SEQUENCE LISTING (3 sequences)

SEQ ID NO: 1 - DNA, 22 bp, synthetic construct
  source 1..22: mol_type = other DNA
catccagcaaatacaccatcca

SEQ ID NO: 2 - DNA, 28 bp, synthetic construct
  source 1..28: mol_type = other DNA
ttctgcacatcataattaggagtatcaa

SEQ ID NO: 3 - DNA, 15 bp, synthetic construct
  source 1..15: mol_type = other DNA
cggagcacaggagat

(19) United States
(12) Patent Application Publication
(10) Pub. No.: US 2024/0309028 A1
(43) Pub. Date: Sep. 19, 2024

(54) PHOSPHOLIPID COMPOUNDS AND METHODS OF MAKING AND USING THE SAME

(71) Applicant: Gilead Sciences, Inc., Foster City, CA (US)

(72) Inventors: Byoung-Kwon Chun, Pleasanton, CA (US); Deeba Ensan, Foster City, CA (US); Rao V. Kalla, Foster City, CA (US); Devan Naduthambi, San Bruno, CA (US); Dustin S. Siegel, Half Moon Bay, CA (US)

(21) Appl. No.: 18/441,123

(22) Filed: Feb. 14, 2024

Related U.S. Application Data
(60) Provisional application No. 63/485,482, filed on Feb. 16, 2023.

(51) Int. Cl. C07F 9/06; A61K 31/675

(52) U.S. Cl. CPC C07F 9/062; A61K 31/675

(57) ABSTRACT

Phospholipid compounds and methods of using the same, singly or in combination with additional agents, and pharmaceutical formulations of said compounds for the treatment of viral infections are disclosed.

### CROSS-REFERENCE TO RELATED APPLICATIONS

This application claims priority to U.S. Provisional Application No. 63/485,482, filed Feb. 16, 2023, which is incorporated herein in its entireties for all purposes.

### SEQUENCE LISTING

The instant application contains a Sequence Listing which has been submitted electronically in .XML file format and is hereby incorporated by reference in its entirety. Said .XML copy, created on Jan. 11, 2024, is named 1461-US-NP.xml and is 3,491 bytes in size.

### BACKGROUND

There is a need for compounds, pharmaceutical formulations, and methods for treating viral infections, for example, Paramyxoviridae, Pneumoviridae, Picornaviridae, Flaviviridae, Filoviridae, and Orthomyxovirus infections. Embodiments of the present disclosure can address these and other needs.

### SUMMARY

In some embodiments, the present disclosure provides a compound of Formula I:

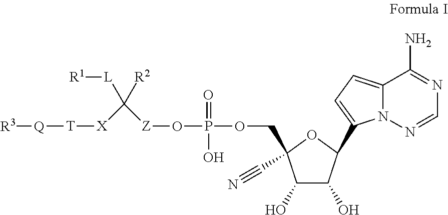

or a pharmaceutically acceptable salt thereof, wherein:
R¹ is H, C₃₋₁₀ cycloalkyl, C₆₋₁₀ aryl, or 5-10 membered heteroaryl containing one, two, or three N; the cycloalkyl, aryl, or heteroaryl of R¹ is optionally substituted with one, two, or three groups independently selected from R^{1A} and —NR^{13A}R^{14A},
   each R^{1A} is independently halo, —CN, C₁₋₃ alkyl, C₁₋₃ haloalkyl, C₁₋₃ alkoxy, C₁₋₃ haloalkoxy, C₃₋₆ cycloalkyl, or 5-10 membered heteroaryl containing one, two, or three heteroatoms selected from N and O;
   each R^{13A} is independently H or C₁-C₃ alkyl optionally substituted with NR^{13A1}R^{14A1}, R^{13A1} is H or C₁₋₃ alkyl; R^{14A1} is H or C₁₋₃ alkyl; and
   each R^{14A} is independently H or C₁₋₃ alkyl;
R² is H or C₁₋₃ alkyl;

R³ is C₁₋₃ haloalkyl;

Q is C₁₀₋₂₁ alkylene or C₁₀₋₂₁ alkenylene; the alkylene or alkenylene of Q is optionally substituted with 1 to 6 Q^{1A}; each Q^{1A} is independently halo;
L is a bond, —O—, or —O(CR^{12A}R^{12B})ₘ—;
   each R^{12A} is independently H or C₁₋₆ alkyl;
   each R^{12B} is independently H or C₁₋₆ alkyl; and
   n is 1 or 2;
X is a bond or C₁₋₃ alkylene;
T is a bond or —O—; and
Z is C₁₋₆ alkylene;
with the proviso that when X is a bond and Tis —O—, then L is a bond.

In some embodiments, the present disclosure provides a compound of Formula I, (Ia), (Ib), (Ic), (Id), or (Ie), or a pharmaceutically acceptable salt thereof.

In some embodiments, the present disclosure provides pharmaceutical formulations comprising a pharmaceutically effective amount of a compound disclosed herein, or a pharmaceutically acceptable salt thereof, and a pharmaceutically acceptable carrier or excipient.

In some embodiments, the present disclosure provides methods of treating or preventing a viral infection in a subject in need thereof, wherein the method comprises administering to the subject a compound disclosed herein, or a pharmaceutically acceptable salt thereof.

In some embodiments, the present disclosure provides a method of treating or preventing a viral infection in a human in need thereof, wherein the method comprises administering to the human a compound disclosed herein, or a pharmaceutically acceptable salt thereof.

In some embodiments, the present disclosure provides a method for manufacturing a medicament for treating or preventing a viral infection in a subject in need thereof, characterized in that a compound disclosed herein, or a pharmaceutically acceptable salt thereof, is used.

In some embodiments, the present disclosure provides a method for manufacturing a medicament for treating or preventing a viral infection in a human in need thereof, characterized in that a compound disclosed herein, or a pharmaceutically acceptable salt thereof, is used.

In some embodiments, the present disclosure provides use of a compound disclosed herein, or a pharmaceutically acceptable salt thereof, for the manufacture of a medicament for the treatment or prevention of a viral infection in a human in need thereof.

### DETAILED DESCRIPTION

### I. General

The disclosure relates generally to methods and compounds for treating or preventing viral infections, for example Paramyxoviridae, Pneumoviridae, Picornaviridae, Flaviviridae, Filoviridae, and Orthomyxovirus infections. The following description sets forth exemplary methods, parameters and the like. It should be recognized, however, that such description is not intended as a limitation on the scope of the present disclosure but is instead provided as a description of exemplary embodiments.

### II. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —CONH₂ is attached through the carbon atom. A dash at the front or end of a chemical group is a matter of convenience; chemical groups can be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

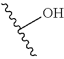 [SMILES: *O]

A squiggly line on a chemical group as shown below, for example, indicates a point of attachment, i.e., it shows the broken bond by which the group is connected to another described group.

As used herein, “a compound of the disclosure” can mean a compound of any of the Formulas I-XIIIb or a pharmaceutically acceptable salt, thereof. Similarly, the phrase “a compound of Formula (number)” means a compound of that formula and pharmaceutically acceptable salts thereof.

The prefix “Cᵤ-Cᵥ” or “Cᵤ₋ᵥ” indicates that the following group has from u to v carbon atoms. For example, “C₁-C₈ alkyl” or “C₁₋₈ alkyl” indicates that the alkyl group has from 1 to 8 carbon atoms.

“Alkyl” refers to an unbranched or branched saturated hydrocarbon chain. For example, an alkyl group can have 1 to 20 carbon atoms (i.e., C₁-C₂₀ alkyl), 1 to 8 carbon atoms (i.e., C₁-C₈ alkyl), 1 to 6 carbon atoms (i.e., C₁-C₆ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (1-Bu, /-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. Other alkyl groups include, but are not limited to, heptyl, octyl, nonyl, decyl, undecyl, dodecyl, pentadcyl, hexadecyl, heptadecyl and octadecyl.

“Alkylene” refers to an unbranched and branched divalent saturated hydrocarbon chain. As used herein, alkylene has 1 to 20 carbon atoms (i.e., C₁₋₂₀ alkylene), 1 to 12 carbon atoms (i.e., C₁₋₁₂ alkylene), 1 to 8 carbon atoms (i.e., C₁₋₈ alkylene), 1 to 6 carbon atoms (i.e., C₁₋₆ alkylene), 1 to 4 carbon atoms (i.e., C₁₋₄ alkylene), 1 to 3 carbon atoms (i.e., C₁₋₃ alkylene), or 1 to 2 carbon atoms (i.e., C₁₋₂ alkylene). Examples of alkyl groups include, but are not limited to, methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons can be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

“Alkenyl” refers to an unbranched or branched hydrocarbon chain containing at least two carbon atoms and at least one carbon-carbon double bond. As used herein, alkenyl can have from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Alkenyl can include any number of carbons, such as C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, or any range therein. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkoxy” means a group having the formula-O-alkyl, in which an alkyl group, as defined above, is attached to the parent molecule via an oxygen atom. The alkyl portion of an alkoxy group can have 1 to 20 carbon atoms (i.e., C₁-C₂₀ alkoxy), 1 to 12 carbon atoms (i.e., C₁-C₁₂ alkoxy), 1 to 8 carbon atoms (i.e., C₁-C₈ alkoxy), 1 to 6 carbon atoms (i.e., C₁-C₆ alkoxy) or 1 to 3 carbon atoms (i.e., C₁-C₃ alkoxy). Examples of suitable alkoxy groups include, but are not limited to, methoxy (—O—CH₃ or —OMe), ethoxy (—OCH₂CH₃ or -OEt), isopropoxy (—O—CH(CH₃)₂), t-butoxy (—O—C(CH₃)₃ or -OtBu) and the like. Other examples of suitable alkoxy groups include, but are not limited to, sec-butoxy, tert-butoxy, pentoxy, hexoxy, and the like. Alkoxy groups can be substituted or unsubstituted.

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁-C₂₀ haloalkyl), 1 to 12 carbon atoms (i.e., C₁-C₁₂ haloalkyl), 1 to 8 carbon atoms (i.e., C₁-C₈ haloalkyl), 1 to 6 carbon atoms (i.e., C₁-C₆ alkyl) or 1 to 3 carbon atoms (i.e., C₁-C₃ alkyl). Examples of suitable haloalkyl groups include, but are not limited to, —CF₃, —CHF₂, —CFH₂, —CH₂CF₃, fluorochloromethyl, difluorochloromethyl, 1,1,1-trifluoroethyl and pentafluoroethyl.

“Halo” or “halogen” as used herein refers to fluoro (—F), chloro (—Cl), bromo (—Br) and iodo (—I).

“Haloalkoxy” is an alkoxy group, as defined above, in which one or more hydrogen atoms of the alkoxy group is replaced with a halogen atom. The alkoxy portion of a haloalkoxy group can have 1 to 20 carbon atoms (i.e., C₁-C₂₀ haloalkoxy), 1 to 12 carbon atoms (i.e., C₁-C₁₂ haloalkoxy), 1 to 8 carbon atoms (i.e., C₁-C₈ haloalkoxy), 1 to 6 carbon atoms (i.e., C₁-C₆ alkoxy) or 1 to 3 carbon atoms (i.e., C₁-C₃ alkoxy). Examples of suitable haloalkoxy groups include, but are not limited to, —OCF₃, —OCHF₂, —OCFH₂, —OCH₂CF₃, and the like.

“Hydroxy” refers to —OH.

“Aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 10 carbon atoms. Exemplary aryl groups include, but are not limited to, radicals derived from benzene (e.g., phenyl), naphthalene, anthracene, biphenyl, and the like.

“Cycloalkyl” refers to a saturated or partially saturated cyclic alkyl group having a single ring or multiple rings including fused, bridged, and spiro ring systems. As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include, but are not limited to, cyclopropyl, cyclobutyl, cyclopentyl, cyclohexyl, cycloheptyl, and cyclooctyl. Cycloalkyl groups also include partially unsaturated ring systems containing one or more double bonds, including fused ring systems with one aromatic ring and one non-aromatic ring, but not fully aromatic ring systems.

“Heteroaryl” refers to an aromatic group, including groups having an aromatic tautomer or resonance structure, having a single ring, multiple rings, or multiple fused rings, with at least one heteroatom in the ring, i.e., one or more ring heteroatoms independently selected from nitrogen, oxygen, and sulfur, wherein the nitrogen or sulfur can be oxidized. Thus, the term includes rings having one or more annular O, N, S, S(O), S(O)₂, and N-oxide groups. The term includes rings having one or more annular C(O) groups. As used herein, heteroaryl include 5 to 20 ring atoms (i.e., 5- to 20-membered heteroaryl), 5 to 12 ring atoms (i.e., 5- to 12-membered heteroaryl), or 5 to 10 ring atoms (i.e., 5- to 10-membered heteroaryl), and 1 to 5 heteroatoms independently selected from nitrogen, oxygen, and sulfur, and oxidized forms of the heteroatoms. Examples of heteroaryl groups include, but are not limited to, pyridin-2(1H)-one, pyridazin-3(2H)-one, pyrimidin-4(3H)-one, quinolin-2(1H)-one, pyrimidinyl, purinyl, pyridyl, pyridazinyl, benzothiazolyl, and pyrazolyl. Heteroaryl does not encompass or overlap with aryl as defined above.

“Heterocycle”, “heterocyclyl”, or “heterocycloalkyl” as used herein refers to a single saturated or partially unsaturated non-aromatic ring or a multiple ring system having at least one heteroatom in the ring (i.e., at least one annular heteroatom selected from oxygen, nitrogen, and sulfur) wherein the multiple ring system includes at least non-aromatic ring containing at least one heteroatom. The multiple ring system can also include other aromatic rings and non-aromatic rings. Unless otherwise specified, a heterocyclyl group has from 3 to 20 annular atoms, for example from 3 to 12 annular atoms, for example from 3 to 10 annular atoms, or 3 to 8 annular atoms, or 3 to 6 annular atoms, or 3 to 5 annular atoms, or 4 to 6 annular atoms, or 4 to 5 annular atoms. Thus, the term includes single saturated or partially unsaturated rings (e.g., 3, 4, 5, 6 or 7-membered rings) having from 1 to 6 annular carbon atoms and from 1 to 3 annular heteroatoms selected from the group consisting of oxygen, nitrogen and sulfur in the ring. The heteroatoms can optionally be oxidized to form —N(—OH)—, ═N(—O—)—, —S(—O)— or —S(═O)₂—. The rings of the multiple condensed ring (e.g., bicyclic heterocyclyl) system can be connected to each other via fused, spiro and bridged bonds when allowed by valency requirements. Heterocycles include, but are not limited to, azetidine, aziridine, imidazolidine, morpholine, oxirane (epoxide), oxetane, thietane, piperazine, piperidine, pyrazolidine, piperidine, pyrrolidine, pyrrolidinone, tetrahydrofuran, tetrahydrothiophene, dihydropyridine, tetrahydropyridine, quinuclidine, 2-oxa-6-azaspiro[3.3]heptan-6-yl, 6-oxa-1-azaspiro[3.3]heptan-1-yl, 2-thia-6-azaspiro[3.3]heptan-6-yl, 2,6-diazaspiro[3.3]heptan-2-yl, 2-azabicyclo[3.1.0]hexan-2-yl, 3-azabicyclo[3.1.0]hexanyl, 2-azabicyclo[2.1.1]hexanyl, 2-azabicyclo[2.2.1]heptan-2-yl, 4-azaspiro[2.4]heptanyl, 5-azaspiro[2.4]heptanyl, and the like.

Heterocycloalkyl rings also include 9 to 15 membered fused ring heterocycloalkyls having 2, 3, or more rings wherein at least one ring is an aryl ring and at least one ring is a non-aromatic ring containing at least one heteroatom. Representative fused bicyclic heterocycloalkyls include, but are not limited to, indoline (dihydroindole), isoindoline (dihydroisoindole), indazoline (dihydroindazole), benzo[d]imidazole, dihydroquinoline, dihydroisoquinoline, dihydrobenzofuran, dihydroisobenzofuran, benzo[d][1,3]dioxol, dihydrobenzo[b]dioxine, dihydrobenzo[d]oxazole, dihydrobenzo[b]thiophene, dihydroisobenzo[c]thiophene, dihydrobenzo[d]thiazole, dihydrobenzo[c]isothiazole, and benzo[b][1,4]thiazine, as shown in the structures below:

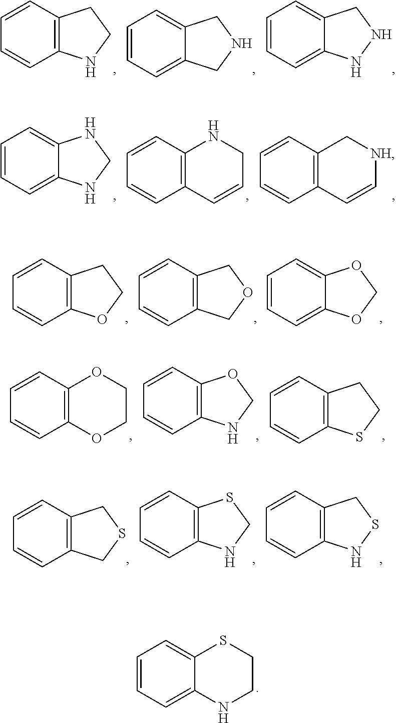

The term “optionally substituted” in reference to a particular moiety of the compound disclosed herein (e.g., an optionally substituted aryl group) refers to a moiety wherein all substituents are hydrogen or wherein one or more of the hydrogens of the moiety can be replaced by the listed substituents.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, formulations, dosage forms and other materials which are useful in preparing a pharmaceutical formulation that is suitable for veterinary or human pharmaceutical use.

The compounds described herein can be prepared and/or formulated as pharmaceutically acceptable salts or when appropriate as a free base. Pharmaceutically acceptable salts are non-toxic salts of a free base form of a compound that possess the desired pharmacological activity of the free base. These salts can be derived from inorganic or organic acids or bases. For example, a compound that contains a basic nitrogen can be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in REMINGTON: THE SCIENCE AND PRACTICE OF PHARMACY, 21^{st} Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

Provided are also compounds described herein or pharmaceutically acceptable salts, isomers, or a mixture thereof, in which from 1 to n hydrogen atoms attached to a carbon atom can be replaced by a deuterium atom or D, in which n is the number of hydrogen atoms in the molecule. As known in the art, the deuterium atom is a non-radioactive isotope of the hydrogen atom. Such compounds can increase resistance to metabolism, and thus can be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” TRENDS PHARMACOL. SCI., 5(12):524-527 (1984). Such compounds are synthesized by means well known in the art, for example by employing starting materials in which one or more hydrogen atoms have been replaced by deuterium. The compounds disclosed herein can be deuterated at various positions, including (but not limited to), the following positions:

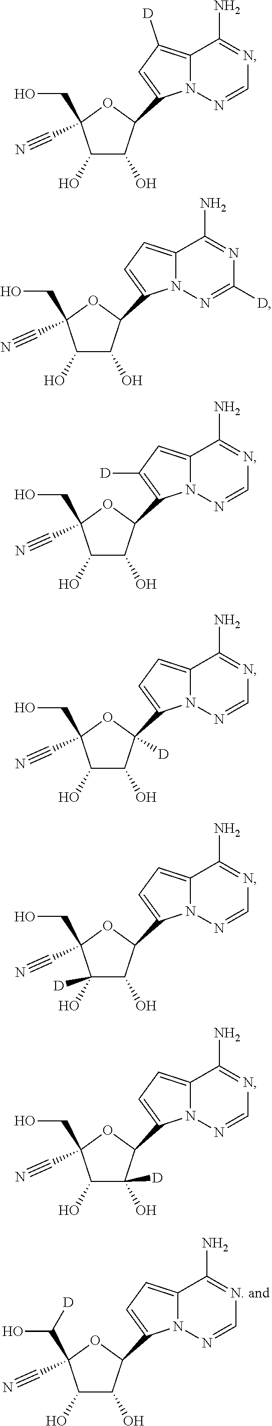

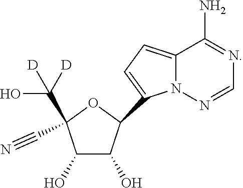 [SMILES: [2H]C([2H])(O)[C@@]1(C#N)O[C@@H](c2ccc3c(N)ncnn23)[C@H](O)[C@@H]1O]

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula I-XIIIb, can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1.1.

The terms “prevention” or “preventing” means any treatment of a disease or condition that causes the clinical symptoms of the disease or condition not to develop. The term “prevention” or “preventing” also encompasses the administration of a compound or composition according to the embodiments disclosed herein post-exposure of the subject to the virus but before the appearance of symptoms of the disease, and/or prior to the detection of the virus in the blood, to prevent the appearance of symptoms of the disease and/or to prevent the virus from reaching detectable levels in the blood, and the administration of a compound or composition according to the embodiments disclosed herein to prevent perinatal transmission of viral infection from mother to baby, by administration to the mother before giving birth and to the child within the first days of life.

“Racemates” refers to a mixture of enantiomers. The mixture can comprise equal or unequal amounts of each enantiomer.

“Stereoisomer” and “stereoisomers” refer to compounds that differ in the chirality of one or more stereocenters. Stereoisomers include enantiomers and diastereomers. The compounds can exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of ADVANCED ORGANIC CHEMISTRY, 4th ed., J. March, John Wiley & Sons, New York, 1992).

A “subject” or “patient” is meant to describe a human or vertebrate animal including a dog, cat, pocket pet, marmoset, horse, cow, pig, sheep, goat, elephant, giraffe, chicken, lion, monkey, owl, rat, squirrel, slender loris, and mouse. A “pocket pet” refers to a group of vertebrate animals capable of fitting into a commodious coat pocket such as, for example, hamsters, chinchillas, ferrets, rats, guinea pigs, gerbils, rabbits and sugar gliders.

“Tautomer” refers to alternate forms of a compound that differ in the position of a proton, such as enol-keto and imine-enamine tautomers, or the tautomeric forms of heteroaryl groups containing a ring atom attached to both a ring —NH— and a ring ═N— such as pyrazoles, imidazoles, benzimidazoles, triazoles, and tetrazoles.

Unless defined otherwise, all technical and scientific terms used herein have the same meaning as commonly understood by one of ordinary skill in the art. A dash at the front or end of a chemical group is a matter of convenience; chemical groups can be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both can be connected in either direction. Similarly, an “arylalkyl” group, for example, can be attached to the remainder of the molecule at either an aryl or an alkyl portion of the group. A prefix such as “Cᵤ₋ᵥ\” or (Cᵤ-Cᵥ) indicates that the following group has from u to v carbon atoms. For example, “C₁₋₆alkyl” and “C₁-C₆ alkyl” both indicate that the alkyl group has from 1 to 6 carbon atoms. Likewise, the term “x-y membered” rings, wherein x and y are numerical ranges, such as “3 to 12-membered heterocyclyl”, refers to a ring containing x-y atoms (e.g., 3-12), of which up to 80% may be heteroatoms, such as N, O, S, and the remaining atoms are carbon.

Unless otherwise specified, the carbon atoms of the compounds of Formulas I-XIIIb are intended to have a valence of four. If in some chemical structure representations, carbon atoms do not have a sufficient number of variables attached to produce a valence of four, the remaining carbon substituents needed to provide a valence of four should be assumed to be hydrogen.

The terms “treating” and “treatment” as used herein are intended to mean the administration of a compound or composition according to the embodiments disclosed herein to alleviate or eliminate symptoms of a viral infection and/or to reduce viral load in a subject.

The term “therapeutically effective amount,” as used herein, is the amount of compound disclosed herein present in a formulation described herein that is needed to provide a desired level of drug in the secretions and tissues of the airways and lungs, or alternatively, in the bloodstream of a subject to be treated to give an anticipated physiological response or desired biological effect when such a formulation is administered by the chosen route of administration. The precise amount will depend upon numerous factors, for example, the particular compound disclosed herein, the specific activity of the formulation, the delivery device employed, the physical characteristics of the formulation, its intended use, as well as subject considerations such as severity of the disease state, subject cooperation, etc., and can readily be determined by one skilled in the art based upon the information provided herein. The term “therapeutically effective amount” or “effective amount” also means amounts that eliminate or reduce the subject's viral burden and/or viral reservoir.

The term “adjacent carbons” as used herein refers to consecutive carbons atoms that are directly attached to each other. For example, in

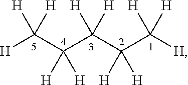

C₁ and C₂ are adjacent carbons, C₂ and C₃ are adjacent carbons, C₃ and C₄ are adjacent carbons, and C₄ and C₅ are adjacent carbons. Similarly, in

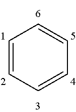

C₁ and C₂ are adjacent carbons, C₂ and C₃ are adjacent carbons, C₃ and C₄ are adjacent carbons, and C₄ and C₅ are adjacent carbons, C₅ and C₆ are adjacent carbons and C₆ and C₁ are adjacent carbons.

“Solvate” as used herein refers to the result of the interaction of a solvent and a compound. Solvates of salts of the compounds described herein are also provided. Hydrates of the compounds described herein are also provided.

“Prodrug” as used herein refers to a derivative of a drug that upon administration to the human body is converted to the parent drug according to some chemical or enzymatic pathway.

As used herein, “pharmaceutically acceptable carrier” or “pharmaceutically acceptable excipient” includes, but is not limited to, any and all solvents, dispersion media, coatings, antibacterial and antifungal agents, isotonic and absorption delaying agents, and combinations thereof. The use of pharmaceutically acceptable carriers and pharmaceutically acceptable excipients for pharmaceutically active substances is well known in the art. Except insofar as any conventional media or agent is incompatible with the active ingredient, its use in the therapeutic formulations is contemplated. Supplementary active ingredients can also be incorporated into the formulations. The carrier(s) must be “acceptable” in the sense of being compatible with the other ingredients of the formulation and physiologically innocuous to the recipient thereof.

### III. Compounds

In some embodiments, the present disclosure provides a compound of Formula (I):

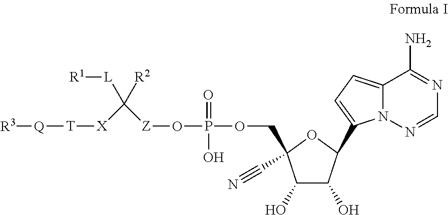

or a pharmaceutically acceptable salt thereof, wherein:
R¹ is H, C₃₋₁₀ cycloalkyl, C₆₋₁₀ aryl, or 5-10 membered heteroaryl containing one, two, or three N; the cycloalkyl, aryl, or heteroaryl of R¹ is optionally substituted with one, two, or three groups independently selected from R^{1A} and —NR^{13A}R^{14A},
   each R^{1A} is independently halo, —CN, C₁₋₃ alkyl, C₁₋₃ haloalkyl, C₁₋₃ alkoxy, C₁₋₃ haloalkoxy, C₃₋₆ cycloalkyl, or 5-10 membered heteroaryl containing one, two, or three heteroatoms selected from N and O;
   each R^{13A} is independently H or C₁-C₃ alkyl optionally substituted with NR^{13A1}R^{14A1}; R^{13A1} is H or C₁₋₃ alkyl; R^{14A1} is H or C₁₋₃ alkyl; and
   each R^{14A} is independently H or C₁₋₃ alkyl;
R² is H or C₁₋₃ alkyl;
R³ is C₁₋₃ haloalkyl;
Q is C₁₀₋₂₁ alkylene or C₁₀₋₂₁ alkenylene; the alkylene or alkenylene of Q is optionally substituted with 1 to 6 Q^{1A}, each Q^{1A} is independently halo;
L is a bond, —O—, or —O(CR^{12A}R^{12B})ₙ—;
   each R^{12A} is independently H or C₁₋₆ alkyl;
   each R^{12B} is independently H or C₁₋₆ alkyl; and
   n is 1 or 2;
X is a bond or C₁₋₃ alkylene;
T is a bond or —O—; and
Z is C₁₋₆ alkylene;
with the proviso that when X is a bond and Tis —O—, then L is a bond.

In some embodiments, the compound of formula (I) has a formula (Ia)

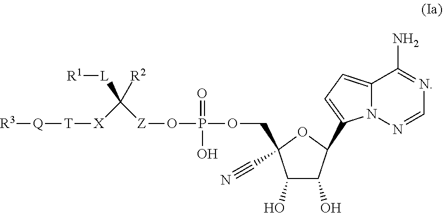

In some embodiments, the compound of formula (I) has a formula (Ib)

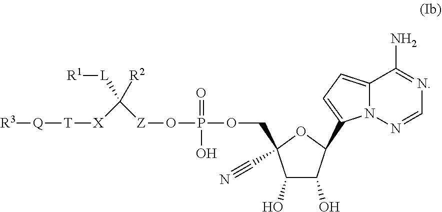

In some embodiments, the compound of formula (I) has a formula (Ic)

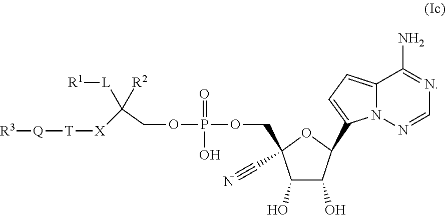

In some embodiments, the compound of formula (I) has a formula (Id)

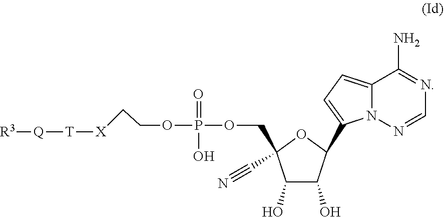

In some embodiments, the compound of formula (I) has a formula (Ie)

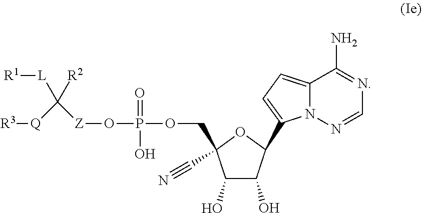

In some embodiments, the compound of Formula (I), (Ia), (Ib), (Ic), (Id), or (Ie), or a pharmaceutically acceptable salt thereof, is the compound wherein R² is H.

In some embodiments, the compound of Formula (I), (Ia), (Ib), or (Ie), or a pharmaceutically acceptable salt thereof, is the compound wherein Z is C₁₋₆ alkylene. In some embodiments, Z is C₁₋₃ alkylene. In some embodiments, Z is —(CH₂)ᵣ, r is 1-6. In some embodiments, Z is —(CH₂)ᵣ—, r is 1-3. In some embodiments, Z is —CH₂—. In some embodiments, Z is —C₂H₄—. In some embodiments, Z is —C₃H₆—.

In some embodiments, the compound of Formula (I), (Ia), (Ib), (Ic), or (Ie), or a pharmaceutically acceptable salt thereof, is the compound wherein L is —O(CR^{12A}R^{12B})ₙ—; each R^{12A} is independently H or C₁₋₆ alkyl; each R^{12B} is independently H or C₁₋₆ alkyl; and n is 1 or 2. In some embodiments, L is —O(CR^{12A}R^{12B})—; R^{12A} is H or C₁₋₆ alkyl; and R^{12B} is H or C₁₋₆ alkyl. In some embodiments, L is —O(CR^{12A}R^{12B})—; R^{12A} is H or C₁₋₃ alkyl; R^{12B} is H or C₁₋₃ alkyl. In some embodiments, L is —OCH₂—. In some embodiments, L is a bond.

In some embodiments, the compound of Formula (I), (Ia), (Ib), (Ic), or (Ie), or a pharmaceutically acceptable salt thereof, is the compound wherein R¹ is C₆₋₁₀ aryl, or 5-10 membered heteroaryl containing one, two, or three N; the aryl, or heteroaryl of R¹ is optionally substituted with one, two, or three groups independently selected from R^{1A} and —NR^{13A}R^{14A}, each R^{1A} is independently halo, —CN, C₁₋₃ alkyl, C₁₋₃ haloalkyl, C₁₋₃ alkoxy, C₁₋₃ haloalkoxy, or 5-10 membered heteroaryl containing one, two, or three heteroatoms selected from N and O; each R^{13A} is independently H or C₁₋₃ alkyl optionally substituted with NR^{13A1}R^{14A1}; R^{13A1} is H or C₁₋₃ alkyl; R^{14A1} is H or C₁₋₃ alkyl; and each R^{14A} is independently H or C₁₋₃ alkyl.

In some embodiments, the compound of Formula (I), (Ia), (Ib), (Ic), or (Ie), or a pharmaceutically acceptable salt thereof, is the compound wherein R¹ is C₆₋₁₀ aryl, or 5-10 membered heteroaryl containing one, two, or three N; the aryl or heteroaryl of R¹ is optionally substituted with one, two, or three groups independently selected from R^{1A}, and each R^{1A} is independently halo, —CN, C₁₋₃ alkyl, C₁₋₃ haloalkyl, C₁₋₃ alkoxy, C₁₋₃ haloalkoxy, or 5-10 membered heteroaryl containing one, two, or three heteroatoms selected from N and O.

In some embodiments, the compound of Formula (I), (Ia), (Ib), (Ic), or (Ie), or a pharmaceutically acceptable salt thereof, is the compound wherein R¹ is C₆₋₁₀ aryl, or 5-10 membered heteroaryl containing one or two N; the aryl or heteroaryl of R¹ is optionally substituted with one, two, or three groups independently selected from R^{1A}, and each R^{1A} is independently halo, —CN, C₁₋₃ alkyl, C₁₋₃ haloalkyl, C₁₋₃ alkoxy, C₁₋₃ haloalkoxy, or 5-10 membered heteroaryl containing one, two, or three heteroatoms selected from N and O.

In some embodiments, the compound of Formula (I), (Ia), (Ib), (Ic), or (Ie), or a pharmaceutically acceptable salt thereof, is the compound wherein R¹ is phenyl or naphthyl; the phenyl or naphthyl of R¹ is optionally substituted with one, two, or three groups independently selected from R^{1A}; and each R^{1A} is independently halo, —CN, C₁₋₃ alkyl, C₁₋₃ haloalkyl, C₁₋₃ alkoxy, C₁₋₃ haloalkoxy, or 5-10 membered heteroaryl containing one, two, or three heteroatoms selected from N and O.

In some embodiments, the compound of Formula (I), (Ia), (Ib), (Ic), or (Ie), or a pharmaceutically acceptable salt thereof, is the compound wherein R¹ is phenyl; the phenyl of R¹ is optionally substituted with one, two, or three groups independently selected from R^{1A}, and each R^{1A} is independently halo, —CN, C₁₋₃ alkyl, C₁₋₃ haloalkyl, C₁₋₃ alkoxy, C₁₋₃ haloalkoxy, or 5-10 membered heteroaryl containing one, two, or three heteroatoms selected from N and O.

In some embodiments, the compound of Formula (I), (Ia), (Ib), (Ic), or (Ie), or a pharmaceutically acceptable salt thereof, is the compound wherein R¹ is phenyl; wherein the phenyl of R¹ is optionally substituted with one or two groups independently selected from R^{1A}, and each R^{1A} is independently halo, —CN, C₁₋₃ alkyl, C₁₋₃ haloalkyl, C₁₋₃ alkoxy, C₁₋₃ haloalkoxy, or 5-10 membered heteroaryl containing one, two, or three heteroatoms selected from N and O.

In some embodiments, the compound of Formula (I), (Ia), (Ib), (Ic), or (Ie), or a pharmaceutically acceptable salt thereof, is the compound wherein R¹ is phenyl; wherein the phenyl of R¹ is optionally substituted with one or two groups independently selected from R^{1A}, and each R¹⁴ is independently halo, —CN, C₁₋₃ alkyl, C₁₋₃ haloalkyl, C₁₋₃ alkoxy, or 5-10 membered heteroaryl containing one, two, or three N.

In some embodiments, the compound of Formula (I), (Ia), (Ib), (Ic), or (Ie), or a pharmaceutically acceptable salt thereof, is the compound wherein R¹ is phenyl; wherein the phenyl of R¹ is optionally substituted one or two groups independently selected from R^{1A}, and each R^{1A} is independently halo, —CN, C₁₋₃ alkoxy, or 5-6 membered heteroaryl containing one, two, or three N.

In some embodiments, the compound of Formula (I), (Ia), (Ib), (Ic), or (Ie), or a pharmaceutically acceptable salt thereof, is the compound wherein R¹ is phenyl; wherein the phenyl of R¹ is optionally substituted one or two groups independently selected from R^{1A}, and each R^{1A} is independently halo, C₁₋₃ alkoxy, or —CN.

In some embodiments, the compound of Formula (I), (Ia), (Ib), (Ic), or (Ie), or a pharmaceutically acceptable salt thereof, is the compound wherein R¹ is phenyl; wherein the phenyl of R¹ is optionally substituted one or two groups independently selected from R^{1A}, and each R^{1A} is independently F, or —CN.

In some embodiments, the compound of Formula (I), (Ia), (Ib), (Ic), or (Ie), or a pharmaceutically acceptable salt thereof, is the compound wherein R¹ is phenyl; wherein the phenyl of R¹ is substituted with F and —CN. In some embodiments, R¹ is

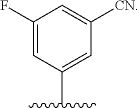 [SMILES: *c1cc(F)cc(C#N)c1]

In some embodiments, the compound of Formula (I), (Ia), (Ib), (Ic), or (Ie), or a pharmaceutically acceptable salt thereof, is the compound wherein R¹ is phenyl; wherein the phenyl of R¹ is substituted with F and —OCH₃. In some embodiments, R¹ is H.

In some embodiments, the compound of Formula (I), (Ia), (Ib), (Ic), or (Ie), or a pharmaceutically acceptable salt thereof, is the compound wherein R¹-L- is

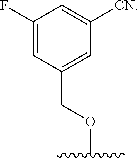 [SMILES: *OCc1cc(F)cc(C#N)c1]

In some embodiments, R¹-L- is H.

In some embodiments, the compound of Formula (I), (Ia), (Ib), (Ic), or (Ie), or a pharmaceutically acceptable salt thereof, is the compound wherein R¹ is 5-10 membered heteroaryl containing one or two N; the heteroaryl of R¹ is optionally substituted with one, two, or three groups independently selected from R^{1A}; and each R^{1A} is independently halo, —CN, or C₁₋₃ alkoxy. In some embodiments, R¹ is quinolinyl optionally substituted with one or two groups independently selected from R^{1A}, and each R^{1A} is independently halo, —CN, or C₁₋₃ alkoxy.

In some embodiments, the compound of Formula (I), (Ia), (Ib), (Ic), or (Id), or a pharmaceutically acceptable salt thereof, is the compound wherein X is a bond. In some embodiments, X is C₁₋₃ alkylene. In some embodiments, X is —CH₂—. In some embodiments, X is —C₂H₄—. In some embodiments, X is —C₃H₆—.

In some embodiments, the compound of Formula (I), (Ia), (Ib), (Ic), or (Id), or a pharmaceutically acceptable salt thereof, is the compound wherein T is a bond. In some embodiments, the compound of Formula (I), (Ia), (Ib), (Ic), or (Id), or a pharmaceutically acceptable salt thereof, is the compound wherein Tis —O—.

In some embodiments, the compound of Formula (I), (Ia), (Ib), (Ic), (Id), or (Ie), or a pharmaceutically acceptable salt thereof, is the compound wherein Q is C₁₀₋₂₁ alkylene optionally substituted with 1 to 6 Q^{1A}; each Q^{1A} is independently halo. In some embodiments, Q is C₁₀₋₂₁ alkylene. In some embodiments, Q is C₁₃₋₂₁ alkylene. In some embodiments, Q is C₁₅₋₂₀ alkylene. In some embodiments, Q is —C₁₆H₃₂—. In some embodiments, Q is —C₁₇H₃₄—. In some embodiments, Q is —C₁₈H₃₆—. In some embodiments, Q is —C₁₉H₃₈—. In some embodiments, Q is —C₂₀H₄₀—. In some embodiments, Q is —(CH₂)ₘ—, m is 10-21. In Some embodiments, m is 13-20. In Some embodiments, m is 15-20. In some embodiments, m is 16. In some embodiments, m is 17. In some embodiments, m is 18. In some embodiments, m is 19. In some embodiments, m is 20.

In some embodiments, the compound of Formula (I), (Ia), (Ib), (Ic), (Id), or (Ie), or a pharmaceutically acceptable salt thereof, is the compound wherein R³ is C₁₋₃ fluoroalkyl. In some embodiments, R³ is —CF₃. In some embodiments, R³ is —CF₂CF₃. In some embodiments, R³ is —CHF₂.

One of skill in the art is aware that each and every embodiment of a group (e.g., R¹) disclosed herein may be combined with any other embodiment of each of the remaining groups (e.g., R², R³, etc.) to generate a complete compound of Formula (I), (Ia), (Ib), (Ic), (Id), or (Ie), or any Formula described herein or a pharmaceutically acceptable salt, stereoisomer, mixture of stereoisomers, or tautomer thereof, each of which is deemed within the ambit of the present disclosure.

In some embodiments, the present disclosure provides a compound as shown in Table 1, or a pharmaceutically acceptable salt thereof.

TABLE 1
Some compounds disclosed herein

| Compound # | structure |
|---|---|
| 1 | 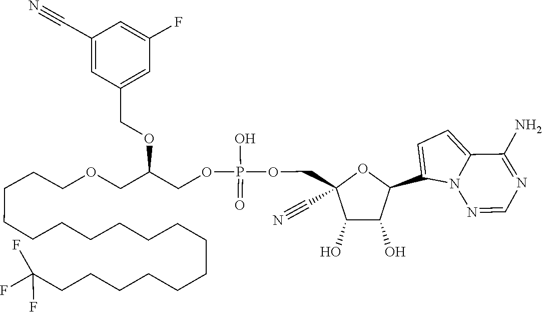 |
| 2 | 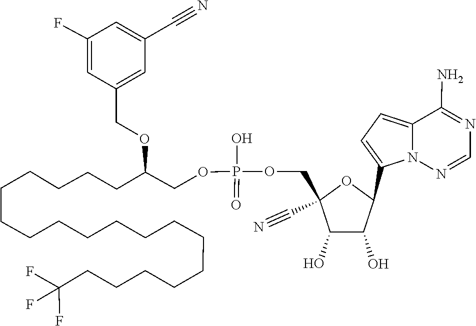 [SMILES: N#Cc1cc(F)cc(CO[C@H](CCCCCCCCCCCCCCCCCCC(F)(F)F)COP(=O)(O)OC[C@@]2(C#N)O[C@@H](c3ccc4c(N)ncnn34)[C@H](O)[C@@H]2O)c1] |
| 3 | 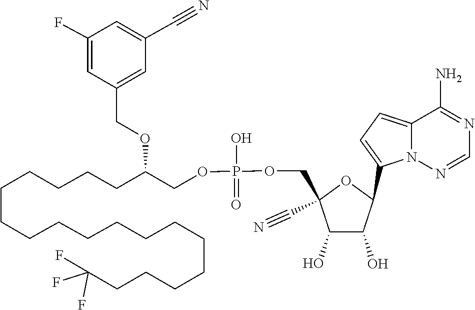 [SMILES: N#Cc1cc(F)cc(CO[C@@H](CCCCCCCCCCCCCCCCCC(F)(F)F)COP(=O)(O)OC[C@@]2(C#N)O[C@@H](c3ccc4c(N)ncnn34)[C@H](O)[C@@H]2O)c1] |
| 4 | 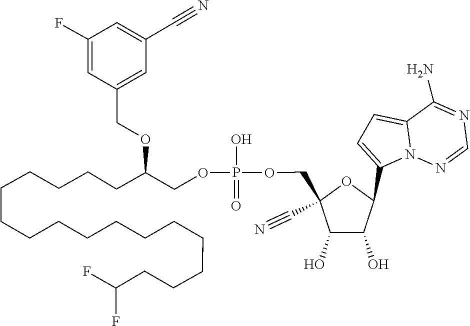 [SMILES: N#Cc1cc(F)cc(CO[C@H](CCCCCCCCCCCCCCCCC(F)F)COP(=O)(O)OC[C@@]2(C#N)O[C@@H](c3ccc4c(N)ncnn34)[C@H](O)[C@@H]2O)c1] |
| 5 | 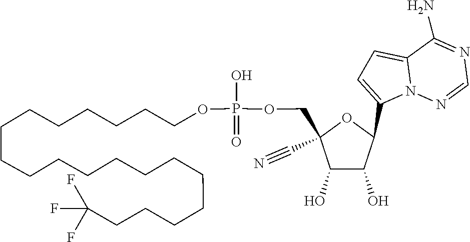 [SMILES: N#C[C@]1(COP(=O)(O)OCCCCCCCCCCCCCCCCCCCC(F)(F)F)O[C@@H](c2ccc3c(N)ncnn23)[C@H](O)[C@@H]1O] |
| 6 | 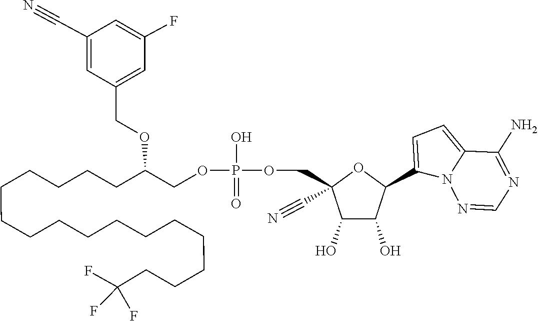 |
| 7 | 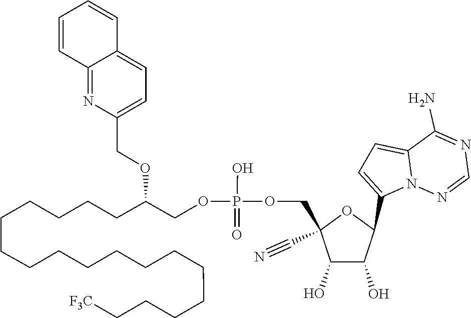 [SMILES: N#C[C@]1(COP(=O)(O)OC[C@H](CCCCCCCCCCCCCCCCCC(F)(F)F)OCc2ccc3ccccc3n2)O[C@@H](c2ccc3c(N)ncnn23)[C@H](O)[C@@H]1O] |
| 8 | 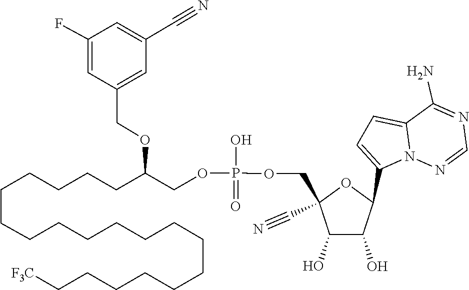 [SMILES: N#Cc1cc(F)cc(CO[C@H](CCCCCCCCCCCCCCCCCCCC(F)(F)F)COP(=O)(O)OC[C@@]2(C#N)O[C@@H](c3ccc4c(N)ncnn34)[C@H](O)[C@@H]2O)c1] |
| 9 | 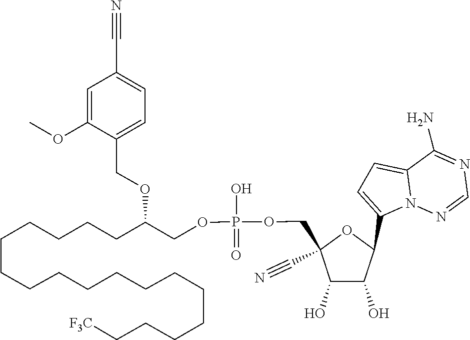 [SMILES: COc1cc(C#N)ccc1CO[C@@H](CCCCCCCCCCCCCCCCCC(F)(F)F)COP(=O)(O)OC[C@@]1(C#N)O[C@@H](c2ccc3c(N)ncnn23)[C@H](O)[C@@H]1O] |
| 10 | 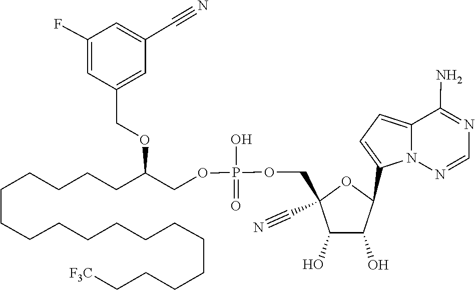 [SMILES: N#Cc1cc(F)cc(CO[C@H](CCCCCCCCCCCCCCCCCC(F)(F)F)COP(=O)(O)OC[C@@]2(C#N)O[C@@H](c3ccc4c(N)ncnn34)[C@H](O)[C@@H]2O)c1] |
| 11 | 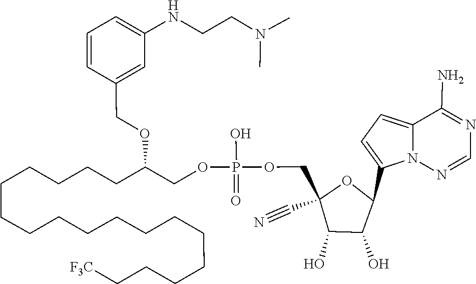 [SMILES: CN(C)CCNc1cccc(CO[C@@H](CCCCCCCCCCCCCCCCCC(F)(F)F)COP(=O)(O)OC[C@@]2(C#N)O[C@@H](c3ccc4c(N)ncnn34)[C@H](O)[C@@H]2O)c1] |

Also falling within the scope herein are the in vivo metabolic products of the compounds described herein, to the extent such products are novel and unobvious over the prior art. Such products may result for example from the oxidation, reduction, hydrolysis, amidation, esterification and the like of the administered compound, primarily due to enzymatic processes. Accordingly, included are novel and unobvious compounds produced by a process comprising contacting a compound with a mammal for a period of time sufficient to yield a metabolic product thereof. Such products typically are identified by preparing a radiolabelled (e.g., ¹⁴C or ³H) compound, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites is done in the same way as conventional drug metabolism studies. The conversion products, so long as they are not otherwise found in vivo, are useful in diagnostic assays for therapeutic dosing of the compounds even if they possess no HSV antiviral activity of their own.

Recipes and methods for determining stability of compounds in surrogate gastrointestinal secretions are known. Compounds are defined herein as stable in the gastrointestinal tract where less than about 50 mole percent of the protected groups are deprotected in surrogate intestinal or gastric juice upon incubation for 1 hour at 37° C. Simply because the compounds are stable to the gastrointestinal tract does not mean that they cannot be hydrolyzed in vivo. The prodrugs typically will be stable in the digestive system but may be substantially hydrolyzed to the parental drug in the digestive lumen, liver, lung or other metabolic organ, or within cells in general. As used herein, a prodrug is understood to be a compound that is chemically designed to efficiently liberate the parent drug after overcoming biological barriers to oral delivery.

### IV. Pharmaceutical Formulations

Also disclosed herein are pharmaceutical formulations comprising a pharmaceutically effective amount of a compound of the present disclosure (e.g., a compound of Formula I, (Ia), (Ib), (Ic), (Id), or (Ie)), or a pharmaceutically acceptable salt thereof, and a pharmaceutically acceptable carrier or excipient. Also provided herein is a pharmaceutical formulation comprising a pharmaceutically effective amount of a compound of the present disclosure, or a pharmaceutically acceptable salt thereof, and a pharmaceutically acceptable carrier or excipient.

The compounds disclosed herein can be formulated with conventional carriers and excipients. Tablets can contain, for instance, excipients, glidants, fillers, binders, or a combination thereof. Aqueous formulations are prepared in sterile form, and when intended for delivery by other than oral administration generally will be isotonic. Exemplary excipients include, but are not limited to, those set forth in the “HANDBOOK OF PHARMACEUTICAL EXCIPIENTS” (1986). Excipients can include, for example, ascorbic acid and other antioxidants, chelating agents such as EDTA, carbohydrates such as dextran, hydroxyalkylcellulose, hydroxyalkylmethylcellulose, stearic acid, and combinations thereof. In some embodiments, the formulation is basic. In some embodiments, the formulation is acidic. In some embodiments, the formulation has a neutral pH. In some embodiments, the pH of the formulations is from 2 to 11 (e.g., 2, 3, 4, 5, 6, 7, 8, 9, 10, 11, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 4-11, 5-6, 5-7, 5-8, 5-9, 5-10, 5-11, 6-7, 6-8, 6-9, 6-10, 6-11, 7-8, 7-9, 7-10, 7-11, 8-9, 8-10, 8-11, 9-10, or 9-11).

In some embodiments, the compounds disclosed herein have pharmacokinetic properties (e.g., oral bioavailability) suitable for oral administration of the compounds. Formulations suitable for oral administration can, for instance, be presented as discrete units such as capsules, cachets or tablets, each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient can also be administered, for instance, as a bolus, electuary, or paste.

A tablet can be made by compression or molding, optionally with one or more accessory ingredients. Compressed tablets can be prepared by compressing in a suitable machine the active ingredient in a free-flowing form such as, for instance, a powder or granules, optionally mixed with a binder, lubricant, inert diluent, preservative, surface active, dispersing agent, or a combination thereof. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom.

For infections of the eye or other external tissues (e.g., mouth and skin), the formulations can be applied as a topical ointment or cream containing the active ingredient(s) in an amount of, for example, 0.075 to 20% w/w (including active ingredient(s) in a range from 0.1% to 20% in increments of 0.1% w/w such as 0.6% w/w, 0.7% w/w, etc.), from 0.2% to 15% w/w, or from 0.5% to 10% w/w. When formulated in an ointment, the active ingredients can be employed in some embodiments with either a paraffinic or a water-miscible ointment base. Alternatively, the active ingredients can be formulated in a cream with an oil-in-water cream base.

In some embodiments, the aqueous phase of the cream base can include, for example, from 30% to 90% (e.g., 35%, 40%, 45%, 50%, 55%, 60%, 65%, 70%, 75%, 80%, 85%) w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. In some embodiments, the cream base can include, for instance, a compound that enhances absorption or penetration of the active ingredient through the skin or other affected areas. Examples of such dermal penetration enhancers include, but are not limited to, dimethyl sulfoxide and related analogs. In some embodiments, the cream or emulsion does not include water.

The oily phase of the emulsions can be constituted from known ingredients in a known manner. In some embodiments, the phase comprises merely an emulsifier (otherwise known as an emulgent). In some embodiments, the phase comprises a mixture of at least one emulsifier with a fat, an oil, or a combination thereof. In some embodiments, a hydrophilic emulsifier is included together with a lipophilic emulsifier that acts as a stabilizer. Together, the emulsifier(s) with or without stabilizer(s) can make up the so-called emulsifying wax, and the wax together with the oil and fat make up the so-called emulsifying ointment base that can form the oily dispersed phase of the cream formulations.

Emulgents and emulsion stabilizers suitable for use in the formulation can include, but are not limited to, TWEEN® 60, TWEEN® 80, SPAN® 80, cetostearyl alcohol, benzyl alcohol, myristyl alcohol, glyceryl mono-stearate, sodium lauryl sulfate, and combinations thereof.

The choice of suitable oils or fats for the formulation can be based on achieving the desired cosmetic properties. In some embodiments, the cream can be a non-greasy, non-staining, and washable product with suitable consistency to avoid leakage from tubes or other containers. In some embodiments, esters can be included, such as, for example, straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate, a blend of branched chain esters known as CRODAMOL® CAP, or a combination thereof. In some embodiments, high melting point lipids such as white soft paraffin and/or liquid paraffin or other mineral oils can be included.

In some embodiments, the compounds disclosed herein are administered alone. In some embodiments, the compounds disclosed herein are administered in pharmaceutical formulations. In some embodiments, the pharmaceutical formulations are for veterinary use. In some embodiments, the pharmaceutical formulations are for human use. In some embodiments, the pharmaceutical formulations disclosed herein include at least one additional therapeutic agent.

Pharmaceutical formulations disclosed herein can be in any form suitable for the intended method of administration. The pharmaceutical formulations disclosed herein can be presented in unit dosage form and can be prepared by any of the methods well known in the art of pharmacy. Exemplary techniques and formulations can be found, for instance, in REMINGTON'S PHARMACEUTICAL SCIENCES (Mack Publishing Co., Easton, PA). Such methods can include the step of bringing into association a compound disclosed herein with the carrier that constitutes one or more accessory ingredients. In general, the formulations can be prepared by uniformly and intimately bringing into association the active ingredient with liquid carriers or finely divided solid carriers or both, and then, if necessary, shaping the product.

When used for oral use for example, tablets, troches, lozenges, aqueous or oil suspensions, dispersible powders or granules, emulsions, hard or soft capsules, solutions, syrups or elixirs can be prepared. Formulations intended for oral use can be prepared according to any method known to the art for the manufacture of pharmaceutical formulations and such formulations can contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients can be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets can be uncoated or can be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax can be employed.

Formulations for oral use can be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin, or olive oil.

Aqueous suspensions can contain the active materials in admixture with excipients suitable for the manufacture of aqueous suspensions. Such excipients can include, for instance, a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension can also contain, for example, one or more preservatives such as ethyl or n-propyl p-hydroxy-benzoate, one or more coloring agents, one or more flavoring agents, one or more sweetening agents (such as sucrose or saccharin), or combinations thereof. Further non-limiting examples of suspending agents include cyclodextrin. In some embodiments, the suspending agent is sulfobutyl ether beta-cyclodextrin (SEB-beta-CD), for example CAPTISOL®.

Oil suspensions can be formulated by suspending the active ingredient in a vegetable oil (e.g., arachis oil, olive oil, sesame oil, coconut oil, or a combination thereof), a mineral oil such as liquid paraffin, or a combination thereof. The oral suspensions can contain, for instance, a thickening agent, such as beeswax, hard paraffin, cetyl alcohol, or a combination thereof. In some embodiments, sweetening agents, such as those set forth above, and/or flavoring agents, are added to provide a palatable oral preparation. In some embodiments, the formulations disclosed herein are preserved by the addition of an antioxidant such as ascorbic acid.

Dispersible powders and granules suitable for preparation of an aqueous suspension by the addition of water can provide the active ingredient in admixture with a dispersing or wetting agent, a suspending agent, a preservative, and combinations thereof. Suitable dispersing or wetting agents and suspending agents are exemplified by those disclosed above. Additional excipients, for example sweetening, flavoring and coloring agents, can also be present.

The pharmaceutical formulations can also be in the form of oil-in-water emulsions. The oily phase can be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion can also contain sweetening and flavoring agents. Syrups and elixirs can be formulated with sweetening agents, such as for instance, glycerol, sorbitol or sucrose. Such formulations can also contain, for instance, a demulcent, a preservative, a flavoring, a coloring agent, or a combination thereof.

The pharmaceutical formulations can be in the form of a sterile injectable or intravenous preparation, such as a sterile injectable aqueous or oleaginous suspension. This suspension can be formulated according to the known art using those suitable dispersing or wetting agents and suspending agents which have been mentioned above. The sterile injectable or intravenous preparation can also be a sterile injectable solution or suspension in a non-toxic parenterally acceptable diluent or solvent, such as a solution in 1,3-butane-diol or prepared as a lyophilized powder. Among the acceptable vehicles and solvents that can be employed are water, Ringer's solution and isotonic sodium chloride solution. In addition, sterile fixed oils can be employed as a solvent or suspending medium. For this purpose, any bland fixed oil can be employed including synthetic mono- or diglycerides. In addition, fatty acids such as oleic acid can likewise be used in the preparation of injectables. Among the acceptable vehicles and solvents that can be employed include, but are not limited to, water, Ringer's solution isotonic sodium chloride solution, and hypertonic sodium chloride solution.

The amount of active ingredient that can be combined with the carrier material to produce a single dosage form will vary depending upon the host treated and the particular mode of administration. For example, a time-release formulation intended for oral administration to humans can contain approximately 1 mg to 1000 mg of active material compounded with an appropriate and convenient amount of carrier material, which can vary from 5% to 95% of the total formulations (weight:weight). The pharmaceutical formulation can be prepared to provide easily measurable amounts for administration. For example, an aqueous solution intended for intravenous infusion can contain from 3 μg to 500 μg of the active ingredient per milliliter of solution in order that infusion of a suitable volume at a rate of 30 mL/hr can occur.

Formulations suitable for topical administration to the eye also include eye drops wherein the active ingredient is dissolved or suspended in a suitable carrier, especially an aqueous solvent for the active ingredient. In some embodiments, the compounds disclosed herein are included in the pharmaceutical formulations disclosed herein in a concentration of 0.5% to 20% (e.g., 0.5% to 10%, 1.5% w/w).

Formulations suitable for topical administration in the mouth include lozenges can comprise an active ingredient (i.e., a compound disclosed herein and/or additional therapeutic agents) in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration can be presented as a suppository with a suitable base comprising for example cocoa butter or a salicylate.

Formulations suitable for vaginal administration can be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions that can contain anti-oxidants, buffers, bacteriostats and solutes that render the formulation isotonic with the blood of the intended recipient; and aqueous and non-aqueous sterile suspensions that can include suspending agents and thickening agents.

The formulations can be presented in unit-dose or multi-dose containers, for example, sealed ampoules and vials, and can be stored in a freeze-dried (lyophilized) condition requiring only the addition of the sterile liquid carrier, for example water for injection, immediately before use. Extemporaneous injection solutions and suspensions are prepared from sterile powders, granules and tablets of the kind previously described. Preferred unit-dosage formulations are those containing a daily dose or unit daily sub-dose, as herein above recited, or an appropriate fraction thereof, of the active ingredient.

It should be understood that in addition to the ingredients particularly mentioned above the formulations can include other agents conventional in the art having regard to the type of formulation in question, for example those suitable for oral administration can include flavoring agents.

Further provided are veterinary formulations comprising a compound disclosed herein together with a veterinary carrier therefor.

Veterinary carriers are materials useful for the purpose of administering the formulation and can be solid, liquid or gaseous materials which are otherwise inert or acceptable in the veterinary art and are compatible with the active ingredient. These veterinary formulations can be administered orally, parenterally, or by any other desired route.

Compounds herein are used to provide controlled release pharmaceutical formulations containing as active ingredient one or more of the compounds (“controlled release formulations”) in which the release of the active ingredient can be controlled and regulated to allow less frequency dosing or to improve the pharmacokinetic or toxicity profile of a given active ingredient.

Effective dose of active ingredient depends at least on the nature of the condition being treated, toxicity, whether the compound is being used prophylactically (lower doses) or against an active viral infection, the method of delivery, and the pharmaceutical formulation, and will be determined by the clinician using conventional dose escalation studies. In some embodiments, the from 0.0001 to 100 mg/kg body weight per day; for instance, from 0.01 to 10 mg/kg body weight per day; from 0.01 to 5 mg/kg body weight per day; from 0.05 to 0.5 mg/kg body weight per day. For example, the daily candidate dose for an adult human of approximately 70 kg body weight can range from 1 mg to 1000 mg (e.g., from 5 mg to 500 mg), and can take the form of single or multiple doses.

### V. Kits

Also provided herein are kits that includes a compound disclosed herein or a pharmaceutically acceptable salt thereof. In some embodiments the kits described herein can comprise a label and/or instructions for use of the compound in the treatment of a disease or condition in a subject (e.g., human) in need thereof. In some embodiments, the disease or condition is viral infection.

In some embodiments, the kit can also comprise one or more additional therapeutic agents and/or instructions for use of additional therapeutic agents in combination with the compound disclosed herein in the treatment of the disease or condition in a subject (e.g., human) in need thereof.

In some embodiments, the kits provided herein comprise individual dose units of a compound as described herein, or a pharmaceutically acceptable salt, racemate, enantiomer, diastereomer, tautomer, polymorph, pseudopolymorph, amorphous form, hydrate or solvate thereof. Examples of individual dosage units can include pills, tablets, capsules, prefilled syringes or syringe cartridges, IV bags, inhalers, nebulizers etc., each comprising a therapeutically effective amount of the compound in question, or a pharmaceutically acceptable salt, racemate, enantiomer, diastereomer, tautomer, polymorph, pseudopolymorph, amorphous form, hydrate or solvate thereof. In some embodiments, the kit can contain a single dosage unit and in others multiple dosage units are present, such as the number of dosage units required for a specified regimen or period.

Also provided are articles of manufacture that include a compound disclosed herein, or a pharmaceutically acceptable salt, stereoisomer, mixture of stereoisomers or tautomer thereof; and a container. In some embodiments, the container of the article of manufacture is a vial, jar, ampoule, preloaded syringe, blister package, tin, can, bottle, box, an intravenous bag, an inhaler, or a nebulizer.

### VI. Administration

One or more compounds of the disclosure are administered by any route appropriate to the condition to be treated. Suitable routes include oral, rectal, inhalation, pulmonary, topical (including buccal and sublingual), vaginal and parenteral (including subcutaneous, intramuscular, intravenous, intradermal, intrathecal and epidural), and the like. In some embodiments, the compounds disclosed herein are administered by inhalation or intravenously. It will be appreciated that the route can vary with for example the condition of the recipient.

In the methods of the present disclosure for the treatment of a viral infection, the compounds of the present disclosure can be administered at any time to a subject who can come into contact with the virus or is already suffering from the viral infection. In some embodiments, the compounds of the present disclosure can be administered prophylactically to subjects coming into contact with subjects suffering from the viral infection or at risk of coming into contact with humans suffering from the viral infection, e.g., healthcare providers. In some embodiments, administration of the compounds of the present disclosure can be to subjects testing positive for the viral infection but not yet showing symptoms of the viral infection. In the methods of the present disclosure for the treatment of a viral infection, the compounds of the present disclosure can be administered at any time to a human who can come into contact with the virus or is already suffering from the viral infection. In some embodiments, the compounds of the present disclosure can be administered prophylactically to humans coming into contact with humans suffering from the viral infection or at risk of coming into contact with humans suffering from the viral infection, e.g., healthcare providers. In some embodiments, administration of the compounds of the present disclosure can be to humans testing positive for the viral infection but not yet showing symptoms of the viral infection. In some embodiments, administration of the compounds of the present disclosure can be to humans upon commencement of symptoms of the viral infection.

In some embodiments, the methods disclosed herein comprise event driven administration of the compound disclosed herein, or a pharmaceutically acceptable salt thereof, to the subject.

As used herein, the terms “event driven” or “event driven administration” refer to administration of the compound of any one of Formulas I-XIIIb, or a pharmaceutically acceptable salt thereof, (1) before an event (e.g., 2 hours, 1 day, 2 days, 5 day, or 7 or more days before the event) that would expose the subject to the virus (or that would otherwise increase the subject's risk of acquiring the viral infection); and/or (2) during an event (or more than one recurring event) that would expose the subject to the virus (or that would otherwise increase the subject's risk of acquiring the viral infection); and/or (3) after an event (or after the final event in a series of recurring events) that would expose the subject to the virus (or that would otherwise increase the subject's risk of acquiring the viral infection). In some embodiments, the event driven administration is performed pre-exposure of the subject to the virus. In some embodiments, the event driven administration is performed post-exposure of the subject to the virus. In some embodiments, the event driven administration is performed pre-exposure of the subject to the virus and post-exposure of the subject to the virus.

In certain embodiments, the methods disclosed herein involve administration prior to and/or after an event that would expose the subject (e.g., human) to the virus or that would otherwise increase the subject's (e.g., human's) risk of acquiring the viral infection, e.g., as pre-exposure prophylaxis (PrEP) and/or as post-exposure prophylaxis (PEP). In some embodiments, the methods disclosed herein comprise pre-exposure prophylaxis (PrEP). In some embodiments, methods disclosed herein comprise post-exposure prophylaxis (PEP).

In some embodiments, a compound disclosed herein, or a pharmaceutically acceptable salt thereof, is administered before exposure of the subject to the virus.

In some embodiments, a compound disclosed herein, or a pharmaceutically acceptable salt thereof, is administered before and after exposure of the subject to the virus.

In some embodiments, a compound disclosed herein, or a pharmaceutically acceptable salt thereof, is administered after exposure of the subject to the virus.

An example of event driven dosing regimen includes administration of a compound disclosed herein, or a pharmaceutically acceptable salt thereof, within 24 to 2 hours before the virus, followed by administration of a compound disclosed herein, or a pharmaceutically acceptable salt, every 24 hours during the period of exposure, followed by a further administration of a compound disclosed herein, or a pharmaceutically acceptable salt thereof, after the last exposure, and one last administration of a compound disclosed herein, or a pharmaceutically acceptable salt thereof, 24 hours later.

A further example of an event driven dosing regimen includes administration of the compound of any one of Formulas I-XIIIb, or a pharmaceutically acceptable salt thereof, within 24 hours before the viral exposure, then daily administration during the period of exposure, followed by a last administration approximately 24 hours later after the last exposure (which can be an increased dose, such as a double dose).

Effective dose of active ingredient depends at least on the nature of the condition being treated, toxicity, whether the compound is being used prophylactically or against an active viral infection, the method of delivery, and the pharmaceutical formulation, and will be determined by the clinician using conventional dose escalation studies. It can be expected to be from 0.0001 mg/kg to 100 mg/kg body weight per day (e.g., from 0.01 mg/kg to 10 mg/kg body weight per day; from 0.01 mg/kg to 5 mg/kg body weight per day; from 0.05 mg/kg to 0.5 mg/kg body weight per day). In some embodiments, the daily candidate dose for an adult human of approximately 70 kg body weight is from 1 mg to 2000 mg (e.g., 5 mg to 500 mg, 500 mg to 1000 mg, 1000 mg to 1500 mg, 1500 mg to 2000 mg) and can take the form of single or multiple doses (e.g., 2 doses per day, 3 doses per day). For example, the daily candidate dose for an adult human of approximately 70 kg body weight can range from 1 mg to 1000 mg (e.g., from 5 mg to 500 mg) and can take the form of single or multiple doses.

Any suitable period of time for administration of the compounds of the present disclosure is contemplated. For example, administration can be for from 1 day to 100 days, including 2, 3, 4, 5, 6, 7, 8, 9, 10, 15, 20, 25, 30, 40, 50, 60, 70, 80, or 90 days. The administration can also be for from 1 week to 15 weeks, including 1, 2, 3, 4, 5, 6, 7, 8, 9, 10, 11, 12, 13, 14, or 15 weeks. Longer periods of administration are also contemplated.

In some embodiments, the compounds disclosed herein are administered once daily. In some embodiments, the compounds disclosed herein are administered twice daily. In some embodiments, the compounds disclosed herein are administered once every alternate day. In some embodiments, the compounds disclosed herein are administered once a week. In some embodiments, the compounds disclosed herein are administered twice a week.

In some embodiments, one or more compounds disclosed herein are administered once daily. The once daily dose can be administered for as long as required, for example for up to 5 days, up to 7 days, up to 10 days, up to 15 days, up to 20 days, up to 25 days, up to a month or longer. In some embodiments, the once daily dose is administered for up to 20 days, up to 15 days, up to 14 days, up to 13 days, up to 12 days, up to 10 days, up to 8 days, up to 6 days, up to 4 days, up to 3 days, up to 2 days, or for one day.

In some embodiments, the one or more compounds disclosed herein are dosed once daily, for 6 to 12 days, for example for 8-10 days. In some embodiments, the one or more compounds are administered once daily for 9 days. In some embodiments, the one or more compounds are administered once daily for 10 days. In some embodiments 50-150 mg of one or more compounds disclosed herein is administered once daily for 5 to 12 days, for e.g., for 5 days, 6 days, 7 days, 8 days, 9 days, 10 days, 11 days, or 12 days. In some embodiments 100 mg of one or more compounds disclosed herein is administered once daily for 5 to 12 days, for e.g., for 5 days, 6 days, 7 days, 8 days, 9 days, 10 days, 11 days, or 12 days. In some embodiments 500-2000 mg (e.g., 500-1000 mg, 1000-1500 mg) of one or more compounds disclosed herein is administered once daily for 5 to 12 days, for e.g., for 5 days, 6 days, 7 days, 8 days, 9 days, 10 days, 11 days, or 12 days.

In some embodiments, one or more compounds disclosed herein are administered twice daily. The twice daily dose can be administered for as long as required, for example for up to 5 days, up to 7 days, up to 10 days, up to 15 days, up to 20 days, up to 25 days, up to a month or longer. In some embodiments, the twice daily dose is administered for up to 20 days, up to 15 days, up to 14 days, up to 13 days, up to 12 days, up to 10 days, up to 8 days, up to 6 days, up to 4 days, up to 3 days, up to 2 days, or for one day.

In some embodiments, the one or more compounds disclosed herein are dosed twice daily, for 6 to 12 days, for example for 8-10 days. In some embodiments, the one or more compounds are administered twice daily for 9 days. In some embodiments, the one or more compounds are administered twice daily for 10 days. In some embodiments 1-1000 mg of one or more compounds disclosed herein is administered twice daily for 5 to 12 days, for e.g., for 5 days, 6 days, 7 days, 8 days, 9 days, 10 days, 11 days, or 12 days. In some embodiments 500-1500 mg (e.g., 500-1000 mg, 1000-1500 mg) of one or more compounds disclosed herein is administered twice daily for 5 to 12 days, for e.g., for 5 days, 6 days, 7 days, 8 days, 9 days, 10 days, 11 days, or 12 days.

### VII. Methods of Use

The present disclosure also provides a method of treating or preventing a viral infection in a subject (e.g., human) in need thereof, the method comprising administering to the subject a compound described herein.

In some embodiments, the present disclosure provides a method of treating a viral infection in a subject (e.g., human) in need thereof, the method comprising administering to a subject in need thereof a compound described herein.

In some embodiments, the present disclosure provides for methods of treating or preventing a viral infection in a subject (e.g., human) in need thereof, the method comprising administering to the subject a compound disclosed herein and at least one additional active therapeutic or prophylactic agent.

In some embodiments, the present disclosure provides for methods of treating a viral infection in a subject (e.g., human) in need thereof, the method comprising administering to the subject a compound disclosed herein, and at least one additional active therapeutic agent.

In some embodiments, the present disclosure provides for methods of inhibiting a viral polymerase in a cell, the methods comprising contacting the cell infected a virus with a compound disclosed herein, whereby the viral polymerase is inhibited.

In some embodiments, the present disclosure provides for methods of inhibiting a viral polymerase in a cell, the methods comprising contacting the cell infected a virus with a compound disclosed herein, and at least one additional active therapeutic agent, whereby the viral polymerase is inhibited.

Also provided here are the uses of the compounds disclosed herein for use in treating or preventing a viral infection in a subject in need thereof. For example, provided herein are uses of the compounds disclosed herein for use in treating a viral infection in a subject in need thereof.

### A. Paramyxoviridae

In some embodiments, the viral infection is a Paramyxoviridae virus infection. As such, in some embodiments, the present disclosure provides methods for treating a Paramyxoviridae infection in a subject (e.g., a human) in need thereof, the method comprising administering to the subject a compound disclosed herein. In some embodiments, the Paramyxoviridae virus includes a BSL4 pathogen. Paramyxoviridae viruses include, but are not limited to, Nipah virus, Hendra virus, measles, mumps, and parainfluenza virus.

In some embodiments, the present disclosure provides a method for manufacturing a medicament for treating a Paramyxoviridae virus infection in a subject (e.g., human) in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a subject (e.g., human) of Paramyxoviridae virus infection.

In some embodiments, the present disclosure provides a compound of the present disclosure, or a pharmaceutically acceptable salt thereof, for use in the treatment of a Paramyxoviridae virus infection in a subject (e.g., human) in need thereof.

### B. Pneumoviridae

In some embodiments, the viral infection is a Pneumoviridae virus infection. In some embodiments, the present disclosure provides a method of treating a Pneumoviridae virus infection in a subject (e.g., human) in need thereof, the method comprising administering to the human a therapeutically effective amount of a compound of the present disclosure, or a pharmaceutically acceptable salt thereof. Pneumoviridae viruses include, but are not limited to, respiratory syncytial virus (RSV) and human metapneumovirus. In some embodiments, the Pneumoviridae virus infection is a respiratory syncytial virus (RSV) infection. In some embodiments, the Pneumoviridae virus infection is human metapneumovirus infection.

In some embodiments, the present disclosure provides a method for manufacturing a medicament for treating a Pneumoviridae virus infection in a subject (e.g., human) in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a subject (e.g., human) of a Pneumoviridae virus infection. In some embodiments, the Pneumoviridae virus infection is a respiratory syncytial virus infection. In some embodiments, the Pneumoviridae virus infection is human metapneumovirus infection.

In some embodiments, the present disclosure provides a method for manufacturing a medicament for treating a Pneumoviridae virus infection in a human in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a human of a Pneumoviridae virus infection. In some embodiments, the Pneumoviridae virus infection is a respiratory syncytial virus infection. In some embodiments, the Pneumoviridae virus infection is human metapneumovirus infection.

In some embodiments, the present disclosure provides a compound of the present disclosure, or a pharmaceutically acceptable salt thereof, for use in the treatment of a Pneumoviridae virus infection in a human in need thereof. In some embodiments, the Pneumoviridae virus infection is a respiratory syncytial virus (RSV) infection. In some embodiments, the Pneumoviridae virus infection is human metapneumovirus infection.

In certain embodiments, the present disclosure provides methods for treating an RSV infection, comprising administering to a subject (e.g., a human) infected with respiratory syncytial virus a therapeutically effective amount a compound of the present disclosure or a pharmaceutically acceptable salt thereof. In some embodiments, the human is suffering from a chronic respiratory syncytial viral infection. In some embodiments, the human is acutely infected with RSV.

In certain embodiments, a method of inhibiting RSV replication is provided, comprising administering a compound of the present disclosure, or a pharmaceutically acceptable salt thereof, to a subject (e.g., a human).

In certain embodiments, the present disclosure provides a method for reducing the viral load associated with RSV infection, wherein the method comprises administering to a subject (e.g., a human) infected with RSV a therapeutically effective amount of a compound of the present disclosure, or a pharmaceutically acceptable salt thereof, wherein the therapeutically effective amount is sufficient to reduce the RSV viral load in the subject.

As described more fully herein, compounds of the present disclosure can be administered with one or more additional therapeutic agent(s) to a subject (e.g., a human) infected with RSV. The additional therapeutic agent(s) can be administered to the infected subject (e.g., a human) at the same time as a compound of the present disclosure or before or after administration of a compound of the present disclosure.

In certain embodiments, a compound of the present disclosure, or a pharmaceutically acceptable salt thereof, for use in treating or preventing an RSV infection is provided. In certain embodiments, a compound of the present disclosure (e.g., a compound of Formula I, (Ia), (Ib), (Ic), (Id), or (Ie)), or a pharmaceutically acceptable salt thereof, for the manufacture of a medicament for treating or preventing an RSV infection is provided.

In some embodiments, a method of inhibiting RSV replication is provided, wherein the method comprises administering to a subject (e.g., human) in need thereof, a compound disclosed herein, wherein the administration is by inhalation.

In some embodiments, the present disclosure provides a method for reducing the viral load associated with RSV infection, wherein the method comprises administering to a human infected with RSV a compound disclosed herein.

### C. Picornaviridae

In some embodiments, the viral infection is a Picornaviridae virus infection. In some embodiments, the present disclosure provides a method of treating a Picornaviridae virus infection in a human in need thereof, the method comprising administering to the subject (e.g., human) a therapeutically effective amount of a compound of the present disclosure, or a pharmaceutically acceptable salt thereof. Picornaviridae viruses are enteroviruses causing a heterogeneous group of infections including herpangina, aseptic meningitis, a common-cold-like syndrome (human rhinovirus infection), a non-paralytic poliomyelitis-like syndrome, epidemic pleurodynia (an acute, febrile, infectious disease generally occurring in epidemics), hand-foot-mouth syndrome, pediatric and adult pancreatitis and serious myocarditis. In some embodiments, the Picornaviridae virus infection is human rhinovirus infection. In some embodiments, the Picornaviridae virus infection is enterovirus infection. In some embodiments, the Picornaviridae virus infection is selected from the group consisting of Coxsackie A virus infection, Coxsackie A virus infection, enterovirus D68 infection, enterovirus B69 infection, enterovirus D70 infection, enterovirus A71 infection, and poliovirus infection. In some embodiments, the Picornaviridae virus is foot and mouth disease virus (FMDV).

In some embodiments, the present disclosure provides a method for manufacturing a medicament for treating a Picornaviridae virus infection in a subject (e.g., human) in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a subject (e.g., human) of a Picornaviridae virus infection. In some embodiments, the Picornaviridae virus infection is human rhinovirus infection.

In some embodiments, the present disclosure provides a compound of the present disclosure, or a pharmaceutically acceptable salt thereof, for use in the treatment of a Picornaviridae virus infection in a subject (e.g., human) in need thereof. In some embodiments, the Picornaviridae virus infection is human rhinovirus infection.

### D. Flaviviridae

In some embodiments, the viral infection is a Flaviviridae virus infection. In some embodiments, the present disclosure provides a method of treating a Flaviviridae virus infection in a subject (e.g., human) in need thereof, the method comprising administering to the subject (e.g., human) a therapeutically effective amount of a compound of the present disclosure, or a pharmaceutically acceptable salt thereof. Representative Flaviviridae viruses include, but are not limited to, dengue, Yellow fever, West Nile, Zika, Japanese encephalitis virus, tick-borne encephalitis virus (TBEV), and Hepatitis C (HCV). In some embodiments, the Flaviviridae virus infection is a dengue virus infection. In some embodiments, the Flaviviridae virus infection is a Yellow fever virus infection. In some embodiments, the Flaviviridae virus infection is a West Nile virus infection. In some embodiments, the Flaviviridae virus infection is a Zika virus infection. In some embodiments, the Flaviviridae virus infection is a Japanese encephalitis virus infection. In some embodiments, the Flaviviridae virus infection is a tick-borne encephalitis virus (TBEV) infection. In some embodiments, the Flaviviridae virus infection is a Hepatitis C virus infection. In some embodiments, the Flaviviridae virus infection is bovine viral diarrhea virus (BVDV). In some embodiments, the Flaviviridae virus infection is swine fever virus (SFV).

In some embodiments, the present disclosure provides a method for manufacturing a medicament for treating a Flaviviridae virus infection in a subject (e.g., human) in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a subject (e.g., human) of a Flaviviridae virus infection. In some embodiments, the Flaviviridae virus infection is a dengue virus infection. In some embodiments, the Flaviviridae virus infection is a Yellow fever virus infection. In some embodiments, the Flaviviridae virus infection is a West Nile virus infection. In some embodiments, the Flaviviridae virus infection is a Zika virus infection. In some embodiments, the Flaviviridae virus infection is a Hepatitis C virus infection.

In some embodiments, the present disclosure provides a compound of the present disclosure, or a pharmaceutically acceptable salt thereof, for use in the treatment of a Flaviviridae virus infection in a human in need thereof. In some embodiments, the Flaviviridae virus infection is a dengue virus infection. In some embodiments, the Flaviviridae virus infection is a Yellow fever virus infection. In some embodiments, the Flaviviridae virus infection is a West Nile virus infection. In some embodiments, the Flaviviridae virus infection is a Zika virus infection. In some embodiments, the Flaviviridae virus infection is a Hepatitis C virus infection.

### E. Filoviridae

In some embodiments, the viral infection is a Filoviridae virus infection. In some embodiments, the present disclosure provides a method of treating a Filoviridae virus infection in a subject (e.g., human) in need thereof, the method comprising administering to the subject (e.g., human) a therapeutically effective amount of a compound of the present disclosure, or a pharmaceutically acceptable salt thereof. Representative Filoviridae viruses include, but are not limited to, Ebola (variants Zaire, Bundibugio, Sudan, Tai forest, or Reston) and Marburg. In some embodiments, the Filoviridae virus infection is an Ebola virus infection. In some embodiments, the Filoviridae virus infection is a Marburg virus infection.

In some embodiments, the present disclosure provides a method for manufacturing a medicament for treating a Filoviridae virus infection in a human in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a human of a Filoviridae virus infection. In some embodiments, the Filoviridae virus infection is an Ebola virus infection.

In some embodiments, the present disclosure provides a compound of the present disclosure, or a pharmaceutically acceptable salt thereof, for use in the treatment of a Filoviridae virus infection in a subject (e.g., human) in need thereof. In some embodiments, the Filoviridae virus infection is an Ebola virus infection. In some embodiments, the Filoviridae virus infection is a Marburg virus infection.

### VIII. Combination Therapy

The compounds described herein can also be used in combination with one or more additional therapeutic agents or prophylactic agents. As such, also provided herein are methods for treatment of viral infections in a subject in need thereof, wherein the methods comprise administering to the subject a compound disclosed herein and a therapeutically effective amount of one or more additional therapeutic or prophylactic agents. In some embodiments, the methods comprise administering to the subject a compound disclosed herein and a therapeutically effective amount of one or more additional therapeutic agents. In some embodiments, the compounds disclosed herein are combined with at least one other active therapeutic agent, wherein the combination is used for treating a viral infection in a subject in need thereof. In some embodiments, the combination can be used to treat multiple separate viral infections (e.g., RSV and HIV) in one subject. In some embodiments, the compounds disclosed herein are combined with at least one other active therapeutic agent to cover a broader spectrum of respiratory viruses in one treatment without need for a diagnostic.

In some embodiments, the combination can be used to treat the same virus (e.g., RSV) in one subject. Active therapeutic agents include, but are not limited to, approved drugs, therapeutic agents currently in clinical trials, therapeutic agents that have shown efficacy in an animal model, therapeutic agents that have shown potency in in vitro assays, or any of the above.

In some embodiments, the additional therapeutic agent is an antiviral agent. Any suitable antiviral agent can be used in the methods described herein. In some embodiments, the antiviral agent is selected from the group consisting of 5-substituted 2′-deoxyuridine analogues, nucleoside analogues, pyrophosphate analogues, nucleoside reverse transcriptase inhibitors, non-nucleoside reverse transcriptase inhibitors, protease inhibitors, integrase inhibitors, entry inhibitors, acyclic guanosine analogues, acyclic nucleoside phosphonate analogues, HCV NS5A/NS5B inhibitors, influenza virus inhibitors, interferons, immunostimulators, oligonucleotides, antimitotic inhibitors, and combinations thereof.

In some embodiments, the additional therapeutic agent is a 5-substituted 2′-deoxyuridine analogue. For example, in some embodiments, the additional therapeutic agent is selected from the group consisting of idoxuridine, trifluridine, brivudine [BVDU], and combinations thereof.

In some embodiments, the additional therapeutic agent is a nucleoside analogue. For example, in some embodiments, the additional therapeutic agent is selected from the group consisting of vidarabine, entecavir (ETV), telbivudine, lamivudine, adefovir dipivoxil, tenofovir disoproxil fumarate (TDF) and combinations thereof. In some embodiments, the additional therapeutic agent is favipiravir, ribavirin, galidesivir, β-D-N4-hydroxycytidine or a combination thereof.

In some embodiments, the additional therapeutic agent is a pyrophosphate analogue. For example, in some embodiments, the additional therapeutic agent is foscarnet or phosphonoacetic acid. In some embodiments, the additional therapeutic agent is foscarnet.

In some embodiments, the additional therapeutic agent is nucleoside reverse transcriptase inhibitor. In some embodiments, the antiviral agent is zidovudine, didanosine, zalcitabine, stavudine, lamivudine, abacavir, emtricitabine, and combinations thereof.

In some embodiments, the additional therapeutic agent is a non-nucleoside reverse transcriptase inhibitor. In some embodiments, the antiviral agent is selected from the group consisting of nevirapine, delavirdine, efavirenz, etravirine, rilpivirine, and combinations thereof.

In some embodiments, the additional therapeutic agent is a protease inhibitor. In some embodiments, the protease inhibitor is a HIV protease inhibitor. For example, in some embodiments, the antiviral agent is selected from the group consisting of saquinavir, ritonavir, indinavir, nelfinavir, amprenavir, lopinavir, atazanavir, fosamprenavir, darunavir, tipranavir, cobicistat, and combinations thereof. In some embodiments, the antiviral agent is selected from the group consisting of saquinavir, ritonavir, indinavir, nelfinavir, amprenavir, lopinavir, atazanavir, fosamprenavir, darunavir, tipranavir, and combinations thereof. In some embodiments, the protease inhibitor is an HCV NS3/4A protease inhibitor. For example, in some embodiments, the additional therapeutic agent is selected from the group consisting of voxilaprevir, asunaprevir, boceprevir, paritaprevir, simeprevir, telaprevir, vaniprevir, grazoprevir, ribavirin, danoprevir, faldaprevir, vedroprevir, sovaprevir, deldeprevir, narlaprevir and combinations thereof. In some embodiments, the additional therapeutic agent is selected from the group consisting of voxilaprevir, asunaprevir, boceprevir, paritaprevir, simeprevir, telaprevir, vaniprevir, grazoprevir, and combinations thereof.

In some embodiments, the additional therapeutic agent is an integrase inhibitor. For example, in some embodiments, the additional therapeutic agent is selected from the group consisting of raltegravir, dolutegravir, elvitegravir, abacavir, lamivudine, and combinations thereof. In some embodiments, the additional therapeutic agent is selected from the group consisting of bictegravir, raltegravir, dolutegravir, cabotegravir, elvitegravir, and combinations thereof. In some embodiments, the additional therapeutic agent is selected from the group consisting of bictegravir, dolutegravir, and cabotegravir, and combinations thereof. In some embodiments, the additional therapeutic agent is bictegravir.

In some embodiments, the additional therapeutic agent is an entry inhibitor. For example, in some embodiments, the additional therapeutic agent is selected from the group consisting of docosanol, enfuvirtide, maraviroc, ibalizumab, fostemsavir, leronlimab, ibalizumab, fostemsavir, leronlimab, palivizumab, respiratory syncytial virus immune globulin, intravenous [RSV-IGIV], varicella-zoster immunoglobulin [VariZIG], varicella-zoster immune globulin [VZIG]), and combinations thereof.

In some embodiments, the additional therapeutic agent is an acyclic guanosine analogue. For example, in some embodiments, the additional therapeutic agent is selected from the group consisting of acyclovir, ganciclovir, valacyclovir (also known as valaciclovir), valganciclovir, penciclovir, famciclovir, and combinations thereof.

In some embodiments, the additional therapeutic agent is an acyclic nucleoside phosphonate analogues. For example, in some embodiments, the additional therapeutic agent is selected from a group consisting of cidofovir, adefovir, adefovir dipivoxil, tenofovir, TDF, emtricitabine, efavirenz, rilpivirine, elvitegravir, and combinations thereof. In some embodiments, the additional therapeutic agent is selected from the group consisting of cidofovir, adefovir, adefovir dipivoxil, tenofovir, TDF, and combinations thereof. In some embodiments, the additional therapeutic agent is selected from the group consisting of cidofovir, adefovir dipivoxil, TDF, and combinations thereof.

In some embodiments, the additional therapeutic agent is an HCV NS5A/NS5B inhibitor. In some embodiments, the additional therapeutic agent is a NS3/4A protease inhibitor. In some embodiments, the additional therapeutic agent is a NS5A protein inhibitor. In some embodiments, the additional therapeutic agent is a NS5B polymerase inhibitor of the nucleoside/nucleotide type. In some embodiments, the additional therapeutic agent is a NS5B polymerase inhibitor of the nonnucleoside type. In some embodiments, the additional therapeutic agent is selected from the group consisting of daclatasvir, ledipasvir, velpatasvir, ombitasvir, elbasvir, sofosbuvir, dasabuvir, ribavirin, asunaprevir, simeprevir, paritaprevir, ritonavir, elbasvir, grazoprevir, and combinations thereof. In some embodiments, the additional therapeutic agent is selected from the group consisting of daclatasvir, ledipasvir, velpatasvir, ombitasvir, elbasvir, sofosbuvir, dasabuvir, and combinations thereof.

In some embodiments, the additional therapeutic agent is an influenza virus inhibitor. In some embodiments, the additional therapeutic agent is a matrix 2 inhibitor. For example, in some embodiments, the additional therapeutic agent is selected from the group consisting of amantadine, rimantadine, and combinations thereof. In some embodiments, the additional therapeutic agent is a neuraminidase inhibitor. For example, in some embodiments, the additional therapeutic agent is selected from the group consisting of zanamivir, oseltamivir, peramivir, laninamivir octanoate, and combinations thereof. In some embodiments, the additional therapeutic agent is a polymerase inhibitor. For example, in some embodiments, the additional therapeutic agent is selected from the group consisting of ribavirin, favipiravir, and combinations thereof. In some embodiments, the additional therapeutic agent is selected from the group consisting of amantadine, rimantadine, arbidol (umifenovir), baloxavir marboxil, oseltamivir, peramivir, ingavirin, laninamivir octanoate, zanamivir, favipiravir, ribavirin, and combinations thereof. In some embodiments, the additional therapeutic agent is selected from the group consisting of amantadine, rimantadine, zanamivir, oseltamivir, peramivir, laninamivir octanoate, ribavirin, favipiravir, and combinations thereof.

In some embodiments, the additional therapeutic agent is an interferon. In some embodiments, the additional therapeutic agent is selected from the group consisting of interferon alfacon 1, interferon alfa 1b, interferon alfa 2a, interferon alfa 2b, pegylated interferon alfacon 1, pegylated interferon alfa 1b, pegylated interferon alfa 2a (PegIFNα-2a), and PegIFNα-2b. e embodiments, the additional therapeutic agent is selected from the group consisting of interferon alfacon 1, interferon alfa 1b, interferon alfa 2a, interferon alfa 2b, pegylated interferon alfa 2a (PegIFNα-2a), and PegIFNα-2b. In some embodiments, the additional therapeutic agent is selected from the group consisting of interferon alfacon 1, pegylated interferon alfa 2a (PegIFNα-2a), PegIFNα-2b, and ribavirin. In some embodiments, the additional therapeutic agent is pegylated interferon alfa-2a, pegylated interferon alfa-2b, or a combination thereof.

In some embodiments, the additional therapeutic agent is an immunostimulatory agent. In some embodiments, the additional therapeutic agent is an oligonucleotide. In some embodiments, the additional therapeutic agent is an antimitotic inhibitor. For example, in some embodiments, the additional therapeutic agent is selected from the group consisting of fomivirsen, podofilox, imiquimod, sinecatechins, and combinations thereof.

In some embodiments, the additional therapeutic agent is selected from the group consisting of besifovir, nitazoxanide, REGN2222, doravirine, sofosbuvir, velpatasvir, daclatasvir, asunaprevir, beclabuvir, FV100, and letermovir, and combinations thereof.

In some embodiments, the additional therapeutic agent is an agent for treatment of RSV. For example, in some embodiments, the antiviral agent is ribavirin, ALS-8112 or presatovir. For example, in some embodiments, the antiviral agent is ALS-8112 or presatovir.

In some embodiments, the additional therapeutic agent is an agent for treatment of picornavirus. In some embodiments, the additional therapeutic agent is selected from the group consisting of hydantoin, guanidine hydrochloride, L.-buthionine sulfoximine, Py-11, and combinations thereof. In some embodiments, the additional therapeutic agent is a picornavirus polymerase inhibitor. In some embodiments, the additional therapeutic agent is rupintrivir.

In some embodiments, the additional therapeutic agent is an agent for treatment of malaria. In some embodiments, the additional therapeutic agent is chloroquine.

In some embodiments, the additional therapeutic agent is selected from the group consisting of hydroxychloroquine, chloroquine, artemether, lumefantrine, atovaquone, proguanil, tafenoquine, pyronaridine, artesunate, artenimol, piperaquine, artesunate, amodiaquine, pyronaridine, artesunate, halofantrine, quinine sulfate, mefloquine, solithromycin, pyrimethamine, MMV-390048, ferroquine, artefenomel mesylate, ganaplacide, DSM-265, cipargamin, artemisone, and combinations thereof.

In some embodiments, the additional therapeutic agent is an agent for treatment of coronavirus. In some embodiments, the additional therapeutic agent is an agent for treatment of COVID-19 (coronavirus disease 2019, a disease caused by a virus named SARS-COV-2). In some embodiments, the additional therapeutic agent is selected from a group consisting of IFX-1, FM-201, CYNK-001, DPP4-Fc, ranpirnase, nafamostat, LB-2, AM-1, anti-viroporins, remdesivir, VV116, GS-441524, GS-5245, and combinations thereof.

In some embodiments, the additional therapeutic agent is an agent for treatment of ebola virus. For example, in some embodiments, the additional therapeutic agent is selected from the group consisting of ribavirin, palivizumab, motavizumab, RSV-IGIV (RespiGam®), MEDI-557, A-60444, MDT-637, BMS-433771, amiodarone, dronedarone, verapamil, Ebola Convalescent Plasma (ECP), TKM-100201, BCX4430 ((2S,3S,4R,5R)-2-(4-amino-5H-pyrrolo[3,2-d]pyrimidin-7-yl)-5-(hydroxymethyl)pyrrolidine-3,4-diol), favipiravir (also known as T-705 or Avigan), T-705 monophosphate, T-705 diphosphate, T-705 triphosphate, FGI-106 (1-N,7-N-bis[3-(dimethylamino)propyl]-3,9-dimethylquinolino[8,7-h]quinolone-1,7-diamine), JK-05, TKM-Ebola, ZMapp, rNAPc2, VRC-EBOADC076-00-VP, OS-2966, MVA-BN filo, brincidofovir, Vaxart adenovirus vector 5-based ebola vaccine, Ad26-ZEBOV, FiloVax vaccine, GOVX-E301, GOVX-E302, ebola virus entry inhibitors (NPC1 inhibitors), rVSV-EBOV, and combinations thereof. In some embodiments, the additional therapeutic agent is ZMapp, mAB114, REGEN-EB3, and combinations thereof.

In some embodiments, the additional therapeutic agent is an agent for treatment of HCV. In some embodiments, the additional therapeutic agent is a HCV polymerase inhibitor. For example, in some embodiments, the additional therapeutic agent is selected from the group consisting of sofosbuvir, GS-6620, PSI-938, ribavirin, tegobuvir, radalbuvir, MK-0608, and combinations thereof. In some embodiments, the additional therapeutic agent is a HCV protease inhibitor. For example, in some embodiments, the additional therapeutic agent is selected from the group consisting of such as GS-9256, vedroprevir, voxilaprevir, and combinations thereof.

In some embodiments, the additional therapeutic agent is a NS5A inhibitor. For example, in some embodiments, the additional therapeutic agent is selected from the group consisting of ledipasvir, velpatasvir, and combinations thereof.

In some embodiments, the additional therapeutic agent is an anti HBV agent. For example, in some embodiments, the additional therapeutic agent is tenofovir disoproxil fumarate and emtricitabine, or a combination thereof. Examples of additional anti HBV agents include but are not limited to alpha-hydroxytropolones, amdoxovir, antroquinonol, beta-hydroxycytosine nucleosides, ARB-199, CCC-0975, ccc-R08, elvucitabine, ezetimibe, cyclosporin A, gentiopicrin (gentiopicroside), HH-003, hepalatide, JNJ-56136379, nitazoxanide, birinapant, NJK14047, NOV-205 (molixan, BAM-205), oligotide, mivotilate, feron, GST-HG-131, levamisole, Ka Shu Ning, alloferon, WS-007, Y-101 (Ti Fen Tai), rSIFN-co, PEG-IIFNm, KW-3, BP-Inter-014, oleanolic acid, HepB-nRNA, cTP-5 (rTP-5), HSK-II-2, HEISCO-106-1, HEISCO-106, Hepbarna, IBPB-006IA, Hepuyinfen, DasKloster 0014-01, ISA-204, Jiangantai (Ganxikang), MIV-210, OB-AI-004, PF-06, picroside, DasKloster-0039, hepulantai, IMB-2613, TCM-800B, reduced glutathione, RO-6864018, RG-7834, QL-007sofosbuvir, ledipasvir, UB-551, and ZH-2N, and the compounds disclosed in US20150210682, (Roche), US 2016/0122344 (Roche), WO2015173164, WO2016023877, US2015252057A (Roche), WO16128335A1 (Roche), WO16120186A1 (Roche), US2016237090A (Roche), WO16107833A1 (Roche), WO16107832A1 (Roche), US2016176899A (Roche), WO16102438A1 (Roche), WO16012470A1 (Roche), US2016220586A (Roche), and US2015031687A (Roche). In some embodiments, the additional therapeutic agent is a HBV polymerase inhibitor. Examples of HBV DNA polymerase inhibitors include, but are not limited to, adefovir (HEPSERA®), emtricitabine (EMTRIVA®), tenofovir disoproxil fumarate (VIREAD®), tenofovir alafenamide, tenofovir, tenofovir disoproxil, tenofovir alafenamide fumarate, tenofovir alafenamide hemifumarate, tenofovir dipivoxil, tenofovir dipivoxil fumarate, tenofovir octadecyloxyethyl ester, CMX-157, tenofovir exalidex, besifovir, entecavir (BARACLUDE®), entecavir maleate, telbivudine (TYZEKA®), filocilovir, pradefovir, clevudine, ribavirin, lamivudine (EPIVIR-HBV®), phosphazide, famciclovir, fusolin, metacavir, SNC-019754, FMCA, AGX-1009, AR-II-04-26, HIP-1302, tenofovir disoproxil aspartate, tenofovir disoproxil orotate, and HS-10234. In some embodiments, the additional therapeutic agent is an HBV capsid inhibitor.

In some embodiments, the additional therapeutic agent is an agent for treatment of HIV. In some embodiments, the additional therapeutic agent is selected from the group consisting of HIV protease inhibitors, HIV integrase inhibitors, entry inhibitors, HIV nucleoside reverse transcriptase inhibitors, HIV nonnucleoside reverse transcriptase inhibitors, acyclic nucleoside phosphonate analogues, and combinations thereof.

In some embodiments, the additional therapeutic agent is selected from the group consisting of HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), and cell therapies (such as chimeric antigen receptor T-cell, CAR-T, and engineered T cell receptors, TCR-T, autologous T cell therapies).

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is a HIV combination drug. Examples of the HIV combination drugs include, but are not limited to ATRIPLA® (efavirenz, tenofovir disoproxil fumarate, and emtricitabine); BIKTARVY® (bictegravir, emtricitabine, and tenofovir alafenamide); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); SYMTUZA® (darunavir, tenofovir alafenamide hemifumarate, emtricitabine, and cobicistat); SYMFI™ (efavirenz, lamivudine, and tenofovir disoproxil fumarate); CIMDU™ (lamivudine and tenofovir disoproxil fumarate); tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dapivirine+levonorgestrel, dolutegravir+lamivudine, dolutegravir+emtricitabine+tenofovir alafenamide, elsulfavirine+emtricitabine+tenofovir disoproxil, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine and lamivudine.

In some embodiments, the additional therapeutic agent is a HIV capsid inhibitor (e.g., lenacapavir).

In some embodiments, the additional therapeutic agent is a HIV protease inhibitor. For example, in some embodiments the additional therapeutic agent is selected from the group consisting of saquinavir, ritonavir, indinavir, nelfinavir, amprenavir, lopinavir, atazanavir, fosamprenavir, darunavir, tipranavir, cobicistat, ASC-09, AEBL-2, MK-8718, GS-9500, GS-1156, and combinations thereof. For example, in some embodiments the additional therapeutic agent is selected from the group consisting of saquinavir, ritonavir, indinavir, nelfinavir, amprenavir, lopinavir, atazanavir, fosamprenavir, darunavir, tipranavir, cobicistat. In some embodiments, the additional therapeutic agent is selected from the group consisting of amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, MK-8122, TMB-607, TMC-310911, and combinations thereof.

In some embodiments, the additional therapeutic agent is a HIV integrase inhibitor. For example, in some embodiments, the additional therapeutic agent is selected from the group consisting of raltegravir, elvitegravir, dolutegravir, abacavir, lamivudine, bictegravir and combinations thereof. In some embodiments, the additional therapeutic agent is bictegravir. In some embodiments, the additional therapeutic agent is selected from a group consisting of bictegravir, elvitegravir, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, dolutegravir, JTK-351, bictegravir, AVX-15567, BMS-986197, cabotegravir (long-acting injectable), diketo quinolin-4-1 derivatives, integrase-LEDGF inhibitor, ledgins, M-522, M-532, NSC-310217, NSC-371056, NSC-48240, NSC-642710, NSC-699171, NSC-699172, NSC-699173, NSC-699174, stilbenedisulfonic acid, T-169, VM-3500, cabotegravir, and combinations thereof.

In some embodiments, the additional therapeutic agent is a HIV entry inhibitor. For example, in some embodiments, the additional therapeutic agent is selected from the group consisting of enfuvirtide, maraviroc, and combinations thereof. Further examples of HIV entry inhibitors include, but are not limited to, cenicriviroc, CCR5 inhibitors, gp41 inhibitors, CD4 attachment inhibitors, DS-003 (BMS-599793), gp120 inhibitors, and CXCR4 inhibitors. Examples of CCR5 inhibitors include aplaviroc, vicriviroc, maraviroc, cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, and vMIP (Haimipu). Examples of CXCR4 inhibitors include plerixafor, ALT-1188, N15 peptide, and vMIP (Haimipu).

In some embodiments, the additional therapeutic agent is a HIV nucleoside reverse transcriptase inhibitor. In some embodiments, the additional therapeutic agent is a HIV nonnucleoside reverse transcriptase inhibitor. In some embodiments, the additional therapeutic agent is an acyclic nucleoside phosphonate analogue. In some embodiments, the additional therapeutic agent is a HIV capsid inhibitor.

In some embodiments, the additional therapeutic agent is a HIV nucleoside or nucleotide inhibitor of reverse transcriptase. For example, the additional therapeutic agent is selected from the group consisting of adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, islatravir, lamivudine, phosphazid, stavudine, zalcitabine, zidovudine, rovafovir etalafenamide (GS-9131), GS-9148, MK-8504, MK-8591, MK-858, VM-2500, KP-1461, and combinations thereof.

In some embodiments, the additional therapeutic agent is a HIV non-nucleoside or non-nucleotide inhibitor of reverse transcriptase. For example, the additional agent is selected from the group consisting of dapivirine, delavirdine, delavirdine mesylate, doravirine, efavirenz, etravirine, lentinan, MK-8583, nevirapine, rilpivirine, TMC-278LA, ACC-007, AIC-292, KM-023, PC-1005, elsulfavirine rilp (VM-1500), combinations thereof.

In some embodiments, the additional therapeutic agents are selected from ATRIPLAR (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, the additional therapeutic agent is selected from the group consisting of colistin, valrubicin, icatibant, bepotastine, epirubicin, epoprosetnol, vapreotide, aprepitant, caspofungin, perphenazine, atazanavir, efavirenz, ritonavir, acyclovir, ganciclovir, penciclovir, prulifloxacin, bictegravir, nelfinavir, tegobuvi, nelfinavir, praziquantel, pitavastatin, perampanel, eszopiclone, and zopiclone.

In some embodiments, the additional therapeutic agent is an inhibitor of Bruton tyrosine kinase (BTK, AGMX1, AT, ATK, BPK, IGHD3, IMD1, PSCTK1, XLA; NCBI Gene ID: 695). For example, in some embodiments, the additional therapeutic agent is selected from the group consisting of (S)-6-amino-9-(1-(but-2-ynoyl)pyrrolidin-3-yl)-7-(4-phenoxyphenyl)-7H-purin-8(9H)-one, acalabrutinib (ACP-196), BGB-3111, CB988, HM71224, ibrutinib (Imbruvica), M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, TAS-5315, AZD6738, calquence, danvatirsen, and combinations thereof. In some embodiments, the additional therapeutic agent is selected from a group consisting of tirabrutinib, ibrutinib, acalabrutinib, and combinations thereof. In some embodiments, the additional therapeutic agent is selected from a group consisting of tirabrutinib, ibrutinib, and combinations thereof. In some embodiments, the additional therapeutic agent is tyrphostin A9 (A9).

In some embodiments, the additional therapeutic agent is a KRAS inhibitor. For example, in some embodiments, the additional therapeutic agent is selected from the group consisting of AMG-510, COTI-219, MRTX-1257, ARS-3248, ARS-853, WDB-178, BI-3406, BI-1701963, ARS-1620 (G12C), SML-8-73-1 (G12C), Compound 3144 (G12D), Kobe0065/2602 (Ras GTP), room temperature11, MRTX-849 (G12C) and KRAS (G12D)-selective inhibitory peptides, including KRpep-2, KRpep-2d, and combinations thereof.

In some embodiments, the additional therapeutic agent is a proteasome inhibitor. For example, in some embodiments, the additional therapeutic agent is selected from a group consisting of ixazomib, carfilzomib, marizomib, bortezomib, and combinations thereof. In some embodiments, the additional therapeutic agent is carfilzomib.

In some embodiments, the additional therapeutic agent is a vaccine. For example, in some embodiments, the additional therapeutic agent is a DNA vaccine, RNA vaccine, live-attenuated vaccine, therapeutic vaccine, prophylactic vaccine, protein-based vaccine, or a combination thereof. In some embodiments, the additional therapeutic agent is mRNA-1273. In some embodiments, the additional therapeutic agent is INO-4800 or INO-4700. In some embodiments, the additional therapeutic agent is live-attenuated RSV vaccine MEDI-559, human monoclonal antibody REGN2222 against RSV, palivizumab, respiratory syncytial virus immune globulin, intravenous [RSV-IGIV], and combinations thereof. In some embodiments, the additional therapeutic agent is a HBV vaccine, for example pediarix, engerix-B, and recombivax HB. In some embodiments, the additional therapeutic agent is a VZV vaccine, for example zostavax and varivax. In some embodiments, the additional therapeutic agent is a HPV vaccine, for example cervarix, gardasil 9, and gardasil. In some embodiments, the additional therapeutic agent is an influenza virus vaccine. For example, a (i) monovalent vaccine for influenza A (e.g., influenza A [H5N1] virus monovalent vaccine and influenza A [H1N1] 2009 virus monovalent vaccines), (ii) trivalent vaccine for influenza A and B viruses (e.g., Afluria, Agriflu, Fluad, Fluarix, Flublok, Flucelvax, FluLaval, Fluvirin, and Fluzone), and (iii) quadrivalent vaccine for influenza A and B viruses (FluMist, Fluarix, Fluzone, and FluLaval). In some embodiments, the additional therapeutic agent is a human adenovirus vaccine (e.g., Adenovirus Type 4 and Type 7 Vaccine, Live, Oral). In some embodiments, the additional therapeutic agent is a rotavirus vaccine (e.g., Rotarix for rotavirus serotype G1, G3, G4, or G9 and RotaTeq for rotavirus serotype G1, G2, G3, or G4). In some embodiments, the additional therapeutic agent is a hepatitis A virus vaccine (e.g., Havrix and Vaqta). In some embodiments, the additional therapeutic agent is poliovirus vaccines (e.g., Kinrix, Quadracel, and Ipol). In some embodiments, the additional therapeutic agent is a yellow fever virus vaccine (e.g., YF-Vax). In some embodiments, the additional therapeutic agent is a Japanese encephalitis virus vaccine (e.g., Ixiaro and JE-Vax). In some embodiments, the additional therapeutic agent is a measles vaccine (e.g., M-M-R II and ProQuad). In some embodiments, the additional therapeutic agent is a mumps vaccine (e.g., M-M-R II and ProQuad). In some embodiments, the additional therapeutic agent is a rubella vaccine (e.g., M-M-R II and ProQuad). In some embodiments, the additional therapeutic agent is a varicella vaccine (e.g., ProQuad). In some embodiments, the additional therapeutic agent is a rabies vaccine (e.g., Imovax and RabAvert). In some embodiments, the additional therapeutic agent is a variola virus (smallpox) vaccine (ACAM2000). In some embodiments, the additional therapeutic agent is a and hepatitis E virus (HEV) vaccine (e.g., HEV239). In some embodiments, the additional therapeutic agent is a SARS-COV-2 vaccine.

In some embodiments, the additional therapeutic agent is an antibody, for example a monoclonal antibody. For example, the additional therapeutic agent is an antibody against SARS-COV-2 selected from the group consisting of the Regeneron antibodies, the Wuxi Antibodies, the Vir Biotechnology Antibodies, antibodies that target the SARS-COV-2 spike protein, antibodies that can neutralize SARS-COV-2 (SARS-COV-2 neutralizing antibodies), and combinations thereof. In some embodiments, the additional therapeutic agent is anti-SARS COV antibody CR-3022. In some embodiments, the additional therapeutic agent is aPD-1 antibody.

In some embodiments, the additional therapeutic agent is recombinant cytokine gene-derived protein injection.

In some embodiments, the additional therapeutic agent is a polymerase inhibitor. In some embodiments, the additional therapeutic agent is a DNA polymerase inhibitor. For example, in some embodiments, the additional therapeutic agent is cidofovir. In some embodiments, the additional therapeutic agent is a RNA polymerase inhibitor. For example, in some embodiments, the additional therapeutic agent is selected from the group consisting of ribavirin, favipiravir, lamivudine, pimodivir and combination thereof.

In some embodiments, the additional therapeutic agent is selected from the group consisting of lopinavir, ritonavir, interferon-alpha-2b, ritonavir, arbidol, hydroxychloroquine, darunavir and cobicistat, abidol hydrochloride, oseltamivir, litonavir, emtricitabine, tenofovir alafenamide fumarate, baloxavir marboxil, ruxolitinib, and combinations thereof.

In some embodiments, the additional therapeutic agent is selected from the group consisting of 6′-fluorinated aristeromycin analogues, acyclovir fleximer analogues, disulfiram, thiopurine analogues, ASC09F, GC376, GC813, phenylisoserine derivatives, neuroiminidase inhibitor analogues, pyrithiobac derivatives, bananins and 5-hydroxychromone derivatives, SSYA10-001, griffithsin, HR2P-M1, HR2P-M2, P21S10, Dihydrotanshinone E-64-C and E-64-D, OC43-HR2P, MERS-5HB, 229E-HRIP, 229E-HR2P, resveratrol, 1-thia-4-azaspiro[4.5] decan-3-one derivatives, gemcitabine hydrochloride, loperamide, recombinant interferons, cyclosporine A, alisporivir, imatinib mesylate, dasatinib, selumetinib, trametinib, rapamycin, saracatinib, chlorpromazine, triflupromazine, fluphenazine, thiethylperazine, promethazine, cyclophilin inhibitors, K11777, camostat, k22, teicoplanin derivatives, benzo-heterocyclic amine derivatives N30, mycophenolic acid, silvestrol, and combinations thereof.

In some embodiments, the additional therapeutic agent is an antibody. In some embodiments, the additional therapeutic agent is an antibody that binds to a coronavirus, for example an antibody that binds to SARS or MERS. In some embodiments, the additional therapeutic agent is a of SARS-COV-2 virus antibody.

Formulations of the disclosure are also used in combination with other active ingredients. For the treatment of SARS-COV-2 virus infections, in some embodiments, the other active therapeutic agent is active against coronavirus infections, for example SARS-COV-2 virus infections. The compounds and formulations of the present disclosure are also intended for use with general care provided subjects with SARS-COV-2 viral infections, including parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication, antiemetic (such as metoclopramide) and/or antidiarrheal agents, vitamin and mineral supplements (including Vitamin K and zinc sulfate), anti-inflammatory agents (such as ibuprofen or steroids), corticosteroids such as methylprednisolone, immonumodulatory medications (e.g., interferon), other small molecule or biologics antiviral agents targeting SARS-COV-2 (such as but not limited to lopinavir/ritonavir, EIDD-1931, favipiravir, ribavirine, neutralizing antibodies, etc.), vaccines, pain medications, and medications for other common diseases in the subject population, such anti-malarial agents (including artemether and artesunate-lumefantrine combination therapy), typhoid (including quinolone antibiotics, such as ciprofloxacin, macrolide antibiotics, such as azithromycin, cephalosporin antibiotics, such as ceftriaxone, or aminopenicillins, such as ampicillin), or shigellosis. In some embodiments, the additional therapeutic agent is dihydroartemisinin/piperaquine.

In some embodiments, the additional therapeutic agent is an immunomodulator. Examples of immune-based therapies include toll-like receptors modulators such as tlr1, tlr2, tlr3, tlr4, tlr5, tlr6, tlr7, tlr8, tlr9, tlr10, tlr11, tlr12, and tlr13; programmed cell death protein 1 (Pd-1) modulators; programmed death-ligand 1 (Pd-L1) modulators; IL-15 modulators; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; polymer polyethyleneimine (PEI); gepon; IL-12; WF-10; VGV-1; MOR-22; BMS-936559; CYT-107, interleukin-15/Fc fusion protein, AM-0015, ALT-803, NIZ-985, NKTR-255, NKTR-262, NKTR-214, normferon, peginterferon alfa-2a, peginterferon alfa-2b, recombinant interleukin-15, Xmab-24306, RPI-MN, STING modulators, RIG-I modulators, NOD2 modulators, SB-9200, and IR-103. In some embodiments, the additional therapeutic agent is fingolimod, leflunomide, or a combination thereof. In some embodiments, the additional therapeutic agent is thalidomide.

In some embodiments, the additional therapeutic agent is an IL-6 inhibitor, for example tocilizumab, sarilumab, or a combination thereof.

In some embodiments, the additional therapeutic agent is an anti-TNF inhibitor. For example, the additional therapeutic agent is adalimumab, etanercept, golimumab, infliximab, or a combination thereof.

In some embodiments, the additional therapeutic agent is a JAK inhibitor, for example the additional therapeutic agent is baricitinib, filgotinib, olumiant, or a combination thereof.

In some embodiments, the additional therapeutic agent is an inflammation inhibitor, for example pirfenidone.

In some embodiments, the additional therapeutic agent is an antibiotic for secondary bacterial pneumonia. For example, the additional therapeutic agent is macrolide antibiotics (e.g., azithromycin, clarithromycin, and Mycoplasma pneumoniae), fluoroquinolones (e.g., ciprofloxacin and levofloxacin), tetracyclines (e.g., doxycycline and tetracycline), or a combination thereof.

In some embodiments, the compounds disclosed herein are used in combination with pneumonia standard of care (see e.g., Pediatric Community Pneumonia Guidelines, CID 2011:53 (1 October)). Treatment for pneumonia generally involves curing the infection and preventing complications. Specific treatment will depend on several factors, including the type and severity of pneumonia, age and overall health of the subjects. The options include: (i) antibiotics, (ii) cough medicine, and (iii) fever reducers/pain relievers (for e.g., aspirin, ibuprofen (Advil, Motrin IB, others) and acetaminophen (Tylenol, others)). In some embodiments, the additional therapeutic agent is bromhexine anti-cough.

In some embodiments, the compounds disclosed herein are used in combination with immunoglobulin from cured COVID-19 subjects. In some embodiments, the compounds disclosed herein are used in combination with plasma transfusion. In some embodiments, the compounds disclosed herein are used in combination with stem cells.

In some embodiments, the additional therapeutic agent is an TLR agonist. Examples of TLR agonists include, but are not limited to, vesatolimod (GS-9620), GS-986, IR-103, lefitolimod, tilsotolimod, rintatolimod, DSP-0509, AL-034, G-100, cobitolimod, AST-008, motolimod, GSK-1795091, GSK-2245035, VTX-1463, GS-9688, LHC-165, BDB-001, RG-7854, telratolimod.RO-7020531.

In some embodiments, the additional therapeutic agent is selected from the group consisting of bortezomid, flurazepam, ponatinib, sorafenib, paramethasone, clocortolone, flucloxacillin, sertindole, clevidipine, atorvastatin, cinolazepam, clofazimine, fosaprepitant, and combinations thereof.

In some embodiments, the additional therapeutic agent is carrimycin, suramin, triazavirin, dipyridamole, bevacizumab, meplazumab, GD31 (rhizobium), NLRP inflammasome inhibitor, or α-ketoamine. In some embodiments, the additional therapeutic agent is recombinant human angiotensin-converting enzyme 2 (rhACE2). In some embodiments, the additional therapeutic agent is viral macrophage inflammatory protein (vMIP).

In some embodiments, the additional therapeutic agent is an anti-viroporin therapeutic. For example, the additional therapeutic agent is BIT-314 or BIT-225. In some embodiments, the additional therapeutic agent is coronavirus E protein inhibitor. For example, the additional therapeutic agent is BIT-009. Further examples of additional therapeutic agents include those described in WO-2004112687, WO-2006135978, WO-2018145148, and WO-2009018609.

It is also possible to combine any compound of the disclosure with one or more additional active therapeutic agents in a unitary dosage form for simultaneous or sequential administration to a subject. The combination therapy can be administered as a simultaneous or sequential regimen. When administered sequentially, the combination can be administered in two or more administrations.

Co-administration of a compound of the disclosure with one or more other active therapeutic agents generally refers to simultaneous or sequential administration of a compound of the disclosure and one or more other active therapeutic agents, such that therapeutically effective amounts of the compound of the disclosure and one or more other active therapeutic agents are both present in the body of the subject.

Co-administration includes administration of unit dosages of the compounds of the disclosure before or after administration of unit dosages of one or more other active therapeutic agents, for example, administration of the compounds of the disclosure within seconds, minutes, or hours of the administration of one or more other active therapeutic agents. For example, a unit dose of a compound of the disclosure can be administered first, followed within seconds or minutes by administration of a unit dose of one or more other active therapeutic agents. Alternatively, a unit dose of one or more other therapeutic agents can be administered first, followed by administration of a unit dose of a compound of the disclosure within seconds or minutes. In some cases, it can be desirable to administer a unit dose of a compound of the disclosure first, followed, after a period of hours (e.g., 1-12 hours), by administration of a unit dose of one or more other active therapeutic agents. In other cases, it can be desirable to administer a unit dose of one or more other active therapeutic agents first, followed, after a period of hours (e.g., 1-12 hours), by administration of a unit dose of a compound of the disclosure.

The combination therapy can provide “synergy” and “synergistic,” i.e., the effect achieved when the active ingredients used together is greater than the sum of the effects that results from using the compounds separately. A synergistic effect can be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect can be attained when the compounds are administered or delivered sequentially, e.g., in separate tablets, pills or capsules, or by different injections in separate syringes. In general, during alternation therapy, an effective dosage of each active ingredient is administered sequentially, i.e., serially, whereas in combination therapy, effective dosages of two or more active ingredients are administered together. A synergistic anti-viral effect denotes an antiviral effect which is greater than the predicted purely additive effects of the individual compounds of the combination.

### A. Combination Therapy for the Treatment of Pneumoviridae Virus Infections

The compounds and pharmaceutically acceptable salts thereof disclosed herein can be used in combination with any of the active therapeutic agents discussed in Section VIII herein and/or with other active therapeutic agents for the treatment of Pneumoviridae virus infections discussed specifically here in Section VIII.A. In some embodiments, the other active therapeutic agent is active against Pneumoviridae virus infections, particularly respiratory syncytial virus infections and/or metapneumovirus infections. As described more fully herein, compounds of the present disclosure can be administered with one or more additional therapeutic agent(s) to an subject (e.g., a human) infected with RSV. Further, in certain embodiments, when used to treat or prevent RSV, a compound of the present disclosure may be administered with one or more (e.g., one, two, three, four or more) additional therapeutic agent(s) selected from the group consisting of RSV combination drugs, RSV vaccines, RSV RNA polymerase inhibitors, immunomodulators toll-like receptor (TLR) modulators, interferon alpha receptor ligands, hyaluronidase inhibitors, respiratory syncytial surface antigen inhibitors, cytotoxic T-lymphocyte-associated protein 4 (ipi4) inhibitors, cyclophilin inhibitors, RSV viral entry inhibitors, antisense oligonucleotide targeting viral mRNA, short interfering RNAs (siRNA) and ddRNAi endonuclease modulators, ribonucelotide reductase inhibitors, farnesoid X receptor agonists, RSV antibodies, CCR2 chemokine antagonists, thymosin agonists, cytokines, nucleoprotein modulators, retinoic acid-inducible gene 1 stimulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, RSV replication inhibitors, arginase inhibitors, and other RSV drugs.

Non-limiting examples of these other active therapeutic agents active against RSV include active monoclonal antibody and nanobody therapeutic agents, agents active against RSV infections, respiratory syncytial virus protein F inhibitors, viral replication inhibitors, RNA polymerase inhibitors, siRNA-based therapies, and combinations thereof. Non-limiting examples of active monoclonal antibody and nanobody therapeutic agents include palivizumab, RSV-IGIV (RESPIGAM®), MEDI-557 (motavizumab), MEDI8897 (nirsevimab), MK-1654, ALX-0171, A-60444 (also known as RSV604), anti-RSV G protein antibodies, and mixtures thereof. Other non-limiting examples of other active therapeutic agents active against respiratory syncytial virus infections include respiratory syncytial virus protein F inhibitors, such as MDT-637, BMS-433771, AK-0529, RV-521 (sisunatovir), JNJ-53718678 (rilematovir), BTA-585, and presatovir; RNA polymerase inhibitors, such as ribavirin, A-60444 (also known as RSV604), JNJ-64417184, ALS-8112 (JNJ-64041575; lumicitabine), and ALS-8112 (the parent nuc of lumicitabine); and viral replication inhibitors, such as EDP-938 and nitazoxanide; siRNA-based therapies, such as ALN-RSV01; and combinations thereof.

In some embodiments, the other active therapeutic agent can be a vaccine for the treatment or prevention of RSV, including but not limited to MVA-BN RSV, RSV-F, MEDI-8897, JNJ-64400141, DPX-RSV, SynGEM, GSK-3389245A, GSK-300389-1A, RSV-MEDI deltaM2-2 vaccine, VRC-RSVRGP084-00VP, Ad35-RSV-FA2, Ad26-RSV-FA2, and RSV fusion glycoprotein subunit vaccine.

Non-limiting examples of other active therapeutic agents active against metapneumovirus infections include sialidase modulators such as DAS-181; RNA polymerase inhibitors, such as ALS-8112; and antibodies for the treatment of Metapneumovirus infections, such as EV-046113.

In some embodiments, the other active therapeutic agent can be a vaccine for the treatment or prevention of metapneumovirus infections, including but not limited to mRNA-1653 and rHMPV-Pa vaccine.

### B. Combination Therapy for the Treatment of Picornaviridae Virus Infections

The compounds and pharmaceutically acceptable salts thereof disclosed herein can be used in combination with any of the active therapeutic agents discussed in Section VIII herein and/or with other active therapeutic agents for the treatment of Picornaviridae virus infections discussed specifically here in Section VIII.B. In some embodiments, the other active therapeutic agent is active against Picornaviridae virus infections, particularly Enterovirus infections. Non-limiting examples of these other active therapeutic agents are capsid binding inhibitors such as pleconaril, BTA-798 (vapendavir) and other compounds disclosed by Wu, et al. (U.S. Pat. No. 7,078,403) and Watson (U.S. Pat. No. 7,166,604); fusion sialidase protein such as DAS-181; a capsid protein VP1 inhibitor such as VVX-003 and AZN-001; a viral protease inhibitor such as CW-33; a phosphatidylinositol 4 kinase beta inhibitor such as GSK-480 and GSK-533; anti-EV71 antibody.

In some embodiments, the other active therapeutic agent can be a vaccine for the treatment or prevention of Picornaviridae virus infections, including but not limited to EV71 vaccines, TAK-021, and EV-D68 adenovector-based vaccine.

### C. Combination Therapy for the Treatment of Respiratory Infections

The compounds and pharmaceutically acceptable salts thereof disclosed herein can be used in combination with any of the active therapeutic agents discussed in Section VIII herein and/or with other active therapeutic agents discussed specifically here in Section VIII.C. Many of the infections of the Pneumoviridae and Picornaviridae viruses are respiratory infections. Therefore, additional active therapeutics used to treat respiratory symptoms and sequelae of infection can be used in combination with the compounds provided herein. The additional agents can be administered orally or by direct inhalation. For example, other additional therapeutic agents in combination with the compounds provided herein for the treatment of viral respiratory infections include, but are not limited to, bronchodilators and corticosteroids.

### Glucocorticoids

Glucocorticoids, which were first introduced as an asthma therapy in 1950 (Carryer, Journal of Allergy, 21, 282-287, 1950), remain the most potent and consistently effective therapy for this disease, although their mechanism of action is not yet fully understood (Morris, J. ALLERGY CLIN. IMMUNOL., 75 (1 Pt) 1-13, 1985). Unfortunately, oral glucocorticoid therapies are associated with profound undesirable side effects such as truncal obesity, hypertension, glaucoma, glucose intolerance, acceleration of cataract formation, bone mineral loss, and psychological effects, all of which limit their use as long-term therapeutic agents (Goodman and Gilman, 10th edition, 2001). A solution to systemic side effects is to deliver steroid drugs directly to the site of inflammation. Inhaled corticosteroids (ICS) have been developed to mitigate the severe adverse effects of oral steroids. Non-limiting examples of corticosteroids that can be used in combinations with the compounds provided herein are dexamethasone, dexamethasone sodium phosphate, fluorometholone, fluorometholone acetate, loteprednol, loteprednol etabonate, hydrocortisone, prednisolone, fludrocortisones, triamcinolone, triamcinolone acetonide, betamethasone, beclomethasone diproprionate, methylprednisolone, fluocinolone, fluocinolone acetonide, flunisolide, fluocortin-21-butylate, flumethasone, flumetasone pivalate, budesonide, halobetasol propionate, mometasone furoate, fluticasone, AZD-7594, ciclesonide; or a pharmaceutically acceptable salts thereof.

### Anti-Inflammatory Agents

Other anti-inflammatory agents working through anti-inflammatory cascade mechanisms are also useful as additional therapeutic agents in combination with the compounds provided herein for the treatment of viral respiratory infections. Applying “anti-inflammatory signal transduction modulators” (referred to in this text as AISTM), like phosphodiesterase inhibitors (e.g., PDE-4, PDE-5, or PDE-7 specific), transcription factor inhibitors (e.g., blocking NFκB through IKK inhibition), or kinase inhibitors (e.g., blocking P38 MAP, JNK, PI3K, EGFR or Syk) is a logical approach to switching off inflammation as these small molecules target a limited number of common intracellular pathways-those signal transduction pathways that are critical points for the anti-inflammatory therapeutic intervention (see review by P. J. Barnes, 2006). These non-limiting additional therapeutic agents include: 5-(2,4-Difluoro-phenoxy)-1-isobutyl-1H-indazole-6-carboxylic acid (2-dimethylamino-ethyl)-amide (P38 Map kinase inhibitor ARRY-797); 3-Cyclopropylmethoxy-N-(3,5-dichloro-pyridin-4-yl)-4-difluorormethoxy-benzamide (PDE-4 inhibitor Roflumilast); 4-[2-(3-cyclopentyloxy-4-methoxyphenyl)-2-phenyl-ethyl]-pyridine (PDE-4 inhibitor CDP-840); N-(3,5-dichloro-4-pyridinyl)-4-(difluoromethoxy)-8-[(methylsulfonyl)amino]-1-dibenzofurancarboxamide (PDE-4 inhibitor Oglemilast); N-(3,5-Dichloro-pyridin-4-yl)-2-[1-(4-fluorobenzyl)-5-hydroxy-1H-indol-3-yl]-2-oxo-acetamide (PDE-4 inhibitor AWD 12-281); 8-Methoxy-2-trifluoromethyl-quinoline-5-carboxylic acid (3,5-dichloro-1-oxy-pyridin-4-yl)-amide (PDE-4 inhibitor Sch 351591); 4-[5-(4-Fluorophenyl)-2-(4-methanesulfinyl-phenyl)-1H-imidazol-4-yl]-pyridine (P38 inhibitor SB-203850); 4-[4-(4-Fluoro-phenyl)-1-(3-phenyl-propyl)-5-pyridin-4-yl-1H-imidazol-2-yl]-but-3-yn-1-ol (P38 inhibitor RWJ-67657); 4-Cyano-4-(3-cyclopentyloxy-4-methoxy-phenyl)-cyclohexanecarboxylic acid 2-diethylamino-ethyl ester (2-diethyl-ethyl ester prodrug of Cilomilast, PDE-4 inhibitor); (3-Chloro-4-fluorophenyl)-[7-methoxy-6-(3-morpholin-4-yl-propoxy)-quinazolin-4-yl]-amine (Gefitinib, EGFR inhibitor); and 4-(4-Methyl-piperazin-1-ylmethyl)-N-[4-methyl-3-(4-pyridin-3-yl-pyrimidin-2-ylamino)-phenyl]-benzamide (Imatinib, EGFR inhibitor).

### β2-Adrenoreceptor Agonist Bronchodilators

Combinations comprising inhaled ß2-adrenoreceptor agonist bronchodilators such as formoterol, albuterol or salmeterol with the compounds provided herein are also suitable, but non-limiting, combinations useful for the treatment of respiratory viral infections.

Combinations of inhaled ß2-adrenoreceptor agonist bronchodilators such as formoterol or salmeterol with ICS's can be used to treat both the bronchoconstriction and the inflammation (SYMBICORT® and ADVAIR®, respectively). The combinations comprising these ICS and ß2-adrenoreceptor agonist combinations along with the compounds provided herein are also suitable, but non-limiting, combinations useful for the treatment of respiratory viral infections.

Other examples of Beta 2 adrenoceptor agonists include, but are not limited to, bedoradrine, vilanterol, indacaterol, olodaterol, tulobuterol, formoterol, abediterol, salbutamol, arformoterol, levalbuterol, fenoterol, and TD-5471.

### Anticholinergics

For the treatment or prophylaxis of pulmonary broncho-constriction, anticholinergics are of potential use and, therefore, useful as an additional therapeutic agent in combination with the compounds provided herein for the treatment of viral respiratory infections. These anticholinergics include, but are not limited to, antagonists of the muscarinic receptor (particularly of the M3 subtype), which have shown therapeutic efficacy in man for the control of cholinergic tone in COPD (Witek, 1999); 1-{4-Hydroxy-1-[3,3,3-tris-(4-fluoro-phenyl)-propionyl]-pyrrolidine-2-carbonyl}-pyrrolidine-2-carboxylic acid (1-methyl-piperidin-4-ylmethyl)-amide; 3-[3-(2-Diethylamino-acetoxy)-2-phenyl-propionyloxy]-8-isopropyl-8-methyl-8-azonia-bicyclo[3.2.1]octane (Ipratropium-N,N-diethylglycinate); 1-Cyclohexyl-3,4-dihydro-1H-isoquinoline-2-carboxylic acid 1-aza-bicyclo[2.2.2]oct-3-yl ester (Solifenacin); 2-Hydroxymethyl-4-methanesulfinyl-2-phenyl-butyric acid 1-aza-bicyclo[2.2.2]oct-3-yl ester (Revatropate); 2-{1-[2-(2,3-Dihydro-benzofuran-5-yl)-ethyl]-pyrrolidin-3-yl}-2,2-diphenyl-acetamide (Darifenacin); (4-Azepan-1-yl-2,2-diphenyl-butyramide (Buzepide); 7-[3-(2-Diethylamino-acetoxy)-2-phenyl-propionyloxy]-9-ethyl-9-methyl-3-oxa-9-azonia-tricyclo[3.3.1.02,4]nonane (Oxitropium-N,N-diethylglycinate); 7-[2-(2-Diethylamino-acetoxy)-2,2-di-thiophen-2-yl-acetoxy]-9,9-dimethyl-3-oxa-9-azonia-tricyclo[3.3.1.02,4]nonane (Tiotropium-N,N-diethylglycinate); Dimethylamino-acetic acid 2-(3-diisopropylamino-1-phenyl-propyl)-4-methyl-phenyl ester (Tolterodine-N,N-dimethylglycinate); 3-[4,4-Bis-(4-fluoro-phenyl)-2-oxo-imidazolidin-1-yl]-1-methyl-1-(2-oxo-2-pyridin-2-yl-ethyl)-pyrrolidinium; 1-[1-(3-Fluoro-benzyl)-piperidin-4-yl]-4,4-bis-(4-fluoro-phenyl)-imidazolidin-2-one; 1-Cyclooctyl-3-(3-methoxy-1-aza-bicyclo[2.2.2]oct-3-yl)-1-phenyl-prop-2-yn-1-ol; 3-[2-(2-Diethylamino-acetoxy)-2,2-di-thiophen-2-yl-acetoxy]-1-(3-phenoxy-propyl)-1-azonia-bicyclo[2.2.2]octane (Aclidinium-N,N-diethylglycinate); or (2-Diethylamino-acetoxy)-di-thiophen-2-yl-acetic acid 1-methyl-1-(2-phenoxy-ethyl)-piperidin-4-yl ester; revefenacin, glycopyrronium bromide, umeclidinium bromide, tiotropium bromide, aclidinium bromide, and bencycloquidium bromide.

### Mucolytic Agents

The compounds provided herein can also be combined with mucolytic agents to treat both the infection and symptoms of respiratory infections. A non-limiting example of a mucolytic agent is ambroxol. Similarly, the compounds can be combined with expectorants to treat both the infection and symptoms of respiratory infections. A non-limiting example of an expectorant is guaifenesin.

Nebulized hypertonic saline is used to improve immediate and long-term clearance of small airways in subjects with lung diseases (Kuzik, J. Pediatrics 2007, 266). Thus, the compounds provided herein can also be combined with nebulized hypertonic saline particularly when the virus infection is complicated with bronchiolitis. The combination of the compound provided herein with hypertonic saline can also comprise any of the additional agents discussed above. In some embodiments, 3% hypertonic saline is used.

### D. Combination Therapy for the Treatment of COPD

The compounds and pharmaceutically acceptable salts thereof disclosed herein can be used in combination with any of the active therapeutic agents discussed in Section VIII herein and/or with other active therapeutic agents for the treatment of respiratory exacerbations of COPD discussed specifically here in Section VIII.D. In some embodiments, the other active therapeutic agents include other active agents against COPD. Non-limiting examples of these other active therapeutic agents include anti-IL5 antibodies, such as benralizumab, mepolizumab; dipeptidyl peptidase I (DPP1) inhibitors, such as AZD-7986 (INS-1007); DNA gyrase inhibitor/topoisomerase IV inhibitors, such as ciprofloxacin hydrochloride; MDR associated protein 4/phosphodiesterase (PDE) 3 and 4 inhibitors, such as RPL-554; CFTR stimulators, such as ivacaftor, QBW-251; MMP-9/MMP-12 inhibitors, such as RBx-10017609; Adenosine A1 receptor antagonists, such as PBF-680; GATA 3 transcription factor inhibitors, such as SB-010; muscarinic receptor modulator/nicotinic acetylcholine receptor agonists, such as ASM-024; MARCKS protein inhibitors, such as BIO-11006; kit tyrosine kinase/PDGF inhibitors such as masitinib; phosphodiesterase (PDE) 4 inhibitors, such as roflumilast, CHF-6001; phosphoinositide-3 kinase delta inhibitors, such as nemiralisib; 5-Lipoxygenase inhibitors, such as TA-270; muscarinic receptor antagonist/beta 2 adrenoceptor agonist, such as batefenterol succinate, AZD-887, ipratropium bromide; TRN-157; elastase inhibitors, such as erdosteine; metalloprotease-12 inhibitors such as FP-025; interleukin 18 ligand inhibitors, such as tadekinig alfa; skeletal muscle troponin activators, such as CK-2127107; p38 MAP kinase inhibitors, such as acumapimod; IL-17 receptor modulators, such as CNTO-6785; CXCR2 chemokine antagonists, such as danirixin; leukocyte elastase inhibitors, such as POL-6014; epoxide hydrolase inhibitors, such as GSK-2256294; HNE inhibitors, such as CHF-6333; VIP agonists, such as aviptadil; phosphoinositide-3 kinase delta/gamma inhibitors, such as RV-1729; complement C₃ inhibitors, such as APL-1; and G-protein coupled receptor-44 antagonists, such as AM-211.

Other non-limiting examples of active therapeutic agents also include, but are not limited to, budesonide, adipocell, nitric oxide, PUR-1800, YLP-001, LT-4001, azithromycin, gamunex, QBKPN, sodium pyruvate, MUL-1867, mannitol, MV-130, MEDI-3506, BI-443651, VR-096, OPK-0018, TEV-48107, doxofylline, TEV-46017, OligoG-COPD-5/20, STEMPEUCEL®, ZP-051, and lysine acetylsalicylate.

In some embodiments, the other active therapeutic agent may be a vaccine that is active against COPD, including but not limited to MV-130 and GSK-2838497A.

### E. Combination Therapy for the Treatment of Flaviviridae Virus Infections

The compounds and pharmaceutically acceptable salts thereof disclosed herein can be used in combination with any of the active therapeutic agents discussed in Section VIII herein and/or with other active therapeutic agents for the treatment of Flaviviridae virus infections discussed specifically here in Section VIII.E. In some embodiments, the other active therapeutic agent is active against Flaviviridae virus infections.

For treatment of the Flaviviridae virus infections, non-limiting examples of the other active therapeutic agents are host cell factor modulators, such as GBV-006; fenretinide ABX-220, BRM-211; alpha-glucosidase 1 inhibitors, such as celgosivir; platelet activating factor receptor (PAFR) antagonists, such as modipafant; cadherin-5/Factor Ia modulators, such as FX-06; NS4B inhibitors, such as JNJ-8359; viral RNA splicing modulators, such as ABX-202; a NS5 polymerase inhibitor; a NS3 protease inhibitor; and a TLR modulator.

In some embodiments, the other active therapeutic agent can be a vaccine for the treatment or prevention of dengue, including but not limited to TETRAVAX-DV, DENGVAXIA®, DPIV-001, TAK-003, live attenuated dengue vaccine, tetravalent dengue fever vaccine, tetravalent DNA vaccine, rDEN2delta30-7169; and DENV-1 PIV.

### F. Combination Therapy for the Treatment of Filoviridae Virus Infections

The compounds and pharmaceutically acceptable salts thereof disclosed herein can be used in combination with any of the active therapeutic agents discussed in Section VIII herein and/or with other active therapeutic agents for the treatment of Filoviridae virus infections discussed specifically here in Section VIII.F. In some embodiments, the other active therapeutic agent is active against Filoviridae virus infections (e.g., marburg virus, ebola virus, Sudan virus, and cueva virus infections). Non-limiting examples of these other active therapeutic agents include:MR186-YTE, remdesivir, ribavirin, palivizumab, motavizumab, RSV-IGIV (RESPIGAMR), MEDI-557, A-60444, MDT-637, BMS-433771, amiodarone, dronedarone, verapamil, Ebola Convalescent Plasma (ECP), TKM-100201, BCX4430 ((2S,3S,4R,5R)-2-(4-amino-5H-pyrrolo[3,2-d]pyrimidin-7-yl)-5-(hydroxymethyl)pyrrolidine-3,4-diol), TKM-Ebola, T-705 monophosphate, T-705 diphosphate, T-705 triphosphate, FGI-106 (1-N,7-N-bis[3-(dimethylamino)propyl]-3,9-dimethylquinolino[8,7-h]quinolone-1,7-diamine), rNAPc2, OS-2966, brincidofovir, remdesivir; RNA polymerase inhibitors, such as galidesivir, favipiravir (also known as T-705 or Avigan), JK-05; host cell factor modulators, such as GMV-006; cadherin-5/factor Ia modulators, such as FX-06; and antibodies for the treatment of Ebola, such as INMAZEB (atoltivimab, maftivimab, and odesivimab), ZMapp, and mAb114 (EBANGA).

Other non-limiting active therapeutic agents active against Ebola include, but are not limited to, an alpha-glucosidase 1 inhibitor, a cathepsin B inhibitor, a CD29 antagonist, a dendritic ICAM-3 grabbing nonintegrin 1 inhibitor, an estrogen receptor antagonist, a factor VII antagonist HLA class II antigen modulator, a host cell factor modulator, a Interferon alpha ligand, a neutral alpha glucosidase AB inhibitor, a niemann-Pick C1 protein inhibitor, a nucleoprotein inhibitor, a polymerase cofactor VP35 inhibitor, a Serine protease inhibitor, a tissue factor inhibitor, a TLR-3 agonist, a viral envelope glycoprotein inhibitor, and an Ebola virus entry inhibitors (NPC1 inhibitors).

In some embodiments, the other active therapeutic agent can be a vaccine for the treatment or prevention of Ebola, including but not limited to VRC-EBOADC076-00-VP, adenovirus-based Ebola vaccine, rVSV-EBOV, rVSVN4CT1-EBOVGP, MVA-BN Filo+Ad26-ZEBOV regimen, INO-4212, VRC-EBODNA023-00-VP, VRC-EBOADC069-00-VP, GamEvac-combi vaccine, SRC VB Vector, HPIV3/EboGP vaccine, MVA-EBOZ, Ebola recombinant glycoprotein vaccine, Vaxart adenovirus vector 5-based Ebola vaccine, FiloVax vaccine, GOVX-E301, and GOVX-E302.

The compounds provided herein can also be used in combination with phosphoramidate morpholino oligomers (PMOs), which are synthetic antisense oligonucleotide analogs designed to interfere with translational processes by forming base-pair duplexes with specific RNA sequences. Examples of PMOs include but are not limited to AVI-7287, AVI-7288, AVI-7537, AVI-7539, AVI-6002, and AVI-6003.

The compounds provided herein are also intended for use with general care provided to subjects with Filoviridae viral infections, including parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication, antiemetic (such as metoclopramide) and/or antidiarrheal agents, vitamin and mineral supplements (including Vitamin K and zinc sulfate), anti-inflammatory agents (such as ibuprofen), pain medications, and medications for other common diseases in the subject population, such anti-malarial agents (including artemether and artesunate-lumefantrine combination therapy), typhoid (including quinolone antibiotics, such as ciprofloxacin, macrolide antibiotics, such as azithromycin, cephalosporin antibiotics, such as ceftriaxone, or aminopenicillins, such as ampicillin), or shigellosis.

### G. Combination Therapy for the Treatment of Influenza

The compounds and pharmaceutically acceptable salts thereof disclosed herein can be used in combination with any of the active therapeutic agents discussed in Section VIII herein and/or with other active therapeutic agents for the treatment of influenza virus infections discussed specifically here in Section VIII.G. In some embodiments, the compounds provided herein are also used in combination with other active therapeutic agents for the treatment of influenza virus infections. The compounds and compositions provided herein are also used in combination with other active therapeutic agents. In some embodiments, the compounds provided herein can also be combined with influenza treatments. In some embodiments, the compounds provided herein are used with influenza treatments when treating influenza viruses. In some embodiments, the compounds provided herein are used with influenza treatments to treat a broader spectrum of respiratory viruses, such as those disclosed herein. In some embodiments, the influenza treatment is a neuraminidase (NA) inhibitor. In some embodiments, the influenza treatment is an M2 inhibitor. Examples of influenza treatments include, but are not limited to, AB-5080, ALS-1, amantadine (GOCOVRI®), AV-001, AV-5124, AVM-0703, baloxavir marboxil (XOFLUZA®), CB-012, CC-42344, CD-388, CT-P27, Codivir, DAS-181, DNK-651, ENOB-FL-01, ENOB-FL-11, favipiravir, GP-584, GP-681, H-015, HC-imAb, HEC-116094HCl·3H2O, HNC-042, histamine glutarimide, IFV-PA, Ingavirin, INI-2004, INNA-051, KYAH01-2019-121, laninamivir, molnupiravir, niclosamide, nitazoxanide, norketotifen, NX-2016, oseltamivir phosphate (TAMIFLU®), peramivir (RAPIVAB®), REVTx-99, rimantadine, S-416, SAB-176, STP-702, T-705IV, TG-1000, TJ-27, TSR-066, 7HP-349, VIR-2482, VIS-410, VIS-FLX, XC-221, zanamivir (RELENZA®), zanamivir-dinitrophenyl conjugate, ZSP-1273, and ZX-7101A.

### IX. Compound Preparation

In some embodiments, the present disclosure provides processes and intermediates useful for preparing the compounds disclosed herein or pharmaceutically acceptable salts thereof.

Compounds disclosed herein can be purified by any of the means known in the art, including chromatographic means, including but not limited to high-performance liquid chromatography (HPLC), preparative thin layer chromatography, flash column chromatography, and ion exchange chromatography. Any suitable stationary phase can be used, including but not limited to, normal and reversed phases as well as ionic resins. In some embodiments, the disclosed compounds are purified via silica gel and/or alumina chromatography.

During any of the processes for preparation of the compounds provided herein, it can be necessary and/or desirable to protect sensitive or reactive groups on any of the molecules concerned. This can be achieved by means of conventional protecting groups as described in standard works, such as T. W. Greene and P. G. M. Wuts, PROTECTIVE GROUPS IN ORGANIC SYNTHESIS, 4^{th} ed., Wiley, New York 2006. The protecting groups can be removed at a convenient subsequent stage using methods known from the art.

Exemplary chemical entities useful in methods of the embodiments will now be described by reference to illustrative synthetic schemes for their general preparation herein and the specific examples that follow. Skilled artisans will recognize that, to obtain the various compounds herein, starting materials can be suitably selected so that the ultimately desired substituents will be carried through the reaction scheme with or without protection as appropriate to yield the desired product. Alternatively, it can be necessary or desirable to employ, in the place of the ultimately desired substituent, a suitable group that can be carried through the reaction scheme and replaced as appropriate with the desired substituent. Furthermore, one of skill in the art will recognize that the transformations shown in the schemes below can be performed in any order that is compatible with the functionality of the particular pendant groups.

The methods of the present disclosure generally provide a specific enantiomer or diastereomer as the desired product, although the stereochemistry of the enantiomer or diastereomer was not determined in all cases. When the stereochemistry of the specific stereocenter in the enantiomer or diastereomer is not determined, the compound is drawn without showing any stereochemistry at that specific stereocenter even though the compound can be substantially enantiomerically or disatereomerically pure.

Representative syntheses of compounds of the present disclosure are described in the schemes below, and the particular examples that follow.

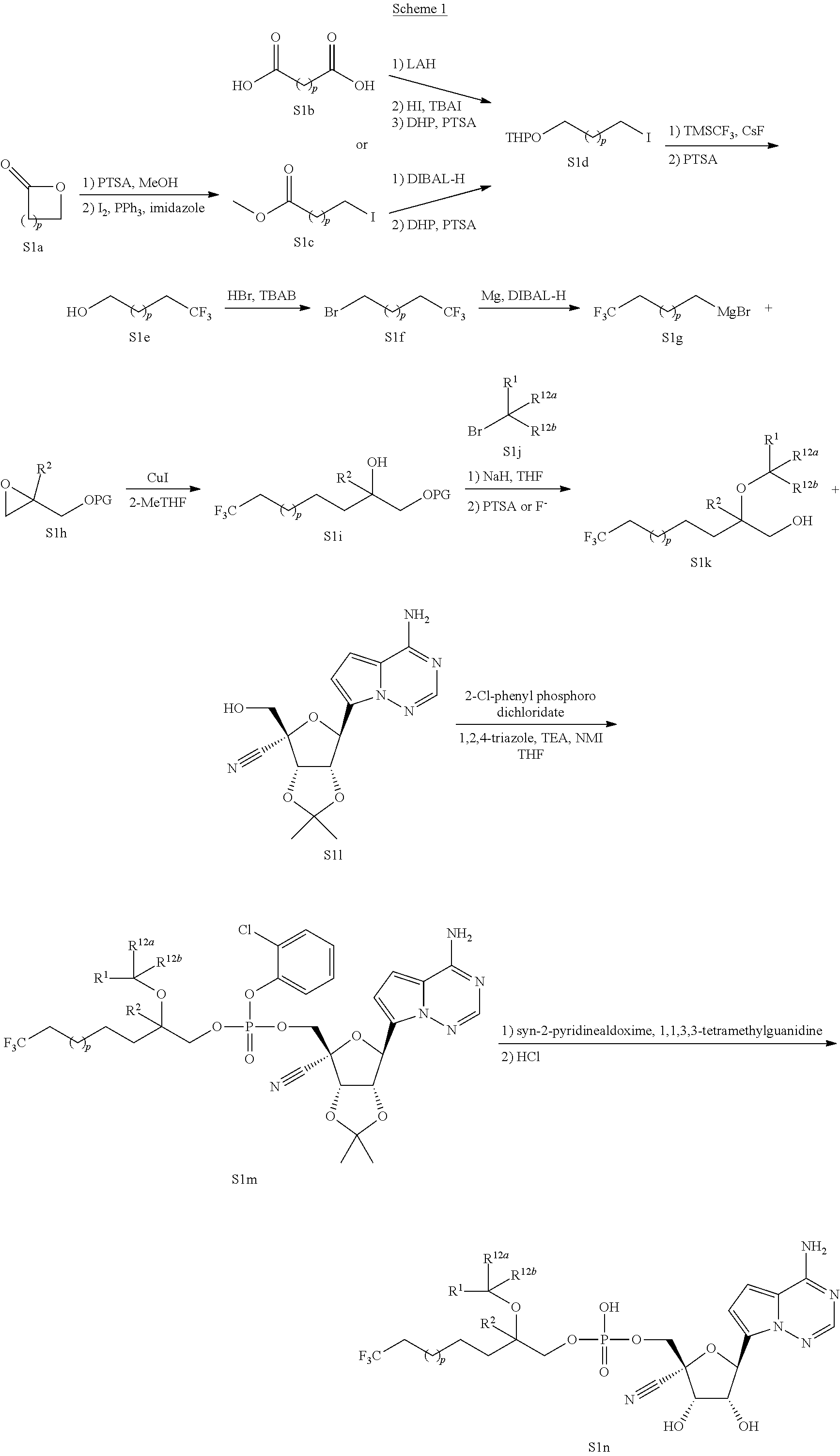

Scheme 1 shows a general synthesis of compounds beginning with the opening of a lactone Sla (where p=C₇₋₂₃ alkylene) to the ester alcohol under acidic conditions (e.g. PTSA, MeOH), which is then converted to the alkyl iodide S1c (e.g. I₂, PPh₃, imidazole). Reduction of the ester (e.g., DIBAL-H) followed by protection (e.g., DHP) of the alcohol affords intermediate S1d, which can be alternatively synthesized via reduction of the dicarboxylic acid S1b (where p=C₇₋₂₃ alkylene) (e.g., LAH), conversion to the alkyl iodide (e.g., HI, TBAI) and protection (e.g., DHP) of the alcohol. A substitution reaction of the alkyl iodide S1d (e.g., TMSCF₃, CsF) followed by deprotection of the alcohol under acidic conditions (e.g., PTSA) affords the trifluoromethyl alcohol S1e. Conversion of S1e to the alkyl bromide S1f (e.g., HBr, TBAB) and subsequent reaction with activated (e.g., DIBAL-H) Mg turnings generates the Grignard S1g. Addition of S1g to an epoxide S1h with PG (e.g., Tr, TBDPS) affords alcohol S1i. A substitution reaction with the halide S1j (e.g., Br) under basic conditions (e.g., NaH) and removal of PG (e.g., PTSA or TBAF) affords alcohol S1k. The alcohol S1k and nucleoside S1l are coupled with 2-Cl-phenyl phosphorodichloridate under basic conditions (e.g., 1,2,4-triazole, TEA, NMI, THF) to afford S1m. Removal of the 2-Cl-phenol (e.g., syn-2-pyridinealdoxime, 1,1,3,3-tetramethylguanidine) and acetonide (e.g., HCl) affords final compounds (e.g., Compounds 2, 3 and 6 of Table 1) of the type S1n.

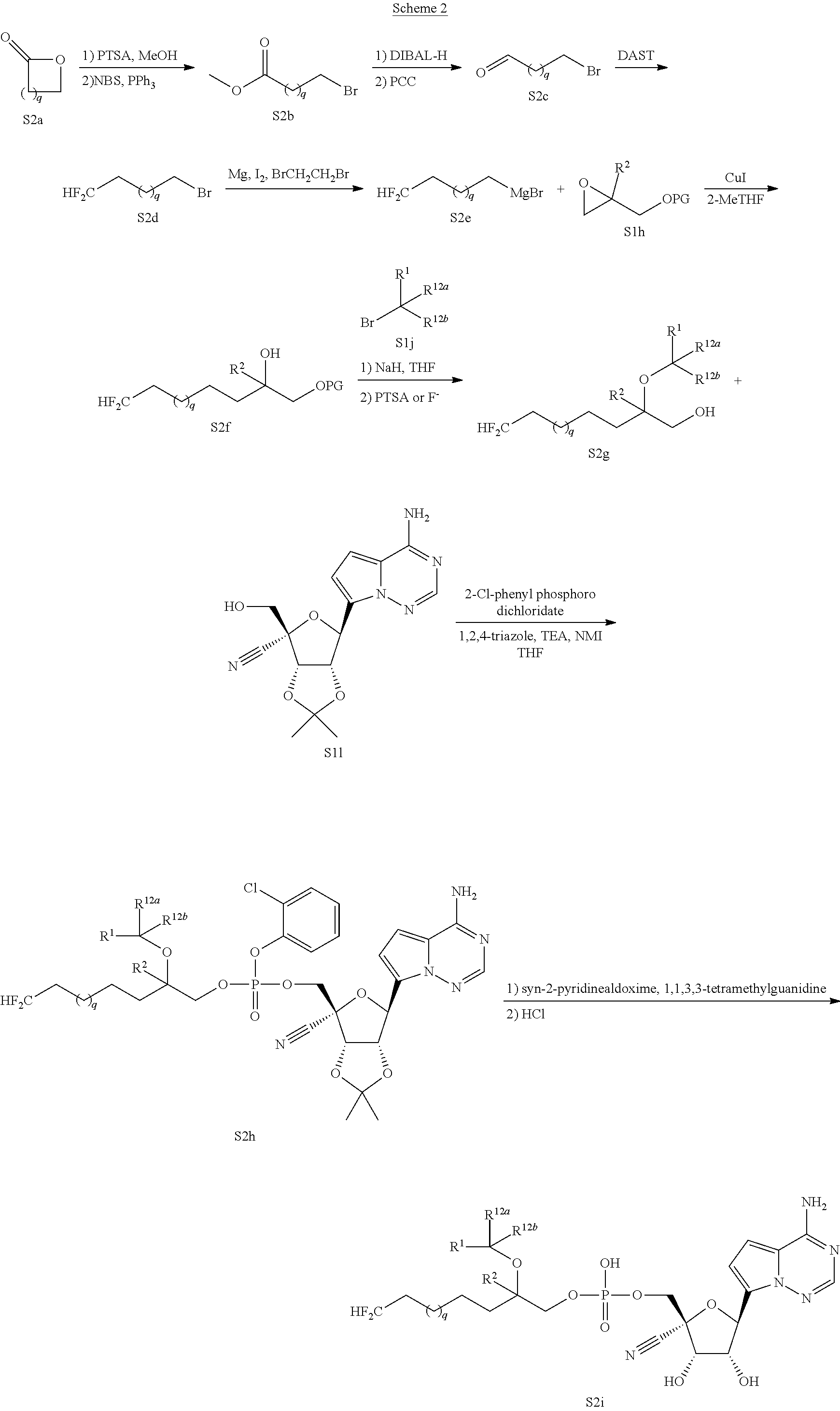

Scheme 2 shows a general synthesis of compounds beginning with the opening of a lactone S2a (where q=C₇₋₂₃ alkylene) to the ester alcohol under acidic conditions (e.g., PTSA, MeOH), which is then converted to the alkyl bromide S2b (e.g., NBS, PPh₃). Reduction of the ester (ex. DIBAL-H) followed by oxidation of the alcohol (e.g., PCC) affords intermediate S2c. Transformation of the aldehyde S2c using a fluorinating reagent (e.g., DAST) generates the difluoromethyl alkyl bromide S2d. Reaction of S2d with activated (e.g., I₂, BrCH₂CH₂Br) Mg turnings generates the Grignard S2e. Addition of S2e to an epoxide S1h with PG (e.g., Tr, TBDPS) affords alcohol S2f. A substitution reaction with the halide S1j (e.g., Br) under basic conditions (e.g., NaH) and removal of PG (e.g., PTSA or TBAF) affords alcohol S2g. The alcohol S2g and nucleoside S1l are coupled with 2-Cl-phenyl phosphorodichloridate under basic conditions (e.g., 1,2,4-triazole, TEA, NMI, THF) to afford S2h. Removal of the 2-Cl-phenol (e.g., syn-2-pyridinealdoxime, 1,1,3,3-tetramethylguanidine) and acetonide (e.g., HCl) affords final compounds (e.g., Compound 4 of Table 1) of the type S2i.

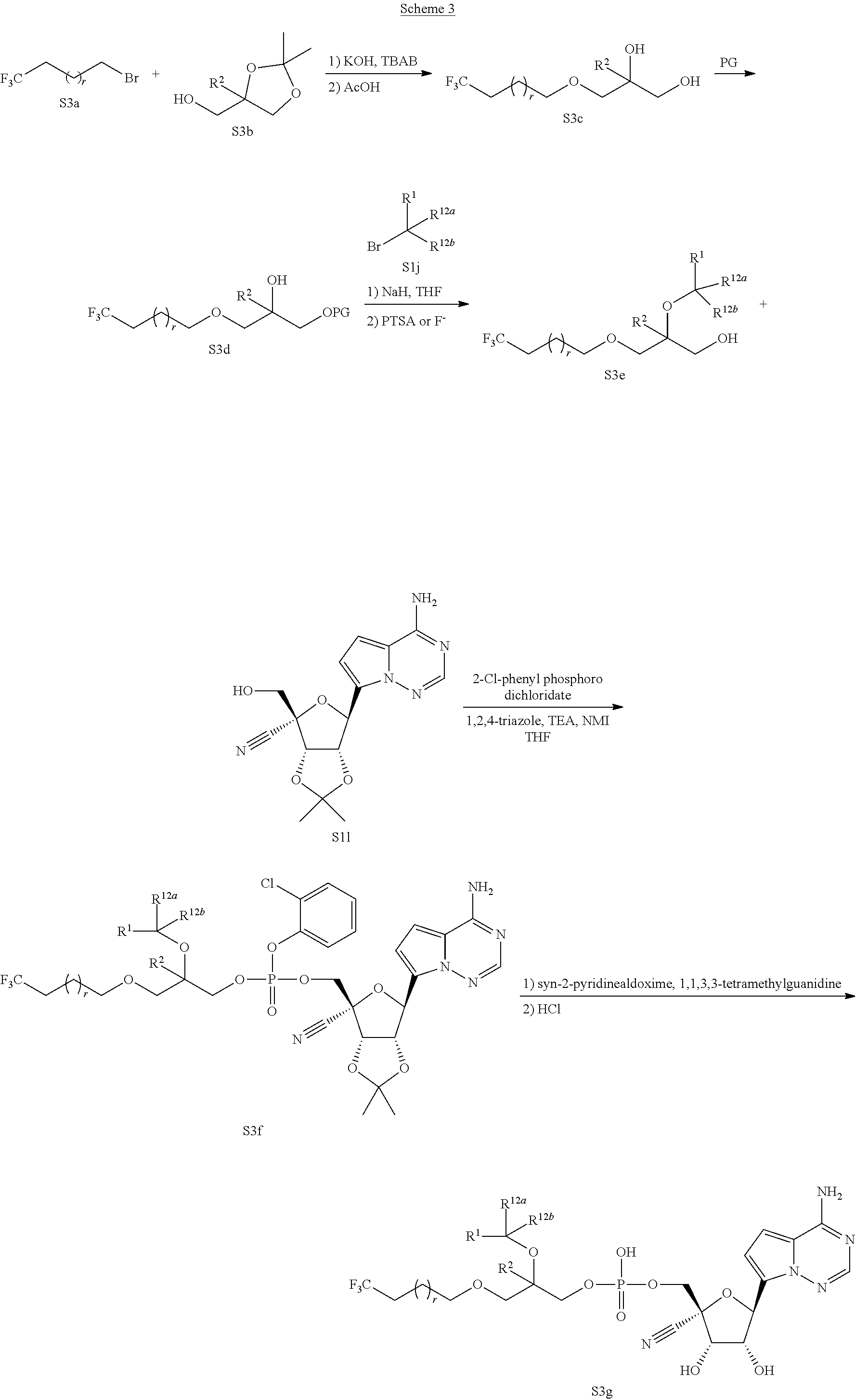

Scheme 3 shows a general synthesis of compounds beginning with a substitution reaction with the alkyl bromide S3a (where r=C₈₋₂₁ alkylene) and the alcohol S3b under basic conditions (e.g., KOH) followed by deprotection of the acetonide under acidic conditions (e.g., AcOH) to afford the diol S3c. S3a is prepared in a similar manner to S1f. Protection of the diol (e.g., TrCl, TEA) generates intermediate S3d, which can undergo a substitution reaction with the halide S1j (e.g., Br) under basic conditions (e.g., NaH) and a removal of PG (e.g., PTSA) to generate the alcohol S3e. S3e and nucleoside S1l are coupled with 2-Cl-phenyl phosphorodichloridate under basic conditions (e.g., 1,2,4-triazole, TEA, NMI, THF) to afford S3f. Removal of the 2-Cl-phenol (e.g., syn-2-pyridinealdoxime, 1,1,3,3-tetramethylguanidine) and acetonide (e.g., HCl) affords final compounds (e.g., Compound 1 of Table 1) of the type S3g.

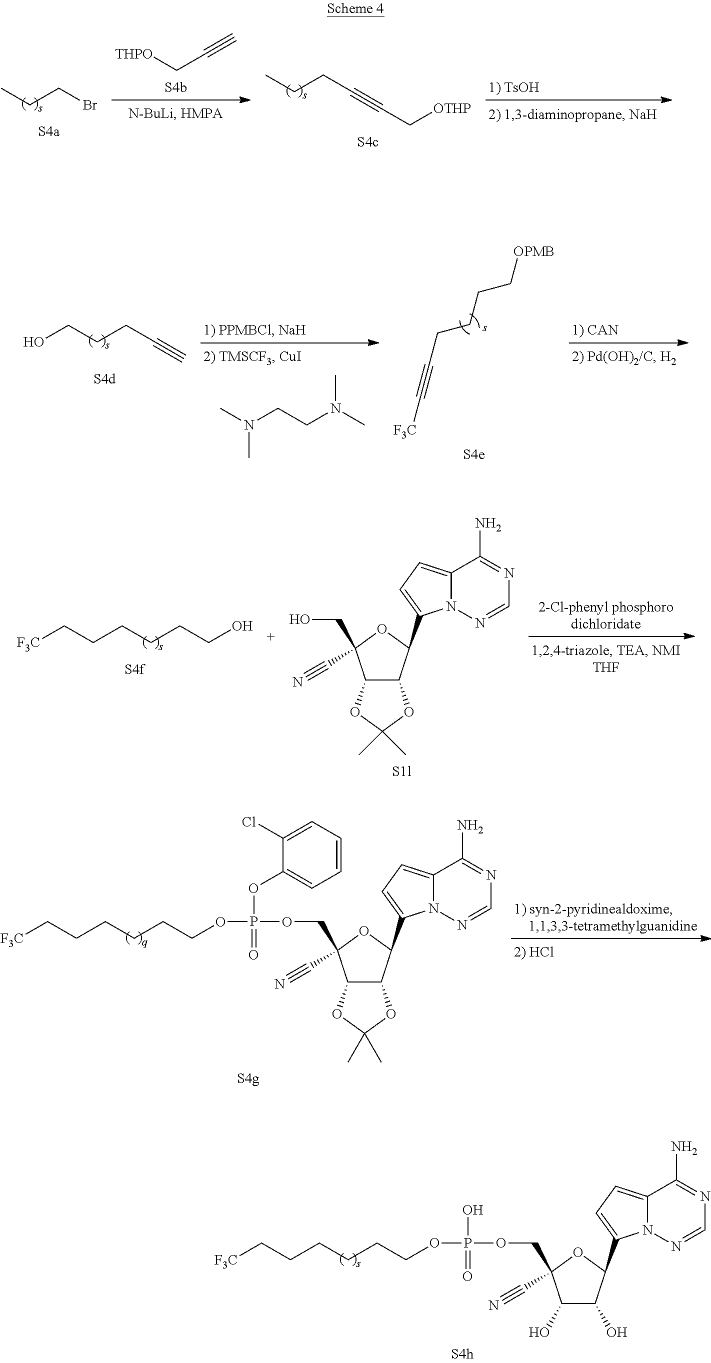

Scheme 4 shows a general synthesis of compounds beginning with a substitution reaction of the alkyl halide S4a (e.g., Br) (where s=C₇₋₂₁ alkylene) and the alkyne S4b with PG (e.g., THP) under basic conditions (e.g., n-BuLi) to afford the intermediate S4c. Deprotection of S4c under acidic conditions (e.g., PTSA) and a rearrangement under basic conditions (e.g., NaH, 1,3-diaminopropane) yields the alcohol S4d. Protection of S4d (e.g., PMBCl, NaH) followed by trifluoromethylation of the terminal alkyne (e.g., TMSCF₃, CuI) generates intermediate S4e. Deprotection of S4e (e.g., CAN) and reduction of the alkyne (e.g., Pd(OH)₂/C, H₂) yields the trifluoromethyl alcohol S4f. S4f and nucleoside S1l are coupled with 2-Cl-phenyl phosphorodichloridate under basic conditions (e.g., 1,2,4-triazole, TEA, NMI, THF) to afford S4g. Removal of the 2-Cl-phenol (e.g., syn-2-pyridinealdoxime, 1,1,3,3-tetramethylguanidine) and acetonide (e.g., HCl) affords final compounds (e.g., Compound 5 of Table 1) of the type S4h.

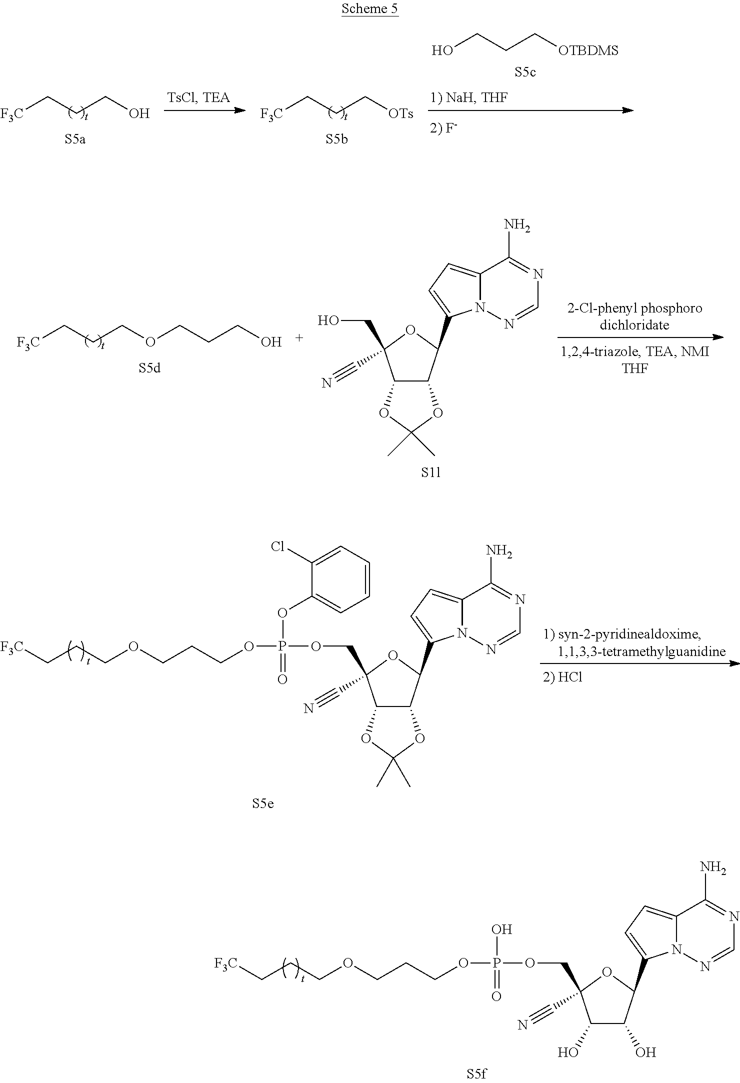

Scheme 5 shows a general synthesis of compounds beginning with the coupling of alcohol S5a (where t=C₈₋₂₁ alkylene) with a leaving group under basic conditions (e.g., TsCl, TEA). S5a is prepared in a similar manner to S1e. A substitution reaction with S5b and the alcohol S5c with PG (e.g., TBDMS) under basic conditions (e.g., NaH) followed by removal of the PG (e.g., TBAF) yields the alcohol S5d. S5d and nucleoside S1l are coupled with 2-Cl-phenyl phosphorodichloridate under basic conditions (e.g., 1,2,4-triazole, TEA, NMI, THF) to afford S5e. Removal of the 2-Cl-phenol (e.g., syn-2-pyridinealdoxime, 1,1,3,3-tetramethylguanidine) and acetonide (e.g., HCl) affords final compounds of the type S5f.

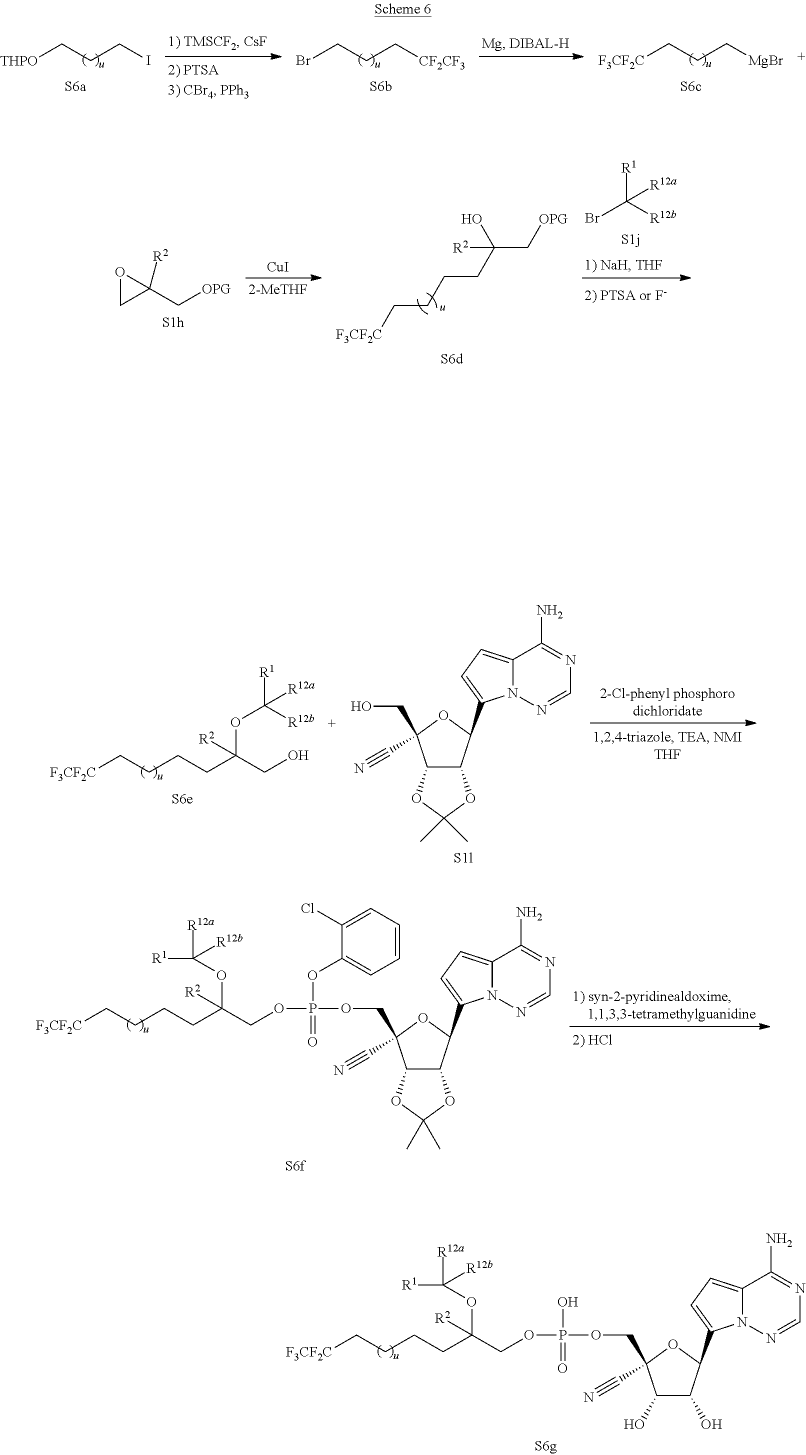

Scheme 6 shows a general synthesis of compounds beginning with the fluorination of the alkyl iodide S6a (where u=C₇₋₂₂ alkylene) (e.g., TMSCF₂CF₃. CsF), deprotection under acidic conditions (e.g., PTSA) and conversion to the corresponding alkyl bromide (e.g., CBr₄, PPh₃) to yield S6b. S6a is prepared in a similar manner to S1d. Reaction of S6b with activated (e.g., DIBAL-H) Mg turnings generates the Grignard S6c. Addition of S6c to an epoxide S1h with PG (e.g., Tr, TBDPS) yields alcohol S6d. A substitution reaction with the halide S1j (e.g., Br) under basic conditions (e.g., NaH) and removal of PG (e.g., PTSA or TBAF) affords alcohol S6e. The alcohol S6e and nucleoside S1l are coupled with 2-Cl-phenyl phosphorodichloridate under basic conditions (e.g., 1,2,4-triazole, TEA, NMI, THF) to give S6f. Removal of the 2-Cl-phenol (e.g., syn-2-pyridinealdoxime, 1,1,3,3-tetramethylguanidine) and acetonide (e.g., HCl) affords final compounds of the type S6g.

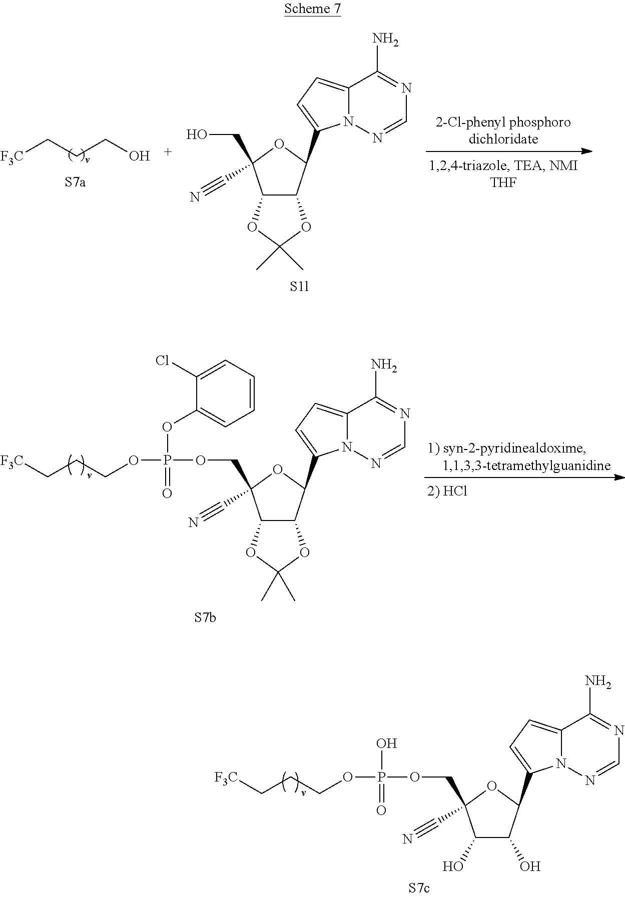

Scheme 7 shows a general synthesis of compounds beginning with the coupling of trifluoromethyl alcohol S7a (where v=C₁₀₋₂₆ alkylene) and nucleoside S1l with 2-C₁-phenyl phosphorodichloridate under basic conditions (e.g., 1,2,4-triazole, TEA, NMI, THF) to afford S7b. S7a is prepared in a similar manner to S1e. Removal of the 2-C₁-phenol (e.g., syn-2-pyridinealdoxime, 1,1,3,3-tetramethylguanidine) and acetonide (e.g., HCl) affords final compounds of the type S7c.

### Examples

### A. Abbreviations

Certain abbreviations and acronyms are used in describing the experimental details. Although most of these would be understood by one skilled in the art, Table 2 contains a list of many of these abbreviations and acronyms.

TABLE 2
List of abbreviations and acronyms.

| Abbreviation | Meaning |
|---|---|
| Ac | acetate |
| ACN | acetonitrile |
| AIBN | azobisisobutyronitrile |
| Bn | benzyl |
| Bu | butyl |
| Bz | benzoyl |
| BzCl | benzoyl chloride |
| CDI | 1,1′-carbonyldiimidazole |
| DAST | diethylaminosulfur trifluoride |
| DCE | 1,2-dichloroethane |
| DCM | dichloromethane |
| DIPEA | N,N-diisopropylethylamine |
| DMAP | 4-dimethylamiopyridine |
| DMDO | dimethydioxirane |
| DMSO | dimethylsulfoxide |
| DMF | dimethylformamide |
| DMTrCl | 4,4′-dimethoxytritylchloride |
| DMTr | 4,4′-dimethoxytrityl |
| EDCI | N-(3-dimethylaminopropyl)-N′- |
| | ethylcarbodiimide hydrochloride |
| Et | ethyl |
| Imid | imidazole |
| KOtBu | potassium tert-butoxide |
| LC | liquid chromatography |
| MCPBA | meta-chloroperbenzoic acid |
| Me | methyl |
| m/z | mass to charge ratio |
| MS or ms | mass spectrum |
| NIS | N-iodosuccinimide |
| NMI | N-Methylimidazole |
| NMP | N-methyl-2-pyrrolidone |
| Ph | phenyl |
| Ph₃P | triphenylphosphine |
| PMB | para-methoxybenzyl |
| PMBCl | para-methoxybenzyl chloride |
| PhOC(S)Cl | phenylchlorothionoformate |
| (PhO)₃PMeI | methyltriphenoxyphosphonium iodide |
| Pyr | pyridine |
| RT | room temperature |
| SFC | supercritical fluid chromatography |
| TBAF | tetrabutylammonium fluoride |
| TBS | tert-butyldimethylsilyl |
| TBSCl | tert-Butyldimethylsilyl chloride |
| TMSN₃ | trimethylsilyl azide |
| TEA | triethylamine |
| TES | triethylsilane |
| TFA | trifluoroacetic acid |
| THF | tetrahydrofuran |
| TMS | trimethylsilyl |
| TMSCl | trimethylsilyl chloride |
| Ts | 4-toluenesulfonyl |
| TsOH | tosylic acid |
| δ | parts per million referenced to residual |
| | non-deuterated solvent peak |

### B. Intermediates

### Intermediate I-1: (3aS,4R,6S,6aS)-6-(4-aminopyrrolo[2,1-f][1,2,4]triazin-7-yl)-4-(hydroxymethyl)-2,2-dimethyltetrahydrofuro[3,4-d][1,3]dioxole-4-carbonitrile

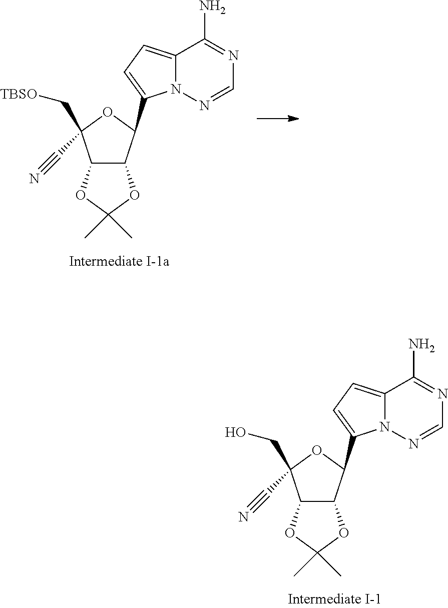 [SMILES: CC1(C)O[C@H]2[C@H](c3ccc4c(N)ncnn34)O[C@](C#N)(CO[Si](C)(C)C(C)(C)C)[C@H]2O1 ; CC1(C)O[C@H]2[C@H](c3ccc4c(N)ncnn34)O[C@](C#N)(CO)[C@H]2O1]

Intermediate I-1a, (3aS,4R,6S,6aS)-6-(4-aminopyrrolo[2,1-f][1,2,4]triazin-7-yl)-4-(((tert-butyldimethylsilyl)oxy)methyl)-2,2-dimethyltetrahydrofuro[3,4-d][1,3]dioxole-4-carbonitrile was prepared according to WO2015/069939. For example, pages 127-138 of WO2015/069939 provide a process for preparing this compound (identified as compound 14k in WO2015/069939).

To a solution of Intermediate I-1a (18.87 mmol) in THF (100 mL). Added TBAF 1.0 M in THF (28.31 mmol) in one portion at ambient temperature. Allowed to stir at ambient temperature for 10 min. The reaction was determined to be complete by LCMS. The reaction mixture was quenched with water and the organics were removed under reduced pressure. The crude was partitioned between EtOAc and Water. The layers were separated and the aqueous was washed with EtOAc. The organics were combined and dried over sodium sulfate. The solids were filtered off and the solvent removed under reduced pressure. The crude was purified by silica gel chromatography 120 g column 0% to 10% CH₃OH in CH₂Cl2 to afford Intermediate I-3a. LC/MS: t_{R}=0.76 min, MS m/z=332.14 [M+1]; LC system: Thermo Accela 1250 UHPLC. MS system: Thermo LCQ Fleet; Column: Kinetex 2.6μ XB-C18 100A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min. ¹H NMR (400 MHZ, DMSO-d₆) δ 7.87-7.80 (m, 3H), 6.85 (d, J=4.5 Hz, 1H), 6.82 (d, J=4.5 Hz, 1H), 5.74 (t, J=5.8 Hz, 1H), 5.52 (d, J=4.2 Hz, 1H), 5.24 (dd, J=6.8, 4.2 Hz, 1H), 4.92 (d, J=6.8 Hz, 1H), 3.65 (dd, J=6.1, 1.7 Hz, 2H), 1.61 (s, 3H), 1.33 (s, 3H).

### Intermediate I-2: heptadecane-1,17-diol

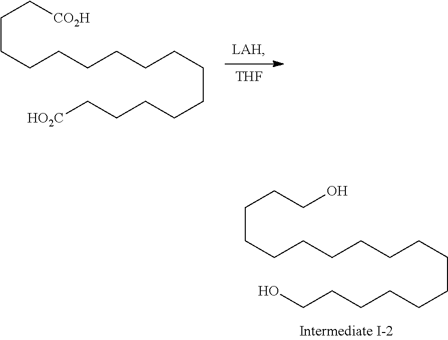

LiAlH₄ (9.4 g, 249.6 mmol, 6 eq) was added into THF (700 mL) at 0° C. to make a suspension. Then heptadecanedioic acid (12.5 g, 41.61 mmol, 1 eq) was added into the above suspension slowly at 0° C. The mixture was stirred at 70° C. for 12 hr, then cooled to 0° C., and the reaction quenched by sequential addition of H₂O (10 mL), 15% NaOH (10 mL), aqNa₂SO₄ solution (20 mL). The mixture was then filtered, washed with THF (5 L), and concentrated under reduced pressure. The crude product was triturated with petroleum ether (80 mL) at 20° C. for 30 min, filtered, washed with petroleum ether (80 mL), and filter cake dried under reduced pressure to give heptadecane-1,17-diol, Intermediate I-2, which was used in next reaction without further purification.

### Intermediate I-3: 17-iodoheptadecan-1-ol

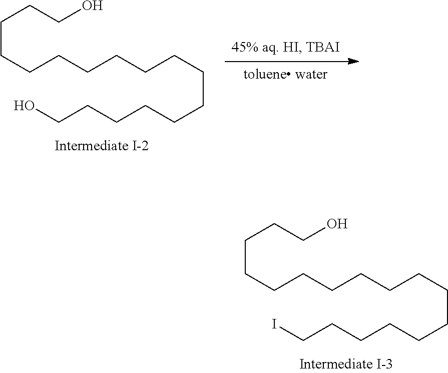 [SMILES: OCCCCCCCCCCCCCCCCCO ; "45% aq. HI, TBAI" ; "toluene· water" ; OCCCCCCCCCCCCCCCCCI]

To a solution of heptadecane-1,17-diol, Intermediate I-2 (10 g, 36.7 mmol, 1 eq) in toluene (180 mL) was added TBAI (542.2 mg, 1.4 mmol, 0.04 eq). Then HI (10.4 g, 36.70 mmol, 6.1 mL, 45% solution, 1 eq) was added to the solution at 90° C. The mixture was stirred at 90° C. for 12 hr, filtered, and concentrated under reduced pressure. The residue was diluted with H₂O (80 mL) and extracted with ethyl acetate (100 mL×3). The combined organic layers were washed with brine (80 mL×3), dried over Na₂SO₄, filtered, and concentrated under reduced pressure to give a residue. The residue was purified by flash silica gel chromatography (ISCO®; 40 g SepaFlash® Silica Flash Column, Eluent of 0˜20% Ethyl acetate/Petroleum ether gradient @ 120 mL/min) to give 17-iodoheptadecan-1-ol, Intermediate I-3.

### Intermediate I-4: 2-((17-iodoheptadecyl)oxy)tetrahydro-2H-pyran

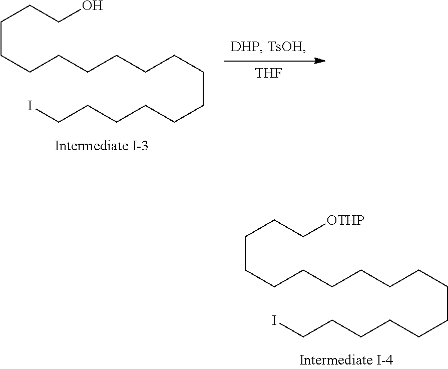

To a solution of 17-iodoheptadecan-1-ol, Intermediate I-3 (2.5 g, 6.5 mmol, 1 eq) in THF (40 mL) were added 3,4-dihydro-2H-pyran (1.3 g, 16.3 mmol, 1.4 mL, 2.5 eq) and PTSA (168.8 mg, 980.7 umol, 0.15 eq). The mixture was stirred at 20° C. for 12 hr and concentrated under reduced pressure. The residue was diluted with H₂O (50 mL) and extracted with ethyl acetate (80 mL×3). The combined organic layers were washed with brine (50 mL×3), dried over Na₂SO₄, filtered, and concentrated under reduced pressure to give a residue. The residue was purified by flash silica gel chromatography (ISCO®; 40 g SepaFlash® Silica Flash Column, Eluent of 0˜6% Ethyl acetate 1 Petroleum ether gradient @ 120 mL/min) to give 2-((17-iodoheptadecyl)oxy)tetrahydro-2H-pyran, Intermediate I-4. ¹H NMR (400 MHZ, CHLOROFORM-d) δ 4.62-4.53 (m, 1H), 3.94-3.82 (m, 1H), 3.79-3.68 (m, 1H), 3.55-3.46 (m, 1H), 3.44-3.34 (m, 1H), 3.20 (t, J=7.0 Hz, 2H), 1.90-1.77 (m, 3H), 1.77-1.67 (m, 1H), 1.65-1.47 (m, 6H), 1.45-1.18 (m, 26H).

### Intermediate I-5: 2-((18,18,18-trifluorooctadecyl)oxy)tetrahydro-2H-pyran

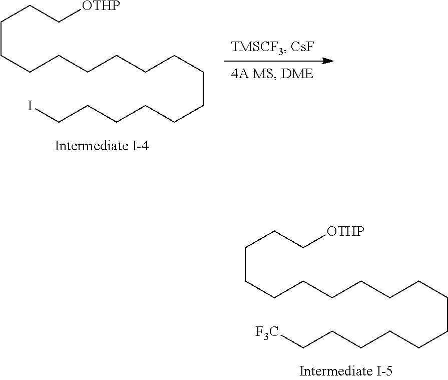

To a solution of CsF (2.6 g, 17.1 mmol, 632.2 uL, 3.2 eq) in 15-CROWN-5 (6.4 g, 29.2 mmol, 5.8 mL, 5.4 eq) were added DME (40 mL) and 4A MS (2 g) and the mixture cooled to −20° C. Then a solution of 2-((17-iodoheptadecyl)oxy)tetrahydro-2H-pyran, Intermediate I-4 (2.5 g, 5.3 mmol, 1 eq) and TMS-CF₃ (3.2 g, 22.7 mmol, 4.2 eq) in DME (40 mL) was added into the above mixture over 15 min. The mixture was stirred at 25° C. for 12 hr, diluted with ethyl acetate (50 mL), washed with brine (50 mL×2), dried over Na₂SO₄, filtered, and concentrated under reduced pressure to give a residue. The residue was purified by column chromatography (SiO₂, Petroleum ether/Ethyl acetate=100/1 to 100/3) to give 2-((18,18,18-trifluorooctadecyl)oxy)tetrahydro-2H-pyran, Intermediate I-5. ¹H NMR (400 MHZ, CHLOROFORM-d) δ 4.58 (br s, 1H), 3.88 (br t, J=9.2 Hz, 1H), 3.80-3.68 (m, 1H), 3.58-3.46 (m, 1H), 3.45-3.33 (m, 1H), 2.14-1.96 (m, 2H), 1.64-1.50 (m, 8H), 1.40-1.15 (m, 28H).

### Intermediate I-6: 18,18,18-trifluorooctadecan-1-ol

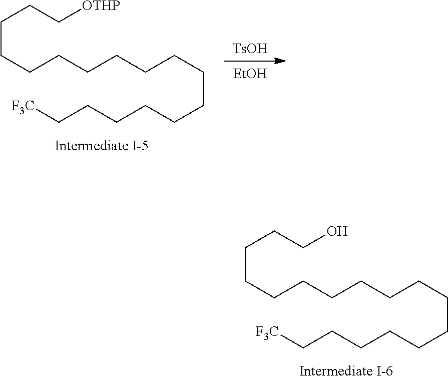

To a solution of 2-((18,18,18-trifluorooctadecyl)oxy)tetrahydro-2H-pyran, Intermediate I-5 (2.1 g, 5.1 mmol, 1 eq) in EtOH (30 mL) was added TsOH (1.6 g, 9.2 mmol, 1.8 eq). The mixture was stirred at 50° C. for 3 hr, diluted with ethyl acetate (80 mL), washed with NaCl (50 mL×3), dried over Na₂SO₄, filtered, and concentrated under reduced pressure to give a residue. The residue was purified by flash silica gel chromatography (ISCO®; 40 g SepaFlash® Silica Flash Column, Eluent of 0˜8% Ethyl acetate/Petroleum ether gradient @ 120 mL/min) to give 18,18,18-trifluorooctadecan-1-ol, Intermediate I-6. ¹H NMR (400 MHZ, CHLOROFORM-d) δ 3.65 (t, J=6.6 Hz, 2H), 2.14-1.97 (m, 2H), 1.66-1.48 (m, 4H), 1.41-1.18 (m, 26H).

### Intermediate I-7: 18-bromo-1,1,1-trifluorooctadecane

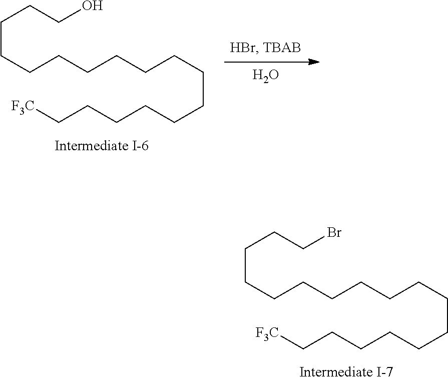

To a solution of 18,18,18-trifluorooctadecan-1-ol, Intermediate I-6 (1.4 g, 4.3 mmol, 1 eq) in HBr (40 mL, in H₂O) was added TBAB (278.1 mg, 862.9 umol, 0.2 eq). The mixture was stirred at 100° C. for 12 hr, diluted with ethyl acetate (80 mL), washed with NaCl (50 mL×3), dried over Na₂SO₄, filtered, and concentrated under reduced pressure to give a residue. The residue was purified by flash silica gel chromatography (ISCO®; 40 g SepaFlash® Silica Flash Column, Eluent of 0˜8% Ethyl acetate/Petroleum ether gradient @ 120 mL/min) to give 18-bromo-1,1,1-trifluorooctadecane, Intermediate I-7. ¹H NMR (400 MHZ, CHLOROFORM-d) δ 3.42 (t, J=6.8 Hz, 2H), 2.14-1.98 (m, 2H), 1.92-1.80 (m, 2H), 1.61-1.50 (m, 2H), 1.49-1.40 (m, 2H), 1.39-1.23 (m, 24H).

### Intermediate I-8: (R)-2,2-dimethyl-4-(((18,18,18-trifluorooctadecyl)oxy)methyl)-1,3-dioxolane

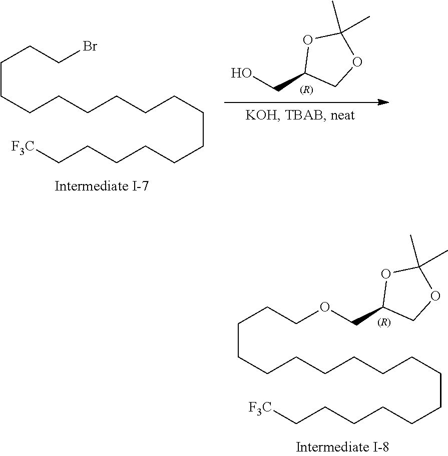

To a solution of 18-bromo-1,1,1-trifluoro-octadecane, Intermediate I-7 (1.4 g, 3.6 mmol, 1 eq) in [(4R)-2,2-dimethyl-1,3-dioxolan-4-yl]methanol (2.8 g, 21.1 mmol, 5.8 eq) were added KOH (506.9 mg, 9.0 mmol, 2.5 eq) and TBAB (233.0 mg, 722.8 umol, 0.2 eq). The mixture was stirred at 40° C. for 12 hr, diluted with H₂O (20 mL), and extracted with ethyl acetate (20 mL×3). The combined organic layers were washed with NaCl (20 mL), dried over Na₂SO₄, filtered, and concentrated under reduced pressure to give a residue. The residue was purified by flash silica gel chromatography (ISCO®; 40 g SepaFlash® Silica Flash Column, Eluent of 0˜8% Ethyl acetate/Petroleum ether gradient @ 100 mL/min) to give (R)-2,2-dimethyl-4-(((18,18,18-trifluorooctadecyl)oxy)methyl)-1,3-dioxolane, Intermediate I-8. ¹H NMR (400 MHZ, CHLOROFORM-d) δ 4.32-4.23 (m, 1H), 4.10-4.03 (m, 1H), 3.77-3.70 (m, 1H), 3.56-3.39 (m, 4H), 2.14-1.97 (m, 2H), 1.63-1.50 (m, 4H), 1.43 (s, 3H), 1.40-1.21 (m, 29H).

### Intermediate I-9: (S)-3-((18,18,18-trifluorooctadecyl)oxy)propane-1,2-diol

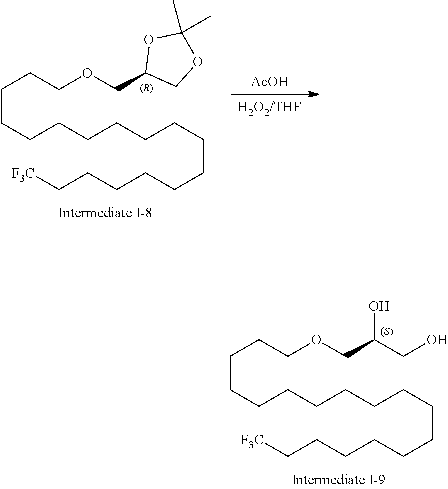

(R)-2,2-dimethyl-4-(((18,18,18-trifluorooctadecyl)oxy)methyl)-1,3-dioxolane, Intermediate I-8 (1.3 g, 2.9 mmol, 1 eq) was dissolved in the solution of THF (25 mL), H₂O (20.0 g, 1.11 mol, 20 mL, 374.5 eq), and AcOH (31.5 g, 524.5 mmol, 30 mL, 176.9 eq). The mixture was stirred at 50° C. for 12 hr and concentrated under reduced pressure to give (S)-3-((18,18,18-trifluorooctadecyl)oxy)propane-1,2-diol, Intermediate I-9 was used in the next step without further purification. 1H NMR (400 MHZ, CHLOROFORM-d) δ 3.90-3.80 (m, 1H), 3.78-3.62 (m, 2H), 3.60-3.40 (m, 4H), 2.77-2.51 (m, 1H), 2.34-2.15 (m, 1H), 2.14-1.99 (m, 2H), 1.66-1.47 (m, 4H), 1.45-1.20 (m, 26H).

### Intermediate I-10: (R)-1-((18,18,18-trifluorooctadecyl)oxy)-3-(trityloxy)propan-2-ol

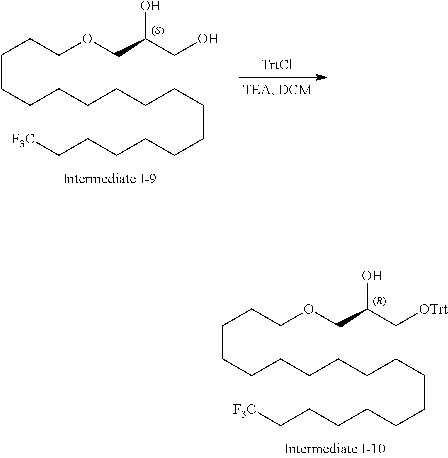

(S)-3-((18,18,18-trifluorooctadecyl)oxy)propane-1,2-diol, Intermediate I-9 (1.3 g, 2.9 mmol, 90% purity, 1 eq) was dissolved in the solution of DCM (30 mL) and TEA (534.7 mg, 5.2 mmol, 735.5 uL, 1.8 eq), then TrtCl (818.4 mg, 2.94 mmol, 1 eq) were added at 0° C. The mixture was stirred at 20° C. for 12 hr, diluted with DCM (10 mL), washed with H₂O (20 mL×3), dried over Na2SO4, filtered, and concentrated under reduced pressure to give a residue. The residue was purified by column chromatography (SiO₂, Petroleum ether/Ethyl acetate=100/1 to 100/6) to give (R)-1-((18,18,18-trifluorooctadecyl)oxy)-3-(trityloxy)propan-2-ol, Intermediate I-10. ¹H NMR (400 MHZ, CHLOROFORM-d) δ 7.50-7.40 (m, 6H), 7.38-7.24 (m, 9H), 4.04-3.94 (m, 1H), 3.62-3.40 (m, 4H), 3.31-3.16 (m, 2H), 2.49-2.31 (m, 1H), 1.68-1.50 (m, 4H), 1.36-1.19 (m, 28H).

### Intermediate I-11: (R)-3-fluoro-5-(((1-((18,18,18-trifluorooctadecyl)oxy)-3-(trityloxy)propan-2-yl)oxy)methyl)benzonitrile

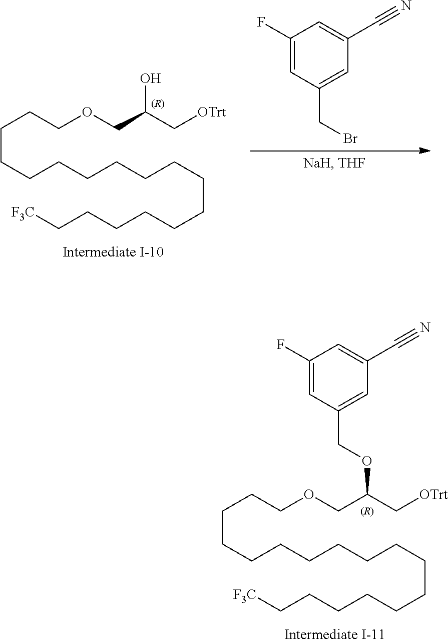

To a solution of (R)-1-((18,18,18-trifluorooctadecyl)oxy)-3-(trityloxy)propan-2-ol, Intermediate I-10 (1 g, 1.56 mmol, 1 eq) in THF (30 mL) was added NaH (187.2 mg, 4.6 mmol, 60% purity, 3 eq) at 0° C. The mixture was stirred at 0° C. for 0.5 hr and then 3-(bromomethyl)-5-fluoro-benzonitrile (601.1 mg, 2.8 mmol, 1.8 eq) was added into the above solution at 0° C. The mixture was stirred at 65° C. for 12 hr. The reaction was quenched by adding into sat NH₄Cl solution (50 mL) at 0° C. The resulting mixture was then extracted with ethyl acetate (20 mL×3). The combined organic layers were washed with NaCl (20 mL×3), dried over Na₂SO₄, filtered, and concentrated under reduced pressure to give a residue. The residue was purified by column chromatography (SiO₂, Petroleum ether/MTBE=100/0 to 100/5) to give (R)-3-fluoro-5-(((1-((18,18,18-trifluorooctadecyl)oxy)-3-(trityloxy)propan-2-yl)oxy)methyl)benzonitrile, Intermediate I-11. ¹H NMR (400 MHZ, CHLOROFORM-d) δ 7.50-7.45 (m, 6H), 7.40 (d, J=9.3 Hz, 1H), 7.37-7.25 (m, 11H), 4.71 (s, 2H), 3.81-3.69 (m, 1H), 3.61 (d, J=5.1 Hz, 2H), 3.45 (t, J=6.6 Hz, 2H), 3.35-3.23 (m, 2H), 1.65-1.52 (m, 4H), 1.38-1.21 (m, 28H).

### Intermediate I-12: (S)-3-fluoro-5-(((1-hydroxy-3-((18,18,18-trifluorooctadecyl)oxy)propan-2-yl)oxy)methyl)benzonitrile

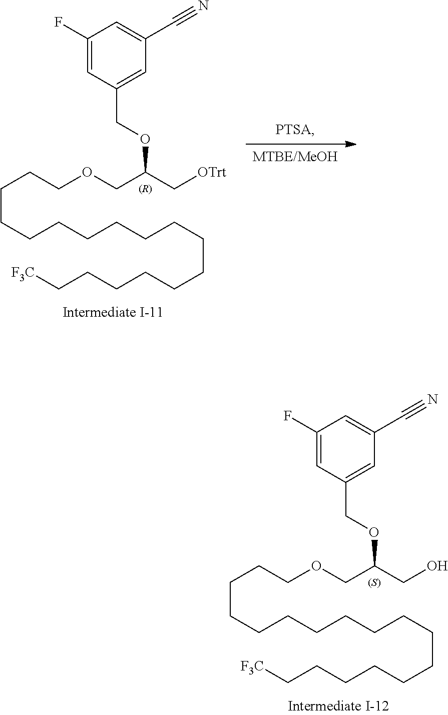

To a solution of (R)-3-fluoro-5-(((1-((18,18,18-trifluorooctadecyl)oxy)-3-(trityloxy)propan-2-1)oxy)methyl)benzonitrile, Intermediate I-11 (400.0 mg, 516.8 umol, 1 eq) in MTBE (15 mL) were added anisole (27.9 mg, 258.4 umol, 28.0 uL, 0.5 eq), MeOH (2.4 mL), and PTSA (44.5 mg, 258.4 umol, 0.5 eq). The mixture was stirred at 50° C. for 3 hr and concentrated under reduced pressure. The residue was diluted with ethyl acetate (20 mL) and washed with NaCl (10 mL×3), dried over Na₂SO4, filtered, and concentrated under reduced pressure to give a residue. The residue was purified by column chromatography (SiO₂, Petroleum ether/Ethyl acetate=20/1 to 4/1). The residue was further purified by prep-HPLC (TFA condition, column: Phenomenex Luna C18 200*40 mm*10 um; mobile phase: [water(TFA)-ACN]; B %: 60%-90%, 8 min) to give (S)-3-fluoro-5-(((1-hydroxy-3-((18,18,18-trifluorooctadecyl)oxy)propan-2-yl)oxy)methyl)benzonitrile, Intermediate I-12. ¹HNMR (400 MHZ, CHLOROFORM-d) δ=7.47 (s, 1H), 7.37 (d, J=8.6 Hz, 1H), 7.29 (s, 1H), 4.79-4.67 (m, 2H), 3.85-3.77 (m, 1H), 3.78-3.55 (m, 2H), 3.66-3.55 (m, 2H), 3.49-3.42 (m, 2H), 2.17-1.97 (m, 3H), 1.65-1.48 (m, 2H), 1.45-1.20 (m, 28H). ¹⁹FNMR (376 MHz, CHLOROFORM-d) δ=−66.38, −109.77. MS (ESI): m/z=532.4 [M+H]⁺.

### Intermediate I-13: 2-chlorophenyl di(1H-1,2,4-triazol-1-yl)phosphinate

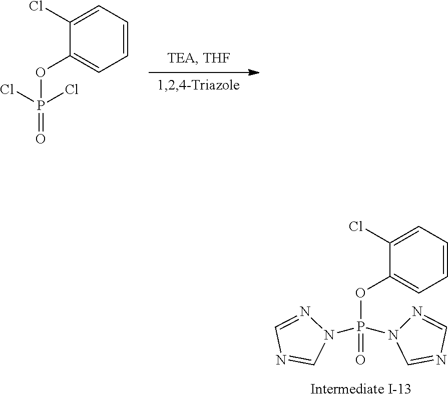 [SMILES: O=P(Cl)(Cl)Oc1ccccc1Cl ; "1,2,4-Triazole" ; "TEA, THF" ; O=P(Oc1ccccc1Cl)(n1cncn1)n1cncn1]

1H-1,2,4-triazole (714 mg, 10.3 mmol) and TEA (1.44 mL, 10.3 mmol) were dissolved in THF (8 mL). To the solution was added 1-chloro-2-dichlorophosphoryloxy-benzene (0.81 mL, 4.92 mmol) dropwise at rt. The reaction mixture was stirred at rt for 30 min and filtered into a graduated tube, the filter cake washed with THF (8 mL), and additional THF added to the filtrate to total volume 20 mL resulting in ca 0.246 M stock solution of Intermediate I-13, which was used in next reactions. ³¹P NMR (162 MHz, Acetonitrile-d₃) 8-16.94.

### Intermediate I-14: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) ((R)-2-((3-cyano-5-fluorobenzyl)oxy)-3-((18,18,18-trifluorooctadecyl)oxy)propyl) phosphate

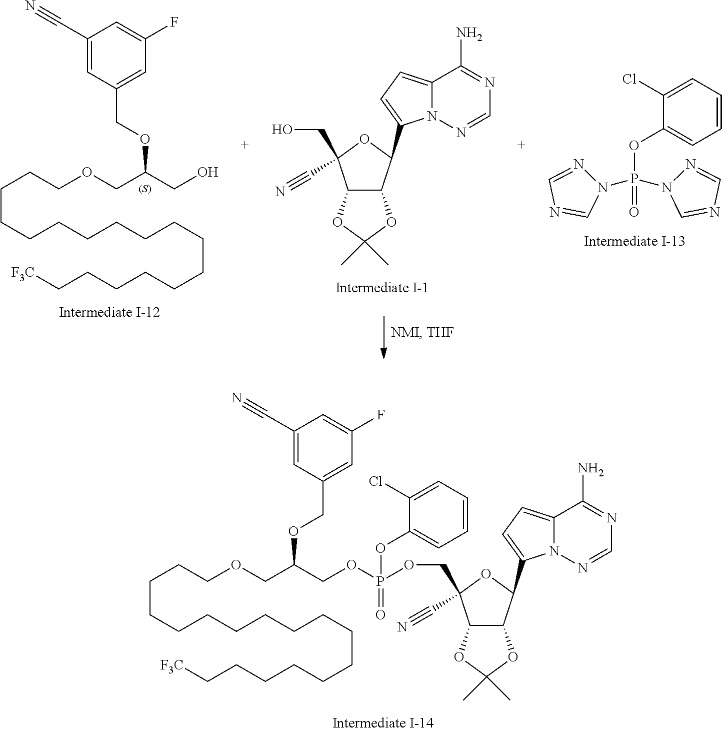

To the solution of Intermediate I-13 (0.246 M in THF, 3.59 mL, 0.883 mmol) were added Intermediate I-1 (225 mg, 0.679 mmol) in one portion and then 1-methylimidazole (0.070 mL, 0.883 mmol). The reaction mixture was stirred at rt for 20 min and then Intermediate I-12 (397 mg, 0.746 mmol) in THF (1 mL) was added dropwise. The resulting mixture was stirred for 1h, diluted with EtOAc (50 mL)-water (40 mL). The resulting mixture was stirred for 10 min, and layers separated, and the aqueous layer extracted with EtOAc (20 mL×3). The combined organic layer was dried with sodium sulfate, concentrated in vacuo, and purified by silica gel (0 to 5% MeOH in DCM) to give Intermediate I-14. ¹H NMR (400 MHZ, Acetonitrile-d3) δ 7.87 (s, 1H), 7.53-7.36 (m, 5H), 7.24-7.08 (m, 2H), 6.81-6.66 (m, 2H), 6.26 (s, 2H), 5.69-5.59 (m, 1H), 5.36-5.18 (m, 1H), 5.17-5.00 (m, 1H), 4.69-4.41 (m, 4H), 4.39-4.13 (m, 2H), 3.83-3.67 (m, 1H), 3.51-3.39 (m, 2H), 3.42-3.30 (m, 2H), 2.31-2.07 (m, 2H), 1.72 (s, 3H), 1.61-1.44 (m, 4H), 1.41-1.00 (m, 29H). ¹⁹F NMR (376 MHz, Acetonitrile-d3) δ −67.51, −112.80. ³¹P NMR (162 MHz, Acetonitrile-d3) δ−7.32, −7.37. MS m/z [M+1]=1035.7.

### Intermediate I-15: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((R)-2-((3-cyano-5-fluorobenzyl)oxy)-3-((18,18,18-trifluorooctadecyl)oxy)propyl) hydrogen phosphate

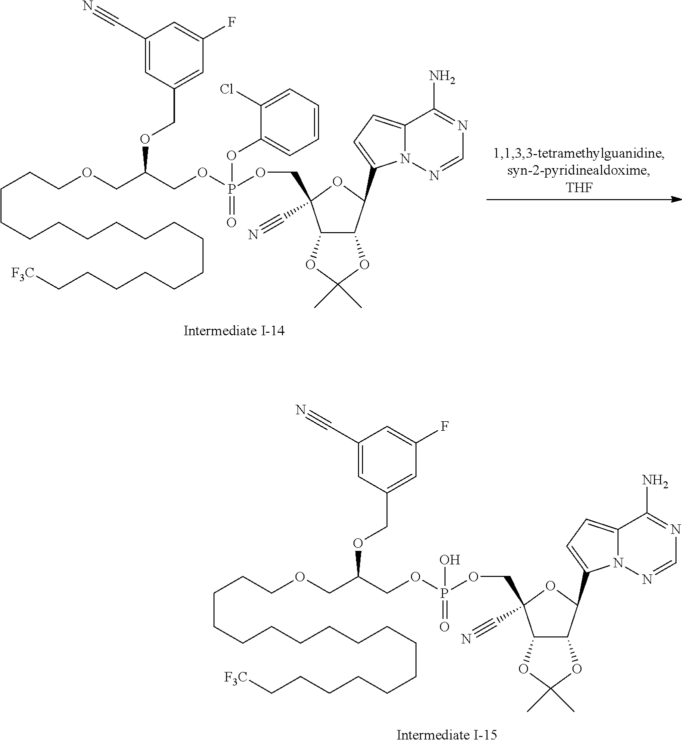 [SMILES: CC1(C)O[C@H]2[C@H](c3ccc4c(N)ncnn34)O[C@](C#N)(COP(=O)(OC[C@@H](COCCCCCCCCCCCCCCCCCC(F)(F)F)OCc3cc(F)cc(C#N)c3)Oc3ccccc3Cl)[C@H]2O1 ; "1,1,3,3-tetramethylguanidine,syn-2-pyridinealdoxime,THF" ; CC1(C)O[C@H]2[C@H](c3ccc4c(N)ncnn34)O[C@](C#N)(COP(=O)(O)OC[C@@H](COCCCCCCCCCCCCCCCCCC(F)(F)F)OCc3cc(F)cc(C#N)c3)[C@H]2O1]

To a solution of Intermediate I-14 (373 mg, 0.36 mmol) in THF (4 mL) were added 1,1,3,3-tetramethylguanidine (0.271 mL, 2.16 mmol) and syn-2-pyridinealdoxime (176 mg, 1.44 mmol). The reaction mixture was stirred at room temperature for 15 h, diluted with EtOAc (100 mL), washed with sat. aqueous ammonium chloride (25 mL×3), dried with sodium sulfate, concentrated in vacuo, and purified by silica gel (0-40% MeOH in DCM) to give Intermediate I-15. ¹H NMR (400 MHZ, Methanol-d4) δ 7.85 (s, 1H), 7.54 (s, 1H), 7.49-4.41 (m, 1H), 7.39-7.31 (m, 1H), 6.85 (d, J=4.5 Hz, 1H), 6.81 (d, J=4.5 Hz, 1H), 5.65 (d, J=3.6 Hz, 1H), 5.28 (dd, J=6.6, 3.6 Hz, 1H), 5.15 (d, J=6.6 Hz, 1H), 4.74 (d, J=13.3 Hz, 1H), 4.65 (d, J=13.3 Hz, 1H), 4.19-4.05 (m, 2H), 4.00-3.86 (m, 2H), 3.79-3.66 (m, 1H), 3.58-3.35 (m, 4H), 2.26-1.92 (m, 2H), 1.71 (s, 3H), 1.61-1.47 (m, 4H), 1.45-1.05 (m, 29H). ¹⁹F NMR (376 MHZ, Methanol-d4) δ −65.87-−72.34, −112.99. ³¹P NMR (162 MHZ, Methanol-d4) δ −0.46. MS m/z [M+1]=925.4.

### Intermediate I-16: 18-bromo-1,1,1-trifluorooctadecane

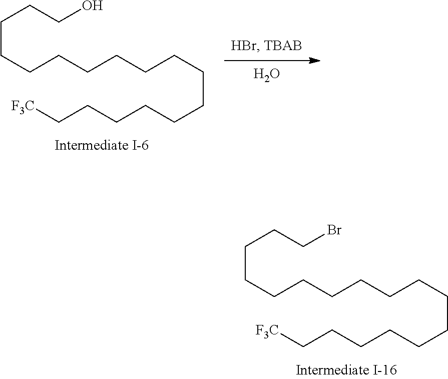

To a solution of TBAB (139.1 mg, 431.5 umol, 0.04 eq) in HBr (18 mL, H₂O) was added Intermediate I-6, 18,18,18-trifluorooctadecan-1-ol (3.5 g, 10.8 mmol, 1 eq) at 20° C. Then the mixture was stirred at 100° C. for 12 hr and concentrated under reduced pressure to remove solvent. The residue was diluted with H₂O (30 mL) and extracted with DCM (20 mL×3). The combined organic layer was washed with H₂O (30 mL×2), dried over Na₂SO₄, filtered, and concentrated under reduced pressure to give a residue. The residue was purified by flash silica gel chromatography (ISCO®; 20 g SepaFlash® Silica Flash Column, Eluent of 1% Ethyl acetate/Petroleum ether gradient @ 45 mL/min) to give compound 18-bromo-1,1,1-trifluorooctadecane, Intermediate I-16. ¹H NMR (400 MHZ, CHLOROFORM-d) δ 3.42 (t, J=6.9 Hz, 2H), 2.12-2.01 (m, 2H), 1.88-1.84 (m, 2H), 1.60-1.52 (m, 2H), 1.53-1.22 (m, 26H).

### Intermediate I-17: (18,18,18-trifluorooctadecyl)magnesium bromide

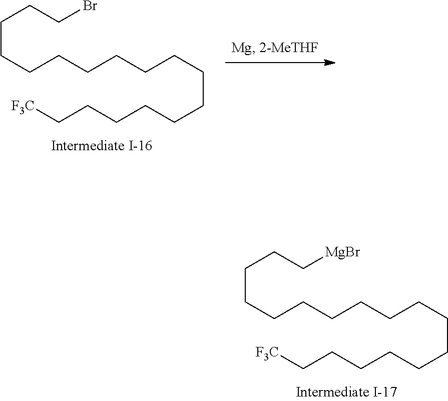

To a solution of Mg (216.5 mg, 8.9 mmol, 1.2 eq) in 2-Me-THF (5 ml) were added I₂ (19.7 mg, 77.5 umol, 15.6 uL, 0.01 eq) and BrCH₂CH₂Br (0.05 mL) under N₂. Then Intermediate I-16, 18-bromo-1,1,1-trifluoro-octadecane (0.3 g, 0.8 mmol) in 2-Me-THF (3 ml) was added dropwise. The mixture was stirred until the color of I₂ was faded to colorless. Then the remaining 18-bromo-1,1,1-trifluoro-octadecane (2.7 g, 7.0 mmol) in 2-Me-THF (27 ml) was added and the mixture was stirred at 25° C. for 4 hr. The reaction mixture was used in the next step without further purification as brown solution in 2-Me-THF.

### Intermediate I-18: (R)-21,21,21-trifluoro-1-(trityloxy)henicosan-2-ol

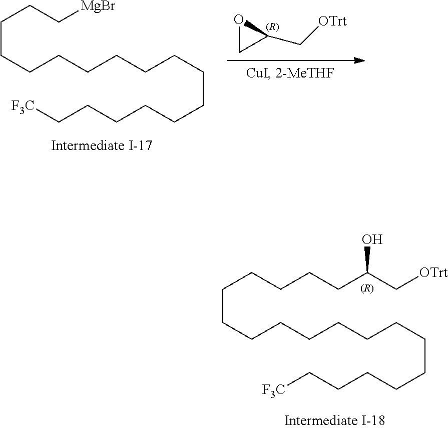

To a mixture of (2R)-2-(trityloxymethyl)oxirane (1.8 g, 5.6 mmol, 1 eq) and CuI (53.4 mg, 280.3 umol, 0.05 eq) in 2-Me-THF (10 mL) was added Intermediate I-18, (18,18,18-trifluorooctadecyl)magnesium bromide (3 g, 7.3 mmol, 1.3 eq) over 10 min via cannula at −20° C. The reaction was stirred vigorously for 5 min and then stirred at 0° C. for 2 h. The reaction was quenched by addition of sat. NH₄Cl solution (50 ml) and then the mixture was extracted with ethyl acetate (35 mL×3). The combined organic layer was washed with H₂O (50 mL×2), dried over Na₂SO₄, filtered, and concentrated under reduced pressure to give a residue. The residue was purified by flash silica gel chromatography (ISCO®; 20 g SepaFlash® Silica Flash Column, Eluent of 0˜4% MTBE/Petroleum ether gradient @ 40 mL/min) to give compound (R)-21,21,21-trifluoro-1-(trityloxy)henicosan-2-ol, Intermediate I-18. ¹H NMR (400 MHZ, CHLOROFORM-d) δ 7.47-7.43 (m, 4H), 7.33-7.29 (m, 11H), 3.80-3.70 (m, 1H), 3.19 (dd, J=3.3, 9.4 Hz, 1H), 3.04 (dd, J=7.7, 9.3 Hz, 1H), 2.30 (d, J=3.4 Hz, 1H), 2.12-2.05 (m, 2H), 1.59-1.53 (m, 2H), 1.48-1.19 (m, 32H).

### Intermediate I-19: (R)-3-fluoro-5-(((21,21,21-trifluoro-1-(trityloxy)henicosan-2-yl)oxy)methyl)benzonitrile

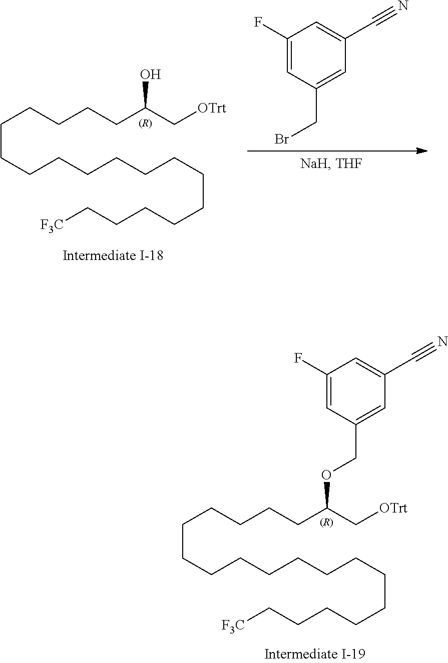

To a solution of NaH (144.0 mg, 3.6 mmol, 60% purity, 2.5 eq) in THF (14 mL) was added (2R)-21,21,21-trifluoro-1-trityloxy-henicosan-2-ol, Intermediate I-18 (900 mg, 1.4 mmol, 1 eq) at 0° C. and the mixture was stirred at 0° C. for 30 min. Then 3-(bromomethyl)-5-fluoro-benzonitrile (369.9 mg, 1.7 mmol, 1.2 eq) was added and the mixture was stirred at 65° C. for 12 hr. The reaction was quenched by addition of sat. NH₄Cl solution (20 ml) at 20° C. and the mixture extracted with ethyl acetate (12 mL×3). The combined organic layer was washed with H₂O (30 mL×2), dried over Na₂SO₄, and concentrated under reduced pressure to give a residue. The residue was purified by flash silica gel chromatography (ISCO®; 12 g SepaFlash® Silica Flash Column, Eluent of 0˜3% MTBE/Petroleum ether gradient @45 mL/min) to give compound (R)-3-fluoro-5-(((21,21,21-trifluoro-1-(trityloxy)henicosan-2-yl)oxy)methyl) benzonitrile, Intermediate I—19. ¹H NMR (400 MHZ, CHLOROFORM-d) δ 7.48-7.41 (m, 7H), 7.31-7.20 (m, 11H), 4.73 (d, J=13.0 Hz, 1H), 4.56 (d, J=12.8 Hz, 1H), 3.57-3.47 (m, 1H), 3.21 (d, J=4.5 Hz, 2H), 2.13-2.00 (m, 2H), 1.59-1.50 (m, 4H), 1.52-1.23 (m, 30H).

### Intermediate I-20: (R)-3-fluoro-5-(((21,21,21-trifluoro-1-hydroxyhenicosan-2-yl)oxy)methyl)benzonitrile

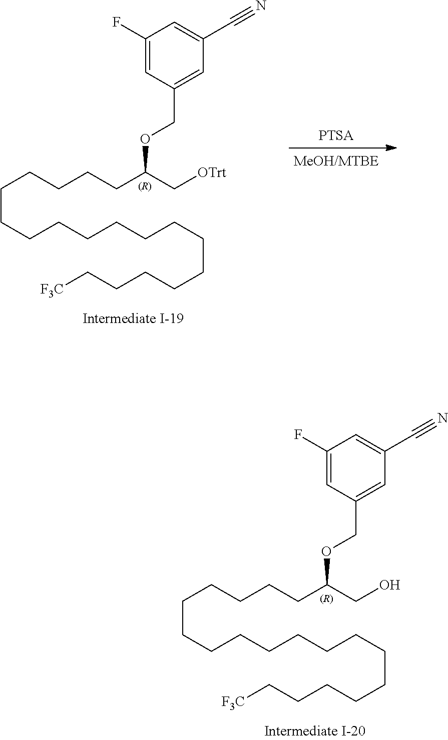

To a solution of 3-fluoro-5-[[(1R)-20,20,20-trifluoro-1-(trityloxymethyl)icosoxy]methyl] benzonitrile, Intermediate I-19 (750 mg, 989.5 umol, 1 eq) in MeOH (2.2 mL) and MTBE (15 mL) were added anisole (53.5 mg, 494.7 umol, 53.8 uL, 0.5 eq) and PTSA (85.2 mg, 494.7 umol, 0.5 eq). The mixture was stirred at 50° C. for 2 hr, diluted with sat. NaHCO₃ (30 mL) and extracted with Ethyl acetate (15 mL×3). The combined organic layer was washed with H₂O (30 mL×2), dried over Na₂SO₄, filtered, and concentrated under reduced pressure to give a residue. The residue was purified by flash silica gel chromatography (ISCO®; 12 g SepaFlash® Silica Flash Column, Eluent of 0˜9% Ethyl acetate/Petroleum ether gradient @ 45 mL/min) to give compound Intermediate I-20. ¹H NMR (400 MHZ, CHLOROFORM-d) δ 7.46 (s, 1H), 7.35-7.20 (m, 2H), 4.64 (s, 2H), 3.79-3.72 (m, 1H), 3.65-3.58 (m, 1H), 3.57-3.50 (m, 1H), 2.13-2.00 (m, 2H), 1.66-1.50 (m, 4H), 1.51-1.25 (m, 30H). MS (ESI): m/z=538.2 [M+Na]⁺.

### Intermediate I-21: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) ((R)-2-((3-cyano-5-fluorobenzyl)oxy)-21,21,21-trifluorohenicosyl) phosphate

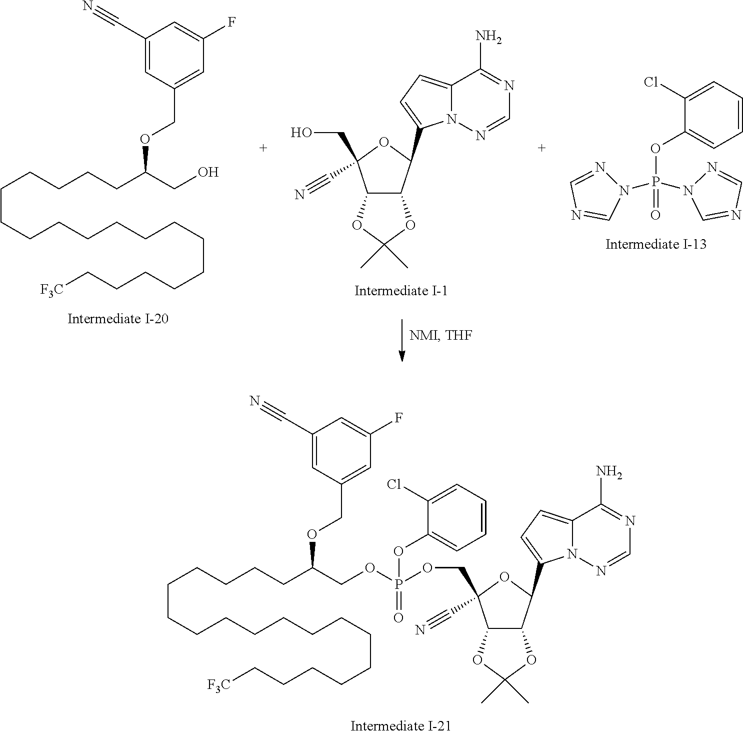 [SMILES: N#Cc1cc(F)cc(CO[C@@H](CO)CCCCCCCCCCCCCCCCCCC(F)(F)F)c1 ; CC1(C)O[C@H]2[C@H](c3ccc4c(N)ncnn34)O[C@](C#N)(CO)[C@H]2O1 ; O=P(Oc1ccccc1Cl)(n1cncn1)n1cncn1 ; CC1(C)O[C@H]2[C@H](c3ccc4c(N)ncnn34)O[C@](C#N)(COP(=O)(OC[C@@H](CCCCCCCCCCCCCCCCCCC(F)(F)F)OCc3cc(F)cc(C#N)c3)Oc3ccccc3Cl)[C@H]2O1]

To the solution of Intermediate I-13 (0.246 M in THF, 3.51 mL, 0.863 mmol) were added Intermediate I-1 (220 mg, 0.664 mmol) in one portion and then 1-methylimidazole (0.069 mL, 0.863 mmol). The reaction mixture was stirred at rt for 10 min and then Intermediate I-20 (376 mg, 0.729 mmol) in THF (1 mL) was added dropwise. The resulting mixture was stirred for 1 h, diluted with EtOAc (50 mL)-water (20 mL)-brine (20 mL), stirred for 10 min, layers separated, the aqueous layer extracted with EtOAc (20 mL×3). The combined organic layer was dried under sodium sulfate, concentrated in vacuo, and purified by silica gel (0 to 5% MeOH in DCM) to give Intermediate I-21. ¹H NMR (400 MHZ, Acetonitrile-d3) δ 7.87 (s, 1H), 7.53-7.21 (m, 5H), 7.26-7.06 (m, 2H), 6.81-6.66 (m, 2H), 6.29 (s, 2H), 5.67 (dd, J=4.9, 3.1 Hz, 1H), 5.33-5.22 (m, 1H), 5.17-4.99 (m, 1H), 4.65-4.39 (m, 4H), 4.34-3.21 (m, 1H), 4.20-4.03 (m, 1H), 3.64-3.50 (m, 1H), 2.27-2.02 (m, 2H), 1.72 (s, 3H), 1.60-1.41 (m, 2H), 1.41-1.11 (m, 35H). ¹⁹F NMR (376 MHz, Acetonitrile-d3) δ−67.52, −112.84. ³¹P NMR (162 MHz, Acetonitrile-d3) δ−7.30, −7.33. MS m/z [M+1]=1019.5.

### Intermediate I-22: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((R)-2-((3-cyano-5-fluorobenzyl)oxy)-21,21,21-trifluorohenicosyl) hydrogen phosphate

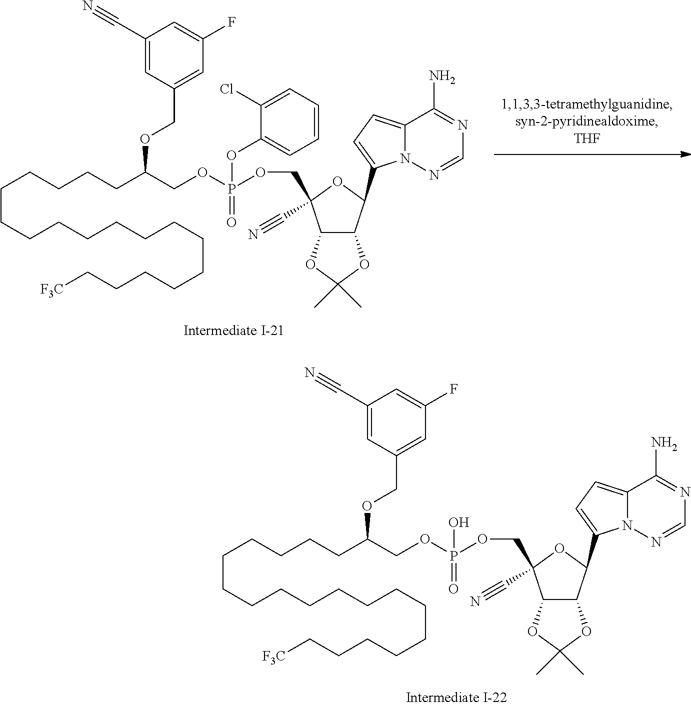 [SMILES: CC1(C)O[C@H]2[C@H](c3ccc4c(N)ncnn34)O[C@](C#N)(COP(=O)(OC[C@@H](CCCCCCCCCCCCCCCCCCC(F)(F)F)OCc3cc(F)cc(C#N)c3)Oc3ccccc3Cl)[C@H]2O1 ; CC1(C)O[C@H]2[C@H](c3ccc4c(N)ncnn34)O[C@](C#N)(COP(=O)(O)OC[C@@H](CCCCCCCCCCCCCCCCCCC(F)(F)F)OCc3cc(F)cc(C#N)c3)[C@H]2O1]

To a solution of Intermediate I-21 (387 mg, 0.38 mmol) in THF (4 mL) were added 1,1,3,3-tetramethylguanidine (0.286 mL, 2.28 mmol) and syn-2-pyridinealdoxime (188 mg, 1.54 mmol). The reaction mixture was stirred at room temperature for 15 h, diluted with EtOAc (100 mL), washed with sat. aqueous ammonium chloride (25 mL×3), dried with sodium sulfate, concentrated in vacuo, and purified by silica gel (0-60% MeOH in DCM) to give Intermediate I-22. ¹H NMR (400 MHZ, Methanol-d4) δ 7.84 (s, 1H), 7.51 (s, 1H), 7.44-7.34 (m, 2H), 6.85 (d, J=4.5 Hz, 1H), 6.80 (d, J=4.5 Hz, 1H), 5.65 (d, J=3.5 Hz, 1H), 5.28 (dd, J=6.6, 3.6 Hz, 1H), 5.15 (d, J=6.6 Hz, 1H), 4.75 (d, J=13.0 Hz, 1H), 4.53 (d, J=13.1 Hz, 1H), 4.20-4.08 (m, 2H), 3.97-3.84 (m, 2H), 3.63-3.51 (m, 1H), 2.21-2.04 (m, 2H), 1.71 (s, 3H), 1.61-1.48 (m, 2H), 1.49-1.20 (m, 35H). ¹⁹F NMR (376 MHz, Methanol-d4) δ −68.50, −112.92. ¹P NMR (162 MHZ, Methanol-d4) δ −0.36. MS m/z [M+1]=909.3.

### Intermediate I-23: methyl 16-hydroxyhexadecanoate

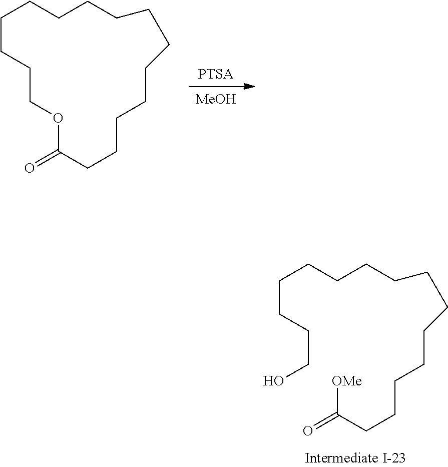

To a solution of oxacycloheptadecan-2-one (10 g, 39.3 mmol, 1 eq) in MeOH (100 mL) was added PTSA (1.0 g, 5.9 mmol, 0.15 eq). The mixture was stirred at 70° C. for 12 hr and concentrated under reduced pressure. The resulting residue was diluted with H₂O (300 mL) and extracted with ethyl acetate (200 mL×3). The combined organic layer was washed with H₂O (300 mL×2), dried over Na₂SO₄, filtered, and concentrated under reduced pressure to give a residue. The residue was purified by flash silica gel chromatography (Silica Flash Column, Eluent of 0˜15% Ethyl acetate/Petroleum ether gradient @ 100 mL/min) to give compound methyl 16-hydroxyhexadecanoate, Intermediate I-23. ¹H NMR (400 MHZ, CHLOROFORM-d) δ 3.72-3.61 (m, 5H), 2.30 (t, J=7.5 Hz, 2H), 1.64-1.54 (m, 4H), 1.35-1.24 (m, 22H).

### Intermediate I-24: methyl 16-iodohexadecanoate

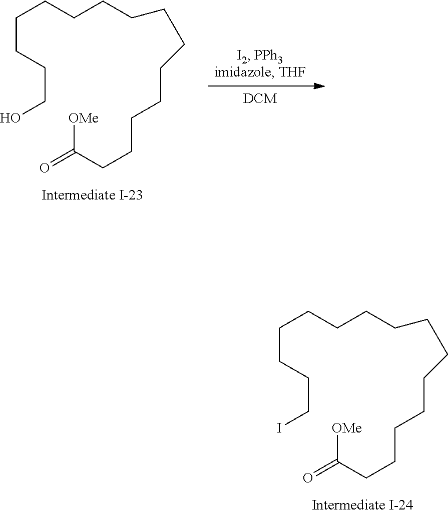

To a solution of methyl 16-hydroxyhexadecanoate, Intermediate I-23 (5 g, 17.5 mmol, 1 eq) in DCM (100 mL) were added imidazole (3.6 g, 52.4 mmol, 3 eq) and PPh₃ (9.2 g, 34.9 mmol, 2 eq). Then the mixture was degassed, purged with N₂ for 3 times, then the temperature was cooled to 0° C., and I₂ (8.9 g, 34.9 mmol, 7.0 mL, 2 eq) in THF (30 mL) added. Then the mixture was stirred at 25° C. for 12 hr, the reaction was quenched by addition 10% Na₂S₂O₃ 300 mL, and then the mixture extracted with DCM (200 mL×3). The combined organic layer was washed with H₂O (300 mL×2), dried over Na₂SO₄, filtered, and concentrated under reduced pressure to give a residue. The residue was purified by flash silica gel chromatography (Silica Flash Column, Eluent of 0˜3% Ethyl acetate/Petroleum ether gradient @ 100 mL/min) to give compound methyl 16-iodohexadecanoate, Intermediate I-24. ¹H NMR (400 MHZ, CHLOROFORM-d) δ 3.66 (d, J=1.9 Hz, 3H), 3.18 (dt, J=1.8, 7.1 Hz, 2H), 2.36-2.27 (m, 2H), 1.86-1.78 (m, 2H), 1.67-1.57 (m, 2H), 1.50-1.20 (m, 22H).

### Intermediate I-25: 16-iodohexadecan-1-ol

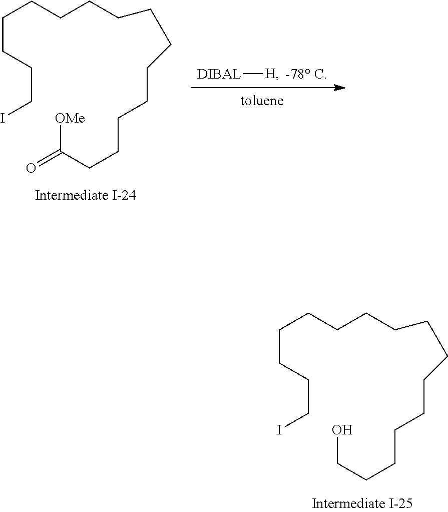

To a solution of methyl 16-iodohexadecanoate, Intermediate I-24 (5 g, 12.6 mmol, 1 eq) in toluene (50 mL) was added dropwise DIBAL-H (1 M, 31.5 mL, 2.5 eq) at −78° C. After addition, the mixture was stirred 0° C. for 2 hr. The reaction was quenched by pouring into sat. NH₄Cl solution (300 ml) at 0° C. The mixture was stirred for 30 min and extracted with ethyl acetate (200 mL×3). The combined organic layer was washed with H₂O (300 mL×2), dried over Na₂SO₄, filtered, and concentrated under reduced pressure to give a residue. The residue was purified by flash silica gel chromatography (Silica Flash Column, Eluent of 0˜15% Ethyl acetate/Petroleum ether gradient @ 100 mL/min) to give compound 16-iodohexadecan-1-ol, Intermediate I-25. ¹H NMR (400 MHZ, CHLOROFORM-d) δ 3.65 (t, J=6.6 Hz, 2H), 3.25-3.15 (m, 2H), 1.88-1.77 (m, 2H), 1.63-1.52 (m, 2H), 1.41-1.36 (m, 2H), 1.37-1.20 (m, 22H).

### Intermediate I-26: 2-((16-iodohexadecyl)oxy)tetrahydro-2H-pyran

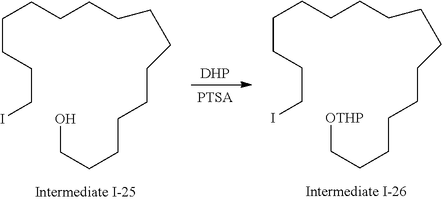

To a solution of 16-iodohexadecan-1-ol, Intermediate I-25 (10 g, 27.1 mmol, 1 eq) in THF (100 mL) were added 3,4-dihydro-2H-pyran (6.8 g, 81.4 mmol, 7.4 mL, 3 eq) and PTSA (701.3 mg, 4.1 mmol, 0.15 eq). The resulting mixture was stirred at 20° C. for 12 hr and concentrated under reduced pressure. The residue was diluted with H₂O 150 mL and extracted with ethyl acetate (100 mL×3). The combined organic layer was washed with H₂O (200 ml×2), dried over Na₂SO₄, filtered, and concentrated under reduced pressure to give a residue. The residue was purified by flash silica gel chromatography (Silica Flash Column, Eluent of 0˜5% Ethyl acetate/Petroleum ether gradient @ 100 mL/min) to give compound 2-((16-iodohexadecyl)oxy)tetrahydro-2H-pyran, Intermediate I-26. ¹H NMR (400 MHZ, CHLOROFORM-d) δ 4.61-4.56 (m, 1H), 3.95-3.87 (m, 1H), 3.80-3.70 (m, 1H), 3.54-3.47 (m, 1H), 3.41-3.40 (m, 1H), 3.19 (t, J=7.1 Hz, 2H), 1.85-1.81 (m, 2H), 1.63-1.51 (m, 6H), 1.42-1.25 (m, 26H).

### Intermediate I-27: 2-((17,17,17-trifluoroheptadecyl)oxy)tetrahydro-2H-pyran

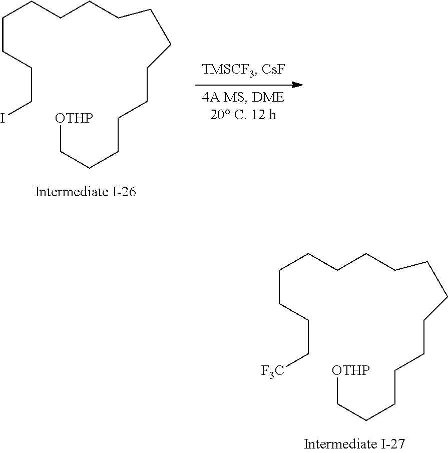

To a solution of CsF (6.4 g, 42.4 mmol, 1.6 mL, 3.2 eq) in 15-CROWN-5 (15.9 g, 72.3 mmol, 14.3 mL, 5.4 eq) were added DME (120 mL) and 4A MS (15 g) and then the mixture cooled to −20° C. Then a solution of 2-(16-iodohexadecoxy)tetrahydropyran, Intermediate I-26 (6 g, 13.3 mmol, 1 eq) and TMS-CF₃ (8.0 g, 56.2 mmol, 4.2 eq) in DME (50 mL) was added into the above solution over 30 min. The mixture was stirred at 20° C. for 12 hr and concentrated under reduced pressure. The residue was diluted ethyl acetate (100 mL), washed with NaCl (50 mL×3), dried over Na₂SO₄, filtered, and concentrated under reduced pressure to give a residue. The residue was purified by column chromatography (SiO₂, Petroleum ether/MTBE=100/0 to 100/3) to give compound 2-((17,17,17-trifluoroheptadecyl)oxy)tetrahydro-2H-pyran, Intermediate I-27.

### Intermediate I-28: 17,17,17-trifluoroheptadecan-1-ol

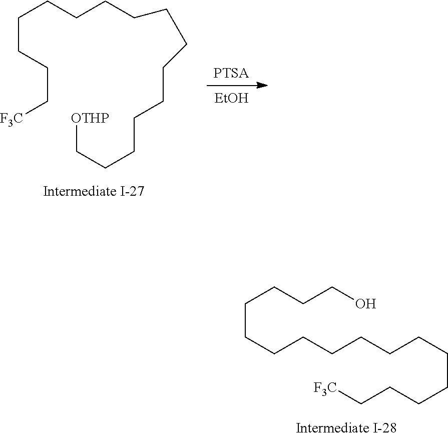

To a solution of 2-(17,17,17-trifluoroheptadecoxy)tetrahydropyran, Intermediate I-27 (5 g, 12.7 mmol, 1 eq) in EtOH (50 mL) was added PTSA (2.2 g, 12.7 mmol, 1 eq). The mixture was stirred at 50° C. for 12 hr and concentrated under reduced pressure. The residue was diluted with H₂O (300 mL) and extracted with ethyl acetate (200 mL×3). The combined organic layer was washed with H₂O (300 mL×2), dried over Na₂SO₄, filtered, and concentrated under reduced pressure to give a residue. The residue was purified by flash silica gel chromatography (Silica Flash Column, Eluent of 0˜15% Ethyl acetate/Petroleum ether gradient @ 120 mL/min) to give compound 17,17,17-trifluoroheptadecan-1-ol, Intermediate I-28. ¹H NMR (400 MHZ, CHLOROFORM-d) δ 3.65 (t, J=6.6 Hz, 2H), 2.13-1.99 (m, 2H), 1.62-1.51 (m, 4H), 1.36-1.26 (m, 24H).

### Intermediate I-29: 17-bromo-1,1,1-trifluoroheptadecane

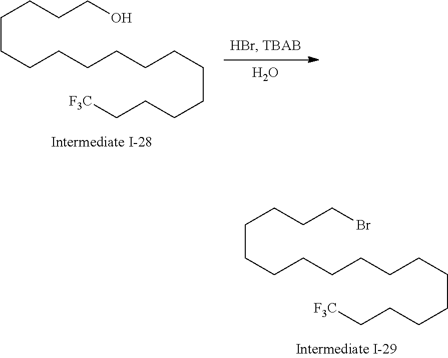

To a solution of 17,17,17-trifluoroheptadecan-1-ol, Intermediate I-28 (5 g, 16.1 mmol, 1 eq) in HBr (40 mL, 30% in H₂O) was added TBAB (207.7 mg, 644.2 umol, 0.04 eq) at 20° C. Then the mixture was stirred at 100° C. for 12 hr and concentrated under reduced pressure. The residue was diluted with H₂O (150 mL) and extracted with DCM (100 mL×3). The combined organic layer was washed with H₂O (300 mL×2), dried over Na₂SO₄, filtered, and concentrated under reduced pressure to give a residue. The residue was purified by flash silica gel chromatography (Silica Flash Column, Eluent of 100% Petroleum ether gradient @ 100 mL/min) to give compound 17-bromo-1,1,1-trifluoroheptadecane, Intermediate I-29. ¹H NMR (400 MHz, CHLOROFORM-d) δ 3.42 (t, J=6.9 Hz, 2H), 2.13-2.00 (m, 2H), 1.86 (td, J=7.1, 14.5 Hz, 2H), 1.60-1.51 (m, 2H), 1.47-1.40 (m, 2H), 1.40-1.25 (m, 22H)

### Intermediate I-30: (17,17,17-trifluoroheptadecyl)magnesium bromide

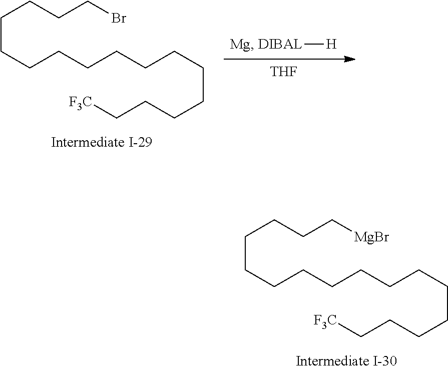

A mixture of Mg (359.4 mg, 14.8 mmol, 1.2 eq) and THF (5 ml) was stirred at 20° C. under N₂. To a above mixture were added 17-bromo-1,1,1-trifluoro-heptadecane, Intermediate I-29 (0.3 g, 0.7 mmol) in THF (0.5 ml) dropwise and then DIBAL-H (1 M, 128.6 uL, 0.01 eq) at 20° C. The remaining 17-bromo-1,1,1-trifluoro-heptadecane (4.5 g, 12.1 mmol) in THF (10 ml) was added and the mixture was stirred at 20° C. for 4 hr. The crude product (17,17,17-trifluoroheptadecyl)magnesium bromide, Intermediate I-30 as brown liquid was used into the next step without further purification.

### Intermediate I-31: (S)-20,20,20-trifluoro-1-(trityloxy)icosan-2-ol

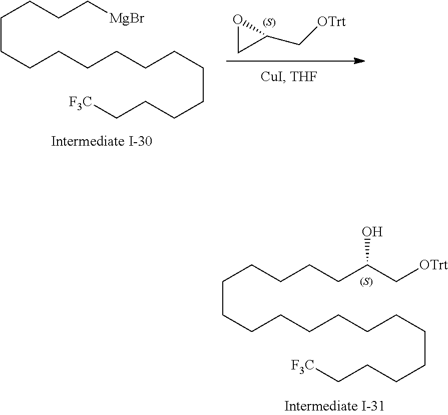

Bromo(17,17,17-trifluoroheptadecyl)magnesium, Intermediate I-30 (4.8 g, 12.1 mmol, 1.3 eq) was added via cannula over 10 min to mixture of (2S)-2-(trityloxymethyl)oxirane (2.9 g, 9.3 mmol, 1.0 eq) and CuI (88.4 mg, 464.3 umol, 0.05 eq) in THF (25 mL) at −20° C. The mixture was stirred vigorously at 20° C. for 5 min, warmed to 0° C., and continuously stirred for 2 h. The reaction was quenched by addition sat. NH₄Cl solution (80 ml), and then the mixture was extracted with ethyl acetate (50 mL×3). The combined organic layers were washed with H₂O (100 mL×2), dried over Na₂SO₄, filtered, and concentrated under reduced pressure to give a residue. The residue was purified by flash silica gel chromatography (Silica Flash Column, Eluent of 0˜3% Ethyl acetate/Petroleum ether gradient @ 45 mL/min) to give compound (S)-20,20,20-trifluoro-1-(trityloxy)icosan-2-ol, Intermediate I-31. ¹H NMR (400 MHZ, CHLOROFORM-d) δ 7.49-7.40 (m, 6H), 7.34-7.24 (m, 9H), 3.85-3.70 (m, 1H), 3.19 (dd, J=3.3, 9.4 Hz, 1H), 3.03 (dd, J=7.6, 9.4 Hz, 1H), 2.33-2.26 (m, 1H), 2.13-2.01 (m, 2H), 1.60-1.40 (m, 2H), 1.41-1.22 (m, 30H).

### Intermediate I-32: (S)-3-fluoro-5-(((20,20,20-trifluoro-1-(trityloxy)icosan-2-yl)oxy)methyl)benzonitrile

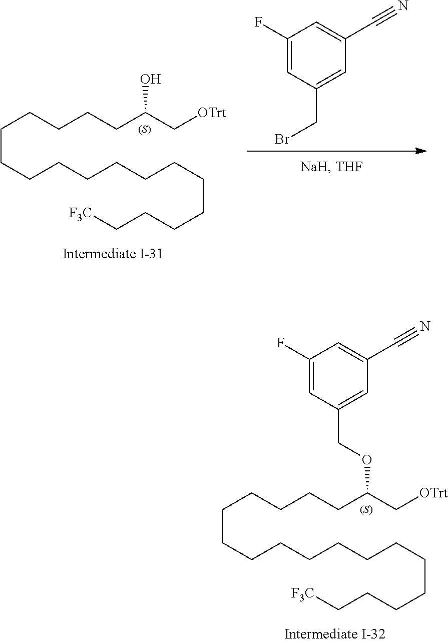

To a solution of (2S)-20,20,20-trifluoro-1-trityloxy-icosan-2-ol, Intermediate I-31 (3.0 g, 4.9 mmol, 1.0 eq) in THF (45 mL) was added NaH (491.1 mg, 12.3 mmol, 60% purity, 2.5 eq) at 0° C. and the mixture was stirred at 0° C. for 30 min. Then 3-(bromomethyl)-5-fluoro-benzonitrile (1.3 g, 6.1 mmol, 1.3 eq) was added and the mixture was stirred at 65° C. for 12 hr. The reaction was quenched by addition sat. NH₄Cl solution (60 ml) and the mixture extracted with ethyl acetate (30 mL×3). The combined organic layer was washed with H₂O (60 mL×2), dried over Na₂SO₄, and concentrated under reduced pressure to give a residue. The residue was purified by flash silica gel chromatography (Silica Flash Column, Eluent of 0˜3% Ethyl acetate/Petroleum ether gradient @ 45 mL/min) to give compound (S)-3-fluoro-5-(((20,20,20-trifluoro-1-(trityloxy)icosan-2-yl)oxy)methyl)benzonitrile, Intermediate I-32. ¹H NMR (400 MHZ, CHLOROFORM-d) § 7.53-7.44 (m, 7H), 7.38-7.25 (m, 11H), 4.77 (d, J=12.8 Hz, 1H), 4.60 (d, J=12.8 Hz, 1H), 3.62-3.49 (m, 1H), 3.26 (d, J=4.8 Hz, 2H), 2.18-2.05 (m, 2H), 1.64-1.54 (m, 4H), 1.48-1.20 (m, 28H).

### Intermediate I-33: (S)-3-fluoro-5-(((20,20,20-trifluoro-1-hydroxyicosan-2-yl)oxy)methyl)benzonitrile

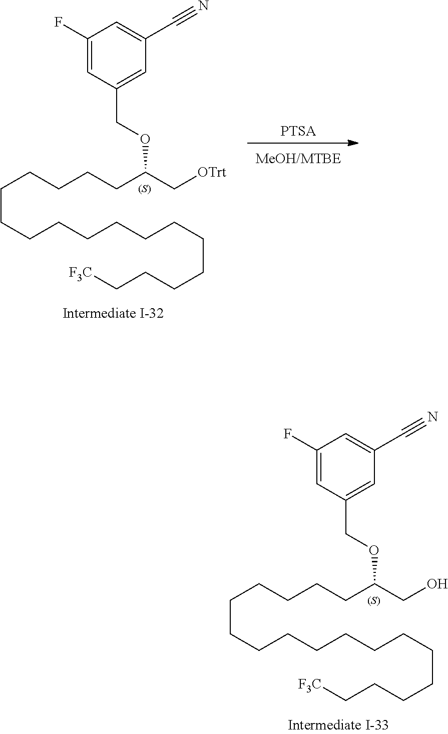

To a solution of 3-fluoro-5-[[(1S)-19,19,19-trifluoro-1-(trityloxymethyl)nonadecoxy]methyl]benzonitrile, Intermediate I-32 (3.0 g, 4.0 mmol, 1.0 eq) in MeOH (9 mL) and MTBE (60 mL) were added anisole (218.0 mg, 2.0 mmol, 219.1 uL, 0.5 eq), and PTSA (347.2 mg, 2.0 mmol, 0.5 eq). The mixture was stirred at 50° C. for 2 hr, diluted with sat. NaHCO₃ (80 mL), and extracted with ethyl acetate (40 mL×3). The combined organic layers were washed with H₂O (50 mL×2), dried over Na₂SO₄, filtered, and concentrated under reduced pressure to give a residue. The residue was purified by flash silica gel chromatography (Silica Flash Column, Eluent of 0˜9% Ethyl acetate/Petroleum ether gradient @ 45 mL/min) to give compound Intermediate I-33. ¹H NMR (400 MHZ, CHLOROFORM-d) δ 7.46 (s, 1H), 7.40-7.22 (m, 2H), 4.64 (s, 2H), 3.75 (dd, J=3.3, 11.5 Hz, 1H), 3.65-3.59 (m, 1H), 3.56-3.50 (m, 1H), 2.13-2.00 (m, 2H), 1.81 (s, 1H), 1.65-1.50 (m, 4H), 1.35-1.25 (m, 28H). 19F NMR (376 MHz, CHLOROFORM-d) δ−109.79, −66.41. MS (ESI): m/z=519.2 [M+H₂O]⁺.

### Intermediate I-34: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) ((S)-2-((3-cyano-5-fluorobenzyl)oxy)-20,20,20-trifluoroicosyl) phosphate

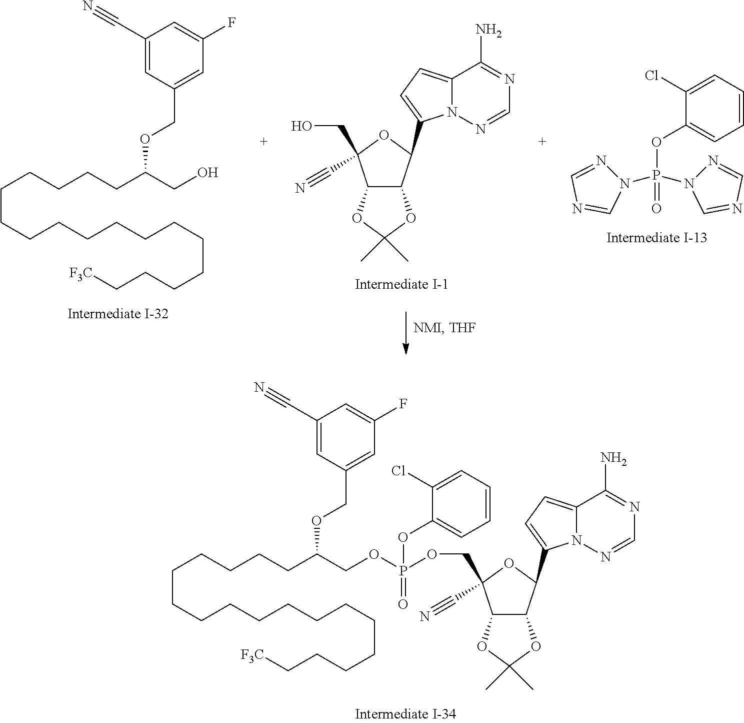 [SMILES: N#Cc1cc(F)cc(CO[C@H](CO)CCCCCCCCCCCCCCCCCC(F)(F)F)c1 ; CC1(C)O[C@H]2[C@H](c3ccc4c(N)ncnn34)O[C@](C#N)(CO)[C@H]2O1 ; O=P(Oc1ccccc1Cl)(n1cncn1)n1cncn1 ; CC1(C)O[C@H]2[C@H](c3ccc4c(N)ncnn34)O[C@](C#N)(COP(=O)(OC[C@H](CCCCCCCCCCCCCCCCCC(F)(F)F)OCc3cc(F)cc(C#N)c3)Oc3ccccc3Cl)[C@H]2O1]

To the stock solution of Intermediate I-13 (0.246 M in THF, 2.95 mL, 0.726 mmol) were added Intermediate I-1 (185 mg, 0.558 mmol) in one portion and then 1-methylimidazole (0.060 mL, 0.726 mmol). The reaction mixture was stirred at rt for 10 min and then Intermediate I-33 (308 mg, 0.613 mmol) in THF (1 mL) added dropwise. The resulting mixture was stirred for 1 h and diluted with EtOAc (50 mL)-water (20 mL)-brine (20 mL). The resulting mixture was stirred for 10 min, layers separated, and the aqueous layer extracted with EtOAc (20 mL×3). The combined organic layer was dried under sodium sulfate, concentrated in vacuo, and purified by silica gel (0 to 5% MeOH in DCM) to give Intermediate I-34. ¹H NMR (400 MHZ, Acetonitrile-d3) δ 7.90-7.70 (m, 1H), 7.53-7.30 (m, 5H), 7.28-7.06 (m, 2H), 6.85-6.61 (m, 2H), 6.26 (s, 2H), 5.71-5.58 (m, 1H), 5.33-5.22 (m, 1H), 5.15-5.01 (m, 1H), 4.64-4.38 (m, 4H), 4.36-4.21 (m, 1H), 4.17-4.03 (m, 1H), 3.66-3.48 (m, 1H), 2.24-2.08 (m, 2H, buried in solvent peak), 1.75-1.60 (m, 3H), 1.61-1.43 (m, 2H), 1.43-1.14 (m, 33H). ¹⁹F NMR (376 MHz, Acetonitrile-d3) δ−67.52, −112.84. ³¹P NMR (162 MHZ, Acetonitrile-d3) δ−7.20, −7.48. MS m/z [M+1]=1005.6.

### Intermediate I-35: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((S)-2-((3-cyano-5-fluorobenzyl)oxy)-20,20,20-trifluoroicosyl) hydrogen phosphate

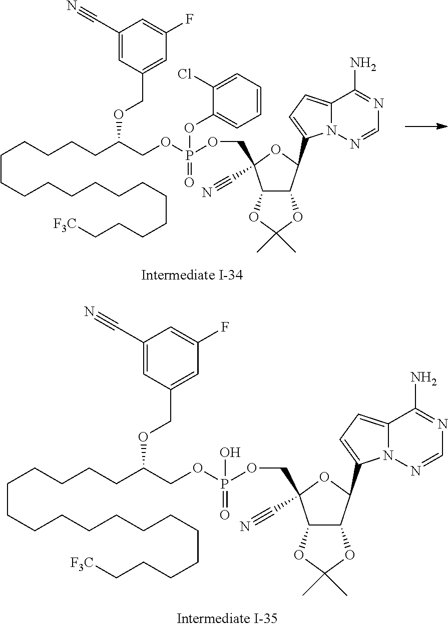 [SMILES: CC1(C)O[C@H]2[C@H](c3ccc4c(N)ncnn34)O[C@](C#N)(COP(=O)(OC[C@H](CCCCCCCCCCCCCCCCCC(F)(F)F)OCc3cc(F)cc(C#N)c3)Oc3ccccc3Cl)[C@H]2O1 ; CC1(C)O[C@H]2[C@H](c3ccc4c(N)ncnn34)O[C@](C#N)(COP(=O)(O)OC[C@H](CCCCCCCCCCCCCCCCCC(F)(F)F)OCc3cc(F)cc(C#N)c3)[C@H]2O1]

To a solution of Intermediate I-34 (387 mg, 0.38 mmol) in THF (4 mL) were added 1,1,3,3-tetramethylguanidine (0.286 mL, 2.28 mmol) and syn-2-pyridinealdoxime (188 mg, 1.54 mmol). The reaction mixture was stirred at room temperature for 15 h, diluted with EtOAc (100 mL), washed with sat. aqueous ammonium chloride (25 mL×3), dried with sodium sulfate, concentrated in vacuo, and purified by silica gel (0-60% MeOH in DCM) to give Intermediate I-35. ¹H NMR (400 MHZ, Methanol-d4) δ 7.87 (s, 1H), 7.51 (s, 1H), 7.45-7.40 (m, 1H), 7.40-7.33 (m, 1H), 6.90 (d, J=4.5 Hz, 1H), 6.84 (d, J=4.6 Hz, 1H), 5.66 (d, J=3.7 Hz, 1H), 5.28 (dd, J=6.6, 3.7 Hz, 1H), 5.15 (d, J=6.6 Hz, 1H), 4.76 (d, J=13.1 Hz, 1H), 4.52 (d, J=12.9 Hz, 1H), 4.13 (d, J=4.3 Hz, 2H), 3.89 (tt, J=11.5, 5.8 Hz, 2H), 3.59 (t, J=5.7 Hz, 1H), 2.30-1.98 (m, 2H), 1.71 (s, 3H), 1.63-1.07 (m, 35H). ¹⁹F NMR (376 MHz, Methanol-d₄) δ −68.57, −113.04. ³¹p NMR (162 MHZ, Methanol-d₄) δ −0.42. MS m/z [M+1]=894.9.

### Intermediate I-37: methyl 16-bromohexadecanoate

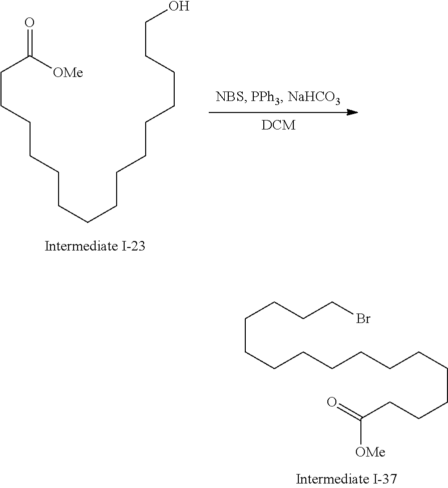

To a solution of methyl 16-hydroxyhexadecanoate, Intermediate I-23 (32.6 g, 114.0 mmol, 1 eq) in CH₂Cl2 (300 mL) was added PPh₃ (37.3 g, 142.5 mmol, 1.2 eq) and NaHCO₃ (816.5 mg, 9.7 mmol, 378.0 uL, 8.52e-2 eq). The solution was cooled to 0° C. and NBS (26.3 g, 148.2 mmol, 1.3 eq) was added to the reaction. The solution was then stirred at 0° C. for 20 min, warmed to 20° C., and stirred for 1 hr. The residue was diluted with Na₂S₂O₃ (500 mL) and extracted with DCM (200 mL×3). The combined organic layers were washed with brine (200 mL×2), dried over Na₂SO₄, filtered, and concentrated under reduced pressure to give a residue. The residue was purified by flash silica gel chromatography (ISCO®; 20 g SepaFlash® Silica Flash Column, Eluent of 0˜10% Ethyl acetate/Petroleum ether gradient @ 120 mL/min) to give methyl 16-bromohexadecanoate, Intermediate I-37. ¹H NMR (400 MHZ, CHLOROFORM-d) δ 3.66 (s, 3H), 3.40 (t, J=6.9 Hz, 2H), 2.30 (t, J=7.5 Hz, 2H), 1.85 (quin, J=7.2 Hz, 2H), 1.65-1.57 (m, 2H), 1.45-1.37 (m, 2H), 1.31-1.23 (m, 20H).

### Intermediate I-38: 16-bromohexadecan-1-ol

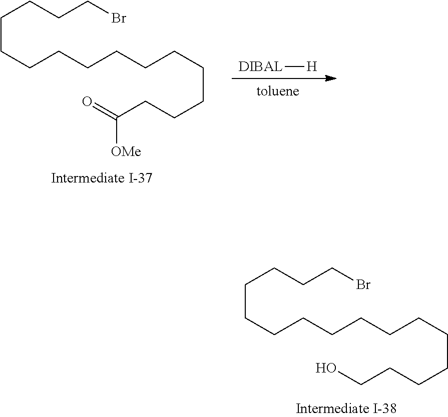

To a solution of methyl 16-bromohexadecanoate, Intermediate I-37 (38.6 g, 110.7 mmol, 1 eq) in Tol. (400 mL), cooled to −78° C., DIBAL-H (1 M, 255.2 mL, 2.3 eq) was added. The reaction was stirred at 0° C., for 1 hr. The residue was diluted with NH₄Cl (500 mL) and extracted with EtOAc (300 mL×3). The combined organic layers were washed with brine (200 mL×2), dried over Na₂SO₄, filtered and concentrated under reduced pressure to give 16-bromohexadecan-1-ol, Intermediate I-38. ¹H NMR (400 MHZ, CHLOROFORM-d) δ 3.62 (t, J=6.7 Hz, 2H), 3.39 (t, J=6.9 Hz, 2H), 1.84 (quin, J=7.2 Hz, 2H), 1.55 (quin, J=6.9 Hz, 2H), 1.46-1.34-1.20-1.37 (m, 2H), (m, 22H).

### Intermediate I-39: 16-bromohexadecanal

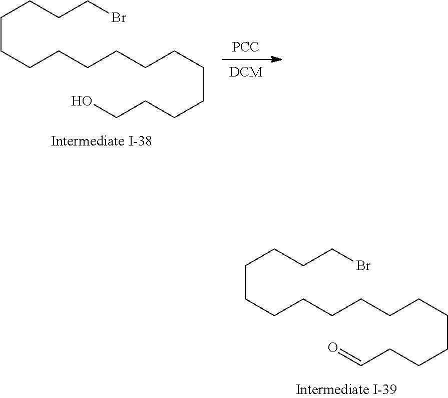

A solution of 16-bromohexadecan-1-ol, Intermediate I-38 (24.0 g, 74.9 mmol, 1 eq) and PCC (32.3 g, 149.8 mmol, 2 eq) in CH₂Cl₂ (200 mL) was stirred at 20° C. for 12 hr. The reaction mixture was filtered and concentrated under reduced pressure to give a residue. The residue was purified by flash silica gel chromatography (ISCO®; 120 g SepaFlash® Silica Flash Column, Eluent of 0˜10% Ethyl acetate/Petroleum ether gradient @ 120 mL/min) to give 16-bromohexadecanal, Intermediate I-39. ¹H NMR (400 MHZ, CHLOROFORM-d) δ 9.76 (t, J=1.8 Hz, 1H), 3.41 (t, J=6.9 Hz, 2H), 2.42 (dt, J=1.8, 7.3 Hz, 2H), 1.85 (td, J=7.1, 14.5 Hz, 2H), 1.63 (quin, J=7.0 Hz, 2H), 1.45-1.39 (m, 2H), 1.31-1.24 (m, 20H).

### Intermediate I-40: 16-bromo-1,1-difluorohexadecane

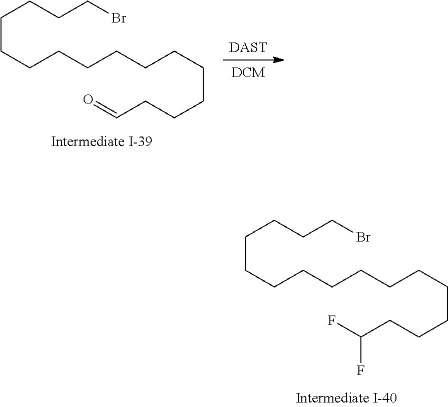

DAST (8.1 g, 50.3 mmol, 6.6 mL, 1.2 eq) in DCM (80 mL) was added to the solution of 16-bromohexadecanal, Intermediate I-39 (13.4 g, 42.0 mmol, 1 eq) in DCM (80 mL), the reaction was stirred at 25° C. for 12 hr. The reaction mixture was concentrated under reduced pressure to remove solvent. The residue was purified by flash silica gel chromatography (ISCO®; 80 g SepaFlash® Silica Flash Column, Eluent of 0˜10% Ethyl acetate/Petroleum ether gradient @ 120 mL/min) to give 16-bromo-1,1-difluorohexadecane, Intermediate I-40. ¹H NMR (400 MHZ, CHLOROFORM-d) δ 5.80 (tt, J=4.6, 57.0 Hz, 1H), 3.42 (t, J=6.8 Hz, 2H), 1.91-1.75 (m, 4H), 1.44 (sxt, J=7.2 Hz, 4H), 1.33-1.22 (m, 20H).

### Intermediate I-41: (16,16-difluorohexadecyl)magnesium bromide

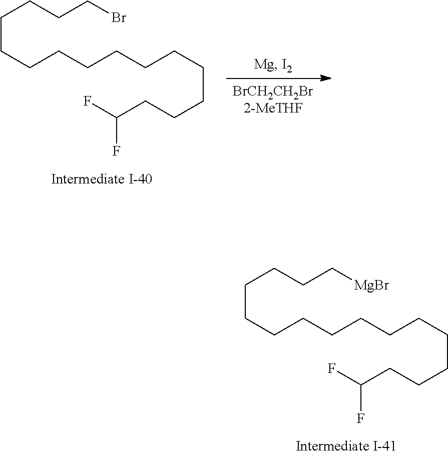

To a mixture of Mg (818.9 mg, 33.6 mmol, 1.1 eq) in 2-Me-THF (2 mL) was added I₂ (74.3 mg, 292.9 umol, 59.0 uL, 0.01 eq) and 16-bromo-1,1-difluorohexadecane (124.5 mg, 662.7 umol, 0.05 mL, 2.26e-2 eq) under N₂. The reaction was stirred until the color of I₂ was faded into a colorless solution. Then 16-bromo-1,1-difluorohexadecane (10 g, 29.3 mmol, 1 eq) in 2-Me-THF (50 mL) was added dropwise to the mixture. The reaction was stirred at 25° C. for 2 hr. The reaction mixture was used into the next step without further purification. The crude product (16,16-difluorohexadecyl)magnesium bromide, Intermediate I-41 (30 mmol, 2-Me-THF solution) was used into the next step without further purification as a gray solution.

### Intermediate I-42: (R)-19,19-difluoro-1-(trityloxy)nonadecan-2-ol

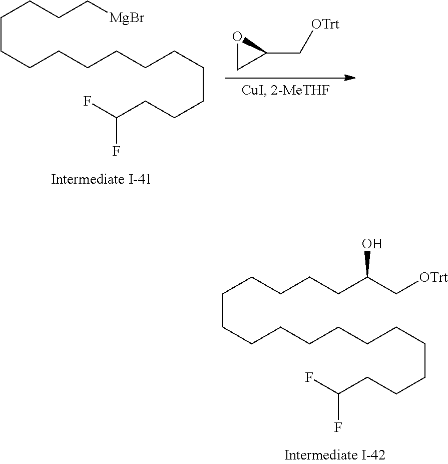

To a solution of (2R)-2-(trityloxymethyl)oxirane (6.6 g, 21.0 mmol, 1 eq) and CuI (200.3 mg, 1.0 mmol, 0.05 eq) in 2-Me-THF (10 mL) was added dropwise (16,16-difluorohexadecyl)magnesium bromide, Intermediate I-41 (2-Me-THF solution, 27.3 mmol, 1.3 eq) at −20° C. The solution was stirred for 10 min, warmed to 0° C., then stirred at 0° C. for 1 hr. The residue was diluted with NH₄Cl (200 mL) and extracted with EtOAc (100 mL×3). The combined organic layers were washed with brine 200 mL (100 mL×2), dried over Na₂SO₄, filtered and concentrated under reduced pressure to give a residue. The residue was purified by flash silica gel chromatography (ISCO®; 40 g SepaFlash® Silica Flash Column, Eluent of 0˜20% Ethyl acetate/Petroleum ether gradient@120 mL/min) to give (R)-19,19-difluoro-1-(trityloxy)nonadecan-2-ol, Intermediate I-42.

### Intermediate I-43: (R)-3-(((19,19-difluoro-1-(trityloxy)nonadecan-2-yl)oxy)methyl)-5-fluorobenzonitrile

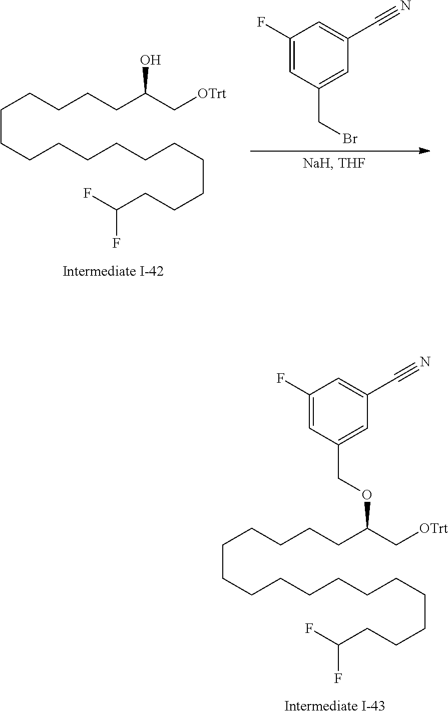

To a solution of (R)-19,19-difluoro-1-(trityloxy)nonadecan-2-ol, Intermediate I-42 (2.6 g, 4.6 mmol, 1 eq) in THF (30 mL) was added NaH (461.2 mg, 11.5 mmol, 60% purity, 2.5 eq) and 3-(bromomethyl)-5-fluoro-benzonitrile (1.1 g, 5.5 mmol, 1.2 eq). The reaction was stirred at 60° C. for 12 hr. The residue was diluted with NH₄Cl (100 Ml) and extracted with EtOAc (100 mL×3). The combined organic layers were washed with brine (100 mL×2), dried over Na₂SO₄, filtered and concentrated under reduced pressure to give a residue. The residue was purified by flash silica gel chromatography (ISCO®; 40 g SepaFlash® Silica Flash Column, Eluent of 0˜10% Ethyl acetate/Petroleum ether gradient @ 120 mL/min) to give (R)-3-(((19,19-difluoro-1-(trityloxy)nonadecan-2-yl)oxy)methyl)-5-fluorobenzonitrile, Intermediate I-43. ¹H NMR (400 MHZ, CHLOROFORM-d) δ 7.39 (br d, J=7.8 Hz, 6H), 7.30 (br s, 10H), 7.20-7.17 (m, 2H), 5.74 (tt, J=4.5, 57.0 Hz, 1H), 4.72-4.46 (m, 2H), 3.47 (quin, J=5.3 Hz, 1H), 3.16 (d, J=4.6 Hz, 2H), 1.85-1.67 (m, 2H), 1.57-1.33 (m, 5H), 1.24-1.16 (m, 25H).

### Intermediate I-44: (R)-3-(((19,19-difluoro-1-hydroxynonadecan-2-yl)oxy)methyl)-5-fluorobenzonitrile

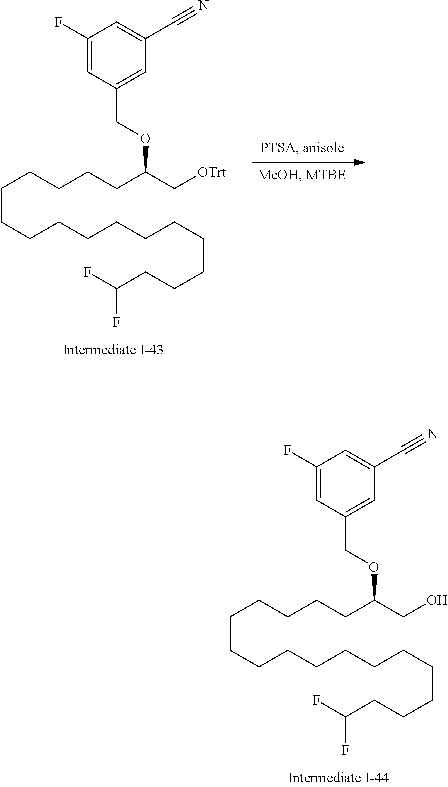

To a solution of (R)-3-(((19,19-difluoro-1-(trityloxy)nonadecan-2-yl)oxy)methyl)-5-fluorobenzonitrile Intermediate I-43 (1.8 g, 2.6 mmol, 1 eq) in MeOH (4.8 mL) and MTBE (36 mL) was added anisole (140.5 mg, 1.3 mmol, 141.2 uL, 0.5 eq) and PTSA (223.7 mg, 1.3 mmol, 0.5 eq) and the mixture was stirred at 50° C. for 2 hr. The residue was diluted with NaHCO₃ (100 mL) and extracted with EtOAc (50 mL×3). The combined organic layers were washed with brine (50 mL×2), dried over Na₂SO₄, filtered and concentrated under reduced pressure to give a residue. The residue was purified by flash silica gel chromatography (ISCO®; 40 g SepaFlash® Silica Flash Column, Eluent of 0˜50% Ethyl acetate/Petroleum ether gradient @ 120 mL/min) to give (R)-3-(((19,19-difluoro-1-hydroxynonadecan-2-yl)oxy)methyl)-5-fluorobenzonitrile, Intermediate I-44. ¹H NMR (400 MHZ, CHLOROFORM-d) δ 7.46 (s, 1H), 7.34 (br d, J=9.0 Hz, 1H), 7.29 (br s, 1H), 5.98-5.61 (m, 1H), 4.64 (s, 2H), 3.78-3.72 (m, 1H), 3.65-3.58 (m, 1H), 3.56-3.49 (m, 1H), 1.89-1.74 (m, 3H), 1.68-1.49 (m, 2H), 1.48-1.40 (m, 2H), 1.35-1.24 (m, 25H). MS (ESI): m/z=468.3 [M+H]⁺.

### Intermediate I-45: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) ((R)-2-((3-cyano-5-fluorobenzyl)oxy)-19,19-difluorononadecyl) phosphate

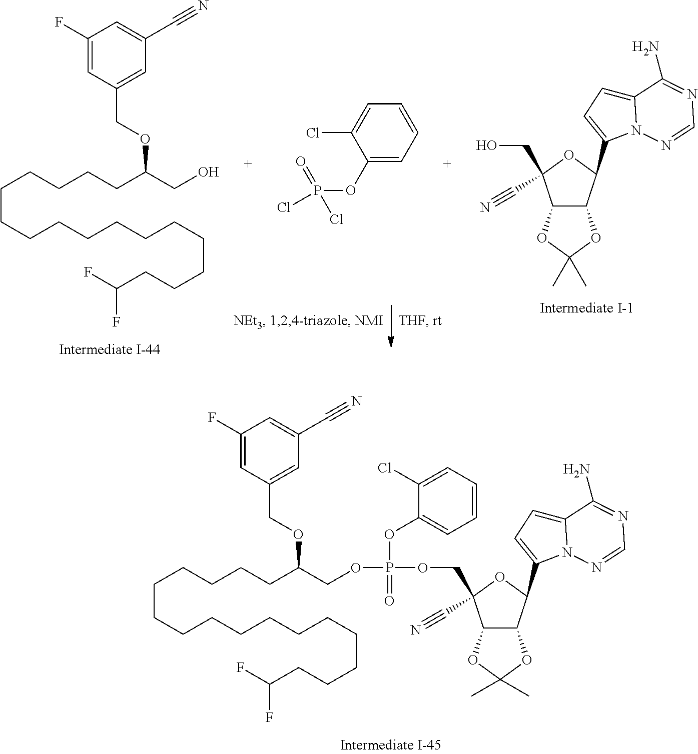 [SMILES: N#Cc1cc(F)cc(CO[C@@H](CO)CCCCCCCCCCCCCCCCC(F)F)c1 ; O=P(Cl)(Cl)Oc1ccccc1Cl ; CC1(C)O[C@H]2[C@H](c3ccc4c(N)ncnn34)O[C@](C#N)(CO)[C@H]2O1 ; CC1(C)O[C@H]2[C@H](c3ccc4c(N)ncnn34)O[C@](C#N)(COP(=O)(OC[C@@H](CCCCCCCCCCCCCCCCC(F)F)OCc3cc(F)cc(C#N)c3)Oc3ccccc3Cl)[C@H]2O1]

1H-1,2,4-triazole (208 mg, 3.02 mmol, 5.0 equiv.) was dissolved in THF (10.0 mL). TEA (0.34 mL, 2.44 mmol, 4.04 equiv.) was added to the solution followed by 2-chlorophenyl phosphorodichloridate (0.18 mL, 1.09 mmol, 1.80 equiv.). The reaction mixture was stirred at rt for 4 min prior to the addition of ((3aS,4R,6aS)-6-(4-aminopyrrolo[2,1-f][1,2,4]triazin-7-yl)-4-(fluoromethyl)-2,2-dimethyltetrahydrofuro[3,4-d][1,3]dioxol-4-yl)methanol, Intermediate I-1 (200 mg, 0.604 mmol, 1.0 equiv.) in one portion followed by 1-methylimidazole (0.26 mL, 3.20 mmol, 5.31 equiv.). The solution was stirred for an additional 10 min before adding (R)-3-(((19,19-difluoro-1-hydroxynonadecan-2-yl)oxy)methyl)-5-fluorobenzonitrile, Intermediate I-44 (283 mg, 0.604 mmol, 1.0 equiv.). After stirring at room temperature overnight, the solution was diluted with EtOAc (150 mL) and water (100 mL). The organic layer was washed with brine (50 mL), dried over Na₂SO₄, filtered, and concentrated in vacuo prior to purification by silica gel chromatography (0-100% EtOAc in hexanes) to afford the Intermediate I-45. ¹H NMR (400 MHz, Methanol-d4) δ 7.81-7.77 (m, 1H), 7.48-7.28 (m, 5H), 7.20-7.07 (m, 2H), 6.86-6.79 (m, 1H), 6.79-6.74 (m, 1H), 6.02-5.67 (m, 1H), 5.66-5.62 (m, 1H), 5.33-5.26 (m, 1H), 5.18-5.12 (m, 1H), 4.64-4.44 (m, 4H), 4.36-4.24 (m, 1H), 4.24-4.09 (m, 1H), 3.64-3.55 (m, 1H), 1.87-1.67 (m, 5H), 1.60-1.20 (m, 33H). ¹⁹F NMR (376 MHz, Methanol-d4) δ−112.69-−112.80 (m), −117.78-−118.21 (m). 31P NMR (162 MHZ, Methanol-d4) δ−7.43-−8.08 (m). MS m/z [M+1]=973.3.

### Intermediate I-46: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((R)-2-((3-cyano-5-fluorobenzyl)oxy)-19,19-difluorononadecyl) hydrogen phosphate

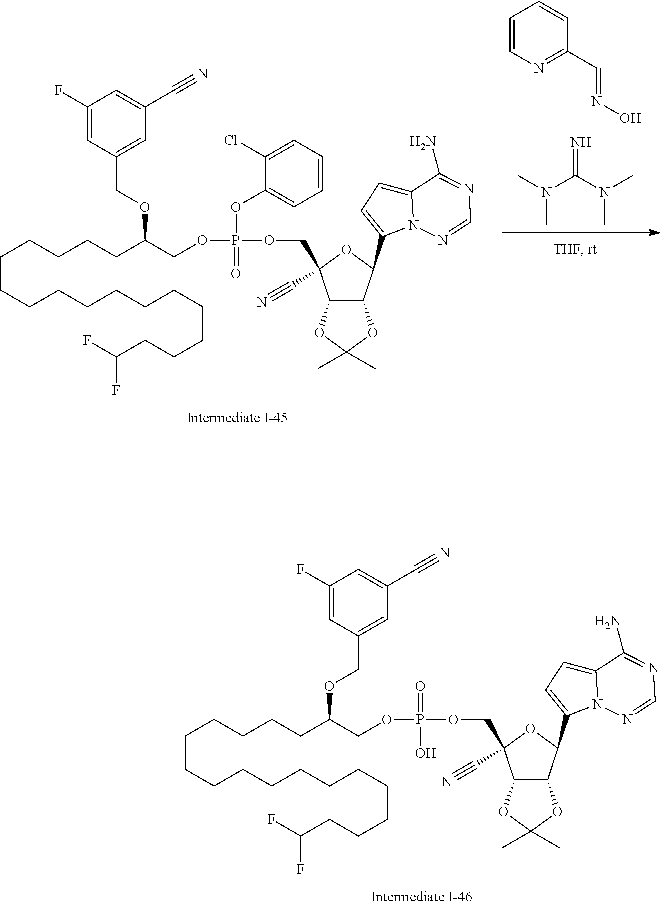

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) ((R)-2-((3-cyano-5-fluorobenzyl)oxy)-19,19-difluorononadecyl) phosphate, Intermediate I-45 (330 mg, 0.339 mmol, 1.0 equiv.) in THF (10.0 mL) was added 1,1,3,3-tetramethylguanidine (0.26 mL, 2.03 mmol, 6.0 equiv.) and syn-2-pyridinealdoxime (317 mg, 2.60 mmol, 7.66 equiv.). The reaction mixture was stirred overnight. The solution was concentrated in vacuo and purified by silica gel (0-40% MeOH in DCM) to afford the title compound, Intermediate I-46. ¹H NMR (400 MHZ, Methanol-d4) δ 7.83 (s, 1H), 7.51-7.47 (m, 1H), 7.43-7.34 (m, 2H), 6.85 (d, J=4.5 Hz, 1H), 6.79 (d, J=4.5 Hz, 1H), 5.84 (tt, J=57.1, 4.5 Hz, 1H), 5.63 (d, J=3.5 Hz, 1H), 5.25 (dd, J=6.6, 3.6 Hz, 1H), 5.13 (d, J=6.6 Hz, 1H), 4.73 (d, J=13.0 Hz, 2H), 4.51 (d, J=13.1 Hz, 1H), 4.17-4.06 (m, 2H), 3.95-3.81 (m, 2H), 3.58-3.50 (m, 1H), 1.87-1.65 (m, 5H), 1.49-1.19 (m, 33H). ¹⁹F NMR (376 MHz, Methanol-d4) δ −112.90-−113.05 (m), −117.84-−118.13 (m). ³¹P NMR (162 MHZ, Methanol-d4) δ−0.19-−0.80 (m). MS m/z [M+1]=863.2.

### Intermediate I-47: 2-(nonadec-2-yn-1-yloxy)tetrahydro-2H-pyran

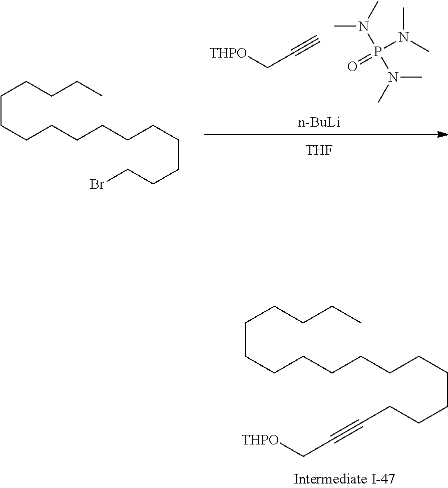

A solution of 2-(prop-2-yn-1-yloxy)tetrahydro-2H-pyran (20.0 g, 321 mmol, 1.00 equiv.) and HMPA (201 g, 3.50 equiv.) in THF (900 mL) was cooled to −70° C. n-BuLi (24.6 g, 385 mmol, 2.50 M in THF, 1.20 equiv.) was added gradually over 1 h to the solution at −70° C. under N₂. 1-bromohexadecane (45.0 g, 321 mmol, 1.00 equiv.) dissolved in THF (200 mL) was then added at −70° C. The reaction mixture was warmed to 20° C. and stirred for 12 h. The reaction was quenched with saturated NH₄Cl (3.0 L) and extracted with EtOAc (2×3.0 L). The organic was concentrated under reduced pressure and purified by silica gel chromatography (petroleum ether/ethyl acetate=I/O to 0/1) to afford the title compound, Intermediate I-47. ¹H NMR: (400 MHz, CDCl₃-d) δ=4.82 (t, J=3.4 Hz, 1H), 4.35-4.16 (m, 2H), 3.86 (ddd, J=3.0, 8.8, 11.4 Hz, 1H), 3.59-3.49 (m, 1H), 2.22 (tt, J=2.2, 7.0 Hz, 2H), 1.91-1.18 (m, 34H), 0.97-0.83 (m, 3H).

### Intermediate I-48: nonadec-2-yn-1-ol

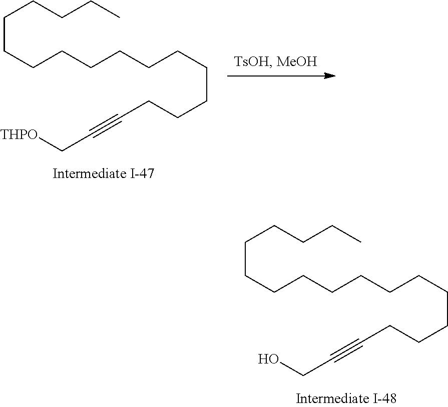

To a solution of 2-(nonadec-2-yn-1-yloxy)tetrahydro-2H-pyran, Intermediate I-47 (30.0 g, 82.2 mmol, 1.00 equiv.) in MeOH (300 mL) was added TsOH H₂O (1.57 g, 0.10 equiv.). The reaction mixture was stirred at room temperature for 12 h prior to filtering and isolating the filter cake to afford the title compound, Intermediate I-48. TLC Information (Petroleum ether/Ethyl acetate=10/1) R_{f}=0.40.

### Intermediate I-49: nonadec-18-yn-1-ol

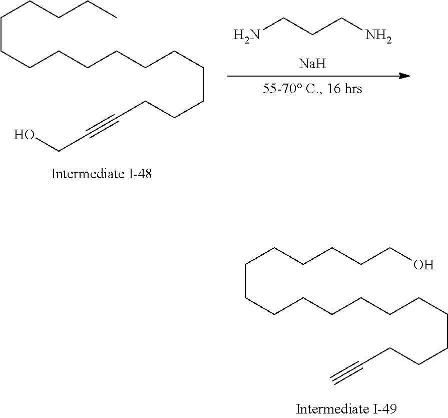

NaH (17.8 g, 445 mmol, 5.00 equiv., 60% by mass) was added to propane-1,3-diamine (325 mL). The solution was stirred at 70° C. for 3 h. Nonadec-2-yn-1-ol (25.0 g, 1.00 equiv.) was added to the mixture. The reaction was stirred at 55° C. for 13 h. The mixture was poured into saturated NH₄Cl (2.00 L) and extracted with EtOAc (2.00 L). The residue was purified by chromatography (SiO₂, Petroleum ether/Ethyl acetate=I/O to 0/1) to afford the title compound, Intermediate I-49. TLC Information (Petroleum ether/Ethyl acetate=5/1) R_{f}=0.30.

### Intermediate I-50: 1-methoxy-4-((nonadec-18-yn-1-yloxy)methyl)benzene

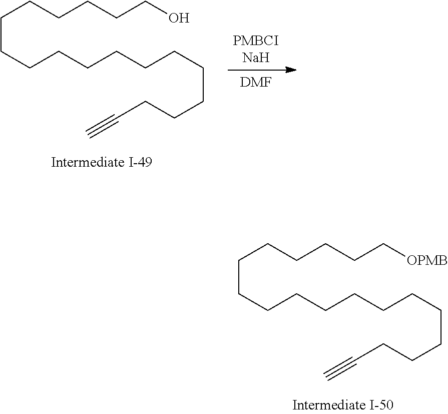

To a solution of nonadec-18-yn-1-ol, Intermediate I-49 (20.0 g, 71.3 mmol, 1.00 equiv.) in DMF (300 mL) was added NaH (7.13 g, 178 mmol, 60.0% purity, 2.50 equiv.) at 0° C. followed by PMBCl (16.7 g, 106 mmol, 14.5 mL, 1.50 equiv.) at 0° C. The reaction was stirred at 50° C. for 12 h. The mixture was poured into saturated NH₄Cl (1.00 L) and the aqueous layer was extracted with EtOAc (1.00 L×2). The organic layers were concentrated under reduced pressure and purified by column chromatography (SiO₂, Petroleum ether/Ethyl acetate=I/O to 0/1) to afford the Intermediate I-50. ¹H NMR: (400 MHz, CDCl₃-d) δ −7.25 (d, J=3.2 Hz, 1H), 6.87 (d, J=8.6 Hz, 2H), 4.43 (s, 2H), 3.80 (s, 3H), 3.43 (t, J=6.6 Hz, 2H), 2.18 (dt, J=2.6, 7.0 Hz, 1H), 1.93 (t, J=2.6 Hz, 1H), 1.78 (t, J=2.6 Hz, 1H), 1.64-1.15 (m, 38H).

### Intermediate I-51: 1-methoxy-4-(((20,20,20-trifluoroicos-18-yn-1-yl)oxy)methyl)benzene

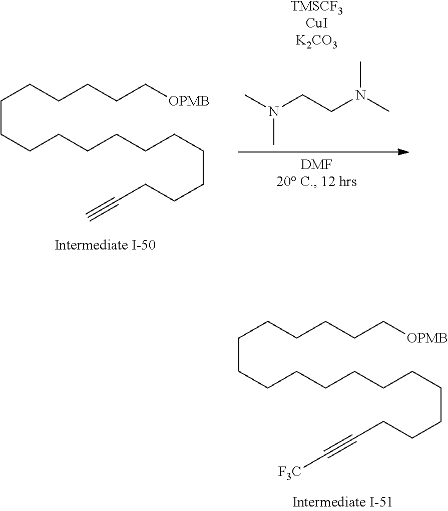

To a solution of CuI (7.13 g, 37.4 mmol, 1.50 eq), K₂CO₃ (10.3 g, 74.8 mmol, 3.00 eq) and N1,N1,N2,N2-tetramethylethane-1,2-diamine (4.35 g, 37.4 mmol, 5.65 mL, 1.50 eq) in DMF (100 mL) was added TMSCF₃ (7.10 g, 49.9 mmol, 2.00 eq) at 0° C. 1-methoxy-4-((nonadec-18-yn-1-yloxy)methyl)benzene, Intermediate I-50 (10.0 g, 24.9 mmol, 1.00 eq) in DMF (100 mL) was then added at 0° C. The reaction mixture was stirred at 20° C. for 12 h. The mixture was poured into water (600 mL) and the aqueous layer was extracted with DCM (600 mL). The organic layer was concentrated under reduced pressure and the residue was purified by column chromatography (SiO₂, Petroleum ether/Ethyl acetate=1/0 to 0/1) to afford the title compound Intermediate I-51. ¹H NMR: (400 MHz, CDCl₃-d) δ=7.33-7.22 (m, 2H), 6.89 (d, J=8.6 Hz, 2H), 4.44 (s, 2H), 3.81 (s, 3H), 3.44 (t, J=6.6 Hz, 2H), 2.37-1.93 (m, 2H), 1.79 (t, J=2.4 Hz, 1H), 1.67-1.53 (m, 3H), 1.54-1.18 (m, 29H).

### Intermediate I-52: 20,20,20-trifluoroicos-18-yn-1-ol

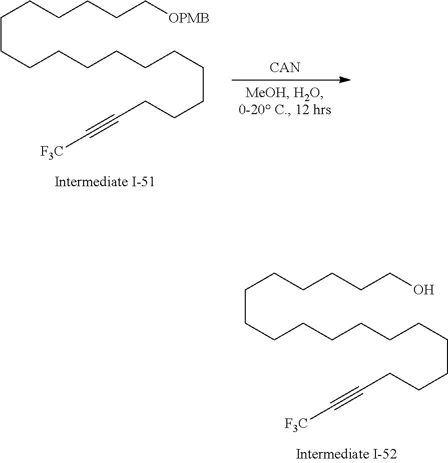

To a solution of 1-methoxy-4-(((20,20,20-trifluoroicos-18-yn-1-yl)oxy)methyl)benzene Intermediate I-51 (5.00 g, 10.6 mmol, 1.00 eq) in MeOH (50.0 mL) and H₂O (5.00 mL) was added CAN (17.5 g, 32.0 mmol, 15.9 mL, 3.00 eq) at 0° C. The reaction was stirred at 20° C. for 12 h. The mixture was poured into H₂O (200 mL) and the aqueous layer was extracted with DCM (200 mL). The organic layer was concentrated under reduced pressure and purified by column chromatography (SiO₂, Petroleum ether/Ethyl acetate=I/O to 0/1) to afford the title compound, Intermediate I-52. TLC Information (Petroleum ether/Ethyl acetate=10/1; R_{f}=0.20).

### Intermediate I-53: 20,20,20-trifluoroicosan-1-ol

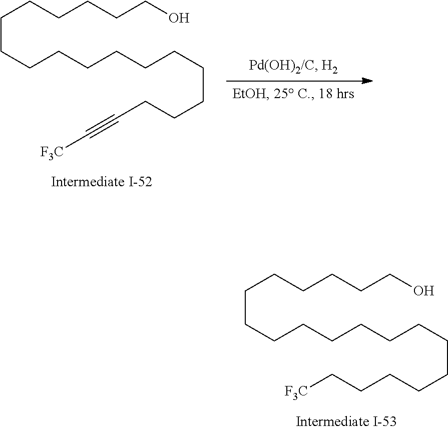 [SMILES: OCCCCCCCCCCCCCCCCCC#CC(F)(F)F ; "EtOH, 25° C., 18 hrs" ; "Pd(OH)2/C, H2" ; OCCCCCCCCCCCCCCCCCCCC(F)(F)F]

To a solution of Pd(OH)₂ (15.0 mg, 0.430 mmol, 0.100 eq) in EtOH (15.0 mL) was added 20,20,20-trifluoroicos-18-yn-1-ol, Intermediate I-52 (1.50 g, 4.30 mmol, 1.00 eq) at 20° C. under argon. The atmosphere was exchanged with H₂ gas and the mixture was stirred under H₂ (15 Psi) at 20° C. for 12 h. The solution was filtered, and the filtrate was concentrated under reduced pressure to afford the title compound Intermediate I-53. ¹H NMR: (400 MHZ, CD₃OD-d₄) δ=3.54 (t, J=6.6 Hz, 2H), 2.20-2.06 (m, 2H), 1.59-1.48 (m, 4H), 1.29 (s, 30H).

### Intermediate I-54: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) (20,20,20-trifluoroicosyl) phosphate

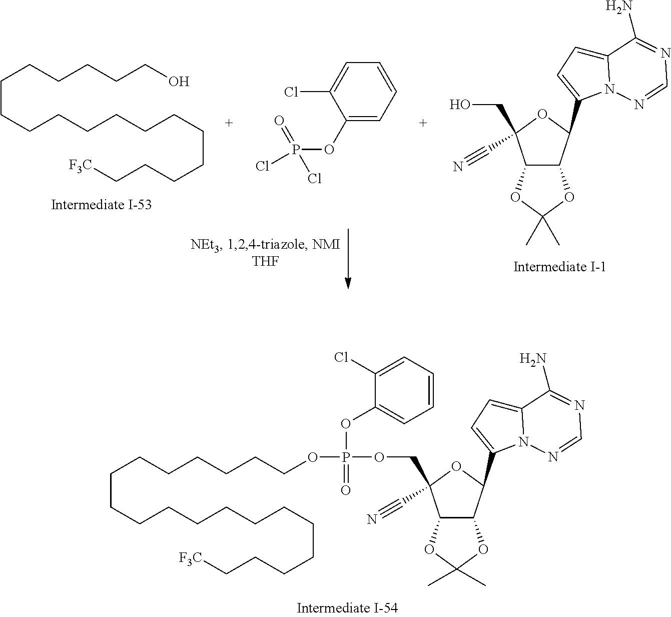 [SMILES: OCCCCCCCCCCCCCCCCCCC(F)(F)F ; O=P(Cl)(Cl)Oc1ccccc1Cl ; CC1(C)O[C@H]2[C@H](c3ccc4c(N)ncnn34)O[C@](C#N)(CO)[C@H]2O1 ; CC1(C)O[C@H]2[C@H](c3ccc4c(N)ncnn34)O[C@](C#N)(COP(=O)(OCCCCCCCCCCCCCCCCCCCC(F)(F)F)Oc3ccccc3Cl)[C@H]2O1]

1H-1,2,4-triazole (156 mg, 2.26 mmol, 3.0 equiv.) was dissolved in THF (10.0 mL). TEA (0.32 mL, 2.26 mmol, 3.0 equiv.) was added to the solution followed by 2-chlorophenyl phosphorodichloridate (0.16 mL, 0.981 mmol, 1.30 equiv.). The reaction mixture was stirred at rt for 5 min prior to the addition of ((3aS,4R,6aS)-6-(4-aminopyrrolo[2,1-f][1,2,4]triazin-7-yl)-4-(fluoromethyl)-2,2-dimethyltetrahydrofuro[3,4-d][1,3]dioxol-4-yl)methanol Intermediate I-1 (250 mg, 0.755 mmol, 1.00 equiv.) in one portion followed by 1-methylimidazole (0.18 mL, 2.26 mmol, 3.0 equiv.). The solution was stirred for an additional 10 min before adding 20,20,20-trifluoroicosan-1-ol Intermediate I-53 (266 mg, 0.755 mmol, 1.0 equiv.). After stirring at room temperature overnight, the solution was diluted with EtOAc (100 mL) and water (100 mL). The organic layer was washed with brine (50 mL), dried over Na₂SO₄, filtered, and concentrated in vacuo prior to purification by silica gel chromatography (0-100% EtOAc in hexanes) to afford the title compound Intermediate I-54. ¹H NMR (400 MHZ, Methanol-d4) δ 7.83-7.78 (m, 1H), 7.48-7.40 (m, 1H), 7.37-7.30 (m, 1H), 7.21-7.12 (m, 2H), 6.87-6.81 (m, 1H), 6.80-6.75 (m, 1H), 5.68-5.62 (m, 1H), 5.36-5.29 (m, 1H), 5.21-5.13 (m, 1H), 4.60-4.45 (m, 2H), 4.25-4.11 (m, 2H), 2.19-2.04 (m, 2H), 1.73 (s, 3H), 1.68-1.48 (m, 4H), 1.43-1.20 (m, 33H). ¹⁹F NMR (376 MHz, Methanol-d4) δ−68.55 (t, J=11.2 Hz). 31P NMR (162 MHz, Methanol-d4) δ −7.59-−8.04 (m). MS m/z [M+1]=856.2

### Intermediate I-55: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (20,20,20-trifluoroicosyl) hydrogen phosphate

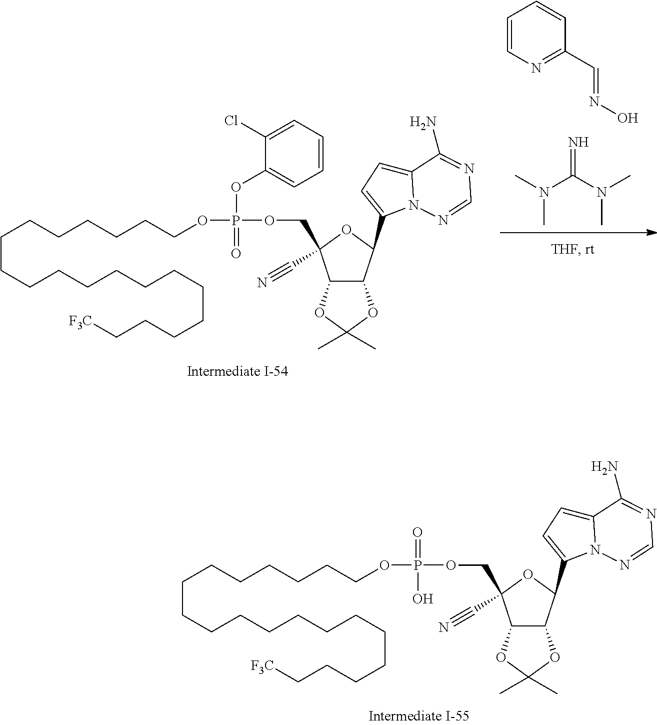

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) (20,20,20-trifluoroicosyl) phosphate Intermediate I-54 (385 mg, 0.450 mmol, 1.0 equiv.) in THF (10.0 mL) was added 1,1,3,3-tetramethylguanidine (0.56 mL, 4.50 mmol, 10 equiv.) and syn-2-pyridinealdoxime (549 mg, 4.50 mmol, 10 equiv.). The reaction mixture was stirred at room temperature overnight. The solution was concentrated in vacuo and purified by silica gel (0-40% MeOH in DCM) to afford the title compound Intermediate I-55. ¹H NMR (400 MHZ, Methanol-d4) δ 7.89 (s, 1H), 6.98 (d, J=4.6 Hz, 1H), 6.87 (d, J=4.6 Hz, 1H), 5.65 (d, J=3.5 Hz, 1H), 5.27 (dd, J=6.6, 3.6 Hz, 1H), 5.15 (d, J=6.6 Hz, 1H), 4.15-4.05 (m, 2H), 3.87-3.79 (m, 2H), 2.20-2.04 (m, 2H), 1.70 (s, 3H), 1.59-1.48 (m, 4H), 1.44-1.21 (m, 33H). ¹⁹F NMR (376 MHZ, Methanol-d4) δ−68.51-−68.60 (m). 31P NMR (162 MHZ, Methanol-d4) δ 0.04-−0.57 (m). MS m/z [M+1]=746.2

### Intermediate I-56: methyl 15-hydroxypentadecanoate

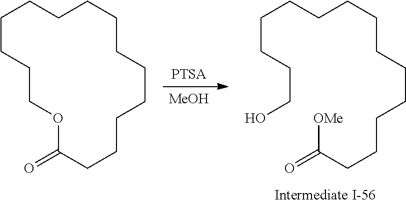

To a solution of oxacyclohexadecan-2-one (7 g, 29.1 mmol, 1 eq) in MeOH (70 mL) was added PTSA (752.2 mg, 4.4 mmol, 0.15 eq). Then the mixture was stirred at 70° C. for 12 hr and concentrated under reduced pressure to remove solvent. The residue was diluted with H₂O (500 mL) and extracted with Ethyl acetate (300 mL×3). The combined organic layer was washed with H₂O (500 mL×2), dried over Na₂SO₄, filtered, and concentrated under reduced pressure to give a residue. The residue was purified by flash silica gel chromatography (Silica Flash Column, Eluent of 0˜15% Ethyl acetate/Petroleum ether gradient @ 120 mL/min) to give compound methyl 15-hydroxypentadecanoate, Intermediate I-56. ¹H NMR (400 MHZ, CHLOROFORM-d) δ 3.70-3.62 (m, 5H), 2.31 (t, J=7.6 Hz, 2H), 1.65-1.54 (m, 4H), 1.34-1.25 (m, 20H).

### Intermediate I-57: methyl 15-iodopentadecanoate

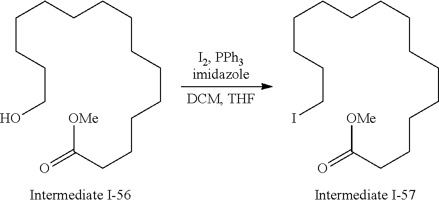

To a solution of methyl 15-hydroxypentadecanoate, Intermediate I-56 (5.0 g, 18.4 mmol, 1 eq) in DCM (100 mL) were added PPh₃ (9.6 g, 36.7 mmol, 2.0 eq) and imidazole (3.8 g, 55.1 mmol, 3.0 eq). Then the mixture was degassed, purged with N₂ for 3 times, cooled to 0° C., and I₂ (9.3 g, 36.7 mmol, 7.4 mL, 2.0 eq) in THF (15 mL) added. The mixture was stirred at 25° C. for 12 h and 10% Na₂S203 (500 mL) added. The mixture was stirred for 5 min, layers separated, and the aqueous layer extracted with DCM (300 mL×3). The combined organic layer was washed with H₂O (500 mL×2), dried over Na₂SO₄, filtered, and concentrated under reduced pressure to give a residue. The residue was purified by flash silica gel chromatography (Silica Flash Column, Eluent of 0˜1% Ethyl acetate/Petroleum ether gradient @ 150 mL/min) to give compound methyl 15-iodopentadecanoate Intermediate I-57. ¹H NMR (400 MHZ, CHLOROFORM-d) δ 3.67 (s, 3H), 3.20 (t, J=7.0 Hz, 2H), 2.31 (t, J=7.5 Hz, 2H), 1.83 (quin, J=7.2 Hz, 2H), 1.67-1.58 (m, 2H), 1.42-1.35 (m, 2H), 1.33-1.24 (m, 18H).

### Intermediate I-58: 15-iodopentadecan-1-ol

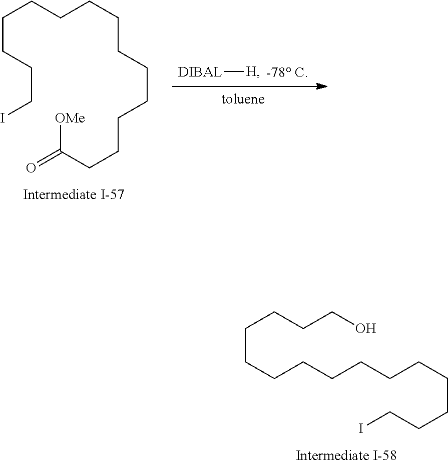

To a solution of methyl 15-iodopentadecanoate, Intermediate I-57 (10.0 g, 26.2 mmol, 1.0 eq) in toluene (100 mL) was added dropwise DIBAL-H (1 M, 65.4 mL, 2.5 eq) at −78° C. The resulting mixture was stirred at 0° C. for 2 h and the reaction mixture quenched by pouring into sat. NH₄Cl solution (800 ml) at 0° C. The mixture was stirred for 30 min and then extracted with ethyl acetate (300 mL×3). The combined organic layer was washed with H₂O (500 mL×2), dried over Na₂SO₄, filtered, and concentrated under reduced pressure to give a residue. The residue was purified by flash silica gel chromatography (Silica Flash Column, Eluent of 0˜15% Ethyl acetate/Petroleum ether gradient @ 120 mL/min) to give compound 15-iodopentadecan-1-ol, Intermediate I-58. ¹H NMR (400 MHZ, CHLOROFORM-d) § 3.64 (t, J=6.6 Hz, 2H), 3.19 (t, J=7.1 Hz, 2H), 1.87-1.79 (m, 2H), 1.57 (quin, J=6.9 Hz, 2H), 1.34-1.25 (m, 22H).

### Intermediate I-59: 2-((15-iodopentadecyl)oxy)tetrahydro-2H-pyran

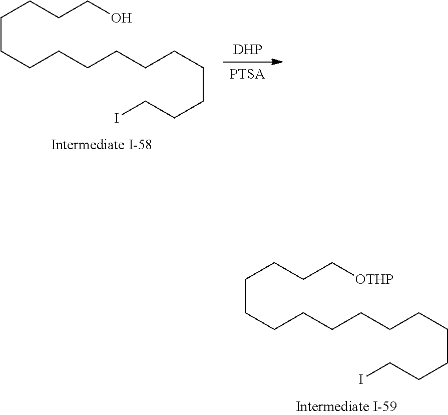

To a solution of 15-iodopentadecan-1-ol, Intermediate I-58 (6.0 g, 16.9 mmol, 1 eq) in THF (130 mL) were added 3,4-dihydro-2H-pyran (4.3 g, 50.8 mmol, 4.6 mL, 3.0 eq) and PTSA (437.4 mg, 2.5 mmol, 0.2 eq). The resulting mixture was stirred at 20° C. for 12 h and concentrated under reduced pressure to remove solvent. The residue was diluted with H₂O (300 mL) and extracted with ethyl acetate (150 mL×3). The combined organic layer was washed with H₂O (200 mL×2), dried over Na₂SO₄, filtered, and concentrated under reduced pressure to give a residue. The residue was purified by flash silica gel chromatography (Silica Flash Column, Eluent of 0˜2% Ethyl acetate/Petroleum ether gradient @ 80 mL/min) to give compound 2-((15-iodopentadecyl)oxy)tetrahydro-2H-pyran, Intermediate I-59. ¹H NMR (400 MHZ, CHLOROFORM-d) δ 4.61-4.55 (m, 1H), 3.88 (ddd, J=3.4, 7.5, 11.1 Hz, 1H), 3.74 (td, J=6.9, 9.5 Hz, 1H), 3.51 (td, J=5.2, 10.9 Hz, 1H), 3.39 (td, J=6.7, 9.5 Hz, 1H), 3.20 (t, J=7.1 Hz, 2H), 1.85-1.80 (m, 2H), 1.62-1.51 (m, 6H), 1.44-1.19 (m, 24H).

### Intermediate I-60: 2-((16,16,16-trifluorohexadecyl)oxy)tetrahydro-2H-pyran

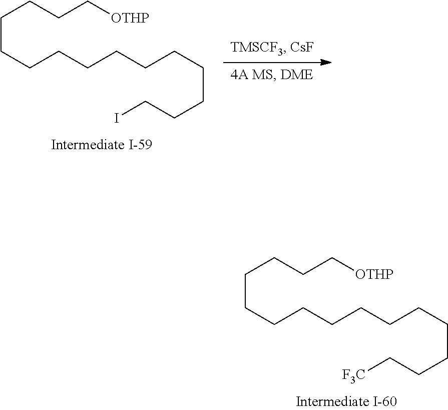

To a solution of CsF (11.1 g, 73.0 mmol, 2.7 mL, 3.2 eq) in 15-CROWN-5 (27.4 g, 124.3 mmol, 24.7 mL, 5.5 eq) were added DME (150 mL) and 4A MS (10.0 g) and then the mixture cooled to −20° C. Then a solution of TMS-CF₃ (13.8 g, 96.7 mmol, 4.2 eq) and 2-(15-iodopentadecoxy)tetrahydropyran, Intermediate I-59 (10.0 g, 22.8 mmol, 1.0 eq) in DME (80 mL) was added into the above solution over 15 min. The mixture was stirred at 25° C. for 12 h, concentrated under reduced pressure, diluted with ethyl acetate (100 mL), washed with NaCl (80 mL×3), dried over Na₂SO₄, filtered, and concentrated under reduced pressure to give a residue. The residue was purified by column chromatography (SiO₂, Petroleum ether/Ethyl acetate=100/1 to 100/3) to give compound 2-((16,16,16-trifluorohexadecyl)oxy)tetrahydro-2H-pyran, Intermediate I-60. ¹H NMR (400 MHZ, CHLOROFORM-d) § 4.58 (br s, 1H), 3.94-3.83 (m, 1H), 3.80-3.68 (m, 1H), 3.57-3.46 (m, 1H), 3.44-3.33 (m, 1H), 2.15-1.98 (m, 2H), 1.90-1.67 (m, 2H), 1.65-1.48 (m, 8H), 1.38-1.24 (m, 22H); ¹⁹F NMR (376 MHz, CHLOROFORM-d) δ −66.49 (t, J=11.66 Hz).

### Intermediate I-61: 16,16,16-trifluorohexadecan-1-ol

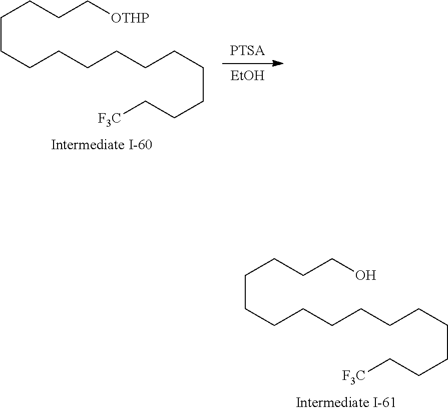

To a solution of 2-(16,16,16-trifluorohexadecoxy)tetrahydropyran Intermediate I-60 (5.0 g, 13.1 mmol, 1.0 eq) in EtOH (50 mL) was added PTSA (2.3 g, 13.1 mmol, 1.0 eq). The mixture was stirred at 50° C. for 12 h, concentrated under reduced pressure, diluted with H₂O (300 mL), and extracted with ethyl acetate (200 mL×3). The combined organic layer was washed with H₂O (300 mL×2), dried over Na₂SO₄, filtered, and concentrated under reduced pressure to give a residue. The residue was purified by flash silica gel chromatography (Silica Flash Column, Eluent of 0˜10% Ethyl acetate/Petroleum ether gradient @ 100 mL/min) to give compound 16,16,16-trifluorohexadecan-1-ol, Intermediate I-61. ¹H NMR (400 MHZ, CHLOROFORM-d) & 3.65 (t, J=6.6 Hz, 2H), 2.14-1.99 (m, 2H), 1.61-1.50 (m, 4H), 1.37-1.25 (m, 22H).

### Intermediate I-62: 16-bromo-1,1,1-trifluorohexadecane

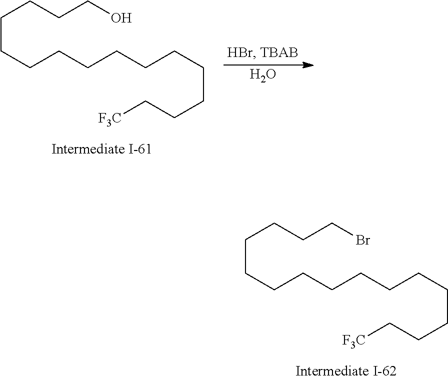 [SMILES: OCCCCCCCCCCCCCCCC(F)(F)F ; O ; FC(F)(F)CCCCCCCCCCCCCCCBr]

To a solution of 16,16,16-trifluorohexadecan-1-ol, Intermediate I-61 (5 g, 16.9 mmol, 1 eq) in HBr (40 mL, 30% in H₂O) was added TBAB (217.5 mg, 674.7 umol, 0.04 eq) at 20° C. Then the mixture was stirred at 100° C. for 12 h and concentrated under reduced pressure to remove solvent. The residue was diluted with H₂O (150 mL) and extracted with DCM (100 mL×3). The combined organic layer was washed with H₂O (300 mL×2), dried over Na₂SO₄, filtered, and concentrated under reduced pressure to give a residue. The residue was purified by flash silica gel chromatography (50 g Silica Flash Column, Eluent of 100% Petroleum ether gradient @ 100 mL/min) to give compound 16-bromo-1,1,1-trifluorohexadecane, Intermediate I-62. ¹H NMR (400 MHZ, CHLOROFORM-d) § 3.42 (t, J=6.9 Hz, 2H), 2.14-2.00 (m, 2H), 1.91-1.81 (m, 2H), 1.61-1.51 (m, 2H), 1.47-1.40 (m, 2H), 1.27 (br s, 20H).

### Intermediate I-63: (16,16,16-trifluorohexadecyl)magnesium bromide

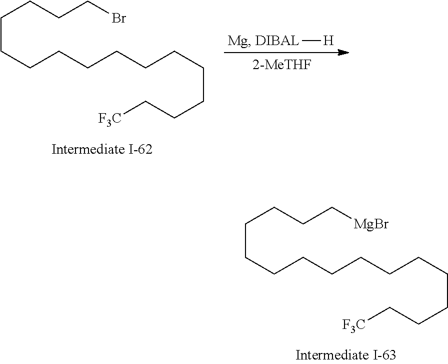

To a solution of Mg (661.2 mg, 27.2 mmol, 1.2 eq) in 2-Me THF (25 ml) was stirred at 20° C. under N₂. Then 16-bromo-1,1,1-trifluoro-hexadecane (0.5 g, 1.4 mmol) in 2-Me THF (1 ml) was added dropwise. Initiation of the reaction was performed through addition of DIBAL-H (1 M, 272.1 uL, 0.12 eq) at 20° C. Then the remaining 16-bromo-1,1,1-trifluoro-hexadecane (8.0 g, 22.3 mmol) in 2-Me THF (16 ml) was added and the mixture stirred at 20° C. for 4 h. The crude product (16,16,16-trifluorohexadecyl)magnesium bromide as brown liquid (in 2-Me THF) was used into the next step without further purification.

### Intermediate I-64: (S)-19,19,19-trifluoro-1-(trityloxy)nonadecan-2-ol

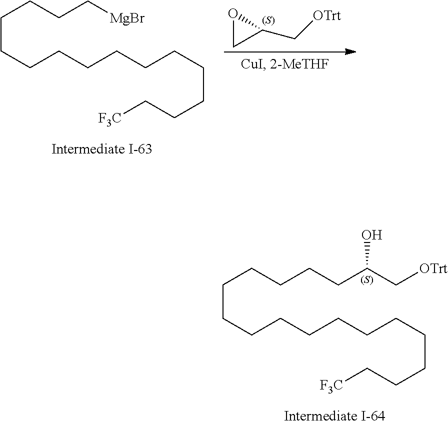

To a mixture of (2S)-2-(trityloxymethyl)oxirane, Intermediate I-63 (5.4 g, 17.0 mmol, 1 eq), CuI (162.3 mg, 852.2 umol, 0.05 eq) in 2-Me THF (30 mL) at −20° C. was added bromo(16,16,16-trifluorohexadecyl)magnesium (8.5 g, 22.2 mmol, 1.3 eq) over 10 min via cannula. The resulting mixture was stirred vigorously 5 min, warmed to 0° C., stirred for 2 h, the reaction quenched by addition sat. NH₄Cl solution (60 ml), and then the mixture extracted with ethyl acetate (40 mL×3). The combined organic layer was washed with H₂O (80 mL×2), dried over Na₂SO₄, filtered, and concentrated under reduced pressure to give a residue. The residue was purified by flash silica gel chromatography (40 g Silica Flash Column, Eluent of 0˜3% Ethyl acetate/Petroleum ether gradient @ 80 mL/min) to give compound (S)-19,19,19-trifluoro-1-(trityloxy)nonadecan-2-ol, Intermediate I-64. ¹H NMR (400 MHZ, CHLOROFORM-d) δ 7.38 (d, J=7.5 Hz, 6H), 7.26-7.16 (m, 9H), 3.76-3.66 (m, 1H), 3.12 (dd, J=3.2, 9.3 Hz, 1H), 2.97 (dd, J=7.8, 9.1 Hz, 1H), 2.24 (br s, 1H), 2.10-−1.75 (m, 2H), 1.52-1.46 (m, 2H), 1.19 (br d, J=9.9 Hz, 28H).

### Intermediate I-65: (S)-3-fluoro-5-(((19,19,19-trifluoro-1-(trityloxy)nonadecan-2-yl)oxy)methyl)benzonitrile

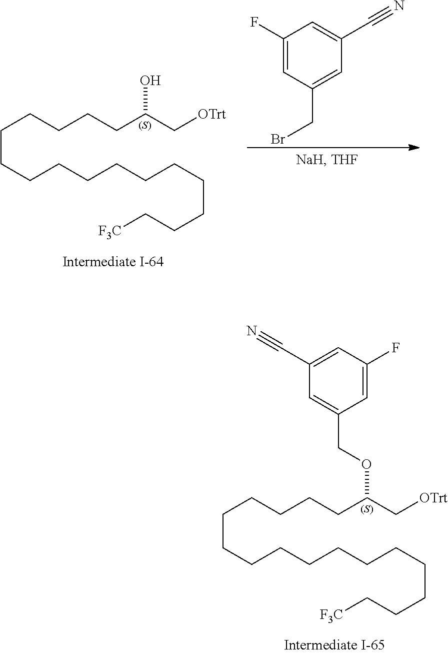

To a solution of (2S)-19,19,19-trifluoro-1-trityloxy-nonadecan-2-ol, Intermediate I-64 (5.0 g, 8.4 mmol, 1.0 eq) in THF (70 mL) was added NaH (837.8 mg, 21.0 mmol, 60% purity, 2.5 eq) at 0° C. and then the mixture was stirred at 0° C. for 30 min. Then 3-(bromomethyl)-5-fluoro-benzonitrile (2.2 g, 10.1 mmol, 1.2 eq) was added and the mixture was stirred at 65° C. for 12 h. The reaction was quenched by addition sat. NH₄Cl solution (100 ml) at 20° C. and the mixture extracted with ethyl acetate (50 mL×3). The combined organic layer was washed with H₂O (120 mL×2), dried over Na₂SO₄, and concentrated under reduced pressure to give a residue. The residue was purified by flash silica gel chromatography (20 g Silica Flash Column, Eluent of 0˜3% Ethyl acetate/Petroleum ether gradient @ 45 mL/min) to give compound (S)-3-fluoro-5-(((19,19,19-trifluoro-1-(trityloxy)nonadecan-2-yl)oxy)methyl)benzonitrile, Intermediate I-65. ¹H NMR (400 MHZ, CHLOROFORM-d) δ 7.48 (br d, J=7.2 Hz, 6H), 7.37-7.25 (m, 12H), 4.80-4.71 (m, 1H), 4.59 (d, J=12.8 Hz, 1H), 3.56 (quin, J=5.3 Hz, 1H), 3.24 (br d, J=4.8 Hz, 2H), 2.15-2.01 (m, 2H), 1.61-1.53 (m, 2H), 1.35-1.25 (m, 28H).

### Intermediate I-66: (S)-3-fluoro-5-(((19,19,19-trifluoro-1-hydroxynonadecan-2-yl)oxy)methyl)benzonitrile

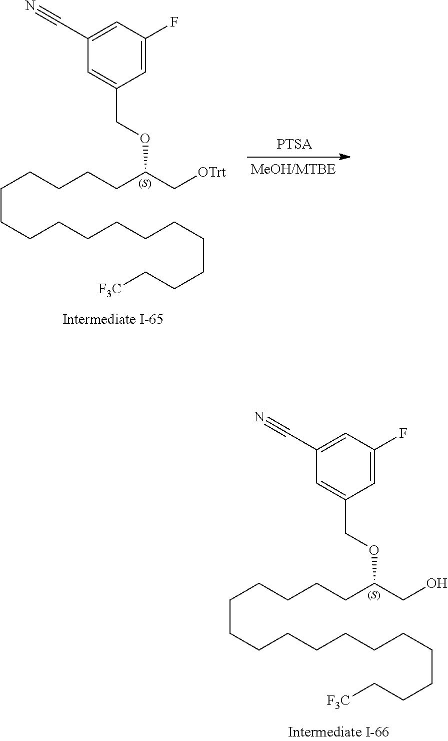

To a solution of 3-fluoro-5-[[(1S)-18,18,18-trifluoro-1-(trityloxymethyl)octadecoxy]methyl] benzonitrile, Intermediate I-65 (2.3 g, 3.2 mmol, 1 eq) in MTBE (46 mL) and MeOH (7 mL) were added anisole (170.4 mg, 1.6 mmol, 171.2 uL, 0.5 eq) and PTSA (271.3 mg, 1.6 mmol, 0.5 eq). The resulting mixture was stirred at 50° C. for 2 h, diluted with sat. NaHCO₃ (80 mL), and extracted with ethyl acetate (40 mL×3). The combined organic layer was washed with H₂O (50 mL×2), dried over Na₂SO₄, filtered, and concentrated under reduced pressure to give a residue. The residue was purified by flash silica gel chromatography (20 g Silica Flash Column, Eluent of 0˜7% Ethyl acetate/Petroleum ether gradient @ 45 mL/min) to give (S)-3-fluoro-5-(((19,19,19-trifluoro-1-hydroxynonadecan-2-yl)oxy)methyl)benzonitrile, Intermediate I-66. ¹H NMR (400 MHZ, CHLOROFORM-d) δ 7.46 (s, 1H), 7.35 (br d, J=8.9 Hz, 1H), 7.30-7.27 (m, 1H), 4.64 (s, 2H), 3.75 (br dd, J=2.4, 11.4 Hz, 1H), 3.65-3.58 (m, 1H), 3.53 (dq, J=3.3, 6.1 Hz, 1H), 2.13-1.99 (m, 2H), 1.79 (br s, 1H), 1.67-1.49 (m, 4H), 1.39-1.24 (m, 26H); MS (ESI): m/z=510.2 [M+Na].

### Intermediate I-67: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) ((S)-2-((3-cyano-5-fluorobenzyl)oxy)-19,19,19-trifluorononadecyl) phosphate

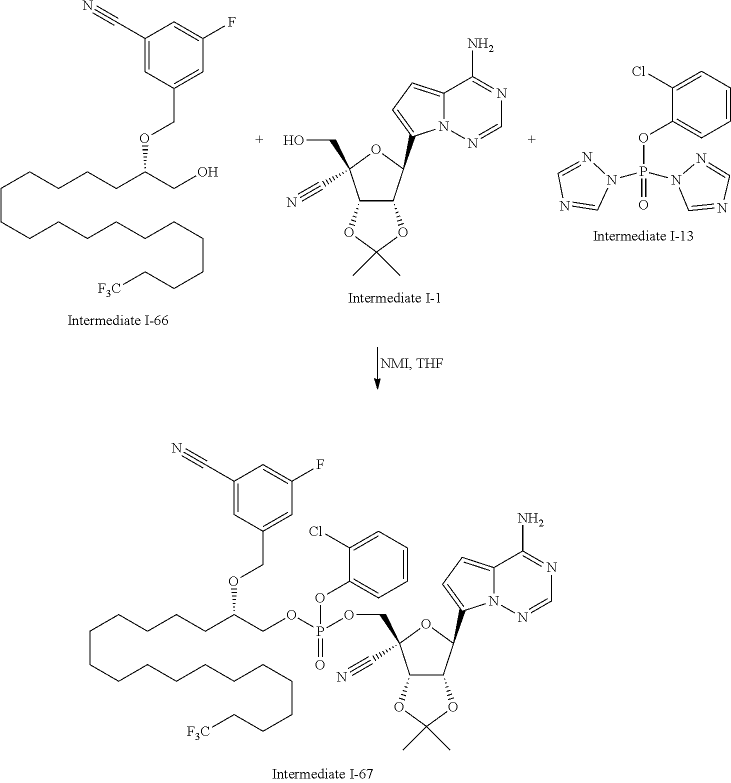 [SMILES: N#Cc1cc(F)cc(CO[C@H](CO)CCCCCCCCCCCCCCCCC(F)(F)F)c1 ; CC1(C)O[C@H]2[C@H](c3ccc4c(N)ncnn34)O[C@](C#N)(CO)[C@H]2O1 ; O=P(Oc1ccccc1Cl)(n1cncn1)n1cncn1 ; CC1(C)O[C@H]2[C@H](c3ccc4c(N)ncnn34)O[C@](C#N)(COP(=O)(OC[C@H](CCCCCCCCCCCCCCCCC(F)(F)F)OCc3cc(F)cc(C#N)c3)Oc3ccccc3Cl)[C@H]2O1]

To the solution M, 0.49 of Intermediate I-13 (1.62 mL, 0.303 mmol) were added Intermediate I-1 (125 mg, 0.377 mmol) in one portion and then 1-methylimidazole (0.0391 mL, 0.49 mmol). The reaction mixture was stirred at rt for 10 min and then Intermediate I-66 (202 mg, 0.415 mmol) was added in one portion. The resulting mixture was stirred for 1 h, diluted with EtOAc (100 mL), washed with water (50 mL), and the aqueous layer extracted with EtOAc (50 mL×3). The combined organic layer was dried under sodium sulfate, concentrated in vacuo, and purified by silica gel (0 to 5% MeOH in DCM) to give Intermediate I-67. ¹H NMR (400 MHZ, Acetonitrile-d3) δ 7.90-7.80 (m, 1H), 7.51-7.29 (m, 5H), 7.29-7.10 (m, 2H), 6.80-6.75 (m, 1H), 6.74-6.70 (m, 1H), 6.25 (s, 2H), 5.69-5.60 (m, 1H), 5.33-5.25 (m, 1H), 5.15-5.00 (m, 1H), 4.77-4.39 (m, 4H), 4.36-4.22 (m, 1H), 4.17-4.01 (m, 1H), 3.66-3.56 (m, 1H), 2.24-2.05 (m, 2H), 1.74-1.65 (m, 3H), 1.59-1.45 (m, 2H), 1.41-1.19 (m, 31H); ¹⁹F NMR (376 MHZ, Acetonitrile-d3) δ−67.52, −112.83, −112.87; 31P NMR (162 MHz, Acetonitrile-d3) δ−7.19, −7.47; MS m/z [M+1]=991.48.

### Intermediate I-68: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((S)-2-((3-cyano-5-fluorobenzyl)oxy)-19,19,19-trifluorononadecyl) hydrogen phosphate

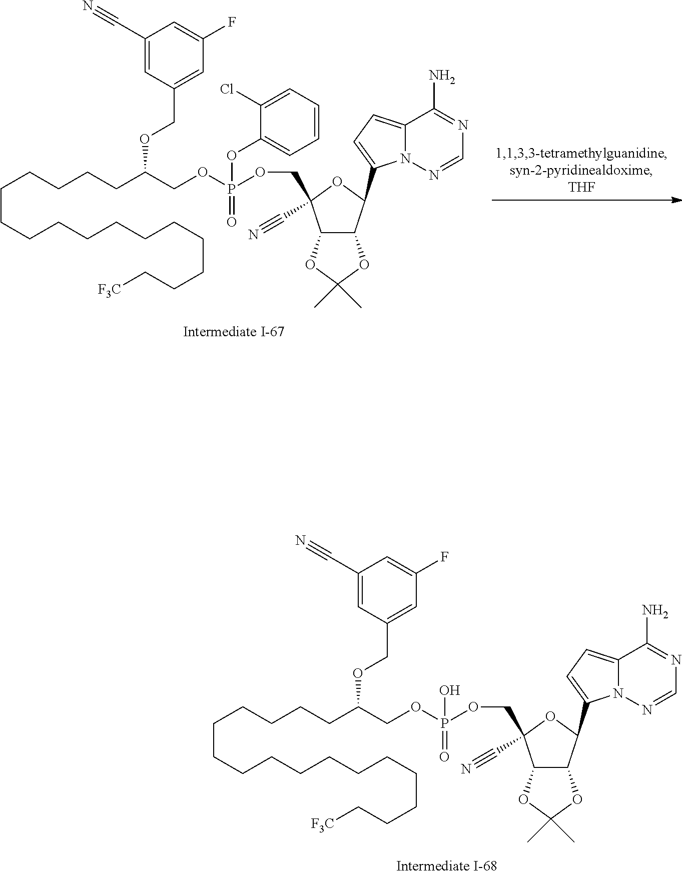 [SMILES: CC1(C)O[C@H]2[C@H](c3ccc4c(N)ncnn34)O[C@](C#N)(COP(=O)(OC[C@H](CCCCCCCCCCCCCCCCC(F)(F)F)OCc3cc(F)cc(C#N)c3)Oc3ccccc3Cl)[C@H]2O1 ; CC1(C)O[C@H]2[C@H](c3ccc4c(N)ncnn34)O[C@](C#N)(COP(=O)(O)OC[C@H](CCCCCCCCCCCCCCCCC(F)(F)F)OCc3cc(F)cc(C#N)c3)[C@H]2O1]

To a solution of Intermediate 67 (232 mg, 0.234 mmol) in THF (5 mL) were added 1,1,3,3-tetramethylguanidine (0.176 mL, 1.40 mmol) and syn-2-pyridinealdoxime (116 mg, 0.95 mmol). The reaction mixture was stirred at room temperature for 15 h, diluted with EtOAc (100 mL), washed with NH4Cl solution (30 mL×2), (add some iPrOH better separation) dried with sodium sulfate, concentrated in vacuo, and purified by silica gel (0-60% MeOH in DCM) to give Intermediate 68. ¹H NMR (400 MHZ, Methanol-d4) δ 7.88 (s, 1H), 7.52 (s, 1H), 7.43 (d, J=9.6 Hz, 1H), 7.38 (d, J=8.5 Hz, 1H), 6.93 (d, J=4.6 Hz, 1H), 6.86 (d, J=4.6 Hz, 1H), 5.66 (d, J=3.7 Hz, 1H), 5.28 (dd, J=6.6, 3.7 Hz, 1H), 5.15 (d, J=6.6 Hz, 1H), 4.76 (d, J=13.0 Hz, 1H), 4.52 (d, J=12.9 Hz, 1H), 4.24-4.04 (m, 2H), 4.02-3.82 (m, 2H), 3.65-3.54 (m, 1H), 2.29-1.99 (m, 2H), 1.70 (s, 3H), 1.60-1.50 (m, 2H), 1.51-1.20 (m, 31H); ¹⁹F NMR (376 MHZ, Methanol-d4) δ−68.57, −113.05; ³¹P NMR (162 MHZ, Methanol-d4) δ−0.42; MS m/z [M+1]=881.4.

### Intermediate I-69: (S)-2-(((20,20,20-trifluoro-1-(trityloxy)icosan-2-yl)oxy)methyl)quinoline

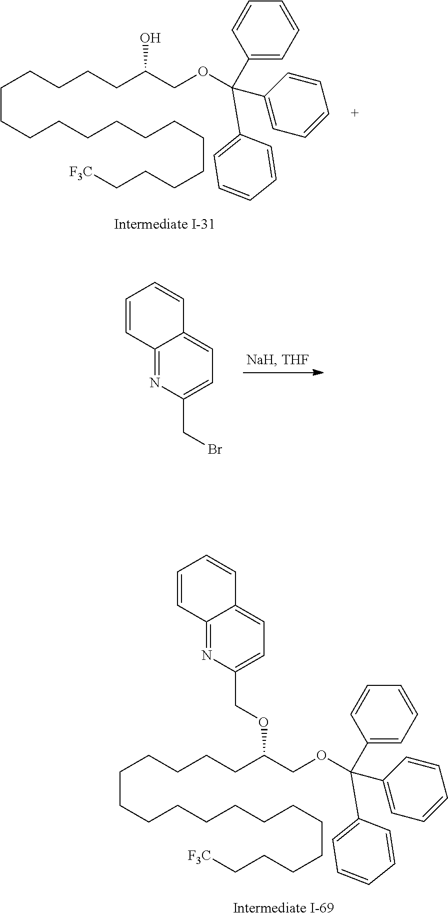 [SMILES: O[C@@H](CCCCCCCCCCCCCCCCCC(F)(F)F)COC(c1ccccc1)(c1ccccc1)c1ccccc1 ; BrCc1ccc2ccccc2n1 ; FC(F)(F)CCCCCCCCCCCCCCCCC[C@@H](COC(c1ccccc1)(c1ccccc1)c1ccccc1)OCc1ccc2ccccc2n1]

To a solution of (2S)-20,20,20-trifluoro-1-trityloxy-icosan-2-ol; 500 mg, 0.82 mmol, 1 eq) in THF (5 mL) was added NaH (78.0 mg, 2 mmol, 60% purity, 2.5 eq) at 0° C. and the mixture was stirred at 0° C. for 30 min. Then 2-(bromomethyl)quinoline (236 mg, 1.1 mmol, 1.2 eq) was added and the mixture was stirred at 65° C. for 12 hr. TLC indicated Reactant was consumed completely and many new spots were formed. The reaction mixture was quenched by addition of sat. NH₄Cl solution (60 ml) at 20° C. and extracted with Ethyl acetate (40 mL×3). The combined organic layers were washed with H₂O (60 mL×2), dried over Na₂SO₄, and concentrated under reduced pressure to give a residue. The residue was purified by flash silica gel chromatography (ISCO®; 20 g SepaFlash® Silica Flash Column. 1H NMR (400 MHZ, Chloroform-d) δ 8.13 (d, J=8.5 Hz, 1H), 8.03 (d, J=8.5 Hz, 1H), 7.80 (dd, J=8.2, 1.4 Hz, 1H), 7.76-7.66 (m, 2H), 7.51 (ddd, J=8.1, 6.8, 1.2 Hz, 1H), 7.49-7.43 (m, 5H), 7.31-7.16 (m, 10H), 4.95 (d, J=13.4 Hz, 1H), 4.85 (d, J=13.5 Hz, 1H), 3.62 (qd, J=5.9, 4.1 Hz, 1H), 3.33-3.12 (m, 2H), 2.14-1.94 (m, 2H), 1.56 (dddd, J=23.8, 15.7, 6.8, 4.3 Hz, 4H), 1.23 (dd, J=16.8, 6.4 Hz, 28H)). ¹⁹F NMR (376 MHz, Chloroform-d) 8-66.95 (t, J=11.0 Hz). MS m/z [M+1]=752.2.

### Intermediate I-70: (S)-20,20,20-trifluoro-2-(quinolin-2-ylmethoxy)icosan-1-ol

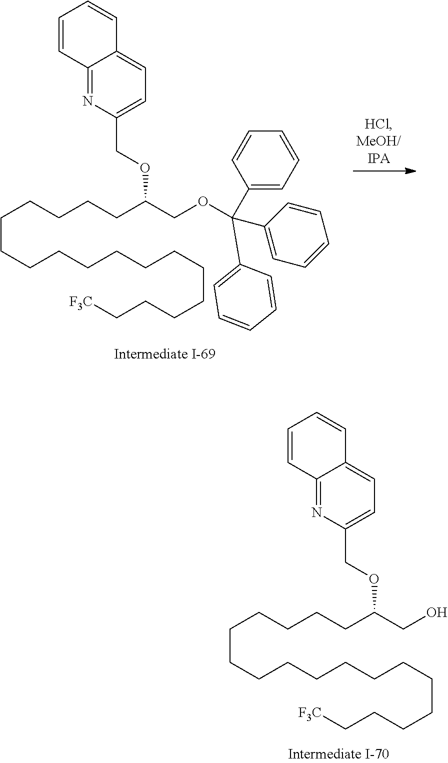 [SMILES: FC(F)(F)CCCCCCCCCCCCCCCCC[C@@H](COC(c1ccccc1)(c1ccccc1)c1ccccc1)OCc1ccc2ccccc2n1 ; OC[C@H](CCCCCCCCCCCCCCCCCC(F)(F)F)OCc1ccc2ccccc2n1]

To a solution of (S)-2-(((20,20,20-trifluoro-1-(trityloxy)icosan-2-yl)oxy)methyl)quinoline (600 mg, 0.8 mmol, 1 eq) in IPA (5 mL) and MeOH (5 mL) was added HCl (12 M, 15 mL, 57.11 eq) and the mixture was stirred at 20° C. for 2 hr. TLC indicated reactant was consumed and two new spots formed. The reaction mixture was quenched by addition sat. NaHCO₃240 mL, and then extracted with Ethyl acetate (120 mL×3). The combined organic layers were washed with H₂O (180 mL×2), dried over Na₂SO₄, filtered, and concentrated under reduced pressure to give a residue. The residue was purified by flash silica gel chromatography to yield the title compound. ¹H NMR (400 MHZ, Chloroform-d) δ 8.16 (d, J=8.5 Hz, 1H), 8.09 (d, J=8.5 Hz, 1H), 7.82 (dd, J=8.1, 1.4 Hz, 1H), 7.72 (ddd, J=8.5, 6.8, 1.5 Hz, 1H), 7.54 (ddd, J=8.1, 6.8, 1.2 Hz, 1H), 7.36 (d, J=8.5 Hz, 1H), 5.55 (s, 1H), 5.09 (d, J=15.0 Hz, 1H), 4.85 (d, J=15.0 Hz, 1H), 3.78-3.62 (m, 3H), 2.13-1.99 (m, 2H), 1.71-1.62 (m, 1H), 1.55 (ddt, J=11.6, 5.8, 2.8 Hz, 3H), 1.26 (d, J=3.9 Hz, 28H). ¹⁹F NMR (376 MHz, Chloroform-d) δ−66.95 (t, J=11.0 Hz). MS m/z [M+1]=510.2.

### Intermediate I-71: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) ((S)-20,20,20-trifluoro-2-(quinolin-2-ylmethoxy)icosyl) phosphate

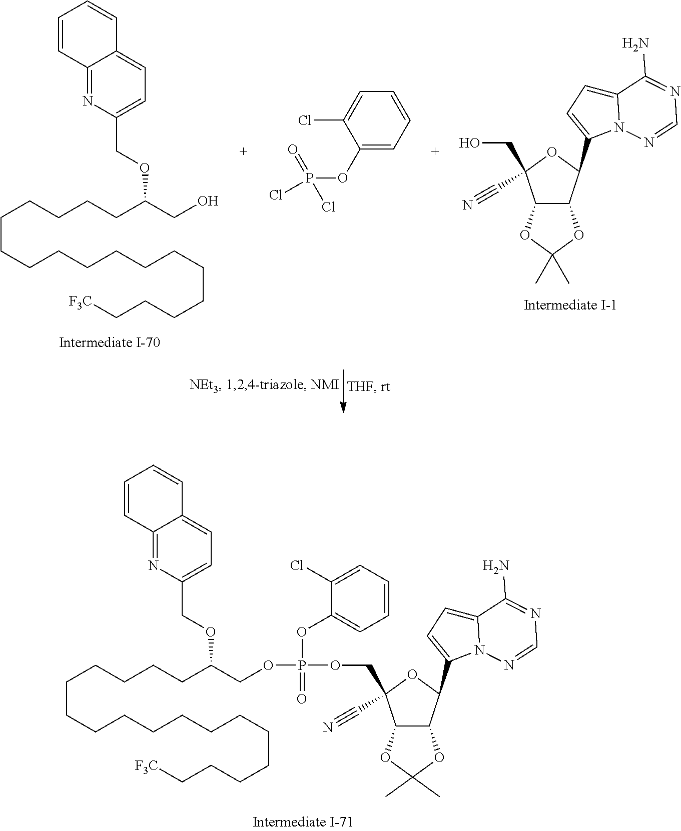 [SMILES: OC[C@H](CCCCCCCCCCCCCCCCCC(F)(F)F)OCc1ccc2ccccc2n1 ; O=P(Cl)(Cl)Oc1ccccc1Cl ; CC1(C)O[C@H]2[C@H](c3ccc4c(N)ncnn34)O[C@](C#N)(CO)[C@H]2O1 ; CC1(C)O[C@H]2[C@H](c3ccc4c(N)ncnn34)O[C@](C#N)(COP(=O)(OC[C@H](CCCCCCCCCCCCCCCCCC(F)(F)F)OCc3ccc4ccccc4n3)Oc3ccccc3Cl)[C@H]2O1]

1H-1,2,4-triazole (106 mg, 1.54 mmol, 3.0 equiv.) was dissolved in THF (10.0 mL). TEA (0.22 mL, 1.54 mmol, 3.0 equiv.) was added to the solution followed by 2-chlorophenyl phosphorodichloridate (0.12 mL, 0.72 mmol, 1.40 equiv.). The reaction mixture was stirred at rt for 6 min prior to the addition of ((3aS,4R,6aS)-6-(4-aminopyrrolo[2,1-f][1,2,4]triazin-7-yl)-4-(fluoromethyl)-2,2-dimethyltetrahydrofuro[3,4-d][1,3]dioxol-4-yl)methanol (170 mg, 0.513 mmol, 1.00 equiv.) in one portion followed by 1-methylimidazole (0.12 mL, 1.54 mmol, 3.0 equiv.). The solution was stirred for an additional 10 min before adding (S)-20,20,20-trifluoro-2-(quinolin-2-ylmethoxy)icosan-1-ol (260 mg, 0.510 mmol, 1.00 equiv.). After stirring at room temperature overnight, the solution was diluted with EtOAc (100 mL) and water (100 mL). The organic layer was washed with brine (50 mL), dried over Na₂SO₄, filtered and concentrated in vacuo prior to purification by silica gel chromatography (0-100% EtOAc in hexanes) to afford the title compound. 1H NMR (400 MHZ, Methanol-d4) δ 8.28-8.21 (m, 1H), 8.00-7.94 (m, 1H), 7.90-7.84 (m, 1H), 7.80-7.70 (m, 2H), 7.68-7.62 (m, 1H), 7.60-7.53 (m, 1H), 7.43-7.28 (m, 2H), 7.15-7.00 (m, 2H), 6.87-6.70 (m, 2H), 5.66-5.60 (m, 1H), 5.30-5.23 (m, 1H), 5.16-5.08 (m, 1H), 4.84-4.78 (m, 2H), 4.61-4.48 (m, 2H), 4.41-4.32 (m, 1H), 4.28-4.14 (m, 1H), 3.78-3.67 (m, 1H), 2.19-2.03 (m, 2H), 1.74-1.66 (m, 3H), 1.65-1.15 (m, 35H). 19F NMR (376 MHz, Methanol-d4) δ−68.54 (t, J=11.3 Hz). MS m/z [M+1]=1013.8.

### Intermediate I-72: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((S)-20,20,20-trifluoro-2-(quinolin-2-ylmethoxy)icosyl) hydrogen phosphate

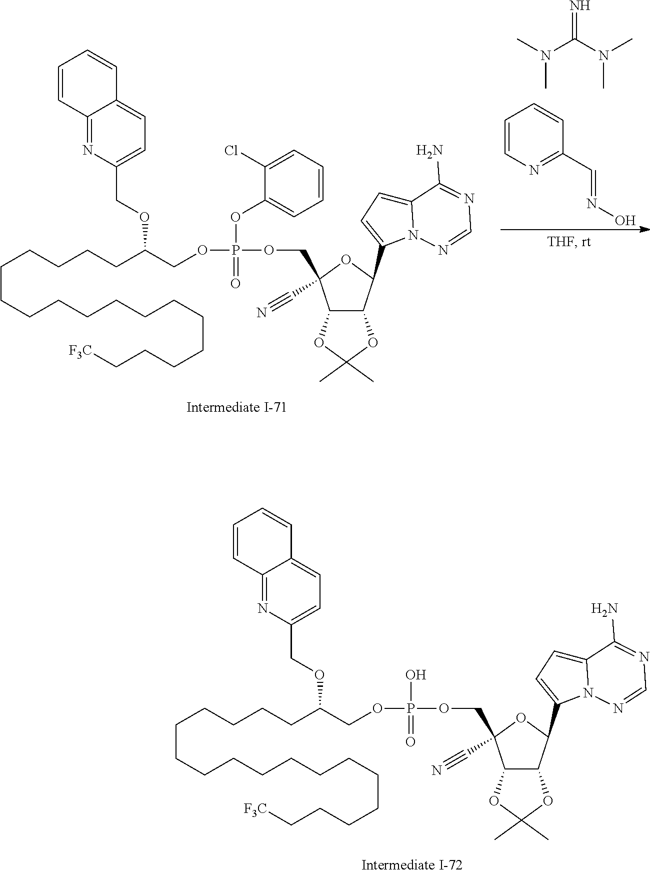

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) ((S)-20,20,20-trifluoro-2-(quinolin-2-ylmethoxy)icosyl) phosphate (233 mg, 0.230 mmol, 1.0 equiv.) in THF (10.0 mL) was added 1,1,3,3-tetramethylguanidine (0.29 mL, 2.30 mmol, 10.0 equiv.) and syn-2-pyridinealdoxime (281 mg, 2.30 mmol, 10.0 equiv.). The reaction mixture was stirred at room temperature overnight. The solution was concentrated in vacuo and purified by silica gel (0-40% MeOH in DCM) to afford the title compound which was used immediately in the next reaction. MS m/z [M+1]=903.4.

### Intermediate I-73: octadecane-1,18-diol

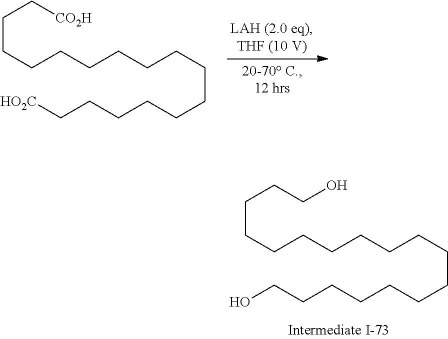 [SMILES: O=C(O)CCCCCCCCCCCCCCCCC(=O)O ; OCCCCCCCCCCCCCCCCCCO]

Two batches of the reaction were run in parallel. LAH (2.50 M, 254 mL, 2.00 eq.) was added into THF (1.00 L) at 20° C. Octadecanedioic acid (100 g, 318.01 mmol, 1.00 eq.) was added to the solution at 70° C. The reaction mixture was stirred at 70° C. for 12 hr. The two reactions were combined for work-up. Na₂SO₄·10 H₂O (100 g) was added at 0° C. The mixture was filtered through a pad of Celite or silica gel and the pad or filter cake was washed with THF (3.00 L×2). The filtrates were concentrated to dryness to give product (80.0 g, 279 mmol, 43.9% yield) as white solid. 1H NMR (400 MHZ, CDCl₃-d): δ 3.70-3.60 (m, 4H), 1.65-1.51 (m, 5H), 1.39-1.24 (m, 28H).

### Intermediate I-74: 18-iodooctadecan-1-ol

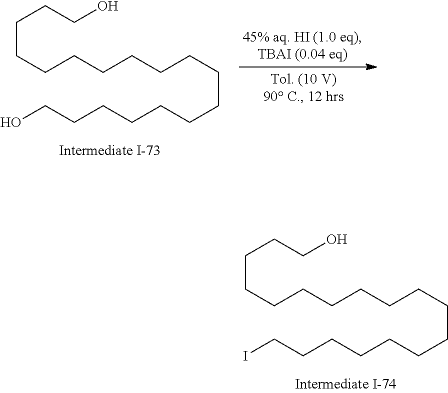 [SMILES: OCCCCCCCCCCCCCCCCCCO ; OCCCCCCCCCCCCCCCCCCI]

Two batches of the reaction were run in parallel. Octadecane-1,18-diol (50.0 g, 174 mmol, 1.00 eq.) was added into toluene (500 mL). HI (49.6 g, 174 mmol, 29.1 mL, 45% purity, 1.00 eq.) and TBAI (2.58 g, 6.98 mmol, 0.04 eq.) were subsequently added. The reaction mixture was stirred at 90° C. for 12 hr. The two reactions were combined for work-up. The mixture was filtered and the filtrate was dissolved in EtOAc (1.00 L) and washed with water (1.00 L×2). The organic phase was concentrated by vacuum and purified by column chromatography (SiO₂, Petroleum ether/Ethyl acetate=100/1 to 0/1) to afford the title compound (40.0 g, 100 mmol, 28.9% yield) as light yellow solid. 1H NMR (400 MHZ, CDCl₃-d) δ 3.65 (t, J=6.6 Hz, 2H), 3.19 (t, J=7.0 Hz, 2H), 1.82 (quin, J=7.2 Hz, 2H), 1.57 (quin, J=6.8 Hz, 2H), 1.41-1.21 (m, 32H).

### Intermediate I-75: 2-((18-iodooctadecyl)oxy)tetrahydro-2H-pyran

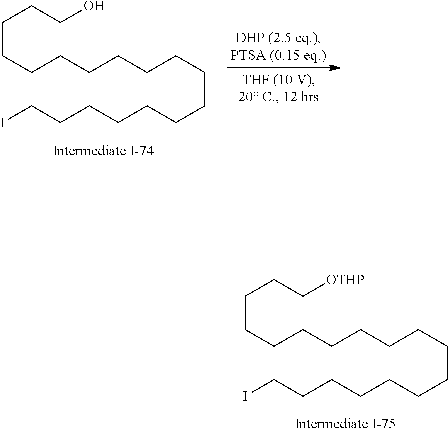

Two batches of the reaction were run in parallel. 18-iodooctadecan-1-ol (20.0 g, 50.4 mmol, 1.00 eq.) was added into THF (200 mL). DHP (10.6 g, 126 mmol, 11.5 mL, 2.50 eq.) and TsOH H₂O (1.44 g, 7.57 mmol, 0.15 eq.) were subsequently added. The reaction mixture was stirred at 20° C. for 12 hr. The two reactions were combined for work-up. The mixture was concentrated, diluted with EtOAc (200 mL) and washed by water (200 mL). The organic layer was concentrated, triturated by MeOH (200 mL) at 0° C. for 0.5 hr and then filtered to obtain the title compound (40.0 g, 83.2 mmol, 82.4% yield) as white solid. 1H NMR (400 MHZ, CDCl₃-d) δ 4.61-4.56 (m, 1H), 3.88 (ddd, J=12.0, 7.6, 3.4 Hz, 1H), 3.74 (dt, J=9.6, 6.8 Hz, 1H), 3.54-3.50 (m, 1H), 3.39 (dt, J=9.6, 6.6 Hz, 1H), 3.20 (t, J=7.0 Hz, 2H), 1.90-1.77 (m, 3H), 1.76-1.68 (m, 1H), 1.64-1.49 (m, 7H), 1.43-1.23 (m, 30H).

### Intermediate I-76: 2-((19,19,19-trifluorononadecyl)oxy)tetrahydro-2H-pyran

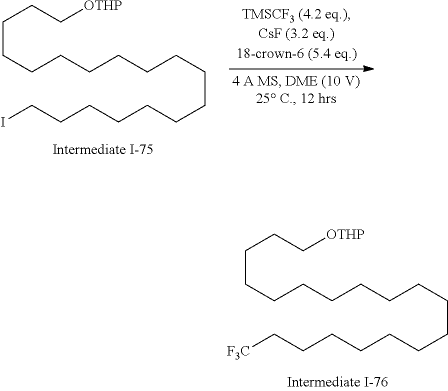

Two batches of the reaction were run in parallel. CsF (20.2 g, 133 mmol, 4.92 mL, 3.20 eq.), 4A MS (20.0 g) and 18-crown-6 (59.4 g, 224 mmol, 5.40 eq.) were added into DME (150 mL). A solution of TMSCF₃ (24.8 g, 174 mmol, 4.20 eq.) and 2-((18-iodooctadecyl)oxy)tetrahydro-2H-pyran (20.0 g, 41.6 mmol, 1 eq.) in DME (50.0 mL) was subsequently added. The reaction mixture was stirred at 25° C. for 12 hr. The two reactions were combined for work-up. The mixture was filtered and the filtrate was poured into water (300 mL) and extracted with EtOAc (300 mL×2). The organic phase was concentrated by vacuum and triturated by MeOH (150 mL) at 0° C. for 1 hr to obtain the title compound (30.0 g, 70.9 mmol, 85.2% yield) as yellow solid. 1H NMR (400 MHZ, CDCl₃-d) δ 4.62-4.55 (m, 1H), 3.92-3.84 (m, 1H), 3.74 (dt, J=9.6, 6.8 Hz, 1H), 3.55-3.47 (m, 1H), 3.39 (dt, J=9.6, 6.6 Hz, 1H), 2.14-1.99 (m, 2H), 1.91-1.78 (m, 1H), 1.77-1.67 (m, 1H), 1.65-1.48 (m, 9H), 1.43-1.20 (m, 30H). ¹⁹F NMR (376 MHz, CDCl₃-d) δ=−66.418 ppm.

### Intermediate I-77: 19,19,19-trifluorononadecan-1-ol

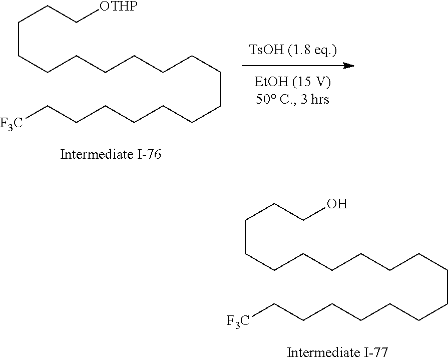

Two batches of the reaction were run in parallel. 2-((19,19,19-trifluorononadecyl)oxy)tetrahydro-2H-pyran (15.0 g, 35.4 mmol, 1.00 eq.) was added into EtOH (225 mL). TsOH H₂O (12.1 g, 63.8 mmol, 1.80 eq.) was subsequently added. The mixture was stirred it at 50° C. for 3 hr. The two reactions were combined for work-up. The mixture was concentrated by vacuum and poured into H₂O (200 mL). The aqueous layer was extracted with EtOAc (200 mL×2). The organic layer was concentrated under reduced pressure and triturated by MeOH (150 mL) at 0° C. for 30 min to afford the title compound (20.0 g, 59.0 mmol, 83.2% yield) as white solid. TLC Information (Eluent: Petroleum ether/Ethyl acetate=3/1) R_{f}=0.40.

### Intermediate I-78: 19-bromo-1,1,1-trifluorononadecane

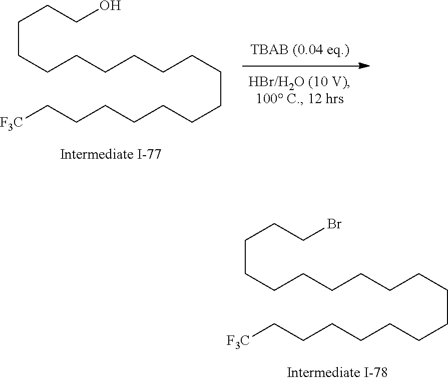 [SMILES: OCCCCCCCCCCCCCCCCCCC(F)(F)F ; FC(F)(F)CCCCCCCCCCCCCCCCCCBr]

Two batches of the reaction were run in parallel. TBAB (285 mg, 886 umol, 0.04 eq) was added into HBr (111 g, 552 mmol, 75.0 mL, 40.0% purity, 24.9 eq.). 19,19,19-trifluorononadecan-1-ol (7.50 g, 22.1 mmol, 1.00 eq.) was subsequently added. The reaction mixture was stirred at 100° C. for 12 hr. The two reactions were combined for work-up. The mixture was poured into EtOAc (50.0 mL) and the organic layer was concentrated under reduced pressure and triturated with MeOH (50.0 mL) at 15° C. for 0.5 hr to afford the title compound (10.0 g, 24.9 mmol, 56.2% yield) as white solid. TLC Information (Eluent: Petroleum ether/Ethyl acetate=5/1) R_{f}=0.65.

### Intermediate I-79: (19,19,19-trifluorononadecyl)magnesium bromide

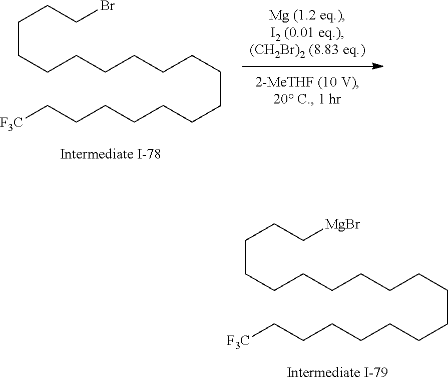

Mg (726 mg, 29.9 mmol, 1.20 eq.) was added into 2-MeTHF (60.0 mL) under N₂ at 20° C. I₂ (63.2 mg, 249 umol, 50.1 μL, 0.01 eq.) and (CH₂Br)₂ (41.3 g, 220 mmol, 16.6 mL, 8.83 eq.) were subsequently added. A solution of 19-bromo-1,1,1-trifluorononadecane (10.0 g, 24.9 mmol, 1.00 eq.) in 2-MeTHF (40.0 mL) was added under N₂ at 20° C. The reaction mixture was stirred for 1 h and was used immediately in the next step. The title compound was obtained (10.0 g in 2-MeTHF, 23.4 mmol, 94.29% yield) as a brown liquid.

### Intermediate I-80: (R)-22,22,22-trifluoro-1-(trityloxy)docosan-2-ol

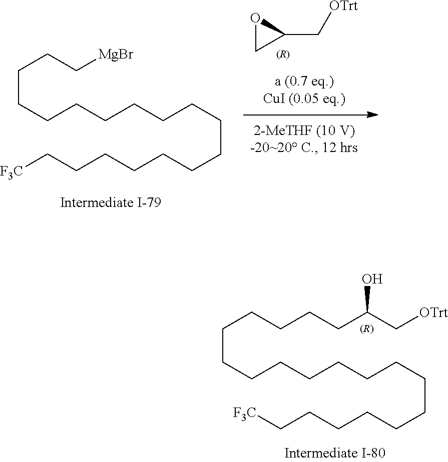

(R)-2-((trityloxy)methyl)oxirane (5.20 g, 16.4 mmol, 0.70 eq.) and CuI (223 mg, 1.17 mmol, 0.05 eq.) were added into 2-MeTHF (100 mL) at 20° C. under N₂. (19,19,19-trifluorononadecyl)magnesium bromide (10.0 g, 23.4 mmol, 1.00 eq.) was subsequently added at −20° C. The reaction mixture was stirred at 20° C. for 12 hr. The mixture was quenched with ice water (200 mL) and extracted with EtOAc (200 mL×2). The organic layer was concentrated under reduced pressure and triturated with MeOH (20.0 mL) at 20° C. for 1 hr to afford the title compound (8.00 g, 12.5 mmol, 53.3% yield) as a white solid. 1H NMR (400 MHZ, CDCl₃-d) δ 7.48 (br d, J=7.8 Hz, 5H), 7.39-7.27 (m, 8H), 3.80 (br d, J=3.2 Hz, 1H), 3.53 (br s, 1H), 3.21 (dd, J=9.2, 2.8 Hz, 1H), 3.11-2.99 (m, 1H), 2.35 (d, J=3.2 Hz, 1H), 2.18-2.00 (m, 2H), 1.58 (dt, J=15.0, 7.6 Hz, 2H), 1.51-1.17 (m, 30H).

### Intermediate I-81: (R)-3-fluoro-5-(((22,22,22-trifluoro-1-(trityloxy)docosan-2-yl)oxy)methyl)benzonitrile

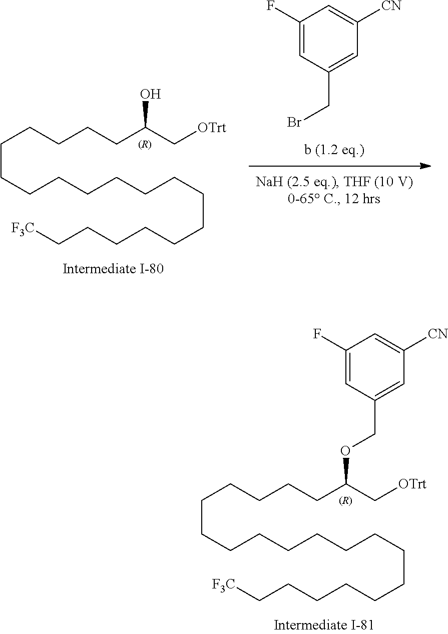

Two batches of the reaction were run in parallel. (R)-22,22,22-trifluoro-1-(trityloxy)docosan-2-ol (4.00 g, 6.26 mmol, 1.00 eq.) was dissolved in THF (40.0 mL) at 20° C. NaH (626 mg, 15.6 mmol, 60.0% purity, 2.50 eq.) was added at 0° C. 3-(bromomethyl)-5-fluorobenzonitrile (1.34 g, 6.26 mmol, 1.00 eq.) was subsequently added at 0° C. The reaction mixture was stirred at 65° C. for 12 hr. The reaction was quenched by NH₄Cl (30.0 mL, 100%) and extracted with EtOAc (30.0 mL×2). The organic layer was concentrated and purified by column chromatography (SiO₂, Petroleum ether/Ethyl acetate=I/O to 10/1) to afford the title compound (5.00 g, 6.48 mmol, 51.7% yield) as a white solid. 1H NMR (400 MHZ, CDCl₃-d) ô 7.48-7.38 (m, 7H), 7.36-7.20 (m, 12H), 4.71 (d, J=12 Hz, 1H), 4.55 (d, J=12 Hz, 1H), 3.57-3.46 (m, 1H), 3.20 (d, J=4.8 Hz, 2H), 2.12-1.96 (m, 2H), 1.60-1.46 (m, 5H), 1.39-1.11 (m, 32H).

### Intermediate I-82: (R)-3-fluoro-5-(((22,22,22-trifluoro-1-hydroxydocosan-2-yl)oxy)methyl)benzonitrile

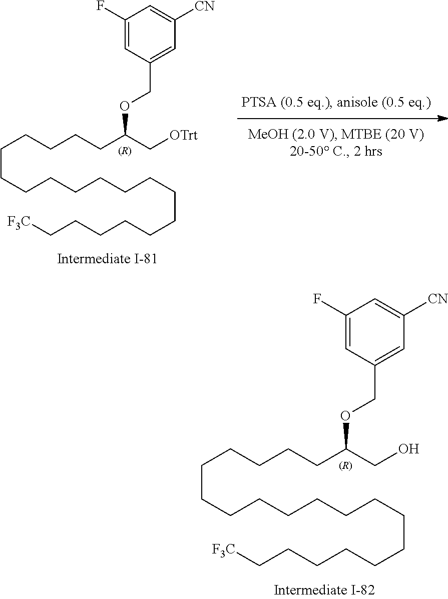

Two batches of the reaction were run in parallel. (R)-3-fluoro-5-(((22,22,22-trifluoro-1-(trityloxy)docosan-2-yl)oxy)methyl)benzonitrile (2.50 g, 3.24 mmol, 1.00 eq.), TsOH H₂O (307 mg, 1.62 mmol, 0.500 eq.) and anisole (175 mg, 1.62 mmol, 175 μL, 0.500 eq.) were added into MeOH (5.00 mL) and MTBE (50.0 mL) at 20° C. The solution was stirred at 50° C. for 2 hr. The two batches were combined for workup. The reaction mixture was poured into NaHCO₃ (100 mL, 100%). The organic layer was concentrated and purified by column chromatography (SiO₂, Petroleum ether/Ethyl acetate=I/O to 10/1) to afford the title compound (2.00 g, 3.61 mmol, 55.7% yield, 95.7% purity) as a white solid. 1H NMR (400 MHZ, CDCl₃-d) ô 7.46 (s, 1H), 7.35 (br d, J=9.0 Hz, 1H), 7.31-7.27 (m, 1H), 4.64 (s, 2H), 3.81-3.70 (m, 1H), 3.62 (dt, J=12, 5.6 Hz, 1H), 3.57-3.46 (m, 1H), 2.15-1.98 (m, 2H), 1.79 (br t, J=5.8 Hz, 1H), 1.67-1.48 (m, 5H), 1.40-1.23 (m, 33H). ¹⁹F NMR (376 MHz, CDCl₃-d) ô=−66.4, −109 ppm.

### Intermediate I-83: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) ((R)-2-((3-cyano-5-fluorobenzyl)oxy)-22,22,22-trifluorodocosyl) phosphate

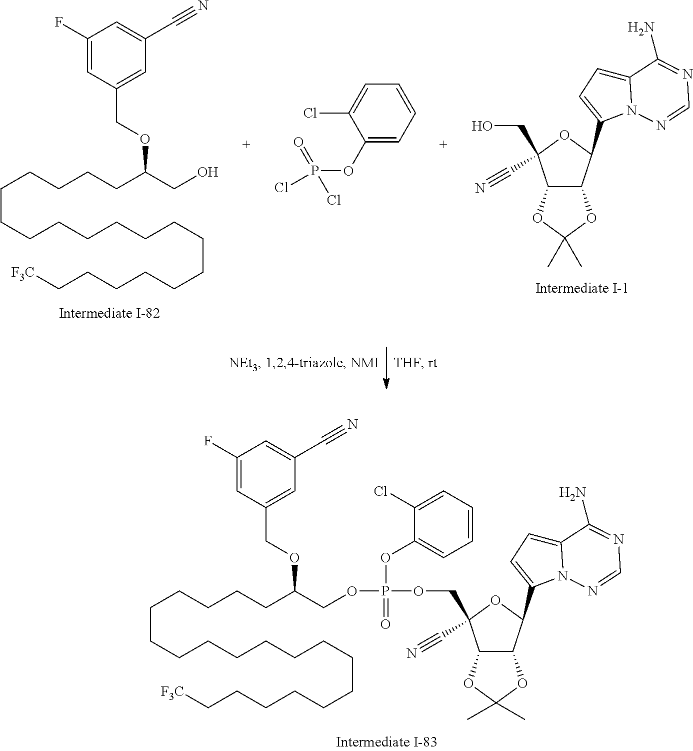 [SMILES: N#Cc1cc(F)cc(CO[C@@H](CO)CCCCCCCCCCCCCCCCCCCC(F)(F)F)c1 ; O=P(Cl)(Cl)Oc1ccccc1Cl ; CC1(C)O[C@H]2[C@H](c3ccc4c(N)ncnn34)O[C@](C#N)(CO)[C@H]2O1 ; CC1(C)O[C@H]2[C@H](c3ccc4c(N)ncnn34)O[C@](C#N)(COP(=O)(OC[C@@H](CCCCCCCCCCCCCCCCCCCC(F)(F)F)OCc3cc(F)cc(C#N)c3)Oc3ccccc3Cl)[C@H]2O1]

1H-1,2,4-triazole (149 mg, 2.16 mmol, 4.2 equiv.) was dissolved in THF (10.0 mL). TEA (0.22 mL, 1.54 mmol, 3.0 equiv.) was added to the solution followed by 2-chlorophenyl phosphorodichloridate (0.12 mL, 0.72 mmol, 1.40 equiv.). The reaction mixture was stirred at rt for 6 min prior to the addition of ((3aS,4R,6aS)-6-(4-aminopyrrolo[2,1-f][1,2,4]triazin-7-yl)-4-(fluoromethyl)-2,2-dimethyltetrahydrofuro[3,4-d][1,3]dioxol-4-yl)methanol (170 mg, 0.513 mmol, 1.00 equiv.) in one portion followed by 1-methylimidazole (0.12 mL, 1.54 mmol, 3.0 equiv.). The solution was stirred for an additional 10 min before adding (R)-3-fluoro-5-(((22,22,22-trifluoro-1-hydroxydocosan-2-yl)oxy)methyl)benzonitrile (270 mg, 0.510 mmol, 1.0 equiv.). After stirring at room temperature overnight, the solution was diluted with EtOAc (100 mL) and water (100 mL). The organic layer was washed with brine (50 mL), dried over Na₂SO₄, filtered and concentrated in vacuo prior to purification by silica gel chromatography (0-100% EtOAc in hexanes) to afford the title compound (290 mg, 0.280 mmol, 55% yield). 1H NMR (400 MHz, Methanol-d4) δ 7.81-7.77 (m, 1H), 7.47-7.29 (m, 5H), 7.20-7.07 (m, 2H), 6.85-6.79 (m, 1H), 6.78-6.74 (m, 1H), 5.68-5.62 (m, 1H), 5.33-5.27 (m, 1H), 5.18-5.11 (m, 1H), 4.64-4.43 (m, 4H), 4.37-4.24 (m, 1H), 4.24-4.07 (m, 1H), 3.64-3.55 (m, 1H), 2.20-2.04 (m, 2H), 1.72 (s, 3H), 1.60-1.20 (m, 39H). ¹⁹F NMR (376 MHz, Methanol-d4) δ−68.55 (t, J=11.6 Hz), −112.69-−112.79 (m). 31P NMR (162 MHZ, Methanol-d4) δ−7.50-−8.00 (m). MS m/z [M+1]=1033.5.

### Intermediate I-84: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((R)-2-((3-cyano-5-fluorobenzyl)oxy)-22,22,22-trifluorodocosyl) hydrogen phosphate

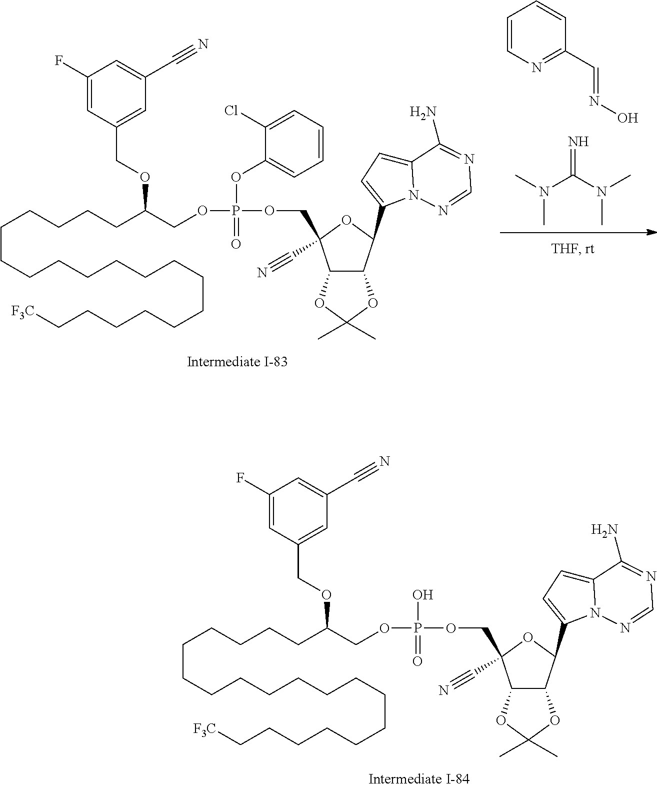

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) ((R)-2-((3-cyano-5-fluorobenzyl)oxy)-22,22,22-trifluorodocosyl) phosphate (285 mg, 0.276 mmol, 1.0 equiv.) in THF (10.0 mL) was added 1,1,3,3-tetramethylguanidine (0.35 mL, 2.76 mmol, 10.0 equiv.) and syn-2-pyridinealdoxime (337 mg, 2.76 mmol, 10.0 equiv.). The reaction mixture was stirred at room temperature overnight. The solution was concentrated in vacuo and purified by silica gel (0-40% MeOH in DCM) to afford the title compound (239 mg, 94% yield). 1H NMR (400 MHZ, Methanol-d4) δ 7.82 (s, 1H), 7.51-7.47 (m, 1H), 7.43-7.33 (m, 2H), 6.83 (d, J=4.5 Hz, 1H), 6.78 (d, J=4.5 Hz, 1H), 5.63 (d, J=3.6 Hz, 1H), 5.26 (dd, J=6.6, 3.6 Hz, 1H), 5.13 (d, J=6.6 Hz, 1H), 4.73 (d, J=13.0 Hz, 1H), 4.51 (d, J=13.0 Hz, 1H), 4.17-4.06 (m, 2H), 3.95-3.81 (m, 2H), 3.58-3.50 (m, 1H), 2.20-2.04 (m, 2H), 1.69 (s, 3H), 1.59-1.48 (m, 2H), 1.48-1.20 (m, 37H). ¹⁹F NMR (376 MHz, Methanol-d4) δ−68.56 (t, J=11.3 Hz), −113.00 (t, J=9.2 Hz). 31P NMR (162 MHz, Methanol-d₄) δ 0.05-−0.91 (m). MS m/z [M+1]=923.3.

### Intermediate I-85: (S)-3-methoxy-4-(((20,20,20-trifluoro-1-(trityloxy)icosan-2-yl)oxy)methyl)benzonitrile

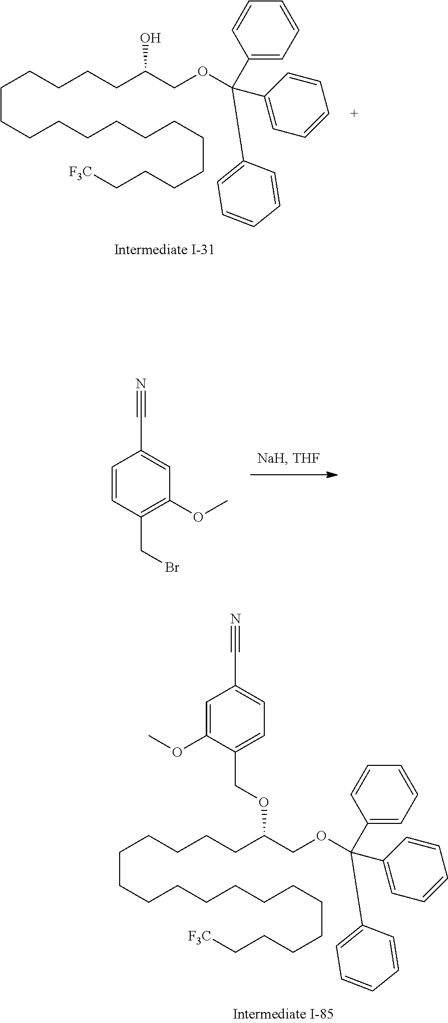 [SMILES: O[C@@H](CCCCCCCCCCCCCCCCCC(F)(F)F)COC(c1ccccc1)(c1ccccc1)c1ccccc1 ; COc1cc(C#N)ccc1CBr ; COc1cc(C#N)ccc1CO[C@@H](CCCCCCCCCCCCCCCCCC(F)(F)F)COC(c1ccccc1)(c1ccccc1)c1ccccc1]

To a solution of (2S)-20,20,20-trifluoro-1-trityloxy-icosan-2-ol; 500 mg, 0.82 mmol, 1 eq) in THF (5 mL) was added NaH (78.0 mg, 2 mmol, 60% purity, 2.5 eq) at 0° C. and the mixture was stirred at 0° C. for 30 min. Then 4-(bromomethyl)-3-methoxy-benzonitrile 241 mg, 1.1 mmol, 1.3 eq) was added and the mixture was stirred at 65° C. for 12 hr. TLC indicated Reactant was consumed completely and many new spots were formed. The reaction mixture was quenched by addition of sat. NH4Cl solution (60 ml) at 20° C. and extracted with Ethyl acetate (40 mL×3). The combined organic layers were washed with H₂O (60 mL×2), dried over Na₂SO₄, and concentrated under reduced pressure to give a residue. The residue was purified by flash silica gel chromatography (ISCO®; 20 g SepaFlash® Silica Flash Column. ¹H NMR (400 MHZ, Chloroform-d) δ 7.61 (d, J=7.7 Hz, 1H), 7.44 (tt, J=6.0, 1.4 Hz, 7H), 7.36-7.21 (m, 14H), 7.05 (d, J=1.4 Hz, 1H), 4.80 (d, J=14.2 Hz, 1H), 4.63 (d, J=14.3 Hz, 1H), 3.83 (s, 3H), 3.63-3.48 (m, 1H), 3.25-3.11 (m, 2H), 2.13-1.98 (m, 3H), 1.24 (q, J=6.8, 6.0 Hz, 32H). ¹⁹F NMR (376 MHz, Chloroform-d) δ-66.95 (t, J=11.0 Hz). MS m/z [M+1]=756.2.

### Intermediate I-86: (S)-3-methoxy-4-(((20,20,20-trifluoro-1-hydroxyicosan-2-yl)oxy)methyl)benzonitrile

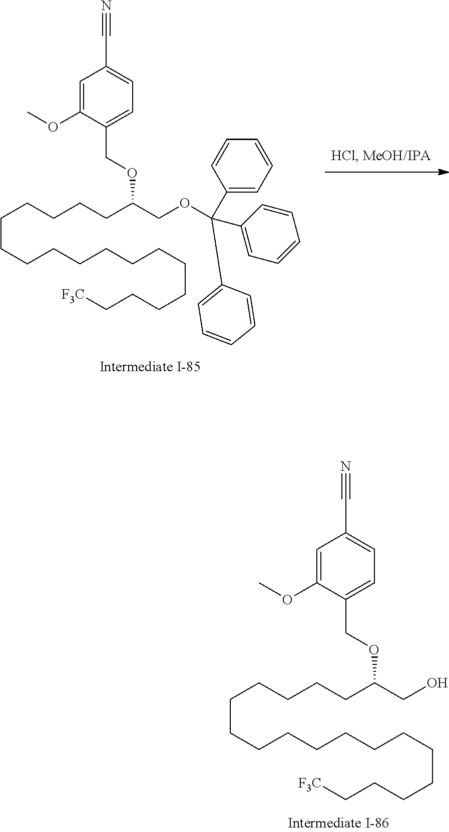 [SMILES: COc1cc(C#N)ccc1CO[C@@H](CCCCCCCCCCCCCCCCCC(F)(F)F)COC(c1ccccc1)(c1ccccc1)c1ccccc1 ; COc1cc(C#N)ccc1CO[C@H](CO)CCCCCCCCCCCCCCCCCC(F)(F)F]

To a solution of (S)-3-methoxy-4-(((20,20,20-trifluoro-1-(trityloxy)icosan-2-yl)oxy)methyl)benzonitrile(600 mg, 0.8 mmol, 1 eq) in IPA (5 mL) and MeOH (5 mL) was added HCl (12 M, 15 mL, 57.11 eq) and the mixture was stirred at 20° C. for 2 hr. TLC indicated reactant was consumed and two new spots formed. The reaction mixture was quenched by addition sat. NaHCO₃240 mL, and then extracted with Ethyl acetate (120 mL×3). The combined organic layers were washed with H₂O (180 mL×2), dried over Na₂SO₄, filtered, and concentrated under reduced pressure to give a residue. The residue was purified by flash silica gel chromatography to yield the title compound. 1H NMR (400 MHZ, Chloroform-d) δ 7.50 (d, J=7.7 Hz, 1H), 7.28 (dd, J=7.8, 1.6 Hz, 1H), 7.09 (d, J=1.6 Hz, 1H), 4.63 (q, J=12.8 Hz, 2H), 3.88 (d, J=1.5 Hz, 3H), 3.72 (q, J=7.2, 6.2 Hz, 1H), 3.54 (q, J=4.5 Hz, 2H), 2.16 (dd, J=7.5, 4.0 Hz, 1H), 2.13-1.98 (m, 2H), 1.56 (tt, J=15.9, 6.4 Hz, 4H), 1.26 (d, J=4.9 Hz, 27H). ¹⁹F NMR (376 MHz, Chloroform-d) δ-66.95 (t, J=11.0 Hz). MS m/z [M+1]=513.9.

### Intermediate I-87: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) ((S)-2-((4-cyano-2-methoxybenzyl)oxy)-20,20,20-trifluoroicosyl) phosphate

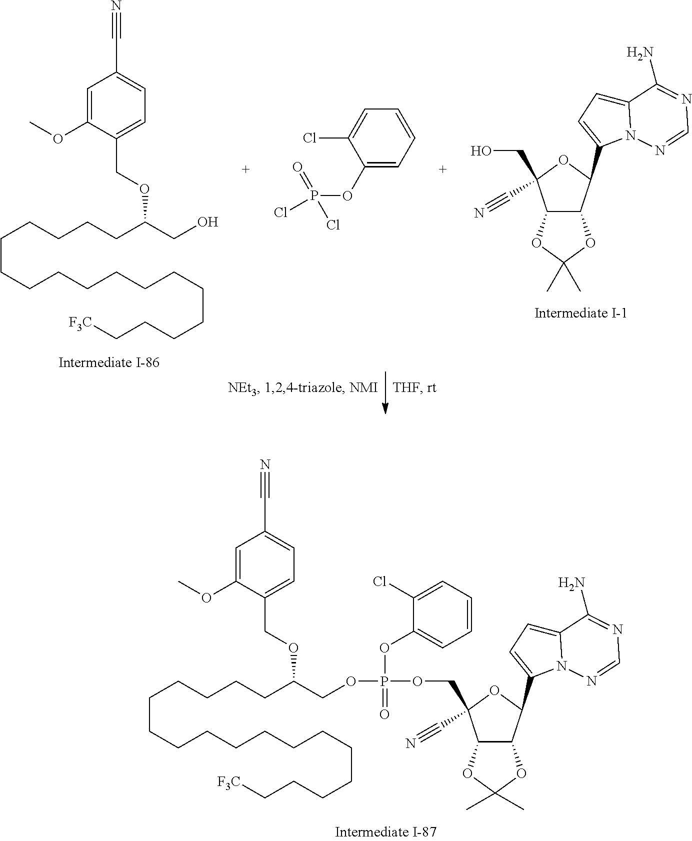 [SMILES: COc1cc(C#N)ccc1CO[C@H](CO)CCCCCCCCCCCCCCCCCC(F)(F)F ; O=P(Cl)(Cl)Oc1ccccc1Cl ; CC1(C)O[C@H]2[C@H](c3ccc4c(N)ncnn34)O[C@](C#N)(CO)[C@H]2O1 ; COc1cc(C#N)ccc1CO[C@@H](CCCCCCCCCCCCCCCCCC(F)(F)F)COP(=O)(OC[C@@]1(C#N)O[C@@H](c2ccc3c(N)ncnn23)[C@@H]2OC(C)(C)O[C@@H]21)Oc1ccccc1Cl]

1H-1,2,4-triazole (69 mg, 0.996 mmol, 3.0 equiv.) was dissolved in THF (10.0 mL). TEA (0.14 mL, 0.996 mmol, 3.0 equiv.) was added to the solution followed by 2-chlorophenyl phosphorodichloridate (0.08 mL, 0.465 mmol, 1.40 equiv.). The reaction mixture was stirred at rt for 6 min prior to the addition of ((3aS,4R,6aS)-6-(4-aminopyrrolo[2,1-f][1,2,4]triazin-7-yl)-4-(fluoromethyl)-2,2-dimethyltetrahydrofuro[3,4-d][1,3]dioxol-4-yl)methanol (110 mg, 0.332 mmol, 1.00 equiv.) in one portion followed by 1-methylimidazole (0.08 mL, 0.996 mmol, 3.0 equiv.). The solution was stirred for an additional 10 min before adding (S)-3-methoxy-4-(((20,20,20-trifluoro-1-hydroxyicosan-2-yl)oxy)methyl)benzonitrile (171 mg, 0.332 mmol, 1.00 equiv.). After stirring at room temperature overnight, the solution was diluted with EtOAc (100 mL) and water (100 mL). The organic layer was washed with brine (50 mL), dried over Na₂SO₄, filtered and concentrated in vacuo prior to purification by silica gel chromatography (0-100% EtOAc in hexanes) to afford the title compound (123 mg, 0.121 mmol, 36% yield). 1H NMR (400 MHz, Methanol-d4) δ 7.83-7.74 (m, 1H), 7.54-7.46 (m, 1H), 7.46-7.37 (m, 1H), 7.36-7.28 (m, 1H), 7.26-7.19 (m, 2H), 7.19-7.03 (m, 2H), 6.86-6.79 (m, 1H), 6.78-6.73 (m, 1H), 5.66-5.60 (m, 1H), 5.33-5.24 (m, 1H), 5.17-5.05 (m, 1H), 4.67-4.45 (m, 4H), 4.36-4.25 (m, 1H), 4.21-4.06 (m, 1H), 3.88-3.79 (m, 3H), 3.69-3.56 (m, 1H), 2.19-2.04 (m, 2H), 1.72 (s, 3H), 1.60-1.16 (m, 35H). ¹⁹F NMR (376 MHz, Methanol-d4) δ−68.55 (t, J=11.2 Hz). 31P NMR (162 MHZ, Methanol-d4) δ−7.49-−8.33 (m). MS m/z [M+1]=1017.4.

### Intermediate I-88: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((S)-2-((4-cyano-2-methoxybenzyl)oxy)-20,20,20-trifluoroicosyl) hydrogen phosphate

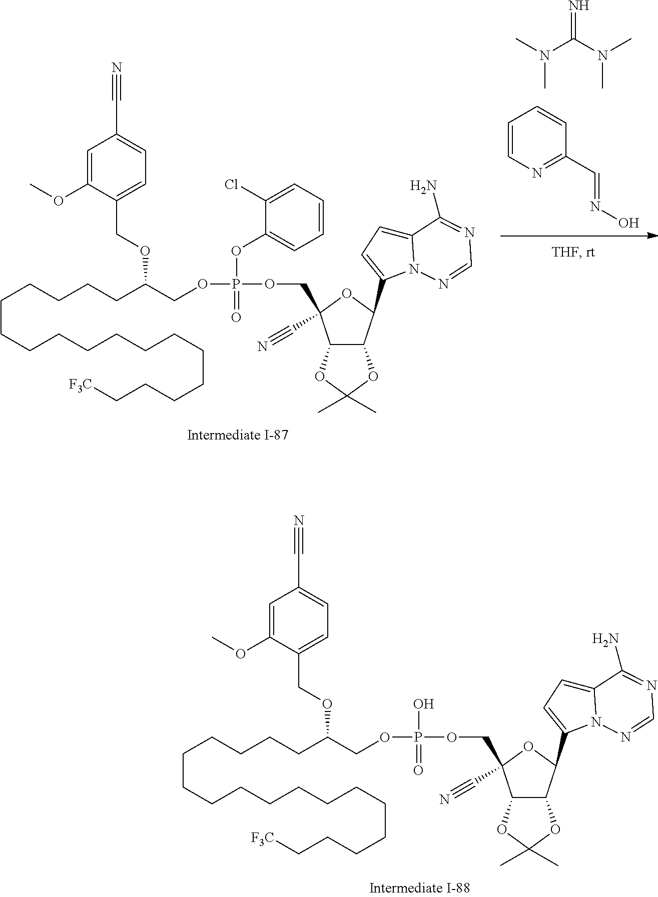

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) ((S)-2-((4-cyano-2-methoxybenzyl)oxy)-20,20,20-trifluoroicosyl) phosphate (120 mg, 0.118 mmol, 1.0 equiv.) in THF (10.0 mL) was added 1,1,3,3-tetramethylguanidine (0.15 mL, 1.18 mmol, 10.0 equiv.) and syn-2-pyridinealdoxime (144 mg, 1.18 mmol, 10.0 equiv.). The reaction mixture was stirred at room temperature overnight. The solution was concentrated in vacuo and purified by silica gel (0-40% MeOH in DCM) to afford the title compound (85 mg, 79% yield). 1H NMR (400 MHZ, Methanol-d4) δ 7.82 (s, 1H), 7.54 (d, J=7.7 Hz, 1H), 7.27-7.16 (m, 2H), 6.85-6.77 (m, 2H), 5.63 (d, J=3.7 Hz, 1H), 5.24 (dd, J=6.6, 3.7 Hz, 1H), 5.13 (d, J=6.6 Hz, 1H), 4.69 (d, J=13.8 Hz, 1H), 4.52 (d, J=13.8 Hz, 1H), 4.16-4.06 (m, 2H), 3.93-3.86 (m, 2H), 3.84 (s, 3H), 3.61-3.52 (m, 1H), 2.20-2.04 (m, 2H), 1.69 (s, 3H), 1.59-1.18 (m, 35H). ¹⁹F NMR (376 MHz, Methanol-d4) δ−68.56 (t, J=11.2 Hz). 31P NMR (162 MHZ, Methanol-d4) δ−0.04-−0.81 (m). MS m/z [M+1]=907.3.

### Intermediate I-89: [(3aS,4R,6S,6aS)-6-(4-aminopyrrolo[2,1-f][1,2,4]triazin-7-yl)-4-cyano-2,2-dimethyl-6,6a-dihydro-3aH-furo[3,4-d][1,3]dioxol-4-yl]methyl (2-chlorophenyl) [(2R)-2-[(3-cyano-5-fluoro-phenyl)methoxy]-20,20,20-trifluoro-icosyl] phosphate

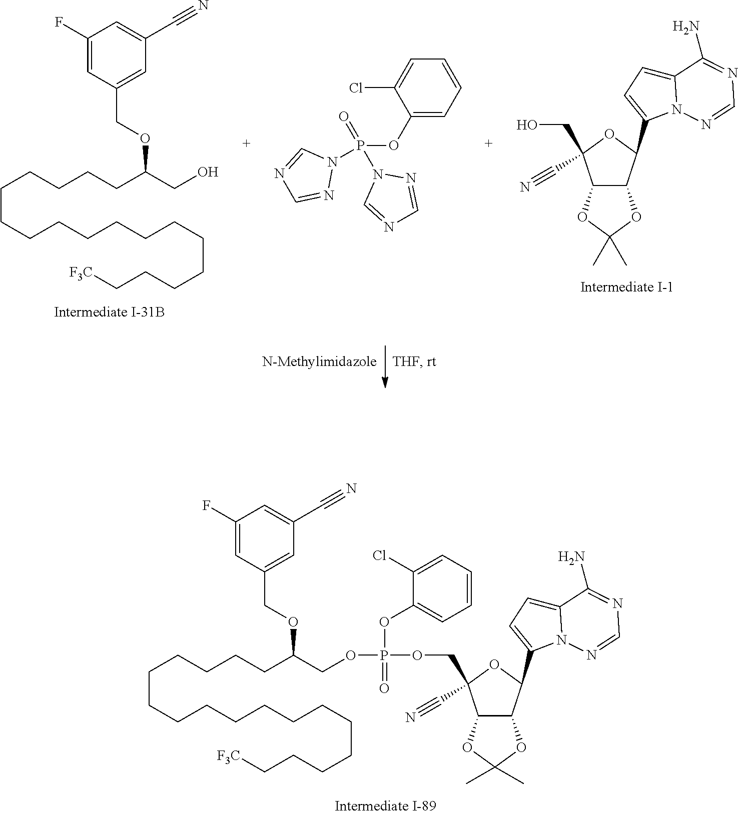 [SMILES: N#Cc1cc(F)cc(CO[C@@H](CO)CCCCCCCCCCCCCCCCCC(F)(F)F)c1 ; O=P(Oc1ccccc1Cl)(n1cncn1)n1cncn1 ; CC1(C)O[C@H]2[C@H](c3ccc4c(N)ncnn34)O[C@](C#N)(CO)[C@H]2O1 ; CC1(C)O[C@H]2[C@H](c3ccc4c(N)ncnn34)O[C@](C#N)(COP(=O)(OC[C@@H](CCCCCCCCCCCCCCCCCC(F)(F)F)OCc3cc(F)cc(C#N)c3)Oc3ccccc3Cl)[C@H]2O1]

To a solution of 1-[(2-chlorophenoxy)-(1,2,4-triazol-1-yl)phosphoryl]-1,2,4-triazole (460 mg, 1.48 mmol) was added intermediate I-1 (35 mg, 1.06 mmol, 1 eq) followed by 1-methylimidazole (113 mg, 1.37 mmol, 1.3 eq) and was stirred at room temperature for 10 min. To the reaction mixture, a solution of lipid alcohol, 3-fluoro-5-[[(1R)-19,19,19-trifluoro-1-(hydroxymethyl)nonadecoxy] methyl]benzonitrile (530 mg, 1.06 mmol, 1 eq) in THF (5 mL) was added slowly and stirred at room temperature for 18 h. The reaction mixture was diluted after completion of the reaction with ethyl acetate (100 mL) and quenched with water (10 mL) and stirred for 2 h. The organic layer was washed with brine (50 mL), dried over Na₂SO₄, filtered and concentrated in vacuo prior to purification by silica gel chromatography (0-100% EtOAc in hexanes) to afford the title compound. MS m/z [M+1]=1005.4.

### Intermediate I-90: [(3aS,4R,6S,6aS)-6-(4-aminopyrrolo[2,1-f][1,2,4]triazin-7-yl)-4-cyano-2,2-dimethyl-6,6a-dihydro-3aH-furo[3,4-d][1,3]dioxol-4-yl]methyl [(2R)-2-[(3-cyano-5-fluoro-phenyl)methoxy]-20,20,20-trifluoro-icosyl] hydrogen phosphate

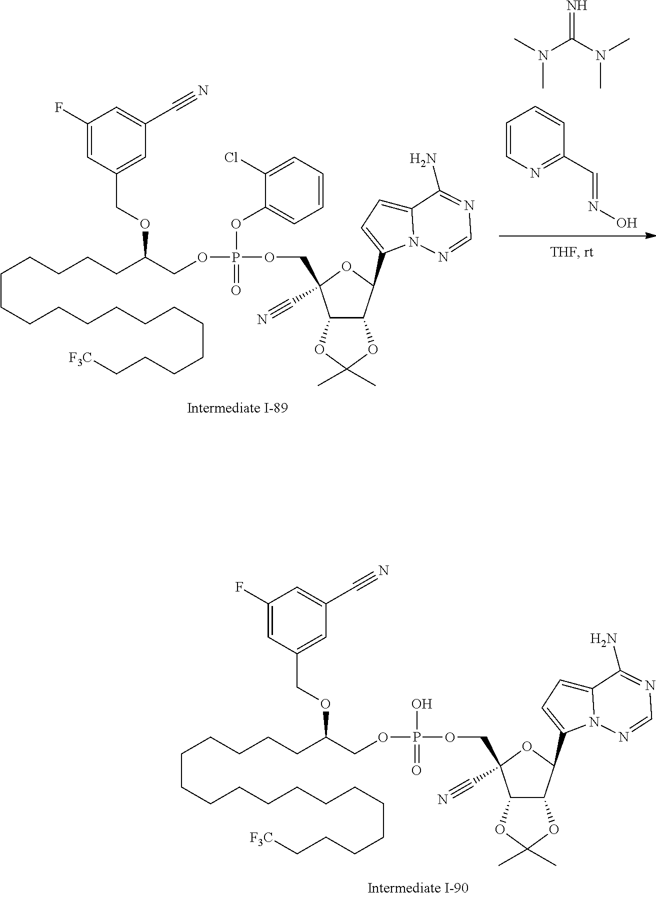

To a solution of [(3aS,4R,6S,6aS)-6-(4-aminopyrrolo[2,1-f][1,2,4]triazin-7-yl)-4-cyano-2,2-dimethyl-6,6a-dihydro-3aH-furo[3,4-d][1,3]dioxol-4-yl]methyl (2-chlorophenyl) [(2R)-2-[(3-cyano-5-fluoro-phenyl)methoxy]-20,20,20-trifluoro-icosyl] phosphate (910 mg, 0.9 mmol, 1.0 equiv.) in THF (10.0 mL) was added 1,1,3,3-tetramethylguanidine (0.68 mL, 5.43 mmol, 6.0 equiv.) and syn-2-pyridinealdoxime (448 mg, 3.67 mmol, 4 equiv.). The reaction mixture was stirred at room temperature overnight. The solution was concentrated in vacuo and purified by silica gel (0-40% MeOH in DCM) to afford the title compound. MS m/z [M+1]=895.3.

### Intermediate I-91: 1-bromo-3-[[(1S)-19,19,19-trifluoro-1-(trityloxymethyl)nonadecoxy]methyl]benzene

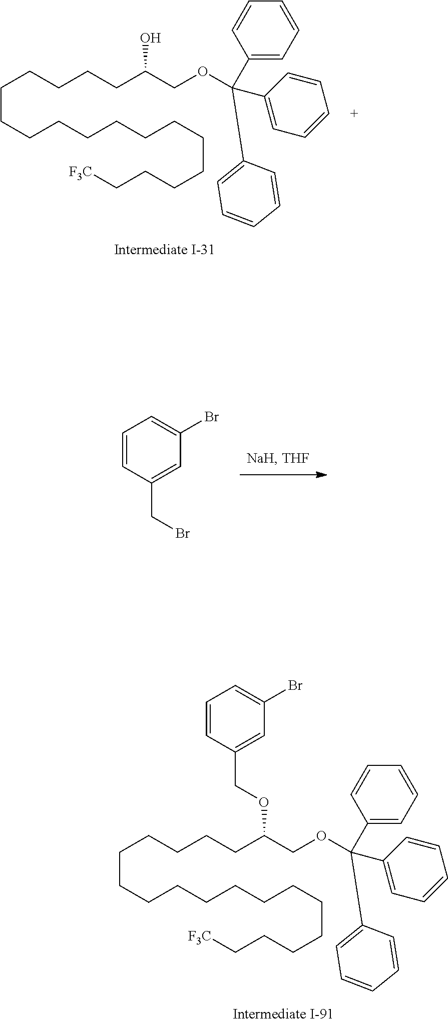 [SMILES: O[C@@H](CCCCCCCCCCCCCCCCCC(F)(F)F)COC(c1ccccc1)(c1ccccc1)c1ccccc1 ; BrCc1cccc(Br)c1 ; FC(F)(F)CCCCCCCCCCCCCCCCC[C@@H](COC(c1ccccc1)(c1ccccc1)c1ccccc1)OCc1cccc(Br)c1]

To a solution of (2S)-20,20,20-trifluoro-1-trityloxy-icosan-2-ol (2000 mg, 3.3 mmol, 1 eq) in THF (5 mL) was added NaH (314 mg, 8.2 mmol, 60% purity, 2.5 eq) at 0° C. and the mixture was stirred at 0° C. for 30 min. Then 1-bromo-3-(bromomethyl)benzene (1064 mg, 4.3 mmol, 1.3 eq) was added and the mixture was stirred at 65° C. for 12 hr. TLC indicated Reactant was consumed completely and many new spots were formed. The reaction mixture was quenched by addition of sat. NH₄Cl solution (60 ml) at 20° C. and extracted with ethyl acetate (40 mL×3). The combined organic layers were washed with H₂O (60 mL×2), dried over Na₂SO₄, and concentrated under reduced pressure to give a residue. The residue was purified by flash silica gel chromatography (ISCOR; 20 g SepaFlash® Silica Flash Column. 1H NMR (400 MHZ, Chloroform-d) δ 7.54 (d, J=1.9 Hz, 1H), 7.51-7.48 (m, 6H), 7.43 (dt, J=7.9, 1.6 Hz, 1H), 7.35-7.27 (m, 9H), 7.24 (dd, J=3.1, 1.7 Hz, 1H), 7.20 (d, J=7.7 Hz, 1H), 4.70 (d, J=12.0 Hz, 1H), 4.59-4.49 (m, 1H), 3.59-3.48 (m, 1H), 3.29-3.11 (m, 2H), 2.18-1.99 (m, 2H), 1.64-1.46 (m, 4H), 1.43-1.19 (m, 31H). ¹⁹F NMR (376 MHz, Chloroform-d) δ-66.94 (t, J=10.9 Hz).

### Intermediate I-92: N′,N′-dimethyl-N-[3-[[(1S)-19,19,19-trifluoro-1-(trityloxymethyl)nonadecoxy] methyl]phenyl]ethane-1,2-diamine

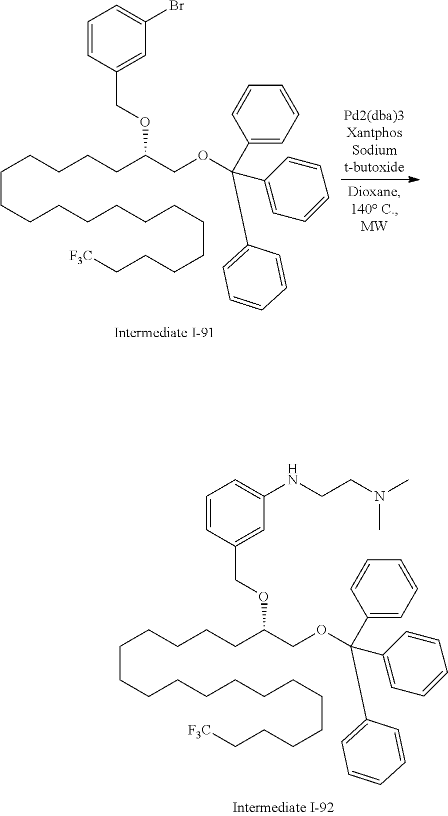 [SMILES: FC(F)(F)CCCCCCCCCCCCCCCCC[C@@H](COC(c1ccccc1)(c1ccccc1)c1ccccc1)OCc1cccc(Br)c1 ; CN(C)CCNc1cccc(CO[C@@H](CCCCCCCCCCCCCCCCCC(F)(F)F)COC(c2ccccc2)(c2ccccc2)c2ccccc2)c1]

To a solution of 1-bromo-3-[[(1S)-19,19,19-trifluoro-1-(trityloxymethyl)nonadecoxy] methyl]benzene (1100 mg, 1.4 mmol, 1 eq) in dioxane (10 mL) in a microwave vial, was added

Pd2(dba)3 (65 mg, 0.07 mmol), 9,9-Dimethyl-4,5-bis(diphenylphosphino)xanthene, Xantphos (98 mg, 0.17 mmol) and Sodium tert-butoxide (271 mg, 2.8 mmol) and irradiated at 140° C. for 45 min. Filtered through celite, concentrated, and purified by flash chromatography using dichloromethane and methanol (20%) as eluent to get the title compound. 1H NMR (400 MHz, Methanol-d4) δ 7.55-7.41 (m, 6H), 7.38-7.19 (m, 9H), 7.10 (t, J=7.8 Hz, 1H), 6.72-6.55 (m, 3H), 4.58 (d, J=11.5 Hz, 1H), 4.45 (d, J=11.6 Hz, 1H), 3.60-3.48 (m, 1H), 3.25-3.10 (m, 4H), 2.56 (t, J=6.7 Hz, 2H), 2.27 (s, 6H), 2.22-2.04 (m, 2H), 1.54 (q, J=7.5 Hz, 4H), 1.27 (d, J=22.0 Hz, 34H). ¹⁹F NMR (376 MHz, Methanol-d4) δ−68.54 (t, J=11.1 Hz).

### Intermediate I-93: (2S)-2-[[3-[2-(dimethylamino)ethylamino]phenyl]methoxy]-20,20,20-trifluoro-icosan-1-ol

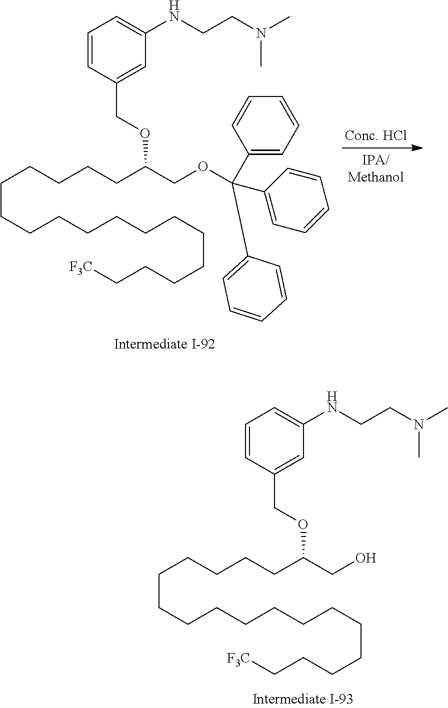 [SMILES: CN(C)CCNc1cccc(CO[C@@H](CCCCCCCCCCCCCCCCCC(F)(F)F)COC(c2ccccc2)(c2ccccc2)c2ccccc2)c1 ; CN(C)CCNc1cccc(CO[C@H](CO)CCCCCCCCCCCCCCCCCC(F)(F)F)c1]

To a solution of N′,N′-dimethyl-N-[3-[[(1S)-19,19,19-trifluoro-1-(trityloxymethyl)nonadecoxy] methyl]phenyl]ethane-1,2-diamine (810 mg, 1 mmol, 1 eq) in IPA (5 mL) and MeOH (5 mL) was added HCl (12 M, 1 mL, 10 eq) and the mixture was stirred at 20° C. for 2 hr. TLC indicated reactant was consumed and two new spots formed. The reaction mixture was quenched by addition sat. NaHCO₃ and then extracted with Ethyl acetate (120 mL×3). The combined organic layers were washed with H₂O (180 mL×2), dried over Na₂SO₄, filtered, and concentrated under reduced pressure to give a residue. The residue was purified by flash silica gel chromatography to yield the title compound.

¹H NMR (400 MHZ, Methanol-d4) δ 7.10 (t, J=7.8 Hz, 1H), 6.70 (t, J=1.9 Hz, 1H), 6.66 (dt, J=7.5, 1.2 Hz, 1H), 6.59 (ddd, J=8.1, 2.4, 1.0 Hz, 1H), 4.59 (d, J=11.6 Hz, 1H), 4.48 (d, J=11.6 Hz, 1H), 3.66-3.52 (m, 2H), 3.50-3.41 (m, 1H), 3.26 (t, J=6.7 Hz, 2H), 2.62 (t, J=6.7 Hz, 2H), 2.34 (s, 6H), 2.21-2.06 (m, 2H), 1.62-1.47 (m, 4H), 1.44-1.24 (m, 29H). ¹⁹F NMR (376 MHz, Methanol-d4) δ−66.79-−71.50 (m). MS m/z [M+1]=545.4.

### Intermediate I-94: [(3aS,4R,6S,6aS)-6-(4-aminopyrrolo[2,1-f][1,2,4]triazin-7-yl)-4-cyano-2,2-dimethyl-6,6a-dihydro-3aH-furo[3,4-d][1,3]dioxol-4-yl]methyl (2-chlorophenyl) [(2S)-2-[[3-[2-(dimethylamino)ethylamino]phenyl]methoxy]-20,20,20-trifluoro-icosyl] phosphate

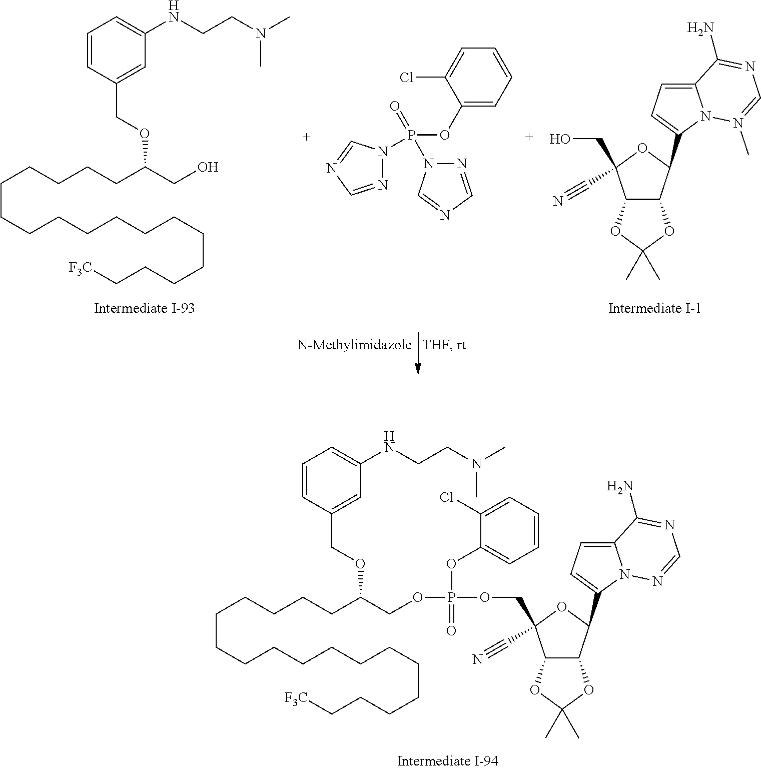

To a solution of 1-[(2-chlorophenoxy)-(1,2,4-triazol-1-yl)phosphoryl]-1,2,4-triazole (352 mg, 1.13 mmol, 1.5 eq) was added intermediate I-1 (250 mg, 0.8 mmol, 1 eq) followed by 1-methylimidazole (80 mg, 1 mmol, 1.3 eq) and was stirred at room temperature for 10 min. To the reaction mixture, a solution of lipid alcohol, (2S)-2-[[3-[2-(dimethylamino)ethylamino]-phenyl]methoxy]-20,20,20-trifluoro-icosan-1-ol (411 mg, 0.75 mmol, 1 eq) in THF (5 mL) was added slowly and stirred at room temperature for 18 h. The reaction mixture was diluted after completion of the reaction with ethyl acetate (100 mL) and quenched with water (10 mL) and stirred for 2 h. The organic layer was washed with brine (50 mL), dried over Na₂SO₄, filtered, and concentrated in vacuo prior to purification by silica gel chromatography (0-40% DCM/Methanol) to afford the title compound. 1H NMR (400 MHZ, Methanol-d4) δ 7.96 (d, J=2.6 Hz, 1H), 7.46 (tdd, J=7.5, 3.1, 1.1 Hz, 1H), 7.41-7.27 (m, 1H), 7.28-7.06 (m, 4H), 6.94 (dd, J=4.7, 2.3 Hz, 1H), 6.78-6.55 (m, 4H), 5.69 (dd, J=3.2, 2.1 Hz, 1H), 5.49 (s, OH), 5.27 (ddd, J=8.1, 6.6, 3.2 Hz, 1H), 5.06 (dd, J=24.0, 6.6 Hz, 1H), 4.64-4.45 (m, 4H), 4.34 (tdd, J=10.7, 7.1, 3.4 Hz, 1H), 4.18 (dddd, J=21.0, 11.0, 8.2, 5.5 Hz, 1H), 3.50 (td, J=6.0, 3.9 Hz, 2H), 3.34 (dt, J=6.0, 2.6 Hz, 3H), 2.91 (d, J=1.1 Hz, 6H), 2.24-2.04 (m, 2H), 1.52 (tdd, J=14.2, 9.9, 6.2 Hz, 4H), 1.28 (q, J=6.4, 4.9 Hz, 28H)). ¹⁹F NMR (376 MHz, Methanol-d4) δ−66.95 (t, J=11.2 Hz). ³¹P NMR (162 MHZ, Methanol-d4) δ−7.65 (p, J=6.48 Hz). MS m/z [M+1]=1048.3.

### Intermediate I-95: (3aS,4R,6S,6aS)-6-(4-aminopyrrolo[2,1-f][1,2,4]triazin-7-yl)-4-cyano-2,2-dimethyl-6,6a-dihydro-3aH-furo[3,4-d][1,3]dioxol-4-yl]methyl [(2S)-2-[[3-[2-(dimethylamino)ethylamino]phenyl]methoxy]-20,20,20-trifluoro-icosyl] hydrogen phosphate

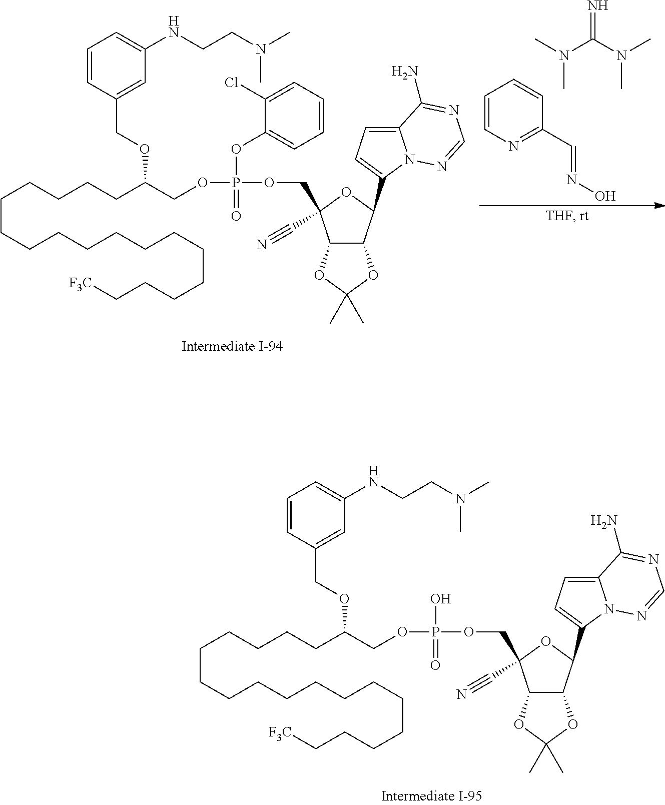

To a solution of [(3aS,4R,6S,6aS)-6-(4-aminopyrrolo[2,1-f][1,2,4]triazin-7-yl)-4-cyano-2,2-dimethyl-6,6a-dihydro-3aH-furo[3,4-d][1,3]dioxol-4-yl]methyl (2-chlorophenyl) [(2S)-2-[[3-[2-(dimethylamino)ethylamino]phenyl]methoxy]-20,20,20-trifluoro-icosyl] phosphate (400 mg, 0.38 mmol, 1.0 equiv.) in THF (10.0 mL) was added 1,1,3,3-tetramethylguanidine (0.3 mL, 2.3 mmol, 6.0 equiv.) and syn-2-pyridinealdoxime (190 mg, 1.55 mmol, 4 equiv.). The reaction mixture was stirred at room temperature overnight. The solution was concentrated in vacuo and purified by silica gel (0-40% MeOH in DCM) to afford the title compound. MS m/z [M+1]=938.4.

### C. Compounds

### Example 1: ((2R,3S,4R,5S)-5-(4-aminopyrrolo[2,1-f][1,2,4]triazin-7-yl)-2-cyano-3,4-dihydroxytetrahydrofuran-2-yl)methyl ((R)-2-((3-cyano-5-fluorobenzyl)oxy)-3-((18,18,18-trifluorooctadecyl)oxy)propyl) hydrogen phosphate

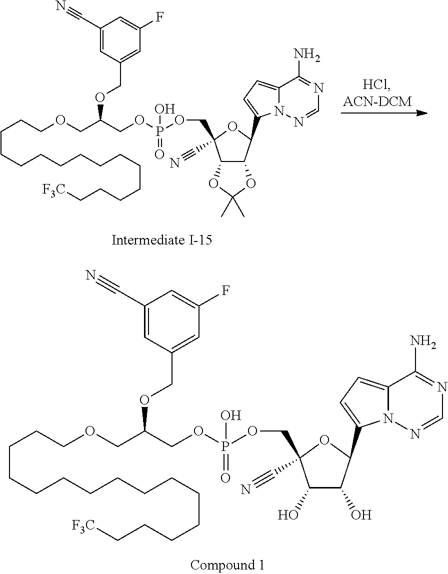 [SMILES: CC1(C)O[C@H]2[C@H](c3ccc4c(N)ncnn34)O[C@](C#N)(COP(=O)(O)OC[C@@H](COCCCCCCCCCCCCCCCC(F)(F)F)OCc3cc(F)cc(C#N)c3)[C@H]2O1 ; N#Cc1cc(F)cc(CO[C@H](COCCCCCCCCCCCCCCCC(F)(F)F)COP(=O)(O)OC[C@@]2(C#N)O[C@@H](c3ccc4c(N)ncnn34)[C@H](O)[C@@H]2O)c1]

To a solution of Intermediate I-15 (327 mg, 0.354 mmol) in ACN (4 ml)-DCM (4 mL) was added 25% HCl (0.3 mL) at rt. The solution was stirred at rt for 3 h, concentrated in vacuo, and purified by silica gel column chromatography (0 to 100% MeOH in DCM) to give title compound ((2R,3S,4R,5S)-5-(4-aminopyrrolo[2,1-f][1,2,4]triazin-7-yl)-2-cyano-3,4-dihydroxytetrahydrofuran-2-yl)methyl ((R)-2-((3-cyano-5-fluorobenzyl)oxy)-3-((18,18,18-trifluorooctadecyl)oxy)propyl) hydrogen phosphate, Compound 1. ¹H NMR (400 MHZ, Methanol-d4) δ 8.05 (s, 1H), 7.54 (s, 1H), 7.47 (d, J=9.5 Hz, 1H), 7.41-7.24 (m, 2H), 7.04 (d, J=4.8 Hz, 1H), 5.55 (d, J=5.1 Hz, 1H), 4.83-4.65 (m, 2H), 4.53-4.46 (m, 1H), 4.45-4.37 (m, 1H), 4.35-4.00 (m, 4H), 3.94-3.72 (m, 1H), 3.65-3.51 (m, 2H), 3.51-3.37 (m, 2H), 2.24-2.00 (m, 2H), 1.63-1.46 (m, 4H), 1.46-0.96 (m, 26H). ¹⁹F NMR (376 MHZ, Methanol-d4) δ −68.53, −112.82. ³¹P NMR (162 MHZ, Methanol-d4) δ−1.08. MS m/z [M+1]=884.8.

### Example 2: ((2R,3S,4R,5S)-5-(4-aminopyrrolo[2,1-f][1,2,4]triazin-7-yl)-2-cyano-3,4-dihydroxytetrahydrofuran-2-yl)methyl ((R)-2-((3-cyano-5-fluorobenzyl)oxy)-21,21,21-trifluorohenicosyl) hydrogen phosphate

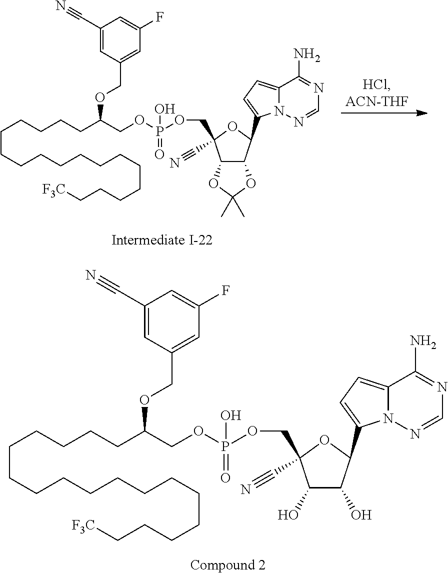 [SMILES: CC1(C)O[C@H]2[C@H](c3ccc4c(N)ncnn34)O[C@](C#N)(COP(=O)(O)OC[C@@H](CCCCCCCCCCCCCCCCCC(F)(F)F)OCc3cc(F)cc(C#N)c3)[C@H]2O1 ; N#Cc1cc(F)cc(CO[C@H](CCCCCCCCCCCCCCCCCC(F)(F)F)COP(=O)(O)OC[C@@]2(C#N)O[C@@H](c3ccc4c(N)ncnn34)[C@H](O)[C@@H]2O)c1]

To a solution of Intermediate I-22 (302 mg, 0.332 mmol) in ACN (4 ml)-THF (4 mL) was added 25% HCl (0.3 mL) at rt. The solution was stirred at rt for 4 h, concentrated in vacuo, and purified by silica gel column chromatography (0 to 100% MeOH in DCM) to give the title compound ((2R,3S,4R,5S)-5-(4-aminopyrrolo[2,1-f][1,2,4]triazin-7-yl)-2-cyano-3,4-dihydroxytetrahydrofuran-2-yl)methyl ((R)-2-((3-cyano-5-fluorobenzyl)oxy)-21,21,21-trifluorohenicosyl) hydrogen phosphate, Compound 2. ¹H NMR (400 MHZ, Methanol-d4) δ 8.05 (s, 1H), 7.51 (s, 1H), 7.46-7.25 (m, 3H), 7.06 (d, J=4.8 Hz, 1H), 5.55 (d, J=5.3 Hz, 1H), 4.79 (d, J=12.9 Hz, 1H), 4.61 (d, J=12.9 Hz, 1H), 4.53-4.46 (m, 1H), 4.43 (d, J=5.3 Hz, 1H), 4.33-4.16 (m, 2H), 4.14-4.05 (m, 1H), 4.05-3.90 (m, 1H), 3.73-3.60 (m, 1H), 2.25-2.01 (m, 2H), 1.63-1.47 (m, 4H), 1.47-1.17 (m, 30H). ¹⁹F NMR (376 MHz, Methanol-d4) δ−68.55, −112.88. ³¹P NMR (162 MHZ, Methanol-d4) δ−0.89. MS m/z [M+1]=869.4.

### Example 3: ((2R,3S,4R,5S)-5-(4-aminopyrrolo[2,1-f][1,2,4]triazin-7-yl)-2-cyano-3,4-dihydroxytetrahydrofuran-2-yl)methyl ((S)-2-((3-cyano-5-fluorobenzyl)oxy)-20,20,20-trifluoroicosyl) hydrogen phosphate

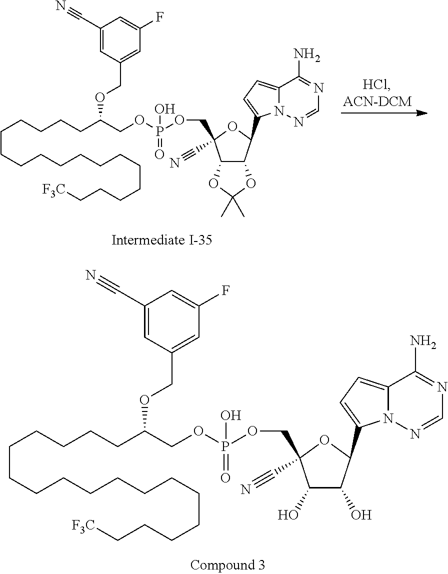 [SMILES: CC1(C)O[C@H]2[C@H](c3ccc4c(N)ncnn34)O[C@](C#N)(COP(=O)(O)OC[C@H](CCCCCCCCCCCCCCCCCC(F)(F)F)OCc3cc(F)cc(C#N)c3)[C@H]2O1 ; N#Cc1cc(F)cc(CO[C@@H](CCCCCCCCCCCCCCCCCC(F)(F)F)COP(=O)(O)OC[C@@]2(C#N)O[C@@H](c3ccc4c(N)ncnn34)[C@H](O)[C@@H]2O)c1]

To a solution of Intermediate I-35 (237 mg, 0.265 mmol) in ACN (2 ml)-DCM (2 mL) was added 25% HCl (1 mL) at rt. The solution was stirred at rt for 4 h, concentrated in vacuo, and purified by silica gel column chromatography (0 to 100% MeOH in DCM) to give the title compound ((2R,3S,4R,5S)-5-(4-aminopyrrolo[2,1-f][1,2,4]triazin-7-yl)-2-cyano-3,4-dihydroxytetrahydrofuran-2-yl)methyl ((S)-2-((3-cyano-5-fluorobenzyl)oxy)-20,20,20-trifluoroicosyl) hydrogen phosphate, Compound 3. ¹H NMR (400 MHZ, Methanol-d4) δ 8.04 (s, 1H), 7.52 (s, 1H), 7.45 (dd, J=9.5, 2.3 Hz, 1H), 7.39-7.34 (m, 2H), 7.07 (dd, J=4.8, 2.0 Hz, 1H), 5.56 (d, J=5.2 Hz, 1H), 4.79 (d, J=13.0 Hz, 1H), 4.62 (d, J=12.9 Hz, 1H), 4.53-4.46 (m, 1H), 4.44 (d, J=5.4 Hz, 1H), 4.34-4.13 (m, 2H), 4.14-4.04 (m, 1H), 4.03-3.91 (m, 1H), 3.73-3.59 (m, 1H), 2.21-2.04 (m, 2H), 1.69-1.12 (m, 32H). ¹⁹F NMR (376 MHz, Methanol-d4) δ −68.55, −112.87. ³¹P NMR (162 MHZ, Methanol-d4) δ−0.80. MS m/z [M+1]=854.8.

### Example 4: ((2R,3S,4R,5S)-5-(4-aminopyrrolo[2,1-f][1,2,4]triazin-7-yl)-2-cyano-3,4-dihydroxytetrahydrofuran-2-yl)methyl ((R)-2-((3-cyano-5-fluorobenzyl)oxy)-19,19-difluorononadecyl) hydrogen phosphate

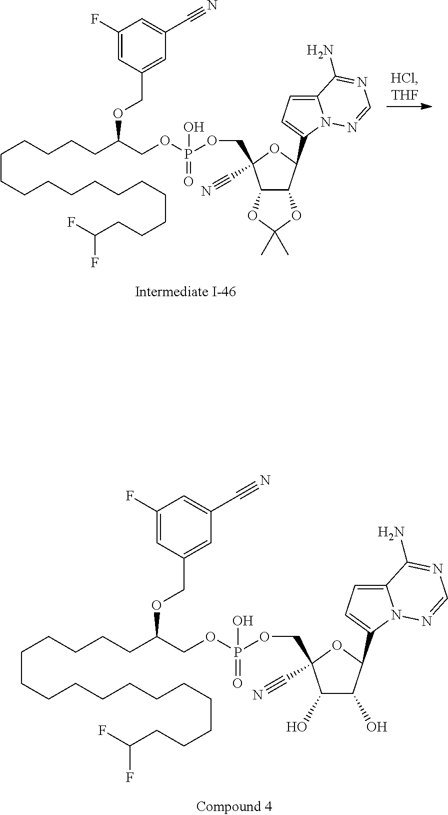 [SMILES: CC1(C)O[C@H]2[C@H](c3ccc4c(N)ncnn34)O[C@](C#N)(COP(=O)(O)OC[C@@H](CCCCCCCCCCCCCCCCC(F)F)OCc3cc(F)cc(C#N)c3)[C@H]2O1 ; N#Cc1cc(F)cc(CO[C@H](CCCCCCCCCCCCCCCCC(F)F)COP(=O)(O)OC[C@@]2(C#N)O[C@@H](c3ccc4c(N)ncnn34)[C@H](O)[C@@H]2O)c1]

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((R)-2-((3-cyano-5-fluorobenzyl)oxy)-19,19-difluorononadecyl) hydrogen phosphate, Intermediate I-46 (307 mg, 0.356 mmol, 1.0 equiv) in THF (4.0 mL) was added concentrated HCl (0.50 mL, 12.0 M, 16.9 equiv.). The reaction mixture was stirred at room temperature for 5 h. The solution was diluted with 4:1 DCM:IPA (100 mL) and water (75 mL). The pH of the aqueous layer was adjusted to around 3-4 using 20 wt % KOH. The layers were separated. The organic layer was washed with an additional 75 mL of water prior to drying over Na₂SO₄, filtering and concentrating in vacuo. The crude residue was purified by silica gel chromatography (0-40% MeOH in DCM) to afford the title compound ((2R,3S,4R,5S)-5-(4-aminopyrrolo[2,1-f][1,2,4]triazin-7-yl)-2-cyano-3,4-dihydroxytetrahydrofuran-2-yl)methyl ((R)-2-((3-cyano-5-fluorobenzyl)oxy)-19,19-difluorononadecyl) hydrogen phosphate, Compound 4. ¹H NMR (400 MHZ, Methanol-d4) δ 7.78 (s, 1H), 7.49-7.44 (m, 1H), 7.41-7.31 (m, 2H), 6.87-6.77 (m, 2H), 5.84 (tt, J=57.1, 4.5 Hz, 1H), 5.54 (d, J=5.0 Hz, 1H), 4.69 (d, J=13.0 Hz, 1H), 4.59-4.43 (m, 3H), 4.20-4.08 (m, 2H), 3.93-3.80 (m, 2H), 3.57-3.49 (m, 1H), 1.88-1.68 (m, 2H), 1.51-1.15 (m, 30H). ¹⁹F NMR (376 MHz, Methanol-d4) δ −112.90-−113.01 (m), −117.83-−118.14 (m). ³¹P NMR (162 MHZ, Methanol-d4) δ−0.02-−0.55 (m). MS m/z [M+1]=823.2.

### Example 5: ((2R,3S,4R,5S)-5-(4-aminopyrrolo[2,1-f][1,2,4]triazin-7-yl)-2-cyano-3,4-dihydroxytetrahydrofuran-2-yl)methyl (20,20,20-trifluoroicosyl) hydrogen phosphate

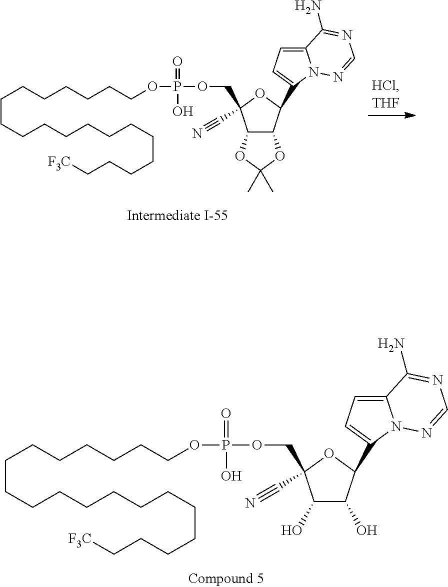 [SMILES: CC1(C)O[C@H]2[C@H](c3ccc4c(N)ncnn34)O[C@](C#N)(COP(=O)(O)OCCCCCCCCCCCCCCCCCCCC(F)(F)F)[C@H]2O1 ; N#C[C@]1(COP(=O)(O)OCCCCCCCCCCCCCCCCCCCC(F)(F)F)O[C@@H](c2ccc3c(N)ncnn23)[C@H](O)[C@@H]1O]

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (20,20,20-trifluoroicosyl) hydrogen phosphate Intermediate I-55 (339 mg, 0.455 mmol, 1.0 equiv) in THF (5.0 mL) was added concentrated HCl (1.0 mL, 12.0 M, 26.4 equiv.). The reaction mixture was stirred at room temperature overnight. The solution was diluted with 4:1 DCM:IPA (100 mL) and water (75 mL). The pH of the aqueous layer was adjusted to between 4 and 5 using 20 wt % KOH and 1M HCl and was further diluted with brine (50 mL). The layers were separated. The organic layer was washed with an additional 75 mL of water and 50 mL of brine prior to drying over Na₂SO₄, filtering and concentrating in vacuo. The crude residue was loaded onto silica gel and purified by silica gel chromatography (0-40% MeOH in DCM) to afford the title compound ((2R,3S,4R,5S)-5-(4-aminopyrrolo[2,1-f][1,2,4]triazin-7-yl)-2-cyano-3,4-dihydroxytetrahydrofuran-2-yl)methyl (20,20,20-trifluoroicosyl) hydrogen phosphate, Compound 5. ¹H NMR (400 MHZ, Methanol-d4) δ 7.84 (s, 1H), 6.98 (d, J=4.6 Hz, 1H), 6.88 (d, J=4.6 Hz, 1H), 5.54 (d, J=5.2 Hz, 1H), 4.58-4.48 (m, 2H), 4.20-4.07 (m, 2H), 3.87-3.80 (m, 2H), 2.19-2.04 (m, 2H), 1.60-1.48 (m, 4H), 1.44-1.18 (m, 30H). ¹⁹F NMR (376 MHZ, Methanol-d4) δ−68.49-−68.62 (m). 31P NMR (162 MHZ, Methanol-d4) δ 0.20-−0.55 (m). MS m/z [M+1]=706.1.

### Example 6: ((2R,3S,4R,5S)-5-(4-aminopyrrolo[2,1-f][1,2,4]triazin-7-yl)-2-cyano-3,4-dihydroxytetrahydrofuran-2-yl)methyl ((S)-2-((3-cyano-5-fluorobenzyl)oxy)-19,19,19-trifluorononadecyl) hydrogen phosphate

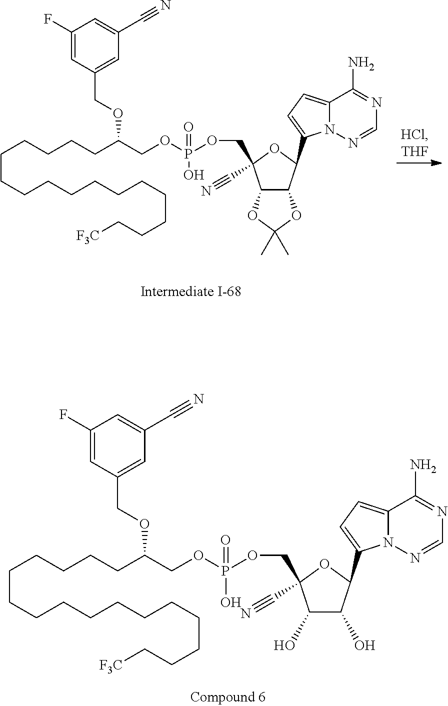 [SMILES: CC1(C)O[C@H]2[C@H](c3ccc4c(N)ncnn34)O[C@](C#N)(COP(=O)(O)OC[C@H](CCCCCCCCCCCCCCCCC(F)(F)F)OCc3cc(F)cc(C#N)c3)[C@H]2O1 ; N#Cc1cc(F)cc(CO[C@@H](CCCCCCCCCCCCCCCCC(F)(F)F)COP(=O)(O)OC[C@@]2(C#N)O[C@@H](c3ccc4c(N)ncnn34)[C@H](O)[C@@H]2O)c1]

To a solution of ((2R,3S,4R,5S)-5-(4-aminopyrrolo[2,1-f][1,2,4]triazin-7-yl)-2-cyano-3,4-dihydroxytetrahydrofuran-2-yl)methyl ((S)-2-((3-cyano-5-fluorobenzyl)oxy)-19,19,19-trifluorononadecyl) hydrogen phosphate Intermediate I-68 (206 mg, 0.34 mmol) in ACN (2 ml) −DCM (2 mL) was added 25% HCl (1 mL) at rt. The solution was stirred at rt for 4 h, concentrated in vacuo, and purified by silica gel column chromatography (0 to 100% MeOH in DCM) to give the title compound ((2R,3S,4R,5S)-5-(4-aminopyrrolo[2,1-f][1,2,4]triazin-7-yl)-2-cyano-3,4-dihydroxytetrahydrofuran-2-yl)methyl ((S)-2-((3-cyano-5-fluorobenzyl)oxy)-19,19,19-trifluorononadecyl) hydrogen phosphate, Compound 6 (123 mg, 63%). 1H NMR (400 MHZ, Methanol-d4) δ 8.06 (s, 1H), 7.52 (s, 1H), 7.45 (d, J=9.4 Hz, 1H), 7.42-7.30 (m, 2H), 7.08 (d, J=4.8 Hz, 1H), 5.56 (d, J=5.2 Hz, 1H), 4.85-4.72 (m, 1H), 4.61 (d, J=12.9 Hz, 1H), 4.50 (t, J=5.3 Hz, 1H), 4.44 (d, J=5.3 Hz, 1H), 4.31-4.12 (m, 2H), 4.12-3.92 (m, 2H), 3.72-3.60 (m, 1H), 2.25-2.05 (m, 2H), 1.66-1.48 (m, 2H), 1.48-1.16 (m, 28H); ¹⁹F NMR (376 MHZ, Methanol-d4) δ−68.56, −112.90; 31P NMR (162 MHZ, Methanol-d4) δ−0.81; MS m/z [M+1]=841.4.

### Example 7: ((2R,3S,4R,5S)-5-(4-aminopyrrolo[2,1-f][1,2,4]triazin-7-yl)-2-cyano-3,4-dihydroxytetrahydrofuran-2-yl)methyl ((S)-20,20,20-trifluoro-2-(quinolin-2-ylmethoxy)icosyl) hydrogen phosphate

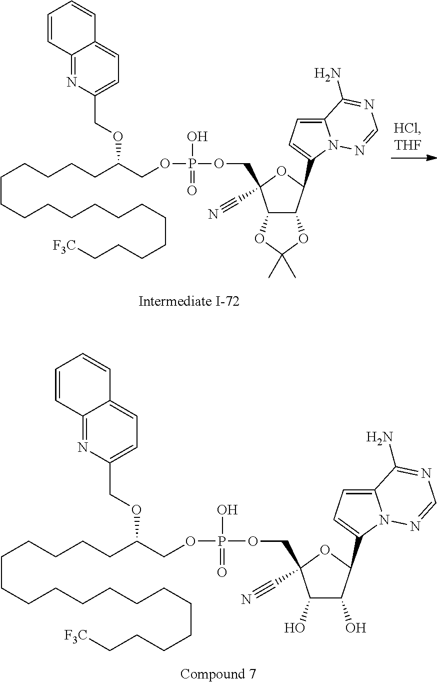 [SMILES: CC1(C)O[C@H]2[C@H](c3ccc4c(N)ncnn34)O[C@](C#N)(COP(=O)(O)OC[C@H](CCCCCCCCCCCCCCCCCC(F)(F)F)OCc3ccc4ccccc4n3)[C@H]2O1 ; N#C[C@]1(COP(=O)(O)OC[C@H](CCCCCCCCCCCCCCCCCC(F)(F)F)OCc2ccc3ccccc3n2)O[C@@H](c2ccc3c(N)ncnn23)[C@H](O)[C@@H]1O]

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((S)-20,20,20-trifluoro-2-(quinolin-2-ylmethoxy)icosyl) hydrogen phosphate (208 mg, 0.230 mmol, 1.0 equiv) in THF (6.0 mL) was added concentrated HCl (1.0 mL, 12.0 M, 52.1 equiv.). The reaction mixture was stirred at room temperature overnight. The solution was diluted with 4:1 DCM:IPA (100 mL) and water (75 mL). The pH of the aqueous layer was adjusted to around 2-3 using 20 wt % KOH. Brine was added to reduce emulsions. The layers were separated. The organic layer was washed with additional water and brine. The organic fraction was dried over Na₂SO₄, filtered, and concentrated in vacuo. The crude residue was loaded onto silica gel and purified by silica gel chromatography (0-40% MeOH in DCM) to afford the title compound as the HCl salt. 1H NMR (400 MHZ, Methanol-d4) δ 8.25 (d, J=8.5 Hz, 1H), 7.97 (d, J=8.5 Hz, 1H), 7.89-7.82 (m, 1H), 7.78-7.66 (m, 3H), 7.59-7.51 (m, 1H), 6.84-6.77 (m, 2H), 5.53 (d, J=5.4 Hz, 1H), 4.91 (d, J=13.2 Hz, 1H), 4.77 (d, J=13.2 Hz, 1H), 4.61-4.47 (m, 2H), 4.23-4.11 (m, 2H), 4.02-3.90 (m, 2H), 3.73-3.64 (m, 1H), 2.20-2.02 (m, 2H), 1.60-1.11 (m, 32H). ¹⁹F NMR (376 MHZ, Methanol-d4) δ−68.54 (t, J=11.2 Hz). 31P NMR (162 MHZ, Methanol-d4) δ 0.07-−1.00 (m). MS m/z [M+1]=863.4.

### Example 8: ((2R,3S,4R,5S)-5-(4-aminopyrrolo[2,1-f][1,2,4]triazin-7-yl)-2-cyano-3,4-dihydroxytetrahydrofuran-2-yl)methyl ((R)-2-((3-cyano-5-fluorobenzyl)oxy)-22,22,22-trifluorodocosyl) hydrogen phosphate

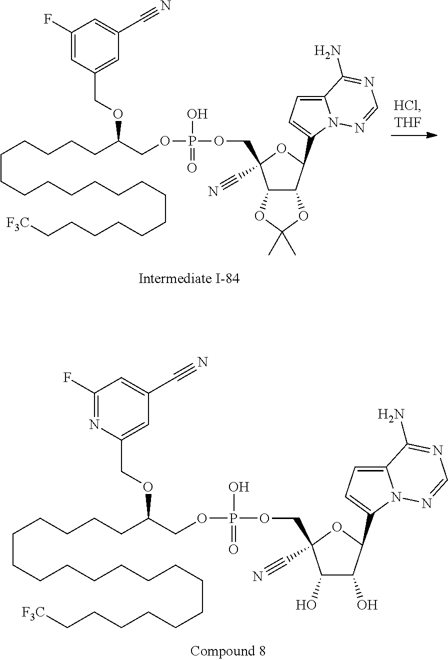 [SMILES: CC1(C)O[C@H]2[C@H](c3ccc4c(N)ncnn34)O[C@](C#N)(COP(=O)(O)OC[C@@H](CCCCCCCCCCCCCCCCCCCC(F)(F)F)OCc3cc(F)cc(C#N)c3)[C@H]2O1 ; N#Cc1cc(F)nc(CO[C@H](CCCCCCCCCCCCCCCCCCCC(F)(F)F)COP(=O)(O)OC[C@@]2(C#N)O[C@@H](c3ccc4c(N)ncnn34)[C@H](O)[C@@H]2O)c1]

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((R)-2-((3-cyano-5-fluorobenzyl)oxy)-22,22,22-trifluorodocosyl) hydrogen phosphate (234 mg, 0.254 mmol, 1.0 equiv) in THF (6.0 mL) was added concentrated HCl (1.0 mL, 12.0 M, 47.3 equiv.). The reaction mixture was stirred at room temperature overnight. The solution was diluted with 4:1 DCM:IPA (100 mL) and water (75 mL). The pH of the aqueous layer was adjusted to around 3 using 20 wt % KOH and 1M HCl. The organic layer was washed with additional water (75 mL). The organic fraction was dried over Na₂SO₄, filtered, and concentrated in vacuo. The crude residue was loaded onto silica gel and purified by silica gel chromatography (0-40% MeOH in DCM) to afford the title compound. 1H NMR (400 MHZ, Methanol-d4) δ 7.85 (s, 1H), 7.50-7.45 (m, 1H), 7.41-7.32 (m, 2H), 6.99 (d, J=4.6 Hz, 1H), 6.89 (d, J=4.6 Hz, 1H), 5.53 (d, J=5.0 Hz, 1H), 4.73 (d, J=13.1 Hz, 1H), 4.56-4.47 (m, 3H), 4.22-4.09 (m, 2H), 3.98-3.84 (m, 2H), 3.62-3.53 (m, 1H), 2.20-2.04 (m, 2H), 1.59-1.18 (m, 36H). ¹⁹F NMR (376 MHz, Methanol-d4) δ−68.37-−68.76 (m), −112.85-−113.03 (m). ³¹P NMR (162 MHz, Methanol-d4) δ−0.12-−0.48 (m). MS m/z [M+1]=883.4.

### Example 9: ((2R,3S,4R,5S)-5-(4-aminopyrrolo[2,1-f][1,2,4]triazin-7-yl)-2-cyano-3,4-dihydroxytetrahydrofuran-2-yl)methyl ((S)-2-((4-cyano-2-methoxybenzyl)oxy)-20,20,20-trifluoroicosyl) hydrogen phosphate

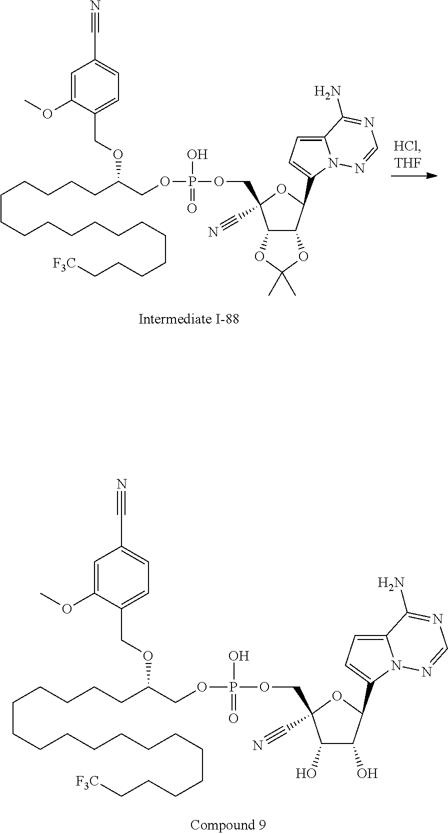 [SMILES: COc1cc(C#N)ccc1CO[C@@H](CCCCCCCCCCCCCCCCCC(F)(F)F)COP(=O)(O)OC[C@@]1(C#N)O[C@@H](c2ccc3c(N)ncnn23)[C@@H]2OC(C)(C)O[C@@H]21 ; COc1cc(C#N)ccc1CO[C@@H](CCCCCCCCCCCCCCCCCC(F)(F)F)COP(=O)(O)OC[C@@]1(C#N)O[C@@H](c2ccc3c(N)ncnn23)[C@H](O)[C@@H]1O]

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((S)-2-((4-cyano-2-methoxybenzyl)oxy)-20,20,20-trifluoroicosyl) hydrogen phosphate (79 mg, 0.0871 mmol, 1.0 equiv) in THF (6.0 mL) was added concentrated HCl (1.0 mL, 12.0 M, 138 equiv.). The reaction mixture was stirred at room temperature overnight. The solution was diluted with 4:1 DCM:IPA (100 mL) and water (75 mL). The pH of the aqueous layer was adjusted to around 3 using 20 wt % KOH and 1M HCl. The layers were separated. The organic layer was washed with additional water. The organic fraction was dried over Na₂SO₄, filtered and concentrated in vacuo. The crude residue was loaded onto silica gel and purified by silica gel chromatography (0-40% MeOH in DCM) to afford the title compound. 1H NMR (400 MHZ, Methanol-d4) δ 7.78 (s, 1H), 7.52 (d, J=7.7 Hz, 1H), 7.25-7.16 (m, 2H), 6.85-6.79 (m, 2H), 5.54 (d, J=5.4 Hz, 1H), 4.68 (d, J=13.8 Hz, 1H), 4.60-4.45 (m, 3H), 4.19-4.07 (m, 2H), 3.94-3.85 (m, 2H), 3.83 (s, 3H), 3.60-3.52 (m, 1H), 2.20-2.05 (m, 2H), 1.60-1.13 (m, 32H). ¹⁹F NMR (376 MHZ, Methanol-d4) δ−68.56 (t, J=11.2 Hz). 31P NMR (162 MHZ, Methanol-d4) δ−0.15-−0.65 (m). MS m/z [M+1]=867.2.

### Example 10: ((2R,3S,4R,5S)-5-(4-aminopyrrolo[2,1-f][1,2,4]triazin-7-yl)-2-cyano-3,4-dihydroxytetrahydrofuran-2-yl)methyl ((R)-2-((3-cyano-5-fluorobenzyl)oxy)-20,20,20-trifluoroicosyl) hydrogen phosphate

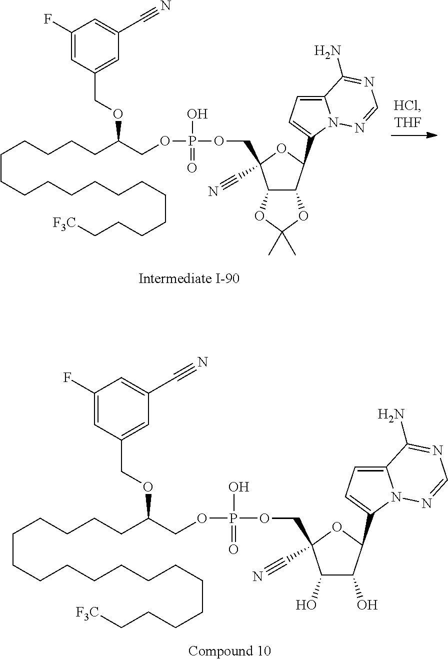 [SMILES: CC1(C)O[C@H]2[C@H](c3ccc4c(N)ncnn34)O[C@](C#N)(COP(=O)(O)OC[C@@H](CCCCCCCCCCCCCCCCCC(F)(F)F)OCc3cc(F)cc(C#N)c3)[C@H]2O1 ; N#Cc1cc(F)cc(CO[C@H](CCCCCCCCCCCCCCCCCC(F)(F)F)COP(=O)(O)OC[C@@]2(C#N)O[C@@H](c3ccc4c(N)ncnn34)[C@H](O)[C@@H]2O)c1]

To a solution of [(3aS,4R,6S,6aS)-6-(4-aminopyrrolo[2,1-f][1,2,4]triazin-7-yl)-4-cyano-2,2-dimethyl-6,6a-dihydro-3aH-furo[3,4-d][1,3]dioxol-4-yl]methyl [(2R)-2-[(3-cyano-5-fluoro-phenyl)methoxy]-20,20,20-trifluoro-icosyl] hydrogen phosphate (810 mg, 0.9 mmol, 1.0 equiv) in THF (6.0 mL) was added concentrated HCl (2.9 mL, 12.0 M, 38 equiv.). The reaction mixture was stirred at room temperature overnight. The solution was diluted with 4:1 DCM:IPA (100 mL) and water (75 mL). The pH of the aqueous layer was adjusted to around 3 using 20 wt % KOH and 1M HCl. The organic layer was washed with additional water (75 mL). The organic fraction was dried over Na₂SO₄, filtered, and concentrated in vacuo. The crude residue was loaded onto silica gel and purified by silica gel chromatography (0-40% MeOH in DCM) to afford the title compound. 1H NMR (400 MHZ, Methanol-d4) δ 7.80 (s, 1H), 7.48 (t, J=1.3 Hz, 1H), 7.43-7.30 (m, 2H), 6.88-6.78 (m, 2H), 5.55 (d, J=5.1 Hz, 1H), 4.71 (d, J=13.0 Hz, 1H), 4.62-4.43 (m, 3H), 4.15 (qd, J=10.9, 4.8 Hz, 2H), 3.99-3.79 (m, 2H), 3.62-3.47 (m, 1H), 3.00 (s, 2H), 2.25-2.01 (m, 2H), 1.63-1.49 (m, 2H), 1.29 (dd, J=15.8, 7.8 Hz, 30H). ¹⁹F NMR (376 MHZ, Methanol-d4) δ−68.56 (t, J=11.3 Hz), −112.71-−113.31 (m). ³¹P NMR (162 MHz, Methanol-d4) δ 1.70-−3.29 (m). MS m/z [M+1]=855.2.

### Example 11: ((2R,3S,4R,5S)-5-(4-aminopyrrolo[2,1-f][1,2,4]triazin-7-yl)-2-cyano-3,4-dihydroxytetrahydrofuran-2-yl)methyl ((S)-2-((3-((2-(dimethylamino)ethyl)amino)benzyl)oxy)-20,20,20-trifluoroicosyl) hydrogen phosphate

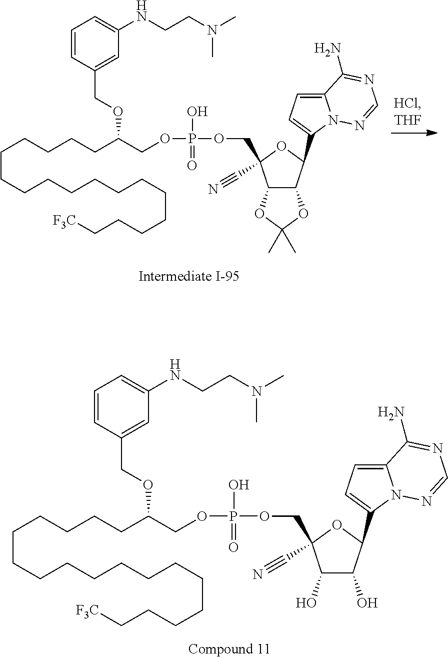 [SMILES: CN(C)CCNc1cccc(CO[C@@H](CCCCCCCCCCCCCCCCCC(F)(F)F)COP(=O)(O)OC[C@@]2(C#N)O[C@@H](c3ccc4c(N)ncnn34)[C@@H]3OC(C)(C)O[C@@H]32)c1 ; CN(C)CCNc1cccc(CO[C@@H](CCCCCCCCCCCCCCCCCC(F)(F)F)COP(=O)(O)OC[C@@]2(C#N)O[C@@H](c3ccc4c(N)ncnn34)[C@H](O)[C@@H]2O)c1]

To a solution of (3aS,4R,6S,6aS)-6-(4-aminopyrrolo[2,1-f][1,2,4]triazin-7-yl)-4-cyano-2,2-dimethyl-6,6a-dihydro-3aH-furo[3,4-d][1,3]dioxol-4-yl]methyl [(2S)-2-[[3-[2-(dimethylamino)ethylamino]phenyl]methoxy]-20,20,20-trifluoro-icosyl] hydrogen phosphate (350 mg, 0.37 mmol, 1.0 equiv) in THF (6.0 mL) was added concentrated HCl (1.2 mL, 12.0 M, 38 equiv.). The reaction mixture was stirred at room temperature overnight. The solution was diluted with 4:1 DCM:IPA (100 mL) and water (75 mL). The pH of the aqueous layer was adjusted to around 3 using 20 wt % KOH and 1M HCl. The organic layer was washed with additional water (75 mL). The organic fraction was dried over Na₂SO₄, filtered, and concentrated in vacuo. The crude residue was loaded onto silica gel and purified by silica gel chromatography (0-40% MeOH in DCM) to afford the title compound. 1H NMR (400 MHZ, Methanol-d4) δ 7.79 (s, 1H), 7.06 (t, J=7.8 Hz, 1H), 6.90 (t, J=1.9 Hz, 1H), 6.86 (d, J=4.5 Hz, 1H), 6.81 (d, J=4.6 Hz, 1H), 6.62-6.53 (m, 2H), 5.55 (d, J=5.2 Hz, 1H), 4.58 (t, J=5.3 Hz, 1H), 4.49 (d, J=5.6 Hz, 3H), 4.22-4.08 (m, 2H), 3.97-3.80 (m, 2H), 3.54 (dt, J=19.2, 6.1 Hz, 3H), 3.18 (td, J=6.4, 2.3 Hz, 2H), 2.81 (s, 6H), 2.22-2.04 (m, 2H), 1.55 (p, J=7.8, 7.3 Hz, 2H), 1.40-1.21 (m, 32H). ¹⁹F NMR (376 MHz, Methanol-d4) δ−68.53 (t, J=11.2 Hz). 31P NMR (162 MHz, Methanol-d4) δ−0.42 (p, J=5.4 Hz). MS m/z [M+1]=898.3.

### D. Biological Examples

### Example A. DENV-2 moDC EC₅₀

Human monocyte-derived dendritic cells (moDCs) were derived from CD14+ monocytes (AllCells) cultured in Human Mo-DC Differentiation medium containing GM-CSF and IL-4 (Miltenyi Biotec). On day 7, moDCs were harvested by mechanical disruption, washed and suspended in serum-free RPMI. moDCs were infected with Vero-derived Dengue 2, New Guinea strain (NGC) at a MOI=0.1 for two hours in serum-free RPMI with gentle agitation at 37° C. Cells were washed and resuspended in 10% serum-containing RPMI (Gibco, supplemented with sodium pyruvate, NEAA, Penicillin-Streptomycin). 10\hat{ }5 cells were plated in triplicate in 96-well plates with compounds dispensed at graded doses (Hewlett-Packard D300 Digital Dispenser). All wells were normalized to 0.25% DMSO. At 48 hours, cells were washed with 1×PBS and all supernatants removed. Total RNA was extracted using RNEasy 96 plates (Qiagen) and used to generate first-strand cDNA using XLT cDNA 5× Supermix (QuantaBio). cDNA was used as a template in a Taqman qPCR duplex reaction specific to DENV2 viral and GAPDH gene expression. EC₅₀ values were determined using Prism Graphpad software, with normalization to a positive control and no compound negative control wells.

### Example B. moDC CC50

Human monocyte-derived dendritic cells (moDCs) were derived from CD14+ monocytes (AllCells) cultured in Human Mo-DC Differentiation medium containing GM-CSF and IL-4 (Miltenyi Biotec). On day 7, moDCs were harvested by mechanical disruption, washed and cultured in triplicate at 1×10\hat{ }5-5\hat{ }10\hat{ }4 cells/well in 96-well plates with compounds dispensed at graded doses (Hewlett-Packard D300 Digital Dispenser). All wells were normalized to 0.25% DMSO. After 48 hours, CellTiter Glo (Promega) was added and incubated for 10 minutes at room temp before reading on a luminometer. % viability curves were calculated against no compound and no cell control wells. CC₅₀ values were determined using Prism Graphpad software.

### Example C. DENV-2 Huh-7 EC₅₀

Huh7 (Human hepatocarcinoma 7) cells were maintained in 10% FCS-containing DMEM complete media. On the day of the assay, cells were trypsinized (0.1% Trypsin-EDTA), washed and infected for 2 hours in serum-free DMEM with Dengue serotype 2 New Guinea C (NGC) strain at MOI=0.1 with gentle agitation at 37° C. After 2 hours, cells were washed with serum-free media and suspended in 10% FCS-containing DMEM (Gibco, supplemented with sodium pyruvate, NEAA, Penicillin-Streptomycin). 10\hat{ }5 cells were plated in triplicate in 96-well plates with compounds dispensed at graded doses (Hewlett-Packard D300 Digital Dispenser). All wells were normalized to 0.25% DMSO. At 48 hours, cells were washed with 1×PBS and all supernatants removed. Total RNA was extracted using RNEasy 96 plates (Qiagen) and used to generate first-strand cDNA using XLT cDNA 5× Supermix (QuantaBio). cDNA was used as a template in a Taqman qPCR duplex reaction specific to DENV2 viral and GAPDH gene expression. EC₅₀ values were determined using Prism Graphpad software, with normalization to a positive control and no compound negative control wells.

### Example D. Huh-7 CC₅₀

Human hepatocarcinoma 7 (Huh7) cells were maintained in 10% FCS-containing complete DMEM. On day of assay, cells were trypsinized with 0.1% Trypsin-EDTA, washed and cultured in triplicate at 1-2×10\hat{ }4 cells/well in 96-well plates with compounds dispensed at graded doses (Hewlett-Packard D300 Digital Dispenser). All wells were normalized to 0.25% DMSO. After 48 hours, CellTiter Glo (Promega) was added and incubated for 10 minutes at room temp before reading on a luminometer. % viability curves were calculated against no compound and no cell control wells. CC₅₀ values were determined using Prism Graphpad software.

### Example E. RSV HEp-2 EC₅₀

Antiviral activity against RSV is determined using an infectious cytopathic cell protection assay in HEp-2 cells. In this assay, compounds inhibiting viral infection and/or replication produce a cytoprotective effect against the virus-induced cell killing that can be quantified using a cell viability reagent. The techniques used here are novel adaptations of methods described in published literature (Chapman et al., ANTIMICROB AGENTS CHEMOTHER. 2007, 51(9):3346-53).

HEp-2 cells are obtained from ATCC (Manassas, VI) and maintained in MEM media supplemented with 10% fetal bovine serum and penicillin/streptomycin. Cells are passaged twice a week and kept at subconfluent stage. Commercial stock of RSV strain A2 (Advanced Biotechnologies, Columbia, MD) is titered before compound testing to determine the appropriate dilution of the virus stock that generates desirable cytopathic effect in HEp-2 cells.

For antiviral tests, HEp-2 cells are grown in large cell culture flasks to near confluency but not fully so. The compounds to be tested are prediluted in DMSO in 384-well compound dilution plates, either in an 8 or 40 sample per plate standardized dose response format. 3-fold serial dilution increments of each test compound are prepared in the plates and test samples are transferred via acoustic transfer apparatus (Echo, Labcyte) at 100 nL per well into cell culture assay 384-well plates. Each compound dilution is transferred in single or quadruplicate samples into dry assay plates, which are stored until assay is ready to go. The positive and negative controls are laid out in opposite on ends of the plate in vertical blocks (1 column).

Subsequently, an infectious mixture is prepared using an appropriate dilution of virus stock previously determined by titration with cells at a density of 50,000/ml and 20 L/well is added to test plates w/compounds via automation (uFlow, Biotek). Each plate includes negative and positive controls (16 replicates each) to create 0% and 100% virus inhibition standards, respectively. Following the infection with RSV, testing plates are incubated for 4 days in a 37° C. cell culture incubator. After the incubation, a cell viability reagent, Cell TiterGlo (Promega, Madison, WI) is added to the assay plates, which are incubated briefly, and a luminescent readout is measured (Envision, Perkin Elmer) in all the assay plates. The RSV-induced cytopathic effect, percentage inhibition, is determined from the levels of remaining cell viability. These numbers are calculated for each tested concentration relative to the 0% and 100% inhibition controls, and the EC₅₀ value for each compound is determined by non-linear regression as a concentration inhibiting the RSV-induced cytopathic effect by 50%. Various potent anti-RSV tool compounds are used as positive controls for antiviral activity.

### Example F. HEp-2 CC₅₀

Cytotoxicity of tested compounds is determined in uninfected HEp-2 cells in parallel with the antiviral activity using the cell viability reagent in a similar fashion as described before for other cell types (Cihlar et al., ANTIMICROB AGENTS CHEMOTHER. 2008, 52(2):655-65). The same protocol as for the determination of antiviral activity is used for the measurement of compound cytotoxicity except that the cells are not infected with RSV. Instead, an uninfected cell mixture at the same density is added at 20 ul/well to plates containing prediluted compounds, also at 100 nL/sample. Assay plates are then incubated for 4 days followed by a cell viability test using the same CellTiter Glo reagent addition and measurement of luminescent readouts. Untreated cell and cells treated at 2 μM puromycin (Sigma, St. Louis, MO) serve as 100% and 0% cell viability control, respectively. The percent of cell viability is calculated for each tested compound concentration relative to the 0% and 100% controls and the CC₅₀ value is determined by non-linear regression as a compound concentration reducing the cell viability by 50%.

### Example G. HEp-2 and MT4 CC₅₀

Cytotoxicity of the compounds was determined in uninfected cells using the cell viability reagent in a similar fashion as described before for other cell types (Cihlar et al., ANTIMICROB AGENTS CHEMOTHER. 2008, 52(2):655-65). HEp-2 (1.5×103 cells/well) and MT-4 (2×103 cells/well) cells were plated in 384-well plates and incubated with the appropriate medium containing 3-fold serially diluted compound ranging from 15 nM to 100,000 nM. Cells were cultured for 4-5 days at 37° C. Following the incubation, the cells were allowed to equilibrate to 25° C., and cell viability was determined by adding Cell-Titer Glo viability reagent. The mixture was incubated for 10 min, and the luminescence signal was quantified using an Envision plate reader. Untreated cell and cells treated at 2 μM puromycin (Sigma, St. Louis, MO) serve as 100% and 0% cell viability control, respectively. The percent of cell viability was calculated for each tested compound concentration relative to the 0% and 100% controls and the CC₅₀ value was determined by non-linear regression as a compound concentration reducing the cell viability by 50%.

### Example H. RSV NHBE EC₅₀

Normal human bronchial epithelial (NHBE) cells were purchased from Lonza (Walkersville, MD, Cat #CC-2540) and cultured in Bronchial Epithelial Growth Media (BEGM) (Lonza, Walkersville, MD, Cat #CC-3170). The cells were passaged 1-2 times per week to maintain <80% confluency. The NHBE cells were discarded after 6 passages in culture.

To conduct the RSV A2 antiviral assay, NHBE cells were plated in 96-well plates at a density of 7,500 cells per well in BEGM and allowed to attach overnight at 37° C. Following attachment, 100 μL of cell culture media was removed and 3-fold serially diluted compound was added using a Hewlett-Packard D300 Digital Dispenser. The final concentration of DMSO was normalized to 0.05%. Following compound addition, the NHBE cells were infected by the addition of 100 μL of RSV A2 at a titer of 1×10^{4.5} tissue culture infectious doses/mL in BEGM and then incubated at 37° C. for 4 days. The NHBE cells were then allowed to equilibrate to 25° C. and cell viability was determined by removing 100 μL of culture medium and adding 100 μL of Cell-Titer Glo viability reagent. The mixtures were incubated for 10 minutes at 25° C., and the luminescence signal was quantified on an Envision luminescence plate reader.

### Example I. RSV NHBE FLuc EC₅₀

Normal human bronchial epithelial (NHBE) cells are purchased from Lonza (Walkersville, MD Cat #CC-2540) and maintained in Bronchial Epithelial Cell Growth Medium (BEGM) (Lonza, Walkersville, MD, Cat #CC-3170) with all provided supplements in the BulletKit. Cells are passaged 2-3 times per week to maintain sub-confluent densities and are used for experiments at passages 2-4.

Recombinant Respiratory Syncytial virus strain A2 containing the firefly luciferase reporter between the P and M genes (RSV-Fluc, 6.3×10⁶ TCID₅₀/mL) is purchased from Viratree (Durham, NC, Cat #R¹⁴⁵).

NHBE cells (5×10³/well) are seeded in 100 μL white wall/clear bottom 96-well plates (Corning) with culture medium and are incubated for 24 hours at 37° C. with 5% CO₂. On the following day, three-fold serial dilutions of compounds prepared in DMSO are added to the wells using the HP D300e digital dispenser with normalization to the highest concentration of DMSO in all wells. The cells are then infected with RSV-Fluc diluted with BEGM media at an MOI of 0.1 for a final volume of 200 μL media/well. Uninfected and untreated wells are included as controls to determine compound efficacy against RSV-Fluc. Following incubation with compound and virus for three days at 37° C. with 5% CO₂, 100 μL of culture supernatant is removed from each well and replaced with 100 μL of ONE-Glo luciferase reagent (Promega, Madison, WI, Cat #E6110). The plates are gently mixed by rocking for 10 minutes at 21° C. and luminescence signal is measured using an Envision plate reader (PerkinElmer). Values are normalized to the uninfected and infected DMSO controls (0% and 100% infection, respectively). Non-linear regression analysis is applied to determine the compound concentration at which 50% luminescence signal is reduced (EC₅₀) using the XLfit4 add-in for MICROSOFT® EXCEL®. All experiments are performed in duplicate with two technical repeats each.

### Example J. RSV NHBE CC₅₀

NHBE cells were seeded in black 384-TC-treated plates (Corning) at 2×10³ cells/well in a final volume of 20 μL BEBM+supplements (Lonza). The next day, add 0.1 μL of compound was added to the assay plates using an Echo acoustic dispenser. Plates were incubated for 3 additional days at 37° C. and 5% CO₂. On day 3 of treatment, 20 μL of CellTiter Glo (Promega) was added to each well using a Biotek dispenser. After a 10-minute incubation, luminescence signal was measured with 0.1 sec integration time using an EnVision (Perkin-Elmer) plate reader. Values were normalized to the DMSO- and puromycin-treated controls (0% and 100% cell death, respectively). Data was fit using non-linear regression analysis and CC₅₀ values were then determined as the concentration reducing the luciferase signal by 50%. The compiled data was generated based on least two independent experimental replicates, each containing technical quadruplicates for each concentration.

### Example K. RSV HAE EC₅₀

HAE cells are cultured at the air-liquid interface and have an apical side that is exposed to the air and a basal side that is in contact with the medium. Prior to experimentation, HAE were removed from their agar-based shipping packaging and were acclimated to 37° C./5% CO₂ overnight in 1 ml of HAE Assay medium (AIR-100-MM, Mattek Corp). HAE were prepared for infection by washing the apical surface twice with 400 μL of PBS (either utilizing direct pipetting methods or by running each transwell through a trough containing PBS) to remove the mucus layer. Apical chambers were drained of PBS and tapped gently onto absorbent material to remove as much PBS as possible. After washing, the cells were transferred to fresh HAE maintenance media containing 4-fold serially diluted compound, delivered to the basal side of the cell monolayer, and apically infected with 100 μL of a 1:600 dilution of RSV A strain A2 1000× stock (ABI, Columbia, MD, cat #10-124-000) in HAE assay medium for 3 hours at 37° C. in 5% CO₂. The virus inoculum was removed and the apical surface of the cells was washed 3 times with PBS using either method previously described. The cells were then cultured in the presence of compound for 3 days at 37° C. Following the incubation, total RNA was extracted from the HAE cells using a MagMAX-96 Viral RNA isolation kit (Applied Biosystems, Foster City, CA, Cat #AM1836) and intracellular RSV RNA was quantified by real-time PCR. Approximately 25 ng of purified RNA was added to a PCR reaction mixture that contained 0.9 μM RSV N Forward and RSV N Reverse primers, 0.2 μM RSV N Probe and 1× Taqman RNA-to-Ct 1-Step Kit (Applied Biosystems, Foster City, CA, Cat #4392938). RNA levels were normalized using a Taqman GAPDH control primer set (Applied Biosystems, Foster City, CA, Cat #402869). Real-Time PCR Primers and Probe Used in the RSV A2 HAE Antiviral Assay: RSV N Forward CATCCAGCAAATACACCATCCA (SEQ ID NO:1), RSV N Reverse TTCTGCACATCATAATTAGGAGTATCAA (SEQ ID NO:2), RSV N Probe FAM-CGGAGCACAGGAGAT-BHQ (SEQ ID NO:3).

### Example L. HRV16 HELA EC₅₀

H1-HeLa cells, cultured in complete DMEM medium containing 10% heat-inactivated FBS and 1% Penicillin/Streptomycin, were seeded in 96 well plates at 3000 cells/well one day prior to compound dosing and infection. The antiviral activity of each compound was measured in triplicate. Compounds were added directly to the cell cultures in serial 3-fold dilutions using the HP300 digital dispenser (Hewlett Packard, Palo Alto, CA) immediately prior to infection. The plates were transferred to BSL-2 containment and the appropriate dilution of virus stock, previously determined by titration and prepared in cell culture media, was added to test plates containing cells and serially diluted compounds. Each plate included 6 wells of infected untreated cells and 6 wells of uninfected cells that served as 0% and 100% virus inhibition control, respectively. Following the infection, test plates were incubated for 96 h in a tissue culture incubator set to 33° C./5% CO₂. Following incubation, the H1-HeLa cells were removed from incubation and allowed to equilibrate to 25° C. Cell viability was determined by removing 100 μL of culture medium and adding 100 μL of Cell-Titer Glo viability reagent. The mixtures were incubated on a shaker for 10 minutes at 25° C., and the luminescence signal was quantified on an Envision luminescence plate reader. The percentage inhibition of virus infection was calculated for each tested concentration relative to the 0% and 100% inhibition controls and the EC₅₀ value for each compound was determined by 4-parametric non-linear regression as the effective concentration of compound that inhibited cytopathic effect by 50%.

### Example M. HRVIA HELA EC₅₀

H1-HeLa cells, cultured in complete RPMI 1640 medium containing 10% heat-inactivated FBS and 1% Penicillin/Streptomycin, were seeded in 96 well plates at 5000 cells/well one day prior to compound dosing and infection. The antiviral activity of each compound was measured in triplicate. Compounds were added directly to the cell cultures in serial 3-fold dilutions using the HP300 digital dispenser (Hewlett Packard, Palo Alto, CA) immediately prior to infection. The plates were transferred to BSL-2 containment and 100 μL of 1/4000 dilution of HRV1a virus stock was added to each well containing cells and serially diluted compounds. Each plate included 6 wells of infected untreated cells and 6 wells of cells containing 5 μM Rupintrivir that served as 0% and 100% virus inhibition control, respectively. Following the infection, test plates were incubated for 96 h in a tissue culture incubator set to 37° C./5% CO₂. Following incubation, the H1-HeLa cells were removed from incubation and allowed to equilibrate to 25° C. Cell viability was determined by removing 100 μL of culture medium and adding 100 μL of Cell-Titer Glo viability reagent. The mixtures were incubated on a shaker for 10 minutes at 25° C., and the luminescence signal was quantified on an Envision luminescence plate reader. The percentage inhibition of virus infection was calculated for each tested concentration relative to the 0% and 100% inhibition controls and the EC₅₀ value for each compound was determined by 4-parametric non-linear regression as the effective concentration of compound that inhibited cytopathic effect by 50%.

### Example N. HRV14 HELA EC₅₀

H1-HeLa cells, cultured in complete RPMI 1640 medium containing 10% heat-inactivated FBS and 1% Penicillin/Streptomycin, were seeded in 96 well plates at 5000 cells/well one day prior to compound dosing and infection. The antiviral activity of each compound was measured in triplicate. Compounds were added directly to the cell cultures in serial 3-fold dilutions using the HP300 digital dispenser (Hewlett Packard, Palo Alto, CA) immediately prior to infection. The plates were transferred to BSL-2 containment and 100 μL of 1/4000 dilution of HRV14 virus stock was added to each well containing cells and serially diluted compounds. Each plate included 6 wells of infected untreated cells and 6 wells of cells containing 5 μM Rupintrivir that served as 0% and 100% virus inhibition control, respectively. Following the infection, test plates were incubated for 96 h in a tissue culture incubator set to 37° C./5% CO₂. Following incubation, the H1-HeLa cells were removed from incubation and allowed to equilibrate to 25° C. Cell viability was determined by removing 100 μL of culture medium and adding 100 μL of Cell-Titer Glo viability reagent. The mixtures were incubated on a shaker for 10 minutes at 25° C., and the luminescence signal was quantified on an Envision luminescence plate reader. The percentage inhibition of virus infection was calculated for each tested concentration relative to the 0% and 100% inhibition controls and the EC₅₀ value for each compound was determined by 4-parametric non-linear regression as the effective concentration of compound that inhibited cytopathic effect by 50%.

### Example O. HRVc15 and HRVc25 EC₅₀

First, HRV replicon RNA is prepared. 5 ug of DNA Template (HRVc15 or HRVc25) is linearized with 2 μL of MluI enzyme in NEB buffer-3 in a final volume of 25 μL for 3 hours at 37° C. Following incubation, linearized DNA is purified on a PCR purification column and the following in vitro transcription is performed using the following conditions: 10 μL of RiboMAX Express T7 2× buffer, 1-8 μL of linear DNA template (1 μg), 0-7 μL nuclease free water, 2 μL enzyme mix T7 express. The final volume of 20 μL is mixed and incubated at 37° C. for 30 min. Following incubation, 1 μL of RQ1 RNase free DNase is added and the mixture is incubated at 37° C. for 15 min. The resulting RNA is then purified with the MegaClear Kit (Gibco Life Technologies cat #11835-030) and is eluted two times with 50 μL of elution buffer at 95° C. H1-HeLa cells cultured in complete RPMI 1640 media containing 10% heat-inactivated FBS and 1% Penicillin/Streptomycin are seeded into T-225 flasks at a concentration of 2E6 cells/flask a day prior to transfection and are incubated at 37° C./5% CO₂ overnight. On the day of transfection, cells are trypsinized following standard cell culture protocols and are washed two times with PBS. Following washes, cells are resuspended at a concentration of 1E7 cells/mL in PBS and the suspension is stored on wet ice. Electroporation is used to introduce replicon RNA into the H1-HeLa cells. A final volume of 10 μL containing 10 μg of replicon c15 or 1 μg of c25 replicon RNA, respectively, are pipetted into a 4 mm electroporation cuvette. The H1-HeLa cell stock is mixed by gently swirling and 0.5 mL of the cell stock previously prepared is transferred into the cuvette containing the replicon RNA. The combined solution is flicked to mix. Following mixing, cells are immediately electroporated using the following settings: 900V, 25 uF, infinite resistance, 1 pulse. Cuvettes are rested on ice for 10 min. Following the 10 min incubation, add 19 mL of ambient temperature, phenol red-free and antiobiotic-free RPMI 1640 containing 10% heat-inactivated FBS per electroporation. 150 μL (4E4 cells) of the electroporated cell suspension are seeded per well into a 96well clear-bottom, white cell culture plate, and are incubated at 25° C. for 30 min. Compounds were added directly to the cell cultures in serial 3-fold dilutions using the HP300 digital dispenser (Hewlett Packard, Palo Alto, CA) and were tested in triplicate. Following the addition of compounds, plates are incubated at 33° C. for 48 hrs. Replicon activity is then measured by a Renilla-Glo Luciferase Assay system. Prior to signal quantification, plates are removed from incubators and are allowed to equilibrate to 25° C. after 50 uL is removed from each well. Following manufacturer's protocol, a 1:100 dilution of Renilla-Glo substrate to buffer is prepared and 100 μL of the Renill-Glo luciferase mix is added to each well. Plates are then incubated for 20 min at 25° C. under gentle agitation and luciferase signal are determined with a 0.1 second detection setting using an EnVision luciferase quantification reader. The percentage inhibition of replicon inhibition was calculated for each tested concentration relative to the 0% and 100% inhibition controls included in the experiments and the EC₅₀ value for each compound was determined by 4-parametric non-linear regression as the effective concentration of compound that inhibited luciferase signal by 50%.

### Example P. DENV-2 Huh-7 Rep EC₅₀

In 384 well plates (Greiner, Cat #781091), compounds were acoustically transferred at 200 nl per well in a 8 compound (4 replicates) or 40 compound dose response format (3 replicates). For all plates tested, Balapiravir, GS-5734 and NITD008 were included as positive inhibition controls alongside 0% inhibition, DMSO-only negative control wells. Following compound addition, Huh-7 cells containing the DENV2 replicon construct were harvested following standard cell culture procedures and were adjusted to a concentration of 1.25E5 cells/mL in cell culture media composed of cDMEM without genticin. 40 μL of the cell stock was then added to each well for a final cell density of 5,000 cells/well. Cell and compound mixtures were incubated at 37° C./5% CO₂ for 48 hours. Prior to harvesting cells, EnduRen Live Cell Substrate (Promega, Cat #E6481) was prepared by suspending 3.4 mg into 100 uL of DMSO to generate a 60 mM stock solution. The stock solution was then diluted 1:200 in pre-warmed cDMEM and 10 uL of this diluted solution was added to each well of the 384 well plates. Plates were then centrifuged at 500 rpm briefly and were placed on a plate shaker for 2 min. Following mixing, plates were incubated at 7° C./5% CO₂ for 1.5 hours prior to measuring luminescence on an Envision luminometer. The percentage inhibition of replicon signal was calculated for each tested concentration relative to the 0% and 100% inhibition controls and the EC₅₀ value for each compound was determined by 4-parametric non-linear regression as the effective concentration of compound that inhibited replicon signal by 50%.

### Example Q. HCV Rep 1B and 2A EC₅₀

Compounds were serially diluted in ten steps of 1:3 dilutions in 384-well plates. All serial dilutions were performed in four replicates per compound within the same 384-well plate. An HCV protease inhibitor ITMN-191 at 100 μM was added as a control of 100% inhibition of HCV replication while puromycin at 10 mM was included as a control of 100% cytotoxicity. To each well of a black polystyrene 384-well plate (Greiner Bio-one, Monroe, NC), 90 μL of cell culture medium (without Geneticin) containing 2000 suspended HCV replicon cells was added with a Biotek uFlow workstation. For compound transfer into cell culture plates, 0.4 μL of compound solution from the compound serial dilution plate was transferred to the cell culture plate on a Biomek FX workstation. The DMSO concentration in the final assay wells was 0.44%. The plates were incubated for 3 days at 37° C. with 5% CO₂ and 85% humidity. The HCV replicon assay was a multiplex assay, able to assess both cytotoxicity and antireplicon activity from the same well. The CC₅₀ assay was performed first. The media in the 384-well cell culture plate was aspirated, and the wells were washed four times with 100 μL of PBS each, using a Biotek ELX405 plate washer. A volume of 50 μL of a solution containing 400 nM calcein AM (Anaspec, Fremont, CA) in 1×PBS was added to each well of the plate with a Biotek uFlow workstation. The plate was incubated for 30 min at room temperature before the fluorescence signal (excitation 490 nm, emission 520 nm) was measured with a Perkin-Elmer Envision plate reader. The EC₅₀ assay was performed in the same wells as the CC₅₀ assay. The calcein-PBS solution in the 384-well cell culture plate was aspirated with a Biotek ELX405 plate washer. A volume of 20 μL of Dual-Glo luciferase buffer (Promega, Madison, WI) was added to each well of the plate with a Biotek uFlow Workstation. The plate was incubated for 10 min at room temperature. A volume of 20 μL of a solution containing a 1:100 mixture of Dual-Glo Stop & Glo substrate (Promega, Madison, WI) and Dual-Glo Stop & Glo buffer (Promega, Madison, WI) was added to each well of the plate with a Biotek uFlow Workstation. The plate was then incubated at room temperature for 10 min before the luminescence signal was measured with a Perkin-Elmer Envision Plate Reader.

### Example R. HEp-2 RSV-Luc5 384-Well Assay (EC₅₀ RSVFLUC Hep2-384)

HEp-2 cell line was purchased from ATCC (Manassas, VA Cat #CCL-23) and maintained in Dulbecco's Minimum Essential Medium (DMEM) (Corning, New York, NY, Cat #15-018 CM) supplemented with 10% fetal bovine serum (FBS) (Hyclone, Logan, UT, Cat #SH30071-03) and 1× Penicillin-Streptomycin-L-Glutamine (Corning, New York, NY, Cat #30-009-CI). Cells were passaged 2 times per week to maintain sub-confluent densities and were used for experiments at passage 5-20. Respiratory syncytial virus recombinant with luciferase (RSV-Luc5) direct pelleted virus (≥1×107 TCID50/ml) was purchased from Microbiologics (Saint Cloud, MN). Viral replication was determined in HEp-2 cells in the following manner.

Compounds are prepared in 384-well polypropylene plates (Greiner, Monroe, NC, Cat #784201) with 8 compounds per plate in grouped replicates of 4 at 10 serially diluted concentrations (1:3).

HEp-2 cells were suspended in DMEM (supplemented with 10% FBS and 1× Penicillin-Streptomycin-L-Glutamine) and 60 uL of 4,000 cells per well were seeded into 384-well plates (Greiner, Monroe, NC, Cat #781080) using Biotek MultiFlo dispenser. After overnight incubation at 37° C. and 5% CO₂, 0.4 uL of three-fold serial dilutions of compound was added to each well using a Biomek FX pipette station. RSV-Luc5 viruses were diluted in DMEM (supplemented with 10% FBS and 1× Penicillin-Streptomycin-L-Glutamine) at an MOI-0.5. Virus suspension was added to each 384-well compound plate at 20 uL per well using a Biotek MultiFlo dispenser. The assay plates were incubated for 3 days at 37° C. and 5% CO₂. At the end of incubation, One-Glo reagent (Promega, Madison, WI, Cat #E6120) was prepared. The assay plate and the reagent were equilibrated to room temperature for 30 minutes. 50 uL per well of medium was removed from assay plate and 40 uL per well of One-Glo reagent was added to each plate by Biomek FX. The plates were sat at room temp for 15 minutes. Viral replication was then assessed by measuring luminescence signal using and Envision plate reader. Remdesivir was used as positive control and DMSO was used as negative control. Values were normalized to the positive and negative controls (as 0% and 100% replication, respectively) and data was fitted using non-linear regression analysis by Gilead's dose response tool. The EC₅₀ value for each compound was then determined as the concentration reducing the viral replication by 50%.

### Example S. HEp-2 and MT4 CC₅₀

Cytotoxicity of the compounds was determined in uninfected cells using the cell viability reagent in a similar fashion as described before for other cell types (Cihlar et al., ANTIMICROB AGENTS CHEMOTHER. 2008, 52(2):655-65). HEp-2 (1.5×103 cells/well) and MT-4 (2× 103 cells/well) cells were plated in 384-well plates and incubated with the appropriate medium containing 3-fold serially diluted compound ranging from 15 nM to 100,000 nM. Cells were cultured for 4-5 days at 37° C. Following the incubation, the cells were allowed to equilibrate to 25° C., and cell viability was determined by adding Cell-Titer Glo viability reagent. The mixture was incubated for 10 min, and the luminescence signal was quantified using an Envision plate reader. Untreated cell and cells treated at 2 μM puromycin (Sigma, St. Louis, MO) serve as 100% and 0% cell viability control, respectively. The percent of cell viability was calculated for each tested compound concentration relative to the 0% and 100% controls and the CC₅₀ value was determined by non-linear regression as a compound concentration reducing the cell viability by 50%.

### Example T. H1-Hela anti-HRV Assay

Both H1-HeLa cells and human rhinovirus 16 (HRV-16) are purchased from ATCC.

H1-HeLa maintenance media is composed of DMEM supplemented with 10% FBS and 1% Penn/Strep. Virus infection medium (VIM) is composed of DMEM+2% FBS.

H1-HeLa cells are seeded into 96-well black/clear bottom plates with 5,000 cells/well in 100 μL/well in H1-HeLa maintenance medium and incubated for 24 hours at 37° C. and 5% CO₂. On the following day medium is aspirated and replaced with 100 μL VIM, next three-fold serial dilutions of compounds prepared in DMSO are added to the wells using the HP D300e digital dispenser with normalization to the highest concentration of DMSO in all wells. HRV-16 is diluted with the VIM to an MOI=0.05 and added to the cells in 100 μL/well. On each plate, uninfected and infected DMSO controls are included to determine compound efficacy against HRV. When extensive cytopathic effect is observed in the positive control (usually 3-6 days post infection) following the incubation at 37° C. and 5% CO₂, the culture plates are cooled to room temperature. The culture medium is removed and 200 μL of CellTiter Glo (1:2 dilution in PBS) is added to each well. The plates are agitated for 10 minutes on a shaker at room temperature and luminescence signal is measured using an EnVision plate reader (PerkinElmer). Values are normalized to the uninfected and infected DMSO controls (0% and 100% infection, respectively). Non-linear regression analysis is applied to determine the compound concentration at which 50% luminescence signal is reduced (EC₅₀) using the XLfit4 add-in for MICROSOFT® EXCEL®. All experiments are performed in duplicate with two technical repeats.

### Example U. NHBE RSV-Luc5 384-Well Assay (EC₅₀ RSVFLUC NHBE-384)

Normal Human Bronchial Epithelial (NHBE) cells were purchased from Lonza (Walkersville, MD Cat #CC2540) and maintained in BEGM Bronchial Epithelial Cell Growth Medium BulletKit (Lonza CC-3170).

Cells were thawed, expanded, and were used for experiments at passage 2. Respiratory syncytial virus recombinant with luciferase (RSV-Luc5) (≥1×107 Infectious Units/ml (IU/ml) determined by TCID₅₀) was purchased from Microbiologics (Saint Cloud, MN). Viral replication was determined in NHBE cells in the following manner.

Compounds are prepared in 100% DMSO in 384-well polypropylene plates (Greiner, Monroe, NC, Cat #784201) with 8 compounds per plate in grouped replicates of 4 at 10 serially diluted concentrations (1:3). The serially diluted compounds were transferred to low dead volume Echo plates (Labcyte, Sunnyvale, CA, Cat #LP-0200).

The test compounds were spotted to 384-well assay plates (Greiner, Monroe, NC, Cat #781091) at 200 nL per well. NHBE cells were harvested and suspended in BEGM Bronchial Epithelial Cell Growth Medium BulletKit and seeded to the pre-spotted assay plates at 5,000 cells per well in 30 μL. RSV-Luc5 virus was diluted in BEGM Bronchial Epithelial Cell Growth Medium BulletKit at 500,000 Infectious Units (IU) per mL and 10 μL per well was added to the assay plates containing cells and compounds, for an MOI of 1. The assay plates were incubated for 3 days at 37° C. and 5% CO₂. At the end of incubation, One-Glo reagent (Promega, Madison, WI, Cat #E6120) was prepared. The assay plates and One-Glo reagent were equilibrated to room temperature for at least 15 minutes. 40 μL per well of One-Glo reagent was added and the plates were incubated at room temperature for 15 minutes before reading the luminescence signal on an En Vision multimode plate reader (Perkin Elmer, Waltham, MA). Remdesivir was used as positive control and DMSO was used as negative control. Values were normalized to the positive and negative controls (as 0% and 100% replication, respectively) and data was fitted using non-linear regression analysis by Gilead's dose response tool. The EC₅₀ value for each compound was defined as the concentration reducing the viral replication by 50%.

### Example V: HEp-2 RSV-Luc5 384-Well Assay (EC₅₀ RSVFLUC Hep2-384 v2

HEp-2 cell line was purchased from ATCC (Manassas, VA Cat #CCL-23) and maintained in Dulbecco's Minimum Essential Medium (DMEM) (Corning, New York, NY, Cat #15-018 CM) supplemented with 10% fetal bovine serum (FBS) (Hyclone, Logan, UT, Cat #SH30071-03) and 1× Penicillin-Streptomycin-L-Glutamine (Corning, New York, NY, Cat #30-009-CI). Cells were passaged 2 times per week to maintain sub-confluent densities and were used for experiments at passage 5-20. Respiratory syncytial virus recombinant with luciferase (RSV-Luc5) (≥1×107 TCID50/ml) was purchased from Microbiologics (Saint Cloud, MN). Viral replication was determined in HEp-2 cells in the following manner.

Compounds are prepared in 100% DMSO in 384-well polypropylene plates (Greiner, Monroe, NC, Cat #784201) with 8 compounds per plate in grouped replicates of 4 at 10 serially diluted concentrations (1:3). The serially diluted compounds were transferred to low dead volume Echo plates (Labcyte, Sunnyvale, CA, Cat #LP-0200).

The test compounds were spotted to 384-well assay plates (Greiner, Monroe, NC, Cat #781091) at 200 nL per well. HEp-2 cells were harvested and suspended in DMEM (supplemented with 10% FBS and 1× Penicillin-Streptomycin-L-Glutamine) and seeded to the pre-spotted assay plates at 4,000 cells per well in 30 μL. RSV-Luc5 viruses were diluted in DMEM (supplemented with 10% FBS and 1× Penicillin-Streptomycin-L-Glutamine) at 200,000 Infectious Units (IU) per mL and 10 μL per well was added to the assay plates containing cells and compounds, for an MOI=0.5. The assay plates were incubated for 3 days at 37° C. and 5% CO₂. At the end of incubation, One-Glo reagent (Promega, Madison, WI, Cat #E6120) was prepared. The assay plates and One-Glo reagent were equilibrated to room temperature for at least 15 minutes. 40 μL per well of One-Glo reagent was added and the plates were incubated at room temp for 15 minutes before reading the luminescence signal on an En Vision multimode plate reader (Perkin Elmer, Waltham, MA). Remdesivir was used as positive control and DMSO was used as negative control. Values were normalized to the positive and negative controls (as 0% and 100% replication, respectively) and data was fitted using non-linear regression analysis by Gilead's dose response tool. The EC₅₀ value for each compound was then determined as the concentration reducing the viral replication by 50%.

### Example W: Dengue Virus-2 Huh-7 EC₅₀

Huh7 hepatoblastoma cells were seeded onto 96-well plates and incubated at 37° C. with 5% CO₂ overnight. The plates were seeded at a cell concentration that will yield >70% confluent monolayers in each well after overnight incubation. Eight 3-fold serial dilutions of compounds were diluted in test media (MEM supplemented with 2% FBS and 50 μg/mL gentamicin). The highest test compound concentration was 50 μg/mL. 100 μL of each concentration was added to 5 test wells on the 96-well plate. Of these 5 test wells, 3 wells of each dilution were infected with dengue virus type 2, diluted in test media (approximately 2000 CCID₅₀ per well for an MOI of 0.07). Test medium (100 μL) with no virus was added to 2 wells to represent the uninfected cytotoxicity controls. Six additional infected wells received 100 μL media alone as untreated virus controls. Further, 100 μL of media alone was added to 6 uninfected wells to serve as uninfected, untreated controls. Cultures were incubated at 37° C.+5% CO₂ until >80% CPE is observed. After cytopathic effect (CPE) is observed microscopically, 0.011% neutral red dye was added for approximately 2 hours. The neutral red dye was removed and wells were rinsed once with PBS to remove residual, unincorporated dye. A 50:50 Sorensen citrate buffer/ethanol solution was added and incubated at room temperature for >30 minutes with agitation, followed by measurement of neutral red dye using a spectrophotometer at 540 nm. The resulting optical density measurement was converted to percent signal of the infected, untreated cell control normalizing to virus controls. EC₅₀ and CC₅₀ values were determined by linear regression analysis.

### Example X: hMPV H1-Hela EC₅₀

The human metapneumoavirus (hMPV) anti-viral assay is an anti-nucleoprotein ELISA performed in infected H1-HeLa cells. H1-HeLa cells were maintained in Dulbecco's Modified Eagle's Medium with high glucose (Gibco, Cat #: 11995073) supplemented with 10% FBS (HyClone, Cat #: SH303396.03), 100 units/mL penicillin and 100 μg/mL streptomycin (Gibco, Cat #: 15140122). One day before infection, H1-HeLa cells were seeded into 96-well plates (Corning, Cat. #: 3903) at 0.1 mL/well at 1.5×10⁵ cells/mL in Opti-MEM (Gibco, Cat #: 31985070) supplemented with 2% FBS, and were incubated at 37° C. in a 100% humid atmosphere containing 5% (v/v) CO₂ overnight. On the next day, CAN97-83-GFP1 (A2a) hMPV was diluted in Opti-MEM medium containing 200 μg/mL CaCl₂) and 2 μg/mL TPCK treated trypsin and distributed to the 96-well plates at 0.1 mL/well for an MOI=0.1. Test compounds were distributed to each well using a HP D300e digital dispenser with a final volume of 200 μL/well. After the plates were centrifuged at 700 g for one hour at room temperature, they were maintained in a humidified chamber at 37° C. with 5% (v/v) CO₂ for 96 hours. After the incubation, the medium was removed, and the plates were fixed with 0.1 mL/well 1% formaldehyde for 15 min at room temperature. The fixative was removed, the plates were air dried for 30-60 minutes, and then permeabilized with 0.1 mL/well 0.5% Triton X-100 in PBS for five minutes at room temperature. Following permeabilization, the plates were washed once with 0.1 mL/well blocking buffer consisting of 10% FBS (HyClone, Cat #: SH303396.03), 5% dry milk (AmericanBio, Cat #: AB10109-01000), and 0.1% Tween 20 (EMD Millipore, Cat #655204) in PBS. The plates were blocked with 0.1 mL/well blocking buffer for 60 minutes at 37° C. afterward. The blocking buffer was removed and 0.05 mL/well human metapneumovirus nucleocapsid antibody (Sigma, Cat #MAB80121) diluted 1:500 in Blocking buffer was added and incubated for two hours at 37° C. The plates were then washed with 0.2 mL/well 0.1% Tween 20/PBS five times, followed by addition of 0.05 mL/well of horseradish peroxidase (HRP) conjugated goat anti-mouse IgG antibody (Fisher Scientific, Cat #501077607) at 1:2000 dilution in the blocking buffer. After one-hour incubation at 37° C., plates were washed five times with 0.2 mL/well 0.1% tween 20 in PBS. The HRP signals were developed by adding 0.1 mL/well of TMB reagent (Thermo Scientific, Cat #: ENN301) and incubating at room until the positive control was apparent. At that point, the reaction was stopped by adding 0.1 mL/well TMB stop solution (SeraCare, Cat #: 5150-0021). The absorbance was measured at 450 nm with an EnVision plate reader. The relative absorbance was calculated by normalizing the absorbance of the compound-treated groups to that of the DMSO-treated groups (set as 100%). EC₅₀ values were calculated using a nonlinear four parameter variable slope regression model.

### Example Y: hMPV H1-Hela CC₅₀

H1-HeLa cell viability was measured using the CellTiter-Glo® Luminescent Cell Viability Assay kit (Promega, Cat #G7573) according to the manufacturer's protocol. H1-HeLa cells were maintained in Dulbecco's Modified Eagle's Medium with high glucose (Gibco, Cat #: 11995073) supplemented with 10% FBS (HyClone, Cat #: SH303396.03), 100 units/mL penicillin and 100 μg/mL streptomycin (Gibco, Cat #: 15140122). One day before compound treatment, H1-HeLa cells were seeded at 1.5×10⁵ cells per well into 96-well plates (Corning, Cat #3904) in Opti-MEM (Gibco, Cat #: 31985070) supplemented with 2% FBS, and were incubated at 37° C. in a 100% humid atmosphere containing 5% (v/v) CO₂. After overnight incubation, the cells were treated with three-fold serial dilutions of the compound. At 96 hours post treatment, CellTiter-Glo reagents were added into each well and luminescence signals were recorded by an En Vision plate reader. The relative cell viability was calculated by normalizing the absorbance of the compound-treated groups to that of the DMSO-treated groups (set as 100% viability) and 10 μM puromycin treated groups (set as 0% viability). The CC₅₀ (compound concentration for reducing 50% of cell viability) was calculated using a nonlinear regression model (four parameters).

### Example Z: H1-HeLa HRV-CTG EC₅₀

H1-Hela cell line (ATCC, Manassas, VA, Cat #CRL-1958) was maintained in Dulbecco's Minimum Essential Medium (DMEM) (Corning, New York, NY, Cat #15-018 CM) supplemented with 10% fetal bovine serum (FBS) (Hyclone, Logan, UT, Cat #SH30071-03), and 1× Penicillin-Streptomycin-L-Glutamine (Corning, New York, NY, Cat #30-009-CI). Cells were passaged 2 times per week to maintain sub-confluent densities and were used for experiments at passage 5-30. The Human Rhinovirus 1B (HRVIB) (ATCC, Manassas, VA, Cat #VR-1645), Human Rhinovirus 14 (HRV14) (ATCC, Manassas, VA, Cat #VR-284), and Human Rhinovirus 16 (HRV16) (ATCC, Manassas, VA, Cat #BR-283) was obtained through ATCC. Viral infection was monitored by determining viability of H1-HeLa cells as described below.

Test molecules are prepared in 100% DMSO in 384-well polypropylene plates (Greiner, Monroe, NC, Cat #784201) with 8 compounds per plate in grouped replicates of 4 at 10 serially diluted concentrations (1:3). The serially diluted compounds were transferred to low dead volume Echo plates (Labcyte, Sunnyvale, CA, Cat #LP-0200).

The test compounds were spotted to 384-well assay plates (Greiner, Monroe, NC, Cat #781091) at 200 nL per well using an Echo acoustic dispenser (Labcyte, Sunnyvale, CA). H1-HeLa cells were harvested and suspended in DMEM (supplemented with 2% FBS and 1× Penicillin-Streptomycin-L-Glutamine) and seeded to the pre-spotted assay plates at 5,000 cells per well in 30 μL. HRVIB, HRV14, and HRV16 was diluted in DMEM (supplemented with 2% FBS and 1× Penicillin-Streptomycin-L-Glutamine) at 97.1 million Infectious Units (IU) per mL, 151 million IU per mL and 221 million IU per mL respectively. 10 μL of virus per well was added to the assay plates containing cells and compounds, for an MOI of 0.5, 1.0, and 0.25 respectively. The assay plates were incubated for 4 days at 37° C. and 5% CO₂. At the end of incubation, Celltiter-Glo (Promega, Madison, WI, Cat #G7573) was prepared. The assay plates and Celltiter-Glo reagent were equilibrated to room temperature for at least 15 minutes. 40 μL per well of Celltiter-Glo reagent was added and the plates were incubated at room temperature for 15 minutes before reading the luminescence signal on an En Vision multimode plate reader (Perkin Elmer, Waltham, MA). Rupintrivir was used as positive control and DMSO was used as negative control. Values were normalized to the positive and negative controls (as 0% and 100% replication, respectively) and data was fitted using non-linear regression analysis by Gilead's dose response tool. The EC₅₀ value for each compound was defined as the concentration reducing viral replication by 50%.

### Example B1: RSV Cellomics Assay for RSV A2 Antiviral Activity (EC₅₀-RSV-Hep2-96-Cellomics)

8×10³ Hep2 cells are seeded into 96 well plates in 100 μL of media (DMEM supplemented with GlutaMAX, 10% FBS and Pen/Strep) and briefly spun at 50×g for 1 minute to attach cells. Cells were further allowed to attach with overnight incubation at 37° C.+5% CO₂. The following day, the media was aspirated and 100 μL of fresh DMEM media containing 2% FBS was added. A three-fold dilution series of compounds of interest are then added to the plate using the Tecan D300e dispenser and the plate is normalized to the well with the highest volume of DMSO. The cells are next infected by adding 100 μL of media containing RSV strain A2 at an MOI=0.05 and the plates incubated for 3 days at 37° C.+5% CO₂. Following the incubation, the media is removed, and the cells are washed once with 250 μL PBS and fixed with 250 μL ice-cold 100% methanol for 10 minutes at 4 C. The methanol is then removed and the plates air dried for 5 minutes and then washed once with 250 μL PBS to remove any residual methanol. The wells are then blocked 60 μL/well of BlockAid (B10710, Thermo-Fisher) for 1 hour at RT on rocker at which point the blocking buffer is carefully aspirated. Primary antibody against the RSV F protein (MAB 858-1) is then diluted 1:2000 in SuperBlock (37515, Thermo Fisher) and 50 μL of the primary antibody solution is added to each well. The plates are incubated at room temperature for 1 hour with gentle agitation and then washed with 250 μL of PBS-Tween 3 times. A goat anti-mouse AlexaFluor 647 conjugated secondary antibody (Sigma, A21235) is then diluted 1:2000 and Hoescht 33342 (H3570) at 1:1000 in SuperBlock and 50 μL added to the wells for 1 hour at 37° C. After incubation the antibody solution is aspirated, and the wells are then washed with 250 μL of PBS-Tween 3 times. To keep cells from drying out, 100 μL of PBS is added to each well and the plates are sealed with black plate seal. Fluorescence at the 647 nm wavelength is measured using a Cellomics CellInsight CX7 machine. Compound activity is determined as a percentage of the % positive cells (or average intensity) compared to DMSO treated RSV infected well controls following subtraction of uninfected controls. The EC₅₀ values are then calculated using nonlinear regression analysis in GraphPad Prism.

### E. Biological Data

Provided below in Table 3 is data related to compounds disclosed herein.

TABLE 3
Biological Data for Compounds Disclosed Herein

| | EC50 RSV | EC50 H1- | EC50 H1- | EC50 H1- | EC50-RSV- |
|---|---|---|---|---|---|
| | FLUC NHBE | HeLa_HRV14_HRV- | HeLa_HRV16_HRV- | HeLa_HRV1B_HRV- | HEp2-96- |
| | 384 V2 | CTG 384-well | CTG 384-well | CTG 384-well | Cellomics |
| Compound | (per Ex. | Assay (per | Assay (per | Assay (per | (nM) |
| # | U) nM | Ex Z) nM | Ex. Z) nM | Ex. Z) nM | Example B1 |
| 1 | 26 | 34 | 34 | 12 | 76 |
| 2 | 30 | 87 | 91 | 62 | 241 |
| 3 | 44 | — | — | — | 421 |
| 4 | 107 | — | — | — | 170 |
| 5 | 4457 | — | — | — | 3418 |
| 6 | 86 | — | — | — | 293 |
| 7 | 714 | — | — | — | 1405 |
| 8 | 134 | — | — | — | 237 |
| 9 | 265 | — | — | — | 215 |
| 10 | 117 | — | — | — | 127 |
| 11 | 4594 | — | — | — | 12587 |

The present disclosure provides reference to various embodiments and techniques. However, it should be understood that many variations and modifications can be made while remaining within the spirit and scope of the present disclosure. The description is made with the understanding that it is to be considered an exemplification of the claimed subject matter, and is not intended to limit the appended claims to the specific embodiments illustrated.

1. A compound of Formula I: 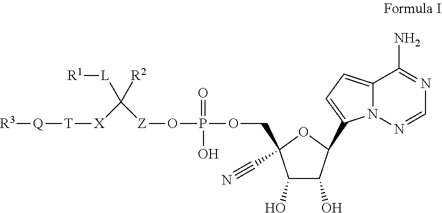
or a pharmaceutically acceptable salt thereof, wherein:
R¹ is H, C₃₋₁₀ cycloalkyl, C₆₋₁₀ aryl, or 5-10 membered heteroaryl containing one, two, or three N; the cycloalkyl, aryl, or heteroaryl of R¹ is optionally substituted with one, two, or three groups independently selected from R^{1A} and —NR^{13A}R^{14A};
each R^{1A} is independently halo, —CN, C₁₋₃ alkyl, C₁₋₃ haloalkyl, C₁₋₃ alkoxy, C₁₋₃ haloalkoxy, C₃₋₆ cycloalkyl, or 5-10 membered heteroaryl containing one, two, or three heteroatoms selected from N and O;
each R^{13A} is independently H or C₁-C₃ alkyl optionally substituted with NR^{13A1}R^{14A1}; R^{13A1} is H or C₁₋₃ alkyl; R^{14A1} is H or C₁₋₃ alkyl; and
each R^{14A} is independently H or C₁₋₃ alkyl;
R² is H or C₁₋₃ alkyl;
R³ is C₁₋₃ haloalkyl;
Q is C₁₀₋₂₁ alkylene or C₁₀₋₂₁ alkenylene; the alkylene or alkenylene of Q is optionally substituted with 1 to 6 Q^{1A}; each Q^{1A} is independently halo;
L is a bond, —O—, or —O(CR^{12A}R^{12B})ₙ—;
each R^{12A} is independently H or C₁₋₆ alkyl;
each R^{12B} is independently H or C₁₋₆ alkyl; and
n is 1 or 2;
X is a bond or C₁₋₃ alkylene;
T is a bond or —O—; and
Z is C₁₋₆ alkylene;
with the proviso that when X is a bond and Tis —O—, then L is a bond.

2-9. (canceled)

10. The compound of claim 1, or a pharmaceutically acceptable salt thereof, wherein the compound is of Formula Ic:

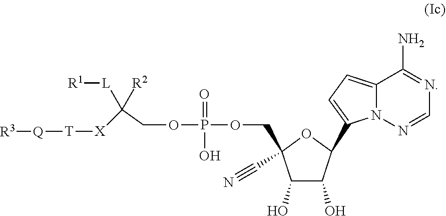

11-13. (canceled)

14. The compound of claim 1, or a pharmaceutically acceptable salt thereof, wherein L is —OCH₂—; and R² is H.

15-23. (canceled)

24. The compound of claim 1, or a pharmaceutically acceptable salt thereof, wherein
R¹ is phenyl optionally substituted with one or two groups independently selected from R^{1A}, and
each R^{1A} is independently halo, C₁₋₃ alkoxy, or —CN.

25. (canceled)

26. The compound of claim 1, or a pharmaceutically acceptable salt thereof, wherein R¹ is phenyl substituted with F and —CN.

27-29. (canceled)

30. The compound of claim 1, or a pharmaceutically acceptable salt thereof, wherein R¹-L- is

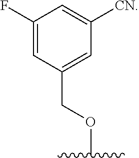 [SMILES: *OCc1cc(F)cc(C#N)c1]

31. (canceled)

32. (canceled)

33. The compound of claim 1, or a pharmaceutically acceptable salt thereof, wherein R¹ is quinolinyl optionally substituted with one or two groups independently selected from R^{1A}, and each R^{1A} is independently halo, —CN, or C₁₋₃ alkoxy.

34. (canceled)

35. The compound of claim 1, or a pharmaceutically acceptable salt thereof, wherein X is a bond.

36. (canceled)

37. The compound of claim 1, or a pharmaceutically acceptable salt thereof, wherein X is —CH₂—.

38. (canceled)

39. The compound of claim 1, or a pharmaceutically acceptable salt thereof, wherein T is a bond.

40. The compound of claim 1, or a pharmaceutically acceptable salt thereof, wherein Tis —O—.

41. The compound of claim 1, or a pharmaceutically acceptable salt thereof, wherein the compound is of Formula Ie:

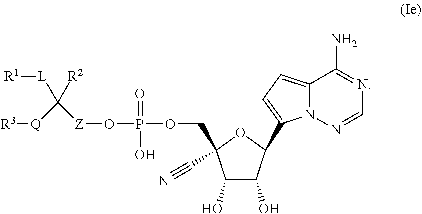

42-44. (canceled)

45. The compound of claim 1, or a pharmaceutically acceptable salt thereof, wherein Q is C₁₅₋₂₀ alkylene, optionally Q is —(CH₂)ₘ—, m is 15-20.

46-51. (canceled)

52. The compound of claim 1, or a pharmaceutically acceptable salt thereof, wherein R³ is —CF₃ or —CHF₂.

53-54. (canceled)

55. A compound as shown in Table 1, or a pharmaceutically acceptable salt thereof.

56. A pharmaceutical composition comprising a pharmaceutically effective amount of a compound of claim 1, or a pharmaceutically acceptable salt thereof, and a pharmaceutically acceptable carrier or excipient.

57-75. (canceled)

76. A compound of claim 55, having the structure 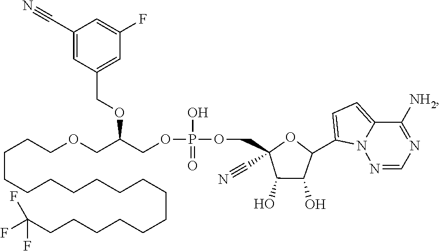 [SMILES: N#Cc1cc(F)cc(CO[C@H](COCCCCCCCCCCCCCCCCCC(F)(F)F)COP(=O)(O)OC[C@@]2(C#N)OC(c3ccc4c(N)ncnn34)[C@H](O)[C@@H]2O)c1]
or a pharmaceutically acceptable salt thereof.

77. A compound of claim 55, having the structure 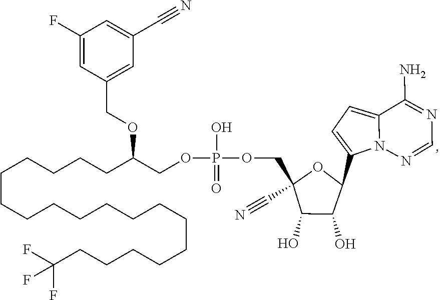 [SMILES: N#Cc1cc(F)cc(CO[C@H](CCCCCCCCCCCCCCCCCCC(F)(F)F)COP(=O)(O)OC[C@@]2(C#N)O[C@@H](c3ccc4c(N)ncnn34)[C@H](O)[C@@H]2O)c1]
or a pharmaceutically acceptable salt thereof.

78. A compound of claim 55, having the structure 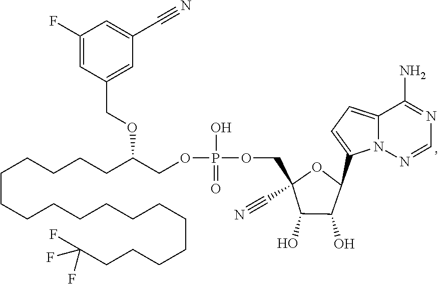 [SMILES: N#Cc1cc(F)cc(CO[C@@H](CCCCCCCCCCCCCCCCCC(F)(F)F)COP(=O)(O)OC[C@@]2(C#N)O[C@@H](c3ccc4c(N)ncnn34)[C@H](O)[C@@H]2O)c1]
or a pharmaceutically acceptable salt thereof.

79. A compound of claim 55, having the structure 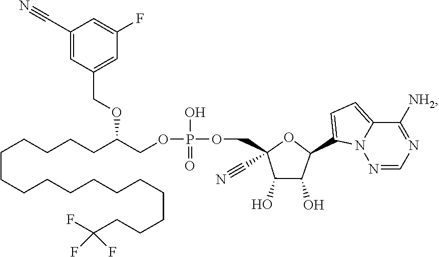 [SMILES: N#Cc1cc(F)cc(CO[C@@H](CCCCCCCCCCCCCCCCC(F)(F)F)COP(=O)(O)OC[C@@]2(C#N)O[C@@H](c3ccc4c(N)ncnn34)[C@H](O)[C@@H]2O)c1]
or a pharmaceutically acceptable salt thereof.
